(19) United States
(12) Patent Application Publication
(10) Pub. No.: US 2024/0287615 A1
(43) Pub. Date: Aug. 29, 2024

(54) CANCER METHODS

(71) Applicants: University College London, London (GB); Università degli Studi di Trento, Trenton (IT); Cornell University, Ithaca, NY (US); The University of British Columbia, Vancouver (CA)

(72) Inventors: Gerhardt Attard, Banstead (GB); Francesca Demichelis, Povo (TN) (IT); Alessandro Romanel, Povo (TN) (IT); Francesco Orlando, Povo (TN) (IT); Mark Rubin, Bolligen (CH); Himisha Beltran, Chestnut Hill, MA (US); Alexander Wyatt, Vancouver (CA)

(73) Assignees: University College London, London (GB); Università degli Studi di Trento, Trenton (IT); Cornell University, Ithaca, NY (US); The University of British Columbia, Vancouver, BC (CA)

(21) Appl. No.: 18/568,220

(22) PCT Filed: Jun. 9, 2022
(86) PCT No.: PCT/GB2022/051447

§ 371 Date: Dec. 7, 2023

(30) Foreign Application Priority Data
Jun. 9, 2021 (GB) 2108237.5

(51) Int. Cl. C12Q 1/6886; C12Q 1/6806; C12Q 1/6827; C12Q 1/686; G16B 20/10; G16B 20/20; G16B 30/10

(52) U.S. Cl. CPC C12Q 1/6886; C12Q 1/6806; C12Q 1/6827; C12Q 1/686; G16B 20/10; G16B 20/20; G16B 30/10; C12Q 2600/106; C12Q 2600/112; C12Q 2600/158

(57) ABSTRACT

The present invention provides an in vitro method, and in vitro assay, for staging, classification, screening, monitoring, stratification, selecting treatment for, ascertaining whether treatment is working in, and/or prognostication of cancer in a subject comprising determining allelic imbalance and copy number of target gene-regions in tumor DNA from a subject as set out in the application. The present invention also provides a set of oligonucleotide probes and kits for use in the method, and assay, of the present invention.

### FIELD OF THE INVENTION

The present invention relates to methods of selecting treatment for, ascertaining whether treatment is working in, and/or prognostication of cancer in a subject. Also provided herein are a set of probes, kits, and an in vitro assay for use in methods of the present invention.

### INTRODUCTION

Clinical implementable molecular stratification underlies the drive for precision medicine in cancer. Prostate cancer is a leading cause of cancer death among men and in the past few years, studies investigating the genomic landscape of metastatic prostate cancer have led to the identification of targetable molecular alterations, emerging resistance mechanisms, and new therapeutic options.

Following the approval of several PARP (poly-ADP ribose polymerase) inhibitors as a therapeutic option for various cancers, such as metastatic castration-resistant prostate cancer (mCRPC) patients with an alteration in BRCA1/2 and in some situations other DNA repair genes (for example ATM, FANCA, CHEK2, PALB2 and HDAC2), biomarker-driven treatment selection is becoming clinically possible (de Bono et al., N Engl J Med. 2020; 382(22):2091-2102). However, archival tissue biopsies, used for biomarker assessment for trial enrollment, have a number of limitations including not being representativeness of current disease, bias from potential intra-patient heterogeneity and a high failure rate (Colomer et al., E Clinical Medicine 2020; 25:100487).

Recently, liquid biopsies have proved to be a promising alternative to tissue biopsy for detecting genomic aberrations and molecular subtype characterization (Maia et al., Nat Rev Urol 2020, Nat Rev Urol. 2020; 17(5):271-291), also allowing for serial testing, through non-invasive blood draws. Liquid biopsies could also help to readily identify possible resistance mechanisms and to detect minimal residual disease (Heitzer et al., Nature Reviews Genetics 2019, Nat Rev Genet. 2019; 20(2):71-88). Further, liquid biopsies provide a more comprehensive characterization of a patient's tumor that is neither temporally nor spatially restricted, as is the case of tissue biopsies (Siravegna et al., Nature Reviews Clinical Oncology 2017, Nat Rev Clin Oncol. 2017; 14(9):531-548), and can serve for the enrollment of patients in umbrella trials. For example, liquid biopsies that involve the analysis of circulating free (cfDNA), and specifically circulating tumor DNA (ctDNA), have shown promise in the stratification and treatment selection of mCRPC patients (Chi et al., Journal of Clinical Oncology 2020 38:15_suppl, 5551-5551).

However, biological and technical issues influence the ability to accurately stratify patients using current methods, especially for copy number change rich cancers, such as prostate cancer (Hieronymus et al., Elife. 2018 Sep. 4; 7:e37294 and U et al., Nature. 2020 February; 578(7793):112-121). Biological and technical challenges for accurate detection of genomic lesions in ctDNA, such as metastatic prostate cancer (mPC) plasma ctDNA, include broad ranges of tumor fractions, intra-patient genomic heterogeneity, frequent aneuploidy, and common imbalanced copy number changes. For example, low ctDNA fractions limit the accurate detection of copy number changes, in particular copy number losses, and further make it hard to discriminate between mono- and biallelic gene loss (Warner et al., Clin. Canc. Res. 2021, Clin Cancer Res. 2021; 27(6):1650-1662).

ctDNA fractions depend on the cancer type and stage of the disease, and can significantly vary between patients. For example, in one study it was found that in a CRPC cohort the median ctDNA fraction in the plasma sample was 18.1% (Romanel et al., Sci Transl Med. 2015; 7(312):312re10). In another study, in the earlier setting of patients with CRPC, a median ctDNA of 11% was found (Vandekerkhove et al., Eur Urol. 2019; 75(4):667-675).

In samples containing low levels of tumor DNA (for example a cfDNA sample comprising <20% ctDNA), current methods display low accuracy in the identification of copy number losses. This greatly limits the amount of information that can be inferred from a sample about the patient's disease. For example, without an accurate measure of allelic imbalance and copy number status, it is not possible to detect genome aberrations such as copy-number neutral losses of heterozygosity. This is important because without an accurate measure of allelic imbalance and copy number status, it is not possible to accurately identify biallelic alterations such as a monoallelic mutation coupled with loss of the other allele.

There therefore remains a significant need for improved diagnostic and analytical methods for the stratification and monitoring of cancers, particularly methods that display greater sensitivity and that provide more detailed information on the genetic aberrations present in a cancer.

### SUMMARY OF INVENTION

The present invention provides an in vitro method for staging, classification, screening, monitoring, stratification, selecting treatment for, ascertaining whether treatment is working in, and/or prognostication of cancer in a subject, wherein said method comprises the steps defined in claim 1.

The present invention provides an in vitro assay for staging, classification, screening, monitoring, stratification, selecting treatment for, ascertaining whether treatment is working in, and/or prognostication of cancer in a subject, wherein said method comprises the steps defined in claim 24.

The present inventors have found that the method and assay of the present invention are surprisingly effective and sensitive at detecting and monitoring allele-specific copy number aberrations (asCNAs) in subjects known or suspected of having a cancer. The method and assay of the present invention utilizes a selected panel of high MAF SNP loci in bespoke gene-regions including the exonic, intronic and flanking regions of a number of selected genes. The combination of the genes, the gene-region designs, and the SNPs selected within each region, have been found by the inventors to lead to the method and assay of the invention being both effective and sensitive for detecting asCNAs. Example 1 below describes a method according to the present invention and an assay according to the present invention, and shows results of the use of the method/assay (“Example 1 SNP panel”). As can be seen from the results section and FIGS. 3A and 19C, when the Example 1 SNP panel was applied to the same prostate cancer samples as an earlier disclosed assay (Annala et al. PMID: 29367197), the Example 1 SNP panel was able to detect asCNAs in line with those reported by Annala et al. (105/109 copy number aberrations, 96.33%), as well as asCNAs not reported by Annala et al., in particular in low tumor fraction samples (for example in samples with 7% tumor fraction for patients #134 (see FIG. 3A). The method/assay of the present invention was also able to detect complex copy number aberrations not reported in Annala et al. (for example, homozygous deletion of PTEN at all time-points for patient #134—see FIG. 3A). This supports that the methods and assays of the present invention provide an accurate and sensitive method able to discriminate between complex asCNAs, for example homozygous deletion asCNAs (which have no imbalance) and hemizygous deletion asCNAs (in which an imbalance is present).

The Example 1 SNP panel method was also applied on serial samples from 3 patients treated with a PARP inhibitor (1 BRCA mutant and 2 ATM mutant patients). For the BRCA mutant patient, the Example 1 SNP panel consistently identified a hemizygous deletion (cnA=1, cnB=0) of BRCA2 gene-region, including detecting this mono-allelic deletion at samples with a low tumor fraction (around 15%). The inventors also found that the deletion was accompanied by a pathogenic missense BRCA2 germline mutation, showing that this patient had biallelic gene loss of function (monoallelic mutation coupled with loss of the other allele). Identification of this biallelic gene loss of function would not have been possible without detailed information about the asCNAs present in the sample. For both ATM mutants, complex asCNAs were detected in plasma samples for both patients. Specifically, both had a complex ATM copy number status (2 copies of one allele and 1 of the other) within aneuploid genome accompanied by a nonsense mutation harboured on the non-gained allele with a CN-corrected VAF=33% (see FIGS. 5C and 21B). Additionally, for one patient, a copy-number neutral loss of heterozygosity (2 copies of one allele and 0 of the other) on TP53 accompanied by the missense mutation on both alleles was identified, suggesting full-impairment of TP53 (see FIG. 21B). The results in these patients further support the accuracy and sensitivity of the method and assay of the invention at determining complex asCNAs, in particular the discrimination between wild-type status (copy call 1,1) and a copy neutral loss of heterozygosity (copy call 2,0) was possible.

Some drugs, for example PARP inhibitors, require full-impairment of genes to be effective cancer treatments. In view of this, the ability to accurately identify complex aberrations leading to gene impairment has important clinical implications. The method and assay of the invention is able to achieve this type of accurate identification. The results in the serial samples from the 1 BRCA mutant patient in FIG. 4 also show how the method and assay of the invention could be used to track treatment effectiveness by monitoring the change in allelic imbalance signal (i.e. depletion of the clone harbouring the BRCA2 loss) and/or tumor fraction at different time points during treatment.

As such, the present inventors have demonstrated that the method of the invention is especially useful for the real-time strategic assessment of drug target biomarkers in cancer patients, including PARP-inhibitors, and thus allows for the optimization of treatments and the monitoring of disease progression/regression in patients undergoing treatments. The present methods find particular utility for the detection and monitoring of asCNAs in subjects known or suspected of having prostate cancer.

The present invention also provides a set of oligonucleotide probes and kits suitable for use in the method of the present invention.

### BRIEF DESCRIPTION OF THE DRAWINGS

FIG. 1A) shows the selection criteria used to determine the Example 1 SNP panel, and FIG. 1B) shows a summary of the statistics for the Example 1 SNP panel. Genomic sizes and number of high minor allele frequency (MAF) SNPs per gene-region of target and control genes are reported. FIG. 1C) shows a schematic summarizing the main blocks of the pipeline. As illustrated in the figure, copy-number assessment and gene-region allelic imbalance detections were independently performed and the results were integrated and corrected for ploidy and tumor content to return allele-specific copy number status of each gene-region (the step in the box labelled “0” requires a set of high-coverage control sample data, and the steps in the two boxes labelled “2” in the top half of the figure require cfDNA and matched control sample data). FIG. 1D) shows a read-depth estimation for each gene-region. Estimations (comprising exonic-intronic regions and upstream/downstream flanking regions) were performed by dividing cfDNA sample data (labelled “cfDNA” on left-hand side of figure) by matched control sample data (labelled “matched control” on left-hand side of figure); i) Log 2R values for each amplicon (Log 2Rampi) were computed by using normalized data (for GC content and for intra-sample mean coverage); ii) the Log 2R_GeneRegion value for the gene-region was then estimated as median across all amplicons within the gene-region. FIG. 1E) shows a schematic of una tantum (per platform) reference model generation for gene-region based allelic imbalance detection method based on control samples. First, for each informative SNP (iSNP, 0.2<AF<0.8) in the panel, mean allelic fraction across the control samples (A̅F̅_{SNP}) was extracted. AFs of informative SNPs were stratified by coverage quantiles and the AF standard deviation for each interval was computed (σ_{AF}^{q}). FIG. 1F) shows schematics of the gene-region based approach for allelic imbalance computation. First, informative SNPs of the patient were inferred by exploiting the matched control sample. Then, a simulated iSNPs AF distribution (D_{β=1}) was obtained by sampling one time per iSNP from a normal distribution. Parameters of the normal distribution corresponding to each iSNP were defined by interrogating the reference model (selected parameters are highlighted in black). Starting from the simulated distribution, distributions mimicking levels of local admixture (P) were obtained. Finally, the observed informative SNPs mirrored AF distribution (in red) was compared with the simulated informative SNPs AF distributions to compute gene-region specific information (i.e. presence of allelic imbalance, beta). This procedure was repeated K times (K=100, by default). FIG. 1G) shows the integration of read-depth estimation and allelic imbalance. The top panel shows the distribution of Log 2R coloured by allele-specific copy-number, and the bottom panel shows the Log 2R-Beta space obtained by integration of read-depth estimations and allelic imbalance. Each point represents a gene-region, and cluster of points are annotated with their expected allele-specific copy numbers.

FIG. 2 shows the assessment of Allelic Imbalance (AI) calls as function of number of iSNPs and gene-region. FIG. 2A) shows the proportion of AI calls on a data (Cornell) set by varying the percentage of iSNPs compared with whole exome sequencing (WES) data (ranges of iSNPs used were reported). Values are shown for both control and cfDNA samples (light and dark shaded boxes at the bottom of each bar, respectively). The proportions of AI reported are relative to the number of AI calls obtained using all the iSNPs available for each gene in the Example 1 SNP panel (i.e. 904). The number of allelic imbalance calls is shown on top of each bar. AI calls were stratified for the number of iSNPs used (bar colors). FIG. 2B) shows evidence of allelic imbalance (E(AI_{T}), y-axis) for representative gene-regions at varying of the percentage of iSNPs used across the patient (Cornell) cohort. The line shading indicates ctDNA level of the sample. Donuts show the proportion of evidence of imbalance per percentage of iSNPs used. The data for all gene-regions are shown in FIG. 15. FIG. 2C shows the proportion of recovered allelic imbalance calls in synthetically diluted samples. ctDNA levels are reported on x-axis. Calls were stratified by allele-specific CN call as established in the real cfDNA samples used for dilutions.

FIG. 3 shows a comparative overview of somatic copy-number aberrations (SCNA) calls on two CRPC serial samples patients using two independent assays (Example 1 SNP Panel (“PCF_SELECT”) and the assay disclosed in Annala et al. (Cancer Discov, 2018 (8) (4) 444-457)). FIG. 3A shows SCNA and SNVs calls detected by the Example 1 SNP panel and by the Annala et al. assay (Cancer Discov, 2018 (8) (4) 444-457). The calls were reported for serial samples with varying ctDNA levels as determined by the Example 1 SNP panel. Gene-regions were grouped by pathways and sorted alphabetically. FIG. 3B shows Log 2R-beta spaces for patient 110. CHD1 and TP53 are highlighted by arrows. The bottom panel shows the mirrored allelic fraction distribution of the iSNPs spanning CHD1 and TP53 for both cfDNA (red) and matched control (green) samples. FIG. 3C shows the Log 2R-beta spaces for patient 134. PTEN and RB1 are highlighted by arrows. The bottom panel shows the mirrored allelic fraction distribution of the iSNPs spanning PTEN and RB1 for both cfDNA (red) and matched control (green) samples.

FIG. 4 shows the BRCA2 status during Niraparib treatment of a CRPC patient. FIG. 4A shows the clinical history of the patient. The stars denote plasma samples sequenced and analyzed using the Example 1 SNP Panel. FIG. 4B shows the mirrored allelic fractions (AF) distribution of patient's BRCA2 gene-region iSNPs. Dashed lines represent the mirrored AF median. The top panel corresponds to PBMCs signal (green); and the following panels correspond to serial plasma samples. FIG. 4C shows the BRCA2 copy number (CN) status of a BRCA2 mutant patient. The left panel shows the Allele-specific CN space of plasma samples with estimated ctDNA level>15%; BRCA2 gene-region corresponding status is highlighted. The right panel shows the variant allele frequency (VAF) of BRCA2 p.E2663V germline mutation. The dotted line furthest to the right of each plot represent the VAF observed in the matched control sample.

FIG. 5 shows the ATM status during sequential treatment (Docetaxel+Niraparib) of a CRPC patient. FIG. 5A shows the clinical history of the patient. The stars denote plasma samples sent for sequencing and analyzed using the Example 1 SNP panel. FIG. 5B shows a summary of the tissue samples collected after death and sent for sequencing (TC=tumor content; asPloidy=allele-specific ploidy). FIG. 5C shows the ATM copy number (CN) status. The left panel shows the allele-specific CN space of plasma and tissue samples; ATM gene-region corresponding status is highlighted; for plasma samples average CN and standard deviations are reported. The middle panel shows the CN-corrected variant allele frequency (VAF) of ATM p.R2763* mutation. The allele-specific ploidy of each sample is reported on the right of the plot. The right panel shows the mirrored allelic fractions (AF) distribution of patient's ATM gene-region iSNPs. The dashed lines represent the mirrored AF median.

FIG. 6 shows a diagram depicting the procedure for focal aberration detection. For each gene-region, an iterative procedure is applied to compute Log 2R values. The gene area (exonic-intronic) is represented as solid color boxes and iteratively wider flanking regions are represented with white boxes and solid lines. Log 2Rn is the Log 2R computed considering all the amplicons spanning the gene-region.

FIG. 7 shows a histogram of Log 2R values per gene-region obtained performing control vs. control segmentation on the Cornell cohort for a high-noise gene-region (A) and a low-noise gene-region (B).

FIG. 8 shows a diagram depicting the workflow to integrate Log 2R values corrected for ploidy and purity (i.e. ctDNA level) and allelic imbalance calls to obtain allele-specific calls.

FIG. 9 shows distribution of iSNPs per gene-region in the Cornell samples. Gene-regions are divided by type and sorted by decreasing median number of iSNPs. Top panel shows the number of SNPs per gene-region included by design in the panel

FIG. 10 shows the distribution of iSNPs per gene-region across 4 healthy donors. Gene-regions are divided by type and sorted by decreasing median number of iSNPs in the Cornell cohort. Top panel shows the number of SNPs per gene-region included by design in the panel.

FIG. 11 shows the effects of GC content normalization on coverage. The top two graphs show distribution of amplicon coverage stratified by GC content in control (left) and cfDNA (right) sample. The lower two graphs show distribution of amplicon coverage after GC content normalization in control (left) and cfDNA (right) sample.

FIG. 12 shows distribution of iSNPs allelic fractions in control (green) and cfDNA (red) samples before and after RMB correction (left and right panels, respectively). Dashed lines show median allelic fraction of iSNPs.

FIG. 13 shows the proportion of allelic imbalance (AI) calls on control samples (i.e. False Positive Rate, FPR) obtained by using reference models built with 10, 20, 30, or 40 control samples. Each model is built by randomly selecting control samples from the Cornell study set; 20 models were built for each cardinality.

FIG. 14 shows beta values computed on samples from 3 healthy volunteers (HV) sequenced at 4 institutions (Cornell, Vancouver, UCL and Trento). Reference models generated pooling Cornell, Vancouver and UCL control samples were used to compute beta values on HV sequenced at the same institution (x-axis) and sequenced in Trento (y-axis). Color and size of the points denote respectively the reference model and the number of iSNPs used. Inset shows discordant beta values observed in gene-regions with a low number of iSNPs.

FIG. 15 shows evidence of allelic imbalance (E(AI_{T}), y-axis) for each gene-regions at varying of the percentage of iSNPs used across the Cornell cohort. Line color indicates ctDNA level of the sample. Donuts show proportion of evidence of imbalance per percentage of SNPs used.

FIG. 16 shows the evidence of allelic imbalance denoted by the box color and beta values annotated inside each box for RB1 and CLU shown for both ctDNA sample (1^{st} and 3^{rd} rows) and matched control sample (2^{nd} and 4^{th} rows) for two patients.

FIG. 17 shows a barplot of the mean proportion of recovered allelic imbalance calls at decreasing ctDNA levels across the synthetically diluted samples.

FIG. 18 shows the proportion of recovered allelic imbalance calls in synthetically diluted samples for ERG_TMPRSS2, NKX3-1, TP53 and RB1 gene-regions. ctDNA levels are reported on the x-axis.

FIGS. 19A) and 19B) are diagrams depicting integration of log 2R and beta in diploid (A) and polyploid sample (B). Dotted and dashed lines represent the expected and the observed log 2R in Wild-Type segments. FIG. 19C) shows a comparative analysis between the Example 1 SNP panel and the assay disclosed in Annala et al (Cancer Discov, 2018 (8) (4) 444-457) on serial polyploid samples. FIG. 19D) shows Log 2R-beta spaces for FOXA1 and PTEN gene-regions highlighted by arrows. The lower panels of FIG. 19D show the mirrored allelic fraction distribution of the iSNPs spanning FOXA1 and PTEN for both cfDNA (red) and matched control (green) samples.

FIG. 20 shows the ERG_TMPRSS2 gene-region in plasma and tissue samples from patient TR067. Panels show allele fractions (AFs) of iSNPs. Dashed lines denote an AF of 0.5. Plasma samples are shown in chronological order.

FIG. 21 shows an overview of the following aspects for a CRPC patient receiving Docetaxel, Niraparib and Radium-223: FIG. 21A) shows the clinical history of the patient. Stars denote plasma samples sent for sequencing and analyzed using the Example 1 SNP panel. FIG. 21B) shows the copy-number status of mutated genes. In particular the top panel of FIG. 21B shows allele-specific CN space. Gene-regions harbouring a non-synonymous SNV in at least two independent plasma samples are highlighted. Average CN and standard deviations across assessable samples are reported for each gene-region. The lower panel of FIG. 21B shows CN-corrected VAF of non-synonymous mutations identified by ABEMUS. Plasma samples are sorted chronologically. Coloured boxes denote treatment administered to the patient at the moment of sample collection.

FIG. 22 shows the proportion of recovered iSNPs in cfDNA samples from the Cornell cohort as function of ctDNA level and beta of gene-regions. Genomic position with 0.2<AF<0.8 and 0.05<AF<0.95 are called as iSNPs in panel A and B, respectively.

FIG. 23 shows information and sequencing statistics of the study samples used in Example 1.

FIG. 24 the ABEMUS parameters used for detection of somatic single nucleotide variants (SNVs)

### DEFINITIONS

Unless defined otherwise, all technical and scientific terms used herein have the same meaning as commonly recognized by one of ordinary skill in the art to which this invention belongs.

As used herein, the term “biological sample” refers to a biological sample derived from a subject to be screened. The biological sample may be any suitable sample known in the art in which the expression of the selected markers can be detected. Included are individual cells and cell populations obtained from bodily tissues or fluids. Examples of suitable body fluids to be tested are plasma, blood, lymph, cerebral fluid, urine and saliva. Examples of suitable body tissues to be tested include prostate tissue, bladder tissue, breast tissue, ovarian tissue and pancreatic tissue.

As used herein, the term “non-tumor DNA” refers to chromosomal genomic DNA present in, or derived from, a non-cancerous cell. For example, non-tumor DNA may be derived from a white blood cell (WBC).

As used herein, the term “target gene” refers to a gene that is known or suspected of being commonly mutated in certain types of cancer and/or frequently altered in signalling pathways known or suspected of being druggable pathways for treating a cancer. Examples of such genes include: AR, AKT1, AKT2, AKT3, APC, ARID1A, ASXL1, ATM, ATR, AURKA, BRAF, BRCA1, BRCA2, CCND1, CDK12, CDK4, CDK6, CDKN1B, CDKN2A, CHD1, CLU, CTNNB1, CUL1, CYLD, ERCC1, KDM6A, ERCC2, ERCC3, MED12, ERCC4, ERCC5, ERG_TMPRSS2, SMARCA1, FAM183B, FAM60A, FANCA, FANCC, FANCD2, FANCE, FANCF, FANCG, FBXW7, FOXA1, FOXP1, GNAS, HSD3B1, IDH1, IDH2, KMT2C, KMT2D, KRAS, MDM2, MDM4, MET, MLH1, MSH2, MSH6, MYC, MYCN, NCOA2, NFE2L2, NKX3-1, PALB2, PIK3CA, PIK3CB, PIK3R1, PTEN, RAD51B, RAD51C, RB1, RNF43, RUNX1, RYBP, SPOP, TP53, ZBTB16 and ZFHX3. Preferred examples of target genes include: AKT1, AKT2, AKT3, APC, ARID1A, ASXL1, ATM, ATR, AURKA, BRAF, BRCA1, BRCA2, CCND1, CDK12, CDK4, CDK6, CDKN1B, CDKN2A, CHD1, CLU, CTNNB1, CUL1, CYLD, ERCC1, ERCC2, ERCC3, ERCC4, ERCC5, ERG_TMPRSS2, FAM183B, FAM60A, FANCA, FANCC, FANCD2, FANCE, FANCF, FANCG, FBXW7, FOXA1, FOXP1, GNAS, HSD3B1, IDH1, KMT2C, KMT2D, KRAS, MDM2, MDM4, MET, MLH1, MSH2, MSH6, MYC, MYCN, NCOA2, NFE2L2, NKX3-1, PALB2, PIK3CA, PIK3CB, PIK3R1, PTEN, RAD51B, RAD51C, RB1, RNF43, RUNX1, RYBP, SPOP, TP53, ZBTB16 and ZFHX3.

As used herein, the term “control gene” is a gene that is known to not be commonly mutated in a cancer and/or to not acquire mutations during the progression of a cancer. Examples of such genes include: ADAM2, APBB11P, AQP8, ATG14, ATXN7, C9orf47, CACNG5, CST7, CSTF2T, DRD1, ENPEP, EPN1, FAM183B, FAM60A, FCER1A, HDAC8, GRHPR, IL1RL2, KRT85, L3MBTL1, LRRC17, MESDC2, M1S18A, MTIF3, NAPG, NFXL1, N1T2, NOTCH3, OR3A3, PTAFR, PTGER4, PXDNL, RNF125, RSF1, SPDYA, TEX11, TK2, TOP3B, UGT2B17, VNN3 and ZBTB9. Preferred examples of control genes include: ADAM2, APBB11P, AQP8, ATG14, ATXN7, C9orf47, CACNG5, CST7, CSTF2T, DRD1, ENPEP, EPN1, FAM183B, FAM60A, FCER1A, GRHPR, IL1RL2, KRT85, L3MBTL1, LRRC17, MESDC2, MIS18A, MTIF3, NAPG, NFXL1, NIT2, NOTCH3, OR3A3, PTAFR, PTGER4, PXDNL, RNF125, RSF1, SPDYA, TK2, TOP3B, UGT2B17, VNN3 and ZBTB9. The control genes for one type of cancer may be different to the control genes for a different cancer, or they may be the same. For example, control genes for prostate cancer may be one or more control genes selected from the lists of control genes above.

As used herein, the term “gene-region” refers to a region of the genome that covers a gene in the genome, e.g. the genome of a subject, for example a human, and includes a gene and a maximum extension of 200 Kbp per side of the gene. A gene-region may cover the intronic (i.e. non-coding), exonic (i.e. coding) and flanking regions of a gene. Preferably, a gene-region of the invention covers intronic, exonic and flanking regions of a gene. A gene-region may be referred to using the genomic location of the region, for example using the coordinates of the start position and end position of the location in a specific chromosome. For a human subject a genomic region is suitably described by a genomic location, and in particular a genomic location with reference to a reference genome (for example, a digital nucleic acid sequence database, assembled a representative example of a species' set of genes). For example, for a human subject, with reference to the human reference genome GRCh37 (also referred to as Human Genome 19 (hg19)) or human reference genome GRCh38 (also referred to as Human Genome 38 (hg38)). For the present inventions, preferably the reference genome is human reference genome GRCh37 (also known as hg19).

As used herein, the term “target gene-region” refers to a gene-region that includes a target gene (supra), and the term “control gene-region” refers to a gene-region that includes a control target (supra).

As used herein, the term “allelic fraction” or “AF” refers to the number of times a mutated base/variant is observed at a genomic locus, divided by the total number of times any base is observed at the genomic locus. For example, in a sample comprising DNA derived from a diploid genome, an allelic fraction of 0.5 implies that there is one copy of an allele with base X at position Y and one copy of an allele with base Z at position Y. That is to say, that 50% of the DNA in the sample contains the allele with base X at position Y and the other 50% of the DNA in the sample contains the allele having base Z at position Y. In a DNA sample derived from an aneuploid cell (for example a cell that contains three copies of a chromosome), an AF of 0.33 for a first allele and an AF of 0.67 for a second implies that there is one copy of an allele with base X at position Y and two copy of an allele with base Z at position Y. That is to say, that 33% of the DNA in the sample contains the allele with base X at position Y and the other 67% of the DNA in the sample contains the allele having base Z at position Y.

As used herein, the term “single nucleotide polymorphism” or “SNP” refers to a polymorphism that occurs at a polymorphic site occupied by a single nucleotide. The site of the SNP is usually preceded by and followed by highly conserved sequences (e.g., sequences that vary in less than 1/100 or 1/1000 members of a population). As used herein, “SNPs” is the plural of SNP. SNPs are most frequently diallelic. The most common allele of a SNP is called a “major” or “wild-type” allele and an alternative allele of said SNP is called a “minor” or “mutant” allele. A SNP usually arises due to substitution of one nucleotide for another at the polymorphic site. SNPs can also arise from a deletion of a nucleotide or an insertion of a nucleotide relative to a reference allele.

As used herein, the term “SNP location” and “SNP locus” refer to a polymorphic site at which a polymorphism occurs. The term “SNP loci” is the plural form of the term “SNP locus”. A “SNP location” or “SNP locus” can be referred to using its unique dbSNP Reference SNP ID. (also referred to as the rsid) in the National Center for Biotechnology Information's dbSNP database. A dbSNP Reference SNP (rsid or RefSNP) number is a locus accession for a variant type assigned by dbSNP. The RefSNP catalog is a non-redundant collection of submitted variants which were clustered, integrated and annotated. RefSNP number is the stable accession regardless of the differences in genomic assemblies. They provide a stable variant notation for mutation and polymorphism analysis, annotation, reporting, data mining, and data integration. A RefSNP is represented by a number preceded by the letters “rs”. A “SNP location” or “SNP locus” can alternatively be referred to using the coordinates of the position of the polymorphic site in a specific chromosome. For a human subject a genomic location is suitably described by reference to a reference genome (for example, a digital nucleic acid sequence database, assembled from a representative example of a species' set of genes). For example, for a human subject, with reference to the human reference genome GRCh37 (also referred to as Human Genome 19 (hg19)) or human reference genome GRCh38 (also referred to as Human Genome 38 (hg38)). For the present inventions preferably the reference genome is human reference genome GRCh37 (also known as hg19).

As used herein, the term “polymorphism” refers to a genetic variation, or the occurrence of two or more genetically determined alternative sequences at a single genetic locus in a population. Each version of the sequence with respect to the polymorphic site is referred to as an “allele” of the polymorphic site. Typically, polymorphisms have two alleles, with the minor allele occurring at a frequency of greater than 1%, and more preferably greater than 5% or 10% of a selected population. The allelic form occurring most frequently in a selected population is sometimes referenced as the “wild-type” form. Diploid organisms may be homozygous or heterozygous for allelic forms. A biallelic polymorphism has two forms. A triallelic polymorphism has three forms. Examples of polymorphisms include restriction fragment length polymorphisms (RFLPs), variable number of tandem repeats (VNTRs), single nucleotide polymorphisms (SNPs), single nucleotide variants (SNVs), dinucleotide repeats, trinucleotide repeats, tetranucleotide repeats, simple sequence repeats, indels, and insertion elements such as Alu.

As used herein, the terms “single nucleotide variant” or “SNV” refer to a single nucleotide variation in a genome sequence. SNVs may be rare or common in a population. If an SNV is present in at least 1% of the population, the SNV may be referred to as a “single nucleotide polymorphism” or “SNP”.

SNPs tend to be evolutionarily stable from generation to generation and, as such, can be used to study specific genetic abnormalities throughout a population. If SNPs occur in the protein coding region (i.e. exonic region) it can lead to the expression of a variant, sometimes defective, form of the protein that may lead to development of a genetic disease. Such SNPs can therefore serve as effective indicators of the genetic disease. Some SNPs may occur in non-coding regions, but nevertheless, may result in differential or defective splicing, or altered protein expression levels. SNPs can therefore be used as diagnostic tools for identifying individuals with a predisposition for certain diseases, genotyping the individual suffering from the disease in terms of the genetic causes underlying the condition, and facilitating drug development based on the insight revealed regarding the role of target proteins in the pathogenesis process.

As used herein, the terms “heterozygous single nucleotide polymorphism” and “heterozygous SNP” refer to a SNP that is present in the DNA of a sample at an AF of between 0.05 to 0.95. In certain embodiments, the terms a heterozygous single nucleotide polymorphism” and “heterozygous SNP” refers a SNP that is present in the DNA of a sample at an AF of between 0.2 to 0.8.

As used herein, the term “minor allele frequency” or “MAF” refers to the frequency at which the second most common allele occurs in a given population. The frequency may be considered high or low. As used here, a “high MAF” is typically an allele that occurs at a frequency of 0.2 to 0.5 in a population, for example at a frequency of 0.2 to 0.5 in the 1000 Genomes Project Genotype Data (release 20130502; PMID: 26432245). As used herein, a “low MAF” is typically an allele that occurs at frequency of less than 0.2, for example less than 0.2 to around 0.01 in a population, for example at a frequency of 0.2 to 0.5 in the 1000 Genomes Project Genotype Data (release 20130502).

As used herein, the term “informative single nucleotide polymorphism”, “informative SNP” or “iSNP” refers to a heterozygous SNP that is present in a gene-region in the genome of a subject. The term “iSNPs” is the plural form of the term “iSNP”.

As used herein, the terms “iSNP location” or “iSNP locus” refer to a polymorphic site at which an iSNP occurs. The term “iSNP loci” is the plural form of the term “iSNP locus”. A “iSNP location” or “iSNP locus” can be referred to using its unique dbSNP Reference SNP ID. (also referred to as the rsid) in the National Center for Biotechnology Information's dbSNP database. A “iSNP location” or “iSNP locus” can alternatively be referred to using the coordinates of the position of the polymorphic site in a specific chromosome as described supra. For the present inventions, preferably the reference genome is human reference genome GRCh37 (also known as hg19).

As used herein, the terms “allele imbalance”, “allelic imbalance” and “AI” refer to an imbalance in the identity of the allele present in the genome of a subject. For AI to be present in a genome there must be at least two different alleles for a gene-region. A genome having identical alleles for a gene-region does not exhibit AI. A loss of heterozygosity (LOH) is a common form of allelic imbalance.

As used herein, the term “copy number” refers to the number of copies of a gene-region, gene, or part thereof, present in the chromosomal DNA of an individual or in chromosomal DNA derived from a cell of an individual. A “normal copy number” when used herein refers to the copy number of a normal or wild-type allele present in a normal cell of a subject. The copy number for any given gene-region, gene or part thereof, may range from 0 to 3 (for example 0, 1, 2 or 3), 0 to 4 (for example 0, 1, 2, 3 or 4), 0 to 5, 0 to 6, or 0 to more than 6. A change in copy number may arise from copy number alteration such as gains or losses of large segments of the genome.

As used herein, the terms “allele-specific copy number aberration”, “allele-specific informed copy number aberration” and “asCNA” refer to alteration in the total copy number of a gene-region, or part thereof, and the specific number of copies of each chromosome. Types of asCNAs include: copy number gain, copy number loss, and copy number-neutral loss of heterozygosity. A gain in copy number may be a gain of either one or both of the two inherited copies. An gain may be referred to as “unbalanced” when the gain differs in magnitude between the alleles (for example a 2,1 gain, 3,2 gain, a 4,2 gain, a 4,3 gain etc.). An gain may be referred to as “balanced” when both alleles gained equally much (for example a 2,2 gain, 3,3 gain, a 4,4, gain, a 5,5, gain, etc.). A copy number loss may be the loss of one of the parental copies of a gene-region, or part thereof (this may be referred to as a mono-allelic copy number loss). Such a loss may result in loss of heterozygosity (LOH) if the parental copies were heterozygous for that gene. A copy number loss may be the loss of both of the parental copies of a gene-region, or part thereof (this may be referred to as a bi-allelic copy number loss). The loss of one parental copy of a gene-region, or part thereof, accompanied by a simultaneous gain of the other parental copy of the same gene-region, or part thereof can also occur. If the parental copies were heterozygous for that gene, such an aberration may result in a copy number-neutral loss of heterozygosity (i.e. a loss of heterozygosity without a change in total copy number). This may also be referred to as a loss of heterozygosity 0,2. The loss of one parental copy of a gene-region, or part thereof, accompanied by a simultaneous gain of multiple copies, for example 2, 3, 4, 5, or more than 5 copies, of the other parental copy of the same gene-region, or part thereof can also occur. Such an aberration can be referred to as a loss of heterozygosity 0,3 when two copies of the other parental copy of the same gene-region are gained; a loss of heterozygosity 0,4 when three copies of the other parental copy of the same gene-region are gained; and a loss of heterozygosity 0, X when X-1 copies of the other parental copy of the same gene-region are gained wherein X is 4, 5, 6, 7, or a higher integer.

As used herein, the term “androgen receptor (AR) associated” means a gene-region that includes a gene associated with the function in the androgen (receptor) signalling pathway. Examples of such genes include: AR, FOXA1, FOXP1, HSD3B1, NCOA2 and ZBTB16. Aberrant activity of the androgen receptor is associated with certain types of prostate cancer. Aberrant activity of the androgen receptor has also been associated with breast cancer, ovarian cancer, pancreatic cancer and bladder cancer. If a genetic aberration, such as an asCNA, or a somatic or germline mutation, is detected in one or more gene-region of this type in a subject, it indicates that a subject would benefit from ceasing or altering treatment with one or more class of drug that is known or suspected of targeting AR function. An example of this class of drug include hormonal agents, such as LHRH agonists (for example leuprolide, goserelin, triptorelin, or histrelin), LHRH antagonists (for example degarelix), androgen blockers (for example abiraterone or ketoconazole), anti-androgens (for example flutamide, bicalutamide, nilutamide, enzalutamide, apalutamide or darolutamide), androgen synthesis inhibitors (for example abiraterone), estrogens and steroids (for example prednisone or dexamethasone); and in particular androgen blockers (for example abiraterone or ketoconazole), anti-androgens (for example flutamide, bicalutamide, nilutamide, enzalutamide, apalutamide or darolutamide) and androgen synthesis inhibitors (for example abiraterone). It also indicates that a subject would benefit from ceasing or altering treatment with one or more class of drug that is known or suspected of targeting AR function from treatment with one or more alternative cancer treatment, for example a chemotherapy such as a taxane (for example docetaxel and cabazitaxel) or a platinum-based antineoplastic drugs (for example carboplatin).

As used herein, the term “cell cycle associated” means a gene-region that includes a gene associated with the process of growth and proliferation of a cell. Examples of such genes include: AURKA, BRAF, CCND1, CDK12, CDK4, CDK6, CDKN1B, CDKN2A, CUL1, FBXW7, KRAS, MDM2, MDM4, MYC, MYCN, RB1 and TP53. Genetic aberrations in genes associated with growth and proliferation of a cell are known to occur in cancer such as prostate cancer, breast cancer, ovarian cancer, pancreatic cancer and bladder cancer, and in metastatic cancer. If a genetic aberration, such as an asCNA, or a somatic or germline mutation, is detected in one or more gene-region of this type in a subject, it indicates that a subject might benefit from treatment with one or more class of drug that is known or suspected of targeting cell cycle perturbations. Examples of such class of drug include: ATR inhibitors (for example Berzosertib), CDK inhibitors (for example Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, and Pyrimidines; and in particular Flavopiridol, Palbociclib, ribociclib and abemaciclib) chemotherapies (for example a taxane (for example docetaxel or cabazitaxel), and c-Met inhibitors (for example cabozantinib)), WEE1 inhibitors (for example adavosertib), Aurora kinase inhibitors (for example Alisertib, ZM447439, hesperidin, and VX-680) and alkylating agents (for example nitrogen mustards (such as cyclophosphamide, chlormethine, uramustine, melphalan, chlorambucil, ifosfamide, and bendamustine), nitrosoureas (such as carmustine, lomustine, and streptozocin) and alkyl sulfonates (such as busulfan)).

As used herein, the term “chromatin remodelling associated” means a gene-region that includes a gene that is associated with cell growth and cell division steps, such as cell-cycle progression and chromosome segregation. Genes associated with chromatin remodelling typically exert a suppressive effect on tumor growth. Examples of such genes include: ARID1A, CHD1, KDM6A, MED12, SMARCA1, KMT2C, KMT2D and RYBP. Genetic aberrations in genes associated with chromatin remodelling functions are known to occur in cancer such as prostate cancer, breast cancer, ovarian cancer, pancreatic cancer and bladder cancer, and in metastatic cancer. If a genetic aberration, such as an asCNA, or a somatic or germline mutation, is detected in one or more gene-region of this type in a subject, it indicates that a subject might benefit from treatment with one or more class of drug that is known or suspected to target chromatin remodelling functions. Examples of such class of drug include DNMT1 inhibitors (for example 5-azacitidine), HDAC inhibitors (for example vorinostat and romidepsin) and BET inhibitors (for example I-BET 151, I-BET 762, OTX-015, TEN-010, CPI-203, CPI-0610, olinone, RVX-208, ABBV-744, AZD5153, MT-1, and MS645).

As used herein, the term “DNA repair associated” means a gene-region that includes a gene associated with the identification and correction of damage to DNA. Examples of such genes include: ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C. Genetic aberrations in genes associated with DNA repair functions are known to occur in cancer such as prostate cancer, breast cancer, ovarian cancer, pancreatic cancer and bladder cancer, and in metastatic cancer. If a genetic aberration, such as an asCNA, or a somatic or germline mutation, is detected in one or more gene-region of this type in a subject, it indicates that a subject might benefit from treatment with one or more class of drug that is known or suspected to target a DNA repair function. Examples of such class of drug include PARP inhibitors (for example example olaparib, rucaparib, niraparib or talazoparib, Veliparib, Pamiparib, Rucaparib, and Veliparib; and in particular example olaparib, rucaparib, niraparib and talazoparib), ATR inhibitors (for example Berzosertib), CDK inhibitors (for example Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, and Pyrimidines; and in particular Flavopiridol, Palbociclib, ribociclib and abemaciclib), DNA-PK inhibitors (for example AZD7648, M3814, CC-122 and CC-115), immune checkpoint therapies (for example PD-1 inhibitors (e.g. pembrolizumab, nivolumab, cemiplimab, and spartalizumab), PD-L1 inhibitors (e.g. atezolizumab, avelumab and durvalumab), or CTLA-4 inhibitord (e.g. ipilimumab)), CHK1 inhibitors (for example V158411, PF-477736 and AZD7762), CHK2 inhibitors (for example CCT241533 and Aminopyridine 7), WEE1 inhibitors (for example adavosertib), platinum-based antineoplastic drugs (for example cisplatin, carboplatin, oxaliplatin, nedaplatin, triplatin tetranitrate, picoplatin, satraplatin and phenanthriplatin, and in particular cisplatin, carboplatin, oxaliplatin, nedaplatin), radionuclide and radiation therapies (for example radium-223 and PSMA-targeting radionuclide therapies (for example 225Ac-Labeled PSMA-617 and 177Lu-Labeled PSMA-617).

As used herein, the term “PI3K Associated” means a gene-region that includes a gene associated with the phosphoinositide 3-kinase (PI3K) signalling pathway. Typically, genes associated with the PI3K signalling pathway are involved in the stimulation of cell proliferation and growth, and the inhibition of cell apoptosis. Examples of such genes include: AKT1, AKT2, AKT3, MET, PIK3C, PIK3CB, PIK3R1 and PTEN. Genetic aberrations in genes associated with the PI3K signalling pathway are known to occur in cancer such as prostate cancer, breast cancer, ovarian cancer, pancreatic cancer and bladder cancer, and in metastatic cancer. If a genetic aberration, such as an asCNA, or a somatic or germline mutation, is detected in one or more gene-region of this type in a subject, it indicates that a subject might benefit from treatment with one or more class of drug that is known or suspected to target the PI3K signalling pathway. Examples of such class of drug include PI3K inhibitors (for example idelalisib, copanlisib, duvelisib, alpelisib, umbralisib, dactolisib, voxtalisib, Taselisib, Idelalisib, Buparlisib, Duvelisib, and Copanlisib and in particular idelalisib, copanlisib, duvelisib, alpelisib, and umbralisib) and mTOR inhibitors (for example rapamycin, deforolimus, dactolisib, voxtalisib, temsirolimus, everolimus, sapanisertib, AZD8055, and AZD2014).

As used herein, the term “Wnt signalling associated” means a gene-region that includes a gene associated with the Wnt signalling pathway. Typically, genes associated with the Wnt signalling pathway are involved in cell fate, cell migration, cell polarity and neural development. Examples of such genes include: APC, CTNNB1 and RNF43. Genetic aberrations in genes associated with the Wnt signalling pathway are known to occur in cancer such as prostate cancer, breast cancer, ovarian cancer, pancreatic cancer and bladder cancer, and in metastatic cancer. If a genetic aberration, such as an asCNA, or a somatic or germline mutation, is detected in one or more gene-region of this type in a subject, it indicates that a subject might benefit from treatment with one or more class of drug that is known or suspected to target the Wnt signalling pathway. Examples of such class of drug include PORCN inhibitors (for example WNT974, ETC-1922159 and CGX1321), FZD antagonists/monoclonal antibodies (for example Vantictumab, Ipafricept, and OTSA101-DTPA-90Y) and Inhibitor of Wnt target genes (for example SM08502).

As used herein, the term “ploidy” refers to the number of copies of each chromosome present in the genome of a cell. Haploid, diploid, triploid, tetraploid, pentaploid and hexaploid are kinds of ploidy. Specifically, the term “haploid” refers to a cell that has one copy of each chromosome. The term “diploid” refers to a cell that has two copies of each chromosome. The term “triploid” refers to a cell that has three copies of each chromosome. The term “tetraploid” refers to a cell that has four copies of each chromosome. The term “pentaploid” refers to a cell that has five copies of each chromosome. The term “hexaploid” refers to a cell that has six copies of each chromosome. A human cell is typically diploid. That is to say that a typical human cell comprises two sets of chromosomes (i.e. 46 chromosomes). However, may types of cancer cells harbour genetic aberrations that result in gain or loss of whole, or parts of, one or more chromosomes. Thus, a human cancer cell cannot be assumed to be diploid. A cell having an abnormal number of chromosomes due to a loss or gain of specific chromosome(s) may be described as aneuploid. A cell having an abnormal number of chromosomes due to a loss or gain of a whole set of chromosome may be described as euploid.

As used herein, the term “Log 2R” refers a function of the percentage of aberrant tumor cells (% AC) that contribute to the copy number alteration and of the copy number in a sample of tumor DNA.

The term “whole exome sequencing” or “WES” refers the nucleotide sequencing of the protein coding regions (i.e. exons) of the genes in the genome.

As used herein, the term “circulating free DNA” (cfDNA) means the DNA fragments that have been released into the blood plasma and are found freely circulating the blood stream, as well as in the urine, released from any cell type in the body. cfDNA is generally double-stranded DNA consisting of small fragments (70 to 200 bp).

As used herein, the term “circulating tumor DNA” (ctDNA) means the DNA fragments that have been released from tumor cells into the blood plasma and are found freely circulating the blood stream, as well as in the urine. ctDNA is generally double-stranded DNA consisting of small fragments (70 bp to 200 bp).

As used herein, the terms “tumor fraction” and “tumor content” or “TC” of a DNA sample refers to the fraction or percentage of DNA molecules derived from tumor cells in the DNA sample compared to the DNA molecules that are not derived from a cancer cell. For example, for a sample comprising cfDNA, the terms “tumor fraction” and “tumor content” of a cfDNA refer to the fraction or percentage of cfDNA molecules derived from tumor cells (i.e. the ctDNA) in a cfDNA sample compared to the cfDNA molecules that are not derived from a cancer cell. cfDNA that is not derived from a cancer cells in a cfDNA sample may be derived from blood cells, for example white blood cells (leukocyte), and other non-cancerous tissues.

As used herein, the term “genomic location” refers to the location of a region of a genome, e.g. the genome of a subject, for example a human. It may be referred to using the coordinates of the start position and end position of the location in a specific chromosome. For a human subject a genomic location is suitably described by reference to a reference genome (for example, a digital nucleic acid sequence database, assembled from a representative example of a species' set of genes). For example, for a human subject, with reference to the human reference genome GRCh37 (also referred to as Human Genome 19 (hg19)) or human reference genome GRCh38 (also referred to as Human Genome 38 (hg38)). For the present inventions, preferably the reference genome is human reference genome GRCh37 (also known as hg19). As such, a genomic location for a human may be described using the coordinates of the start position and end position of the location in a specific chromosome, with reference to the Genome Reference Consortium Human Build 37 (GRCh37) (also referred to as Human Genome 19 (hg19)).

The term “staging” as used herein refers to the process of determining the extent a cancer has developed by growing and/or spreading in a subject. Typically, classification of a cancer involves assigning the cancer a number from I to IV, wherein I is an isolated cancer and IV is a metastatic cancer that has spread to other parts of the body, distal to the primary cancer site.

The term “classification” as used herein refers to the process of classifying the type of cancer in a subject by determining the type of tissue in a subject from which a cancer originates and/or by determining the primary site in the body wherein the cancer first developed. For example, a cancer classified based on the type of tissue in the subject from which a cancer originates may be classified as a carcinoma (for example a colon, prostate or bladder carcinoma), sarcoma, myeloma, leukemia, lymphoma or mixed type (for example, adenosquamous carcinoma, mixed mesodermal tumor, carcinosarcoma, teratocarcinoma).

The term “screening” as used herein refers to the process of checking for a cancer in a subject not known to have cancer. For example, checking for a copy number change or aberration within gene-regions commonly associated with a particular cancer type.

The term “prognostication” as used herein refers to the process of estimating/predicting the likely course and outcome of a cancer, and/or chance that a subject has of recovering from a cancer. For example, a subject whose cancer is not regressing in response to certain cancer treatment(s), as determined, for example by using a method of the present invention, may be considered to have a poor prognosis.

The term “stratification” as used herein refers to the process of stratifying a subject into a molecular group based on the molecular profile the subject's cancer and the predicted response of the subject to a certain treatment. For example stratifying by DNA repair gene associated alteration, such as BRCA1/2 alteration, for predicted response to treatment with a PARP inhibitor or other drugs targeting DNA repair.

As used herein, a “subject” refers to an animal, including mammals such as humans. Preferably, the subject is a human subject. As used herein, an “individual” can be a subject. As used herein, a “patient” refers to a human subject. In one embodiment, the subject is known or suspected to have a cancer (for example prostate cancer), and/or is known or suspected to have a risk of developing cancer (for example prostate cancer), or is known to have cancer and is known or suspected to have metastatic cancer (for example metastatic prostate cancer) or to have a risk of developing metastatic cancer (for example metastatic prostate cancer). In some embodiments, the subject is a subject who has been identified as being at risk of developing a cancer, in particular at risk of developing a prostate cancer. A “subject” may be male or female. In certain embodiments, the subject is male (for example wherein the subject is known or suspected to have/have a risk of developing prostate cancer).

As used herein, a “healthy subject” or “healthy volunteer” refers to a subject that has not been diagnosed with a type of cancer (for example prostate cancer), and preferably has not been diagnosed with any type of cancer. Thus, for example, a method of relating to prostate cancer, a “healthy subject” or “healthy volunteer” has no prostate cancer, and preferably no other type of cancer. Preferably, a healthy subject has not been diagnosed with a type of cancer (for example prostate cancer), and is not suspected of having a type of cancer, and suitably has not been diagnosed with any type of cancer (for example prostate cancer), and is not suspected of having any type of cancer.

As used herein, the term “nucleic acid” means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, and fragments thereof. Reference to a polynucleotide(s) is to be similarly understood.

As used herein, the term “oligonucleotide(s)” refers to nucleic acid molecules that usually comprise between 5 and 100 contiguous bases, for example between 5-10, 5-20, 10-20, 10-50, 15-50,15-100, 20-50, or 20-100 contiguous bases. An oligonucleotide may be capable of hybridising to a target of interest, e.g., a sequence that include an iSNP of the present invention (e.g. an iSNP as defined in Table 1 or Table 2). An oligonucleotide for hybridising to a target may comprise at least 5, least 10, at least 15, at least 20, at least 30, at least 40, at least 50 or at least 60 nucleotides. An oligonucleotide can be used as a primer, a probe, included in a microarray, or used in polynucleotide-based identification methods. For example, the oligonucleotide may be a probe that is complementary to, and capable of hybridizes to, a nucleotide sequence of interest. For example, the probe may be capable of hybridizing to a SNP site present in tumor DNA and/or non-tumor DNA obtained from a subject (e.g. capable of hybridizing to a SNP site defined in Table 1 or Table 2).

As used herein, a “subtype of a cancer” (for example a “subtype of prostate cancer”) is a subset of a type of cancer based on characteristics of the cancer cells, and in particular molecular and genetic characteristics of the cells. Different cancer subtypes can have different disease progression and can respond or not respond to different treatments. The subtype of a cancer is, for example, used to assist in planning treatment and determine prognosis of the patient having that cancer subtype. Examples of a subtypes of prostate cancer include hormone sensitive prostate cancer (HSPC) and castration resistant prostate cancer (CRPC).

### DETAILED DESCRIPTION OF THE INVENTION

The present invention provides an in vitro method for staging, classification, screening monitoring, stratification, selecting treatment for, ascertaining whether treatment is working in, and/or prognostication of cancer in a subject.

The in vitro method of the present invention comprises steps i) to v), as described in further detail below:

### Step i):

The in vitro method of the invention comprises a step of providing a biological sample obtained from the subject comprising tumor DNA. The biological sample comprising tumor DNA may further comprises non-tumor DNA (for example, non-tumor DNA in a plasma sample or a tissue sample). That is to say, that the biological sample may contain DNA derived from a cancer cell in a subject and DNA derived from a non-cancerous (i.e. healthy) cell in the subject. If the biological sample comprises both tumor DNA and non-tumor DNA, the same sample can be provided as the sample comprising tumor DNA and the biological sample comprising non-tumor DNA.

Typically, the biological sample is a blood sample (for example a blood sample, plasma sample or a white blood cell sample), urine sample, saliva sample, tissue sample, or cerebral spinal fluid obtained from the subject.

In certain embodiments, the biological sample comprising tumor DNA and the biological sample comprising non-tumor DNA are separate samples. In certain embodiments, the biological sample comprising tumor DNA and the biological sample comprising non-tumor DNA are the same sample, i.e. a sample comprising a mixture of tumor DNA and non-tumor DNA. In certain embodiments, the biological sample comprising non-tumor DNA may be a tissue sample from a healthy, non-cancerous organ or tissue within the subject, a saliva sample, a blood sample, or white blood cell sample from a subject. Typically, the biological sample comprising non-tumor DNA is a saliva sample, white blood cell sample, or blood sample from a subject. A white blood cell sample may be obtained from a blood sample from a subject, and in particular obtained from the buffy coat separated from a blood sample from a subject. The buffy coat may be separated by centrifugation of the blood sample.

In certain embodiments the biological sample comprising tumor DNA may be a tissue sample from a tumor within the subject, or it may be a plasma sample comprising ctDNA, or it may be a blood sample from a subject. Typically, the biological sample comprising non-tumor DNA is a plasma sample comprising ctDNA, or a blood sample from a subject. A plasma sample comprising ctDNA may be obtained from a blood sample from a subject. The plasma sample comprising ctDNA may be obtained by separating the plasma from the buffy coat in the blood sample, for example by centrifugation of the blood sample.

In certain other embodiments, the biological sample comprising tumor DNA and the biological sample comprising non-tumor DNA are the same sample. For example, the biological sample provided in step i) is a blood sample (for example a plasma sample), urine sample, tissue sample, or cerebral spinal fluid sample comprising both tumor DNA derived from a cancer cell in the subject and non-tumor DNA derived from a non-cancerous cell in the subject. Typically, the tumor DNA present in a blood sample (for example, a plasma sample) or urine sample is circulating tumor DNA (ctDNA).

In embodiments wherein the biological sample comprising tumor DNA and the biological sample comprising non-tumor DNA are the same sample, and the sample is a blood sample, the sample may be a single blood sample that can be separated into a white blood cell sample (for example obtained from the buffy coat separated from the blood sample) to provide the sample comprising non-tumor DNA, and a plasma sample (for example obtained by separating the plasma from a blood sample) to provide the sample comprising non-tumor DNA. Alternatively, or additionally, the blood sample may be a single plasma sample comprising both tumor and non-tumor DNA. For example, the blood sample may be a single plasma sample comprising ctDNA and non-tumor cfDNA.

In certain preferred embodiments, the biological sample comprising tumor DNA is a blood sample comprising circulating tumor DNA (ctDNA). The present inventors have found that the method of the invention is especially effective when using a plasma sample comprising ctDNA obtained from a subject. Thus, preferably, the biological sample comprising tumor DNA is a plasma sample comprising ctDNA. More preferably, the blood sample is a plasma sample that comprises, in addition to the ctDNA, non-tumor cell-free DNA (cfDNA). In certain embodiments, the biological sample comprising tumor DNA is a blood sample, and the biological sample comprising non-tumor DNA is the same blood sample, and the tumor DNA in the sample is ctDNA present in the plasma of the blood sample, and the non-tumor DNA in the sample is genomic DNA from white blood cells present in the blood sample.

Typically, the biological sample comprises a plurality of DNA molecules. For example, at least 10,000, at least 50,000, at least 100,000, at least 500,000, at least 1,000,000 (10⁶), at least 5,000,000 (5×10⁶), at least 10,000,000 (10⁷), at least 100,000,000 (10⁸), at least 1,000,000,000 (10⁹), 5,000,000,000 (5×10⁹), at least 10,000,000,000 (10¹⁰) or at least 15,000,000,000 (1.5×10¹⁰) DNA molecules. Preferably, the biological sample comprises, at least 100,000, at least 500,000, at least 1,000,000 (10⁶), at least 5,000,000 (5×10⁶), at least 10,000,000 (10⁷), at least 100,000,000 (10⁸), at least 1,000,000,000 (10⁹), 5,000,000,000 (5×10⁹), at least 10,000,000,000 (10¹⁰) or at least 15,000,000,000 (1.5×10¹⁰) DNA molecules. More preferably, at least 10,000,000 (10⁷), at least 100,000,000 (10⁸), at least 1,000,000,000 (10⁹) DNA molecules, 5,000,000,000 (5×10⁹), at least 10,000,000,000 (1×10¹⁰) or at least 15,000,000,000 (1.5×10¹⁰).

The quantity of DNA molecules in a sample that are tumor DNA molecules is referred to herein as the percentage tumor content (TC) of a sample. That is to say, that TC refers to the percentage of DNA molecules in a sample that are tumor DNA molecules.

Suitably, the biological sample comprising tumor DNA provided in step i) has a tumor content of at least about 1%, at least about 2%, at least about 3%, at least about 4%, or at least about 5%. For example, the biological sample comprising tumor DNA may have a tumor content of at least about 2%, at least about 3%, at least about 4%, or at least about 5%, at least 7%, at least 10%, at least 15%, at least 20%, at least 30%, at least 40%, at least 50%, at least 50%, at least 60%, at least 70%, at least 80%, at least 90%, or 100%. Preferably, the biological sample comprising tumor DNA provided in step i) has a tumor content of at least about 2%, at least 5%, at least 10%, or at least 15%.

The present inventors have found that the present method is surprising sensitive even when the tumor content of the biological sample is low (for example, about 1% to about 5% tumor content). Thus, in certain embodiments, the biological sample provided in step i) has a tumor content of about 1% to about 5%, for example about 2% to about 5%. In certain embodiments, the biological sample provided in step i) has a tumor content of about 1% to about 15%, for example about 2% to about 15%, or about 5% to about 15%.

In embodiments wherein the biological sample comprising tumor DNA is a tissue sample, the tumor DNA must be extracted from the cells present in the tissue sample. The tumor DNA can be isolated from the biological sample using a variety of techniques known in the art.

In embodiments wherein the biological sample comprising non-tumor DNA is a tissue sample, saliva sample, or white blood cell sample, the non-tumor DNA must be extracted from the cells present in the sample. The non-tumor DNA can be isolated from the biological sample using a variety of techniques known in the art.

In certain embodiments, the tumor DNA and/or non-tumor DNA, is amplified before analysis. Amplification techniques are known to those of ordinary skill in the art and include, but are not limited to, cloning, polymerase chain reaction (PCR), polymerase chain reaction of specific alleles (PASA), polymerase chain ligation, nested polymerase chain reaction, and so forth. The preferred amplification technical for use in the present invention is PCR.

### Step ii):

The method of the present invention comprises a step of detecting the presence of single nucleotide polymorphisms (SNPs) in the non-tumor DNA at:
at least 5% of the SNP loci defined in Table 1 for each of at least 5 target gene-regions defined in Table 1,
and optionally at least 5% of the SNP loci defined in Table 2 for each of at least 3 control gene-regions defined in Table 2;

Tables 1 and 2 are provided below. The genomic locations recited in Tables 1 and 2 are locations with reference to the human reference genome GRCh37 (also known as hg19). The SNP loci defined in Tables 1 and 2 are collectively referred to herein as the “Example 1 SNP panel”.

TABLE 1
target gene-regions and SNPs.

| gene-region | |
|---|---|
| ref/gene- | |
| region loci^{†}/ | |
| number of | |
| SNPs | SNPs (rsids) |
| ARID1A | rs4659442, rs4659443, rs4659444, rs36121084, rs6669111, rs3122646, |
| 1:27022524- | rs11806427, rs11806829, rs12086684, rs6662955, rs1975798, |
| 27108595 | rs6673072, rs4970498, rs12045800, rs74061187, rs11247961, |
| 90 SNPs | rs4970514, rs12046497, rs12094989, rs4970466, rs61692526, |
| | rs12093376, rs4402166, rs7539320, rs12046614, rs11589734, rs4970483, |
| | rs737465, rs1002488, rs6685701, rs1466286, rs4970486, rs12723936, |
| | rs17162190, rs2278978, rs4443935, rs2278979, rs7543005, rs4970489, |
| | rs2290589, rs873151, rs897641, rs3790644, rs3790645, rs34062688, |
| | rs444482, rs3127013, rs389548, rs392814, rs396997, rs9438508, |
| | rs369385, rs383913, rs364977, rs282175, rs282176, rs282177, |
| | rs190737, rs282178, rs406985, rs4970501, rs6598857, rs4970503, |
| | rs566421, rs6598858, rs4970476, rs4970510, rs6666156, rs56304095, |
| | rs12408288, rs17162257, rs72879054, rs12410305, rs76908673, |
| | rs141439288, rs10902643, rs6598860, rs56163802, rs4589135, |
| | rs11247593, rs11247594, rs72879080, rs2273015, rs56147926, |
| | rs11247599, rs113802958, rs10902645, rs7518079, rs115121583, |
| | rs3010106. |
| HSD3B1 | rs838518, rs1935248, rs587728908, rs587606382, rs57393186, |
| 1:120049821- | rs6686552, rs3753264, rs3753263, rs3753262, rs7513419, rs10923804, |
| 120057681 | rs2224995, rs2298028, rs6428816, rs6667679, rs6670497, rs1417604, |
| 217 SNPs | rs10923805, rs10802092, rs3765949, rs10923807, rs12035411, |
| | rs12740687, rs2005390, rs879334, rs879333, rs12038498, rs4659169, |
| | rs4659170, rs4659171, rs6658331, rs12038241, rs1856887, rs12409531, |
| | rs1341018, rs12735092, rs4659175, rs2793132, rs2854962, rs2793133, |
| | rs2854963, rs2854964, rs947129, rs12752713, rs10923821, rs2050891, |
| | rs4659176, rs12722964, rs2362809, rs6670499, rs10923826, rs1341011, |
| | rs7536326, rs7528420, rs2885611, rs2000105, rs2000106, rs6665724, |
| | rs12741083, rs7554698, rs1986371, rs6672903, rs1856884, rs1856883, |
| | rs9651075, rs11584793, rs2096063, rs12748734, rs12755511, |
| | rs12736827, rs10802097, rs10802098, rs2275609, rs7515792, rs7547046, |
| | rs2362810, rs3949342, rs947130, rs10802100, rs2064901, rs12139993, |
| | rs911241, rs2011178, rs2011191, rs10754397, rs1856888, rs10737751, |
| | rs4579805, rs4478857, rs4275492, rs4659188, rs4659189, rs139626219, |
| | rs10923848, rs10754404, rs10802108, rs1885001, rs951693, rs2362815, |
| | rs10754405, rs6657934, rs3737653, rs3737652, rs10754406, rs4659009, |
| | rs4659192, rs2224397, rs2208379, rs10802109, rs6681580, rs5012379, |
| | rs2224398, rs10802110, rs9887874, rs9887882, rs9887805, rs2208382, |
| | rs6668680, rs6668763, rs971058, rs2009240, rs10737754, rs1987612, |
| | rs4659194, rs2224399, rs10923850, rs10923851, rs10923852, |
| | rs10923853, rs13374289, rs2362816, rs2362817, rs2362818, rs2362819, |
| | rs2362820, rs2362821, rs10754408, rs6684302, rs2885613, rs2885614, |
| | rs5009592, rs6663497, rs6697100, rs434523, rs2008921, rs4659202, |
| | rs4659203, rs4659204, rs4659205, rs12745167, rs12727809, rs12746441, |
| | rs375796313, rs370231537, rs374122525, rs376570075, rs189906372, |
| | rs71519736, rs10802113, rs12030462, rs2885796, rs1812822, rs4426020, |
| | rs6680423, rs34827869, rs6704100, rs10923866, rs7551417, rs7537698, |
| | rs910766, rs6428834, rs10802117, rs3862259, rs346669, rs173207, |
| | rs346676, rs346678, rs346680, rs346682, rs1830535, rs1923025, |
| | rs12045785, rs7519907, rs28736838, rs10923875, rs10802118, |
| | rs12023417, rs3862260, rs3862261, rs3850870, rs4659219, rs7531516, |
| | rs838985, rs10754417, rs10802129, rs403218, rs347908, rs10923882, |
| | rs380155, rs481357, rs493427, rs539799, rs598100, rs686362, |
| | rs4659226, rs620741, rs1406917, rs573763, rs646124, rs10802130, |
| | rs6428840, rs10802132, rs541763, rs839613, rs10802133, rs839615, |
| | rs637868. |
| MDM4 | rs11583766, rs4586035, rs4558047, rs4433462, rs4314945, rs4313452, |
| 1:204485511- | rs11582393, rs11582394, rs11585014, rs4575145, rs4523575, rs4437929, |
| 204542871 | rs4245729, rs4245730, rs17333933, rs6703598, rs4304647, rs4077532, |
| 251 SNPs | rs4077533, rs4077534, rs12023102, rs883950, rs4951364, rs4245732, |
| | rs11240724, rs4951365, rs4951068, rs11240726, rs11240727, rs3014614, |
| | rs3125168, rs10900583, rs1996002, rs2942147, rs938128, rs938127, |
| | rs1980052, rs11240732, rs1980051, rs2369094, rs2055939, rs10736846, |
| | rs11240735, rs2942145, rs3014623, rs12068876, rs9661807, rs16853666, |
| | rs3014629, rs2292460, rs3014633, rs374117044, rs3122288, rs3106366, |
| | rs2271427, rs2999492, rs3014637, rs2942127, rs2942130, rs11240746, |
| | rs2055938, rs2942143, rs3014599, rs2999484, rs3014602, rs2271415, |
| | rs12092943, rs6692377, rs6594014, rs7519417, rs10900590, rs4951382, |
| | rs4951383, rs4951384, rs12402641, rs4951385, rs4951386, rs12117054, |
| | rs7556371, rs10494852, rs1398148, rs11240750, rs10900592, rs6594015, |
| | rs11240751, rs4951075, rs16853865, rs10900593, rs10900594, |
| | rs4951389, rs11240754, rs11240755, rs12032733, rs4951077, rs1380576, |
| | rs12136299, rs4951393, rs4951078, rs12043196, rs7556655, rs4252685, |
| | rs4252686, rs4252687, rs2045623, rs4252694, rs10900595, rs4252717, |
| | rs4252718, rs4252725, rs2369244, rs2290855, rs2290854, rs1563828, |
| | rs4252734, rs1460036, rs12741351, rs16853958, rs11240758, |
| | rs11801289, rs12125533, rs4951398, rs4951080, rs6681905, rs6679336, |
| | rs4951402, rs6594016, rs10900599, rs7541589, rs12039454, rs885012, |
| | rs11240760, rs35270244, rs10793765, rs10793766, rs10793767, |
| | rs5010868, rs12139477, rs12130686, rs12138846, rs12133735, |
| | rs12566957, rs4951407, rs4951408, rs10900600, rs6679717, rs4951409, |
| | rs7532236, rs2169136, rs4328119, rs2887429, rs6682208, rs11240762, |
| | rs6594018, rs6594019, rs11240764, rs12143943, rs12567161, rs7541449, |
| | rs1563827, rs868097, rs3795567, rs12027855, rs61817485, rs17335392, |
| | rs7551222, rs7531829, rs7553683, rs7553896, rs7546057, rs875094, |
| | rs875093, rs7515096, rs7542085, rs7541751, rs10751425, rs10900417, |
| | rs915239, rs10793713, rs58651344, rs11240232, rs12024827, rs4951085, |
| | rs4951087, rs4950954, rs4951088, rs4951089, rs12038573, rs11240234, |
| | rs11240235, rs11240236, rs11240237, rs11240238, rs11240239, |
| | rs4279904, rs10900418, rs1411108, rs1329631, rs11240241, rs10900419, |
| | rs4951090, rs4951091, rs4951092, rs12136166, rs12125207, rs12127178, |
| | rs61170670, rs2492986, rs2815831, rs2815830, rs12125624, rs12124793, |
| | rs11240242, rs11240243, rs4143141, rs4143140, rs11240244, rs6663910, |
| | rs6676478, rs1556156, rs6667578, rs2149819, rs1610399, rs2815828, |
| | rs2772235, rs10793714, rs2815827, rs2782525, rs2815826, rs2772241, |
| | rs2772242, rs59053733, rs10793715, rs10751426, rs11240246, |
| | rs11240249, rs1999155, rs11240251, rs4951095, rs12240222, |
| | rs12240223, rs6696549, rs6696552, rs16854288, rs4111548, rs1329634, |
| | rs12085502, rs56342584, rs6593899, rs2012130, rs11240255, rs7553744. |
| AKT3 | rs12074660, rs12079656, rs12091558, rs3943093, rs2490395, rs2484639, |
| 1:243651535- | rs2484640, rs10926985, rs2636321, rs12084733, rs12085949, rs2490391, |
| 244014381 | rs2932634, rs953492, rs10926987, rs12061451, rs11809042, rs6429422, |
| 268 SNPs | rs2783971, rs2783972, rs2636323, rs68192516, rs2451668, rs10158537, |
| | rs2970546, rs10926991, rs2636324, rs2802726, rs2802725, rs2802723, |
| | rs2802722, rs2783968, rs2783967, rs2783966, rs2783965, rs2783964, |
| | rs2636319, rs2636320, rs10926995, rs4658558, rs6695785, rs10803140, |
| | rs12129652, rs57152483, rs2039839, rs6703335, rs10927025, |
| | rs12748751, rs1008173, rs12751210, rs2994329, rs3006923, rs884808, |
| | rs12093576, rs3006933, rs9428576, rs1058305, rs1058304, rs1538773, |
| | rs13376709, rs3930294, rs10927035, rs2291410, rs2220276, rs1352162, |
| | rs12144559, rs10927041, rs2291409, rs6681024, rs2085555, rs12143602, |
| | rs12027563, rs1074657, rs12136847, rs12735854, rs2125233, |
| | rs55778834, rs2125232, rs2345284, rs2125231, rs10927044, rs3856231, |
| | rs61833201, rs10927045, rs6671475, rs12739344, rs6660534, |
| | rs11585986, rs1486472, rs10754807, rs73124169, rs10927052, |
| | rs7547861, rs12691547, rs7517732, rs10927056, rs12117580, rs4658403, |
| | rs320320, rs4658588, rs10927059, rs6686591, rs7517921, rs1973284, |
| | rs6429433, rs1486475, rs320339, rs10927060, rs2034915, rs2125230, |
| | rs1458023, rs10927062, rs9782883, rs9782958, rs4553169, rs4658590, |
| | rs1379700, rs11586029, rs4565696, rs2881275, rs971285, rs6674314, |
| | rs71537335, rs10803158, rs10927067, rs57824597, rs1458024, |
| | rs1531244, rs4132509, rs4610997, rs73128287, rs12142652, rs9287269, |
| | rs10927075, rs9428975, rs12116882, rs7534117, rs7514984, rs34477525, |
| | rs7523822, rs7552659, rs7552820, rs10803161, rs4518884, rs12049228, |
| | rs6659180, rs12045787, rs4430311, rs12753750, rs11579649, rs3008657, |
| | rs2953328, rs3008658, rs3008659, rs2953330, rs2953331, rs3008660, |
| | rs2953327, rs10927084, rs4497192, rs7545692, rs146509887, |
| | rs12134862, rs12083813, rs4412578, rs1516682, rs10737889, rs1914235, |
| | rs4658597, rs4658598, rs10737890, rs6680504, rs12128559, rs10754812, |
| | rs10754813, rs12090270, rs10803166, rs4658599, rs4658600, rs4658601, |
| | rs10927086, rs6670700, rs10927087, rs10803167, rs10803168, |
| | rs4658405, rs4658406, rs950398, rs950399, rs7517940, rs2048864, |
| | rs10927088, rs2206, rs11584607, rs10927090, rs4658604, rs10927091, |
| | rs6429441, rs1568103, rs496927, rs525234, rs559080, rs1223872, |
| | rs482261, rs579388, rs543626, rs544739, rs990794, rs472276, rs500202, |
| | rs495230, rs476426, rs511659, rs495843, rs578085, rs1223870, |
| | rs479829, rs2454224, rs546296, rs6668711, rs693795, rs58305168, |
| | rs479338, rs536441, rs576682, rs563775, rs563904, rs10927097, |
| | rs1711347, rs1798128, rs1690633, rs520971, rs500337, rs500341, |
| | rs523878, rs551361, rs4596868, rs4537536, rs692981, rs505173, |
| | rs6673663, rs6683057, rs512825, rs563745, rs12122824, rs10927101, |
| | rs476141, rs486918, rs10803173, rs551296, rs6429445, rs580980, |
| | rs693150, rs753496, rs12131048, rs2014888, rs2486535, rs1874782, |
| | rs2047137, rs2500489, rs2039789, rs2500487, rs67854820, rs6429446, |
| | rs2131814, rs2220817, rs10927115, rs12027747. |
| PTEN | rs7913974, rs7914120, rs7914810, rs11202458, rs6586081, rs10749553, |
| 10:89622870- | rs10887729, rs10749555, rs4934348, rs4934349, rs4934350, rs4933448, |
| 89731687 | rs12265819, rs7071621, rs10887730, rs12260150, rs7093316, |
| 581 SNPs | rs12252261, rs11202469, rs10887731, rs11202470, rs10887732, |
| | rs12412058, rs12413290, rs11202472, rs61358809, rs10887733, |
| | rs10788556, rs10887734, rs11202473, rs11202474, rs7895494, |
| | rs1324295, rs1324296, rs1324298, rs11202476, rs7893460, rs4934352, |
| | rs4933449, rs11202483, rs2104347, rs1924500, rs1924501, rs1924503, |
| | rs10788558, rs7921697, rs1324299, rs7099810, rs7079400, rs7911924, |
| | rs35382373, rs11592638, rs4933451, rs10887737, rs7913758, rs7070474, |
| | rs7087128, rs1815300, rs1320449, rs2025457, rs791873, rs791874, |
| | rs10437508, rs791881, rs2755434, rs791883, rs791884, rs791885, |
| | rs7092063, rs791888, rs791889, rs11202491, rs1408378, rs2762519, |
| | rs2755432, rs2762520, rs2762521, rs1969821, rs1969822, rs1969823, |
| | rs2762522, rs2755430, rs2038680, rs2147288, rs1609577, rs1609578, |
| | rs1609579, rs2755429, rs2762523, rs2762524, rs2755428, rs3758475, |
| | rs2762527, rs2755425, rs2281803, rs10788559, rs2255683, rs2302402, |
| | rs7078594, rs7904314, rs11202496, rs1980647, rs4934355, rs4244979, |
| | rs1358863, rs1358864, rs7071248, rs12761361, rs7088341, rs6586092, |
| | rs7085195, rs7097212, rs9633742, rs1358865, rs11202499, rs7903568, |
| | rs2077695, rs2077773, rs3824722, rs371639270, rs12413510, |
| | rs12257469, rs11202501, rs11202502, rs4934357, rs4934358, |
| | rs10509410, rs11202503, rs7078618, rs7903516, rs12772633, |
| | rs10887741, rs12412482, rs12781358, rs7908056, rs1535369, rs7072852, |
| | rs35628516, rs12265003, rs6586094, rs6586095, rs1407249, rs12253888, |
| | rs3824721, rs72811966, rs7082207, rs941828, rs941829, rs941830, |
| | rs55720312, rs4934359, rs4933453, rs4933454, rs4934360, rs7097366, |
| | rs12267729, rs12267849, rs10887742, rs4934361, rs4934362, rs4933456, |
| | rs4933457, rs7096803, rs7084129, rs3781191, rs6586097, rs6586098, |
| | rs7923162, rs10887743, rs4933458, rs189808294, rs61855160, |
| | rs11202522, rs7919607, rs7907791, rs7908313, rs7898500, rs3815417, |
| | rs2302406, rs2302407, rs10788564, rs3781190, rs3781189, rs3781188, |
| | rs7896505, rs3781187, rs11202524, rs11202525, rs11202526, rs7079187, |
| | rs7097745, rs7080768, rs7068030, rs7081055, rs11202527, rs12414519, |
| | rs12414218, rs10887745, rs10887746, rs55911146, rs11813070, |
| | rs4933459, rs4933460, rs4934363, rs4934364, rs4934365, rs4934366, |
| | rs4934367, rs10887748, rs11594034, rs1853168, rs7088594, rs7080206, |
| | rs7921017, rs72811988, rs10887749, rs10887750, rs10887751, |
| | rs12256533, rs7073722, rs7073724, rs6586101, rs7920554, rs12762914, |
| | rs35814940, rs1321934, rs7901767, rs35388513, rs11202531, |
| | rs12571331, rs12780234, rs7920778, rs7904344, rs7921889, rs7905932, |
| | rs7090432, rs7902841, rs7913763, rs11202536, rs12776656, rs12782128, |
| | rs3740278, rs1051441, rs7908203, rs12765184, rs11202538, rs12775041, |
| | rs66704193, rs66863455, rs34466534, rs2057358, rs34878240, |
| | rs10082347, rs35254974, rs12413371, rs11818430, rs1923260, |
| | rs1923261, rs11812787, rs10887752, rs12782235, rs1819163, rs1923262, |
| | rs9663978, rs4934369, rs2038554, rs9651491, rs12570639, rs12771188, |
| | rs12573160, rs7090088, rs7068497, rs12770684, rs11202553, |
| | rs12773218, rs1993845, rs7098653, rs7910692, rs11202557, rs6586102, |
| | rs11817759, rs11816798, rs12782454, rs12765681, rs12771844, |
| | rs11498630, rs10887753, rs11202563, rs12570131, rs7085412, |
| | rs12772552, rs1960047, rs17096702, rs12775017, rs12769771, |
| | rs12781171, rs7898915, rs11202564, rs11202565, rs72814123, |
| | rs12780237, rs2067661, rs12779667, rs4443987, rs12779915, rs2306157, |
| | rs12762731, rs3816651, rs1871055, rs11202575, rs7900225, rs6586103, |
| | rs11202576, rs7897862, rs17702687, rs1903862, rs72814134, rs1903861, |
| | rs10788567, rs11202579, rs11202580, rs11202581, rs7078010, |
| | rs10887756, rs10509412, rs7076964, rs10887758, rs10887760, |
| | rs11202584, rs11202585, rs1234212, rs7919108, rs11202586, |
| | rs58599149, rs1234221, rs11202589, rs1903858, rs1234225, rs1234224, |
| | rs1236816, rs1234213, rs1234214, rs2735343, rs2248293, rs532678, |
| | rs478839, rs10887768, rs10887769, rs10430641, rs11202615, |
| | rs10788572, rs11202622, rs71477180, rs12416244, rs10887774, |
| | rs2785071, rs662115, rs2673829, rs2785070, rs2785078, rs2244092, |
| | rs2673825, rs2673824, rs1360950, rs2735350, rs719426, rs12412656, |
| | rs1414892, rs10788577, rs1414893, rs1414894, rs10887776, rs4933464, |
| | rs11202633, rs2049814, rs12414916, rs12413500, rs72816127, |
| | rs7901647, rs7916888, rs7908382, rs6586115, rs7068968, rs10788578, |
| | rs7910126, rs2248456, rs2673822, rs2673821, rs2673820, rs2673819, |
| | rs2673818, rs11202634, rs11202635, rs2673817, rs2673815, rs760177, |
| | rs2673814, rs7907255, rs2735352, rs2673813, rs624597, rs7094771, |
| | rs2673826, rs12240758, rs10887777, rs563675, rs588307, rs812629, |
| | rs2471891, rs479727, rs559783, rs534182, rs10159827, rs677422, |
| | rs1537876, rs1122686, rs2785066, rs4934376, rs2785067, rs2785068, |
| | rs494437, rs618024, rs489027, rs2785069, rs644260, rs567061, |
| | rs545138, rs2975113, rs4344399, rs7084813, rs11202642, rs945559, |
| | rs4934377, rs6586116, rs2311740, rs57633183, rs10788580, rs12247999, |
| | rs7079271, rs11202645, rs1889918, rs7085473, rs4244980, rs1855969, |
| | rs12259196, rs7914055, rs9630112, rs4933466, rs2184697, rs2184698, |
| | rs10887778, rs1981150, rs10788581, rs7070956, rs7070980, rs1013594, |
| | rs10749563, rs10887779, rs7914010, rs10788582, rs12416046, |
| | rs11202655, rs7901779, rs6586119, rs6586120, rs10887780, rs10159513, |
| | rs10160109, rs10887782, rs10159903, rs74146654, rs11202656, |
| | rs17108932, rs1360952, rs10887783, rs10160194, rs10749564, |
| | rs4934378, rs10788583, rs12411862, rs1932532, rs1932531, rs1932530, |
| | rs1577661, rs59204786, rs4934379, rs7899035, rs60034915, rs56375327, |
| | rs4147056, rs17322521, rs7079754, rs2225796, rs17322612, rs7905605, |
| | rs72816194, rs35718624, rs7079675, rs12573738, rs4933470, rs4933471, |
| | rs1109173, rs1109174, rs7069053, rs945562, rs1537875, rs945563, |
| | rs10749565, rs9325599, rs11202661, rs4934380, rs10887787, |
| | rs10887788, rs7072702, rs11202662, rs10887789, rs7077272, rs7077409, |
| | rs10444136, rs7358173, rs78824448, rs10509536, rs7096303, rs7080878, |
| | rs10887790, rs10788585, rs10887791, rs7082474, rs12572780, |
| | rs7096356, rs7082696, rs9787705, rs11202669, rs1929318, rs1943988, |
| | rs756244, rs4934381, rs475618, rs510559, rs1147648, rs535541, |
| | rs567645, rs508901, rs545874, rs533722, rs537546, rs645516, rs629959, |
| | rs485396, rs1943987, rs543338, rs551265, rs647730, rs7924020, |
| | rs7901103, rs10788587, rs11202683, rs1556382, rs4934383, rs4934384, |
| | rs3899355, rs11202684, rs2039305, rs472678, rs497099, rs10887793, |
| | rs7906812, rs1888641, rs2871984, rs2026914, rs7095034, rs10509539, |
| | rs477311, rs55983186, rs11202691, rs670472, rs11816865, rs955033, |
| | rs606424, rs286482, rs286493. |
| FANCF | rs340960, rs340957, rs2671124, rs2593672, rs2593673, rs2671118, |
| 11:22644079- | rs2671114, rs2665678, rs2593675, rs2665676, rs2671116, rs2665675, |
| 22647387 | rs2671121, rs2665674, rs2671123, rs608466, rs11820613, rs1399096, |
| 235 SNPs | rs1514077, rs1160513, rs57372865, rs11026580, rs11026581, |
| | rs10833759, rs340983, rs10833760, rs12293217, rs10833761, rs1022260, |
| | rs12361939, rs974837, rs1514088, rs4551772, rs1514087, rs35324600, |
| | rs12421463, rs11026605, rs10833764, rs201405460, rs7120717, |
| | rs9299965, rs10766948, rs2403742, rs7932502, rs10833768, rs1606615, |
| | rs1399098, rs10833769, rs10741939, rs10833770, rs55645341, |
| | rs10833772, rs1514084, rs4511278, rs4597070, rs4511279, rs4603304, |
| | rs4603305, rs7483898, rs7938987, rs1514094, rs10766950, rs10766951, |
| | rs16909616, rs1533316, rs4498996, rs2063708, rs11026626, rs11026636, |
| | rs2048668, rs11026637, rs2219862, rs991433, rs12366112, rs2170079, |
| | rs10833779, rs7942740, rs6483869, rs11026657, rs12223349, rs1606619, |
| | rs1567863, rs1567864, rs10833781, rs7936769, rs7950479, rs11026663, |
| | rs12807105, rs7936962, rs7937743, rs7937449, rs7941154, rs10833782, |
| | rs10833783, rs10833784, rs10833785, rs10833786, rs408199, |
| | rs12574330, rs439032, rs10741943, rs10833798, rs380243, rs2030159, |
| | rs2062449, rs2062450, rs1874978, rs10741944, rs421953, rs1993964, |
| | rs1993966, rs2046053, rs10766961, rs1125899, rs1125898, rs1125897, |
| | rs1121196, rs1121197, rs1121198, rs1032681, rs1125485, rs2279240, |
| | rs10741945, rs10741946, rs10734315, rs10734316, rs10734317, |
| | rs10734318, rs10741947, rs4606476, rs4301790, rs4444081, rs4312064, |
| | rs16909908, rs4366484, rs1391976, rs2061930, rs7113084, rs437734, |
| | rs431127, rs443661, rs424500, rs439540, rs439404, rs385907, rs430864, |
| | rs400782, rs404833, rs434643, rs2928345, rs7125750, rs408197, |
| | rs426876, rs2928339, rs2928338, rs1695520, rs7926692, rs2956868, |
| | rs400862, rs380792, rs372669, rs373751, rs598393, rs671057, rs395810, |
| | rs601069, rs612567, rs378431, rs448264, rs395398, rs1499245, |
| | rs337471, rs337473, rs91295, rs337474, rs91293, rs337475, rs382766, |
| | rs58707724, rs337445, rs10833804, rs959736, rs10833805, rs9633816, |
| | rs7929747, rs2270755, rs1566378, rs4923025, rs11026770, rs10766966, |
| | rs67808088, rs12422017, rs7942050, rs12289982, rs11026774, |
| | rs12269991, rs1566381, rs2896652, rs1032682, rs2009228, rs11026783, |
| | rs11026784, rs1499241, rs4307722, rs1499243, rs10833811, rs1499244, |
| | rs10500943, rs10833813, rs12575051, rs11026787, rs7115745, |
| | rs12575129, rs7941772, rs34684546, rs7129006, rs4604881, rs4278513, |
| | rs2058372, rs1990423, rs7101469, rs2058371, rs7941671, rs12789792, |
| | rs12270952, rs12271316, rs7105105, rs2041466, rs7926714, rs4922625, |
| | rs11026795, rs10833817, rs10833818, rs2159668, rs12419860. |
| CCND1 | rs4334037, rs4980628, rs7928827, rs7394746, rs7395513, rs7395791, |
| 11:69455855- | rs12806952, rs7103831, rs7109934, rs4506680, rs61452413, rs7130691, |
| 69469242 | rs7130932, rs4980550, rs10796819, rs1938684, rs10908184, rs4980554, |
| 263 SNPs | rs10752562, rs10737153, rs10737154, rs3740643, rs631695, rs7945917, |
| | rs10796820, rs654829, rs4980661, rs506516, rs4980662, rs540233, |
| | rs596146, rs623110, rs486564, rs659058, rs665172, rs676742, rs647340, |
| | rs3015960, rs2507824, rs592081, rs559638, rs609079, rs478482, |
| | rs666849, rs620817, rs589191, rs501129, rs598828, rs493786, rs676856, |
| | rs665095, rs2015489, rs670170, rs476679, rs602690, rs510754, |
| | rs573073, rs673958, rs495496, rs624894, rs1893721, rs596209, |
| | rs604586, rs631683, rs504214, rs686488, rs659612, rs546597, rs661178, |
| | rs72930549, rs582635, rs1485993, rs634812, rs657432, rs498136, |
| | rs497356, rs694397, rs868089, rs2001780, rs2001671, rs2006905, |
| | rs505724, rs9651783, rs504293, rs565600, rs2046494, rs4354713, |
| | rs640013, rs11263498, rs1960217, rs11263499, rs660963, rs638640, |
| | rs652190, rs11263501, rs4082429, rs565477, rs564650, rs7946918, |
| | rs4980566, rs625237, rs625625, rs668196, rs625697, rs638385, |
| | rs55811218, rs477409, rs638751, rs480921, rs1385873, rs60879439, |
| | rs17318844, rs11263503, rs672488, rs518418, rs6606648, rs6606649, |
| | rs10908192, rs7944992, rs7939467, rs7948678, rs61881633, rs7939944, |
| | rs7928863, rs7949172, rs7940094, rs10908193, rs10908194, rs10796824, |
| | rs10796825, rs7109242, rs7109260, rs7130162, rs7103009, rs7112989, |
| | rs7109237, rs7109338, rs7121742, rs7117072, rs10796826, rs10737155, |
| | rs7113676, rs7118126, rs7121435, rs7939870, rs7122472, rs7118790, |
| | rs7950206, rs7929871, rs7930159, rs11263509, rs7946645, rs7950525, |
| | rs7933440, rs7933453, rs7118649, rs2087016, rs7948546, rs11263511, |
| | rs636800, rs11263513, rs10908196, rs10908197, rs647648, rs618777, |
| | rs1982774, rs594024, rs594025, rs10796827, rs592483, rs611003, |
| | rs653810, rs654240, rs655089, rs667515, rs1683847, rs2450254, |
| | rs2510460, rs1385874, rs2930975, rs2450255, rs1385875, rs2510461, |
| | rs1944129, rs1352075, rs586459, rs647451, rs649392, rs3212891, |
| | rs6606652, rs58439903, rs1201515, rs1192921, rs1192923, rs3737463, |
| | rs1192927, rs1789364, rs882796, rs10796829, rs10796830, rs11263534, |
| | rs12804611, rs6606657, rs67863122, rs58815028, rs10796833, |
| | rs10796834, rs4980684, rs11263541, rs948989, rs7937957, rs7947358, |
| | rs531992112, rs72940385, rs61882843, rs61882844, rs7479018, |
| | rs4551801, rs1944134, rs4320970, rs4462371, rs10908208, rs9735736, |
| | rs10737157, rs9736229, rs12575066, rs11263556, rs35038801, |
| | rs4459333, rs9667226, rs3886167, rs9665935, rs57709936, rs3897660, |
| | rs10908218, rs9667021, rs4246965, rs4980692, rs4121888, rs4121889, |
| | rs10908219, rs10908220, rs10796848, rs10796849, rs55926574, |
| | rs10796850, rs10752568, rs10752569, rs4980571, rs10908224, |
| | rs10908225, rs4980695, rs4980696, rs4980698, rs9667380, rs3897662, |
| | rs4980699, rs9330211, rs9330212, rs4611233, rs4582995, rs4246966, |
| | rs4246968. |
| ATM | rs10890777, rs11212445, rs11212446, rs4414189, rs2062878, |
| 11:108093211- | rs10890779, rs76708922, rs10890780, rs4754279, rs4633427, |
| 108239829 | rs12422151, rs12789994, rs12791284, rs4753823, rs3950985, rs7113897, |
| 504 SNPs | rs963620, rs963619, rs4754281, rs10890784, rs10890785, rs11212459, |
| | rs7120994, rs12576453, rs61906955, rs6588985, rs12224968, |
| | rs61906957, rs10890786, rs6588989, rs4554861, rs11600938, rs2089175, |
| | rs57630641, rs6588990, rs12295681, rs12791422, rs1847565, rs7935612, |
| | rs11212470, rs10890790, rs12285904, rs12574834, rs4753825, |
| | rs4753826, rs4754284, rs7951157, rs12575489, rs724787, rs724788, |
| | rs11212476, rs4385865, rs4028257, rs3950982, rs2355838, rs7107189, |
| | rs4312032, rs4028262, rs11212481, rs12577263, rs4028251, rs10890795, |
| | rs7930830, rs3950984, rs4754286, rs4754287, rs11212488, rs4568970, |
| | rs4568971, rs10789651, rs10890798, rs11212490, rs10890800, |
| | rs7130999, rs11212491, rs7935911, rs7947410, rs12574572, rs2271852, |
| | rs10890802, rs10890803, rs3858395, rs3858396, rs3911815, rs4503496, |
| | rs4508171, rs7120705, rs7104942, rs4575229, rs10890804, rs10890805, |
| | rs7105163, rs7120711, rs10890807, rs68076088, rs10890808, |
| | rs11212508, rs10890809, rs4430476, rs12792042, rs3736509, |
| | rs12808406, rs10890810, rs10890811, rs2056266, rs12360727, |
| | rs12807544, rs4550189, rs1519073, rs1550609, rs10890813, rs7128286, |
| | rs7102368, rs10890814, rs7114794, rs7123836, rs11212515, rs34971817, |
| | rs10890816, rs7931681, rs10890817, rs7926222, rs9666209, rs10789655, |
| | rs10749914, rs12794010, rs11212524, rs10890819, rs10890820, |
| | rs12361905, rs11212525, rs4754297, rs2280332, rs11212527, rs7928764, |
| | rs10890821, rs4754298, rs6589004, rs10789656, rs4754299, rs10890822, |
| | rs11212532, rs6589006, rs12787445, rs12801154, rs920679, rs6589007, |
| | rs7129527, rs11212538, rs4754301, rs1850730, rs4623864, rs11212542, |
| | rs7395528, rs4753833, rs7118967, rs10890825, rs3781869, rs3781868, |
| | rs11212546, rs2083707, rs4754305, rs12272183, rs11605442, rs609557, |
| | rs228606, rs228608, rs183459, rs183460, rs228589, rs625120, rs228590, |
| | rs228591, rs641605, rs228594, rs228595, rs623860, rs228597, rs228598, |
| | rs228599, rs679782, rs600931, rs599406, rs694376, rs599164, rs228592, |
| | rs172927, rs672655, rs627418, rs637064, rs228593, rs651030, rs665293, |
| | rs664677, rs641312, rs620815, rs618499, rs11212579, rs4987982, |
| | rs1003623, rs1003624, rs624366, rs654005, rs592955, rs609261, |
| | rs601111, rs582157, rs647681, rs1150201, rs583725, rs680113, |
| | rs582297, rs645485, rs619972, rs1150203, rs650173, rs650128, |
| | rs611646, rs599558, rs660429, rs673281, rs606275, rs676729, rs608086, |
| | rs620613, rs634268, rs595747, rs662218, rs662578, rs609429, rs186591, |
| | rs609655, rs227060, rs227061, rs227062, rs227063, rs227064, rs227067, |
| | rs227068, rs227069, rs227070, rs227072, rs227073, rs227074, rs172896, |
| | rs227075, rs11212592, rs227076, rs425538, rs425061, rs373759, |
| | rs419716, rs371406, rs396552, rs374443, rs227041, rs227040, rs664982, |
| | rs664143, rs652541, rs227053, rs227094, rs170548, rs652311, rs227090, |
| | rs605394, rs227087, rs227086, rs227078, rs186595, rs573890, rs227077, |
| | rs179110, rs10789659, rs113995, rs597788, rs60683001, rs227055, |
| | rs172894, rs227056, rs186593, rs227058, rs186594, rs172895, |
| | rs7101497, rs7931930, rs11212610, rs7943165, rs9667658, rs7108522, |
| | rs11212614, rs11212615, rs11212616, rs2884030, rs2356801, rs2356802, |
| | rs11212617, rs35244261, rs7946955, rs10890834, rs7127490, rs7112136, |
| | rs80348089, rs75005171, rs76859507, rs10431061, rs11212619, |
| | rs11212620, rs11212621, rs1960006, rs4335507, rs1583598, rs10890839, |
| | rs11212635, rs4753842, rs2118308, rs7131306, rs6589019, rs4754324, |
| | rs7942014, rs12796446, rs7110127, rs17108024, rs12792979, rs5023001, |
| | rs12226046, rs7943063, rs10749917, rs7934719, rs3824987, rs11212661, |
| | rs11212662, rs75542713, rs11212663, rs7126588, rs12419828, |
| | rs11212666, rs3901851, rs3765632, rs12223381, rs10749918, |
| | rs10789665, rs11212672, rs12801988, rs10890848, rs11212676, |
| | rs10890849, rs893279, rs751343, rs2118309, rs10431062, rs12361570, |
| | rs7943820, rs11212689, rs11212691, rs10890851, rs4753844, |
| | rs11824250, rs7118229, rs2846411, rs10789666, rs10890852, |
| | rs10890855, rs4754326, rs3900699, rs11212700, rs2640784, rs2852183, |
| | rs2640786, rs2846414, rs2640773, rs1940211, rs1940212, rs2852189, |
| | rs2016291, rs4278482, rs10890860, rs11212704, rs2852192, rs2640758, |
| | rs2852193, rs2155440, rs2256083, rs2640757, rs2852194, rs2640756, |
| | rs2155441, rs2186783, rs2256362, rs2256372, rs2852195, rs2852196, |
| | rs2640755, rs2846407, rs55645834, rs2640753, rs767664, rs11212711, |
| | rs12277367, rs17693854, rs2852199, rs2640752, rs2640750, rs949284, |
| | rs949285, rs949286, rs2155439, rs2640749, rs1074014, rs1072877, |
| | rs10466558, rs2846416, rs11820265, rs2846417, rs80303440, rs7105022, |
| | rs10890863, rs2086592, rs10890865, rs11212723, rs55732976, |
| | rs34403786, rs2846409, rs12799294, rs12575739, rs1564582, |
| | rs10890866, rs962757, rs11212726, rs1482269, rs12791136, rs985548, |
| | rs2852202, rs10789671, rs2846421, rs2846420, rs2846419, rs2640742, |
| | rs2640744, rs2640745, rs2846418, rs922804, rs7930018, rs900243, |
| | rs11212733, rs4539291, rs2852205, rs6589025, rs7121973, rs7125567, |
| | rs7125479, rs7934388, rs4754331, rs9667002, rs7934561, rs900242, |
| | rs10890869, rs10890870, rs2640751, rs2852206, rs2640759, rs2624129, |
| | rs4754332, rs12363922, rs2852207, rs11606123, rs2640761, rs990253, |
| | rs7952583, rs2624130, rs10789674, rs2624131, rs4754334, rs4754335, |
| | rs2640763, rs12146575, rs878189, rs878190, rs2128243, rs11212737, |
| | rs2640764, rs2624133, rs60010354, rs7935508, rs2624134, rs2624135, |
| | rs2624136, rs4754338, rs2062116, rs10890876, rs2640767, rs2624144, |
| | rs2624145, rs2640768, rs2852186, rs2624146, rs2640781, rs2640778, |
| | rs2615748, rs2640766, rs2640765, rs2615747, rs2615746, rs2126569. |
| ZBTB16 | rs869451, rs2008008, rs61907884, rs11214767, rs10891613, rs1379170, |
| 11:113930315- | rs7104411, rs10891615, rs4938066, rs3894248, rs3894247, rs10789988, |
| 114121398 | rs7118530, rs2085421, rs2045367, rs10891621, rs11214848, rs1456889, |
| 228 SNPs | rs11214855, rs2735211, rs2509220, rs897688, rs2846629, rs2735199, |
| | rs2852793, rs1672718, rs2852800, rs1672712, rs2846630, rs10891629, |
| | rs12290032, rs238929, rs238926, rs238925, rs238924, rs238914, |
| | rs238913, rs238912, rs238911, rs238910, rs1630468, rs238904, |
| | rs238903, rs238899, rs537280, rs238897, rs238896, rs238895, rs238892, |
| | rs238891, rs238890, rs1989278, rs763857, rs2735207, rs238889, |
| | rs238887, rs2735209, rs909167, rs1318331, rs2005664, rs909168, |
| | rs1318332, rs1810382, rs2106234, rs2250721, rs4936291, rs10891630, |
| | rs10891631, rs623944, rs509402, rs595641, rs11214878, rs575654, |
| | rs648116, rs648044, rs636406, rs1184748, rs513039, rs585287, |
| | rs663516, rs648181, rs512113, rs542273, rs2852796, rs2519196, |
| | rs2852795, rs583329, rs112381618, rs544126, rs682155, rs486044, |
| | rs1178075, rs667182, rs641566, rs634575, rs634213, rs633721, |
| | rs546406, rs621388, rs663230, rs620523, rs61904663, rs2846021, |
| | rs2846022, rs619046, rs493327, rs606925, rs678552, rs487690, |
| | rs486898, rs604377, rs591242, rs591210, rs589916, rs566013, rs598161, |
| | rs542292, rs542270, rs683900, rs537847, rs670728, rs629922, rs645349, |
| | rs655121, rs499878, rs624452, rs3782005, rs505736, rs583200, |
| | rs1186196, rs1633539, rs651579, rs552080, rs656269, rs7129772, |
| | rs552007, rs494181, rs672455, rs424337, rs454802, rs375620, rs421535, |
| | rs651748, rs398055, rs573264, rs400920, rs390538, rs378331, rs403104, |
| | rs364729, rs394800, rs370634, rs11214909, rs402311, rs526484, |
| | rs530532, rs670984, rs35728782, rs503768, rs2511153, rs683271, |
| | rs114138581, rs538075, rs560931, rs611048, rs9326242, rs10082585, |
| | rs10891640, rs10891641, rs7105175, rs6589418, rs6589419, rs7930540, |
| | rs67227065, rs10891642, rs10891643, rs6589421, rs6589422, |
| | rs17116793, rs4936293, rs2156782, rs10891644, rs2604294, rs10400393, |
| | rs2852427, rs2244175, rs11214934, rs2847492, rs2852432, rs67174547, |
| | rs2256292, rs1941404, rs2852426, rs2847476, rs949373, rs7940850, |
| | rs11214945, rs10789996, rs2852444, rs2604300, rs60237448, |
| | rs59752276, rs2852429, rs2509620, rs3100691, rs2365882, rs10891655, |
| | rs10790005, rs6589426, rs2155805, rs3887314, rs12789793, rs10790006, |
| | rs766783, rs3741302, rs3863297, rs12791529, rs10790008, rs10790009, |
| | rs2365627, rs3851572, rs12282520, rs4938101, rs4938102, rs4938103, |
| | rs7108748, rs4597098, rs7108506. |
| CDKN1B | rs10845572, rs3815591, rs11054953, rs2041927, rs10743990, |
| 12:12867992- | rs10772572, rs10772573, rs10772574, rs4763841, rs11054970, |
| 12875305 | rs10845576, rs4763843, rs35623558, rs11054978, rs10845580, |
| 251 SNPs | rs4763845, rs10772575, rs888544, rs881613, rs7132921, rs10845582, |
| | rs11054988, rs56309863, rs7980170, rs1986164, rs10845584, |
| | rs10772576, rs10845585, rs7358597, rs10492241, rs12308051, |
| | rs10845586, rs55881200, rs6488533, rs4763290, rs7132530, rs35414284, |
| | rs17211947, rs10743991, rs10845588, rs12811932, rs73063495, |
| | rs10845589, rs34725378, rs61915599, rs117238070, rs10772582, |
| | rs10772583, rs12831831, rs10772585, rs10772586, rs17819158, |
| | rs12822507, rs6488539, rs4763858, rs2284794, rs34946234, rs34975957, |
| | rs4763859, rs12580006, rs12830917, rs11609280, rs34051318, |
| | rs7131825, rs35229871, rs61915606, rs741540, rs2900298, rs759748, |
| | rs7309021, rs722780, rs7978272, rs7963653, rs10492239, rs7965458, |
| | rs7973428, rs2111941, rs2417221, rs74700440, rs7299416, rs10845604, |
| | rs12816452, rs9971854, rs10772591, rs10772592, rs10845608, |
| | rs11055015, rs2160972, rs7132848, rs10845611, rs7965148, rs2417222, |
| | rs2417223, rs9804886, rs2080747, rs3759216, rs36228499, rs34328, |
| | rs34327, rs34325, rs34324, rs34323, rs34322, rs6488548, rs12828438, |
| | rs59768035, rs2024385, rs6488549, rs10845623, rs6488550, rs2110598, |
| | rs11055040, rs12320929, rs12321031, rs2058607, rs10845626, |
| | rs7962772, rs1473168, rs11055052, rs10744002, rs7488089, rs10744003, |
| | rs4763871, rs10744004, rs7297457, rs7300798, rs4763872, rs11055057, |
| | rs2417232, rs1079738, rs7133313, rs6488556, rs10744005, rs10744006, |
| | rs7956578, rs1548838, rs7964001, rs7964002, rs4514516, rs1476951, |
| | rs1476952, rs10845630, rs10845631, rs61913633, rs80216939, |
| | rs111661370, rs113872429, rs113782949, rs11055070, rs11055071, |
| | rs4763878, rs10772601, rs2058603, rs2058604, rs2058605, rs12810453, |
| | rs11055074, rs10845633, rs12318909, rs10845636, rs10845639, |
| | rs10734857, rs2417231, rs7972332, rs7973204, rs7973332, rs10744008, |
| | rs10845641, rs7975123, rs9943824, rs11834394, rs7978823, rs3741826, |
| | rs6488559, rs71433476, rs11055091, rs10047556, rs12425031, |
| | rs3741831, rs10845646, rs10845648, rs4762306, rs2287021, rs10845649, |
| | rs4762307, rs2024384, rs7131716, rs61916067, rs61916068, rs12809196, |
| | rs2417234, rs7303734, rs7961795, rs4763890, rs11055107, rs10845656, |
| | rs7978505, rs11055116, rs11055126, rs4462437, rs7300360, rs10845661, |
| | rs3863345, rs3863347, rs7968801, rs4763305, rs4271465, rs3851599, |
| | rs3863350, rs2241228, rs10772609, rs12296954, rs10772610, rs4763306, |
| | rs11055133, rs58452497, rs61092426, rs4763896, rs4763897, rs1984437, |
| | rs7965482, rs11055136, rs2417236, rs11055137, rs3782578, rs3782577, |
| | rs2300643, rs113629527, rs7313660, rs1548836, rs3782572, rs7311128, |
| | rs7311242, rs200440570, rs850938, rs850937, rs850936, rs850935, |
| | rs850934, rs2417238, rs7488715, rs1861182, rs1861183, rs1642203, |
| | rs10845670, rs1684387, rs1684388, rs11055151, rs1684353, |
| | rs199678051, rs2111398. |
| KRAS | rs10842447, rs10842448, rs10842449, rs10771158, rs10771159, |
| 12:25357723- | rs699050, rs699051, rs699052, rs699053, rs859203, rs859202, rs860884, |
| 25403870 | rs859199, rs859198, rs7135717, rs2883033, rs7316549, rs61914766, |
| 260 SNPs | rs715989, rs1497259, rs979553, rs11047799, rs1683168, rs11047800, |
| | rs11047801, rs1497249, rs10842455, rs10842456, rs2054767, rs860877, |
| | rs10743534, rs4963600, rs10842458, rs10743536, rs12315725, |
| | rs1683164, rs1683163, rs7976028, rs6487453, rs16928445, rs859158, |
| | rs859157, rs859154, rs859153, rs2306701, rs11047823, rs7964195, |
| | rs7977670, rs7303669, rs11047824, rs1497253, rs10771165, rs10842463, |
| | rs11832421, rs11832456, rs10842466, rs7960092, rs11047833, |
| | rs6487456, rs4963850, rs2291365, rs10842475, rs12819236, rs12424685, |
| | rs2352782, rs12828609, rs11047855, rs11047856, rs12830058, |
| | rs10771177, rs7306769, rs566624893, rs200798385, rs199734654, |
| | rs200853334, rs12423443, rs7299998, rs11047876, rs112184005, |
| | rs78372860, rs7955844, rs11611468, rs10842501, rs10842502, |
| | rs7312739, rs7302922, rs7137734, rs12814233, rs10842504, rs10842505, |
| | rs11047894, rs11047895, rs11047896, rs11047897, rs4246228, |
| | rs4246229, rs4963859, rs12578773, rs12822857, rs4963860, rs11608657, |
| | rs12579073, rs11047901, rs12368504, rs12424283, rs4963861, |
| | rs11047902, rs10842512, rs11047905, rs7137630, rs12815546, |
| | rs12579942, rs5009909, rs34527025, rs6487464, rs11047915, |
| | rs10505980, rs12813551, rs12226937, rs11047919, rs10842518, |
| | rs4397950, rs2955407, rs6487465, rs11612828, rs4529966, rs4556643, |
| | rs17330302, rs4564422, rs7132980, rs2955404, rs4963605, rs4528424, |
| | rs11615576, rs10771187, rs10771189, rs10771191, rs10842522, |
| | rs10842523, rs12229879, rs10842525, rs7137641, rs7974119, rs4592503, |
| | rs7137301, rs10771198, rs7137395, rs7137611, rs7295713, rs10771199, |
| | rs12319364, rs4963866, rs7300820, rs4963867, rs4963868, rs4963869, |
| | rs4257069, rs59307134, rs4264235, rs7314324, rs7302911, rs11047943, |
| | rs60655178, rs11047944, rs11047945, rs11047946, rs11047947, |
| | rs11047948, rs10842528, rs10842529, rs4257070, rs4379918, rs4277200, |
| | rs4539422, rs12810616, rs12811282, rs12366504, rs12228660, |
| | rs58325490, rs61603343, rs7485340, rs7307496, rs3885680, rs3885678, |
| | rs12828533, rs12579869, rs4601875, rs10505972, rs6487475, rs7961171, |
| | rs4130755, rs4406911, rs4397949, rs573484083, rs11047971, rs2029452, |
| | rs4963875, rs7296810, rs1994581, rs11047976, rs4433651, rs7953479, |
| | rs10842542, rs1873242, rs10842544, rs10842546, rs10842547, |
| | rs11047982, rs55659311, rs11047985, rs10771207, rs4963876, |
| | rs1472036, rs11047990, rs7953386, rs12230608, rs11047991, |
| | rs11047992, rs4963882, rs11047993, rs11047994, rs4578491, rs1387690, |
| | rs10842549, rs10842550, rs113182512, rs12423298, rs10771208, |
| | rs4963613, rs7295052, rs2029449, rs4451808, rs1489907, rs1489908, |
| | rs10842556, rs11048009, rs7953042, rs11048011, rs12369745, |
| | rs10771210, rs28380691, rs28697044, rs28593519, rs11609056, |
| | rs9669368, rs4963884, rs2340375, rs2879758, rs4486713, rs10743562, |
| | rs11048019, rs10842558, rs11615653, rs61924602, rs68101172, |
| | rs4963616, rs7952914, rs10842560, rs7959944, rs7975781. |
| KMT2D | rs2070615, rs2453469, rs10875878, rs2453471, rs2453472, rs2446972, |
| 12:49412758- | rs3782360, rs10783285, rs4760597, rs10783286, rs10875882, rs4760639, |
| 49453557 | rs2453474, rs2926805, rs3104163, rs2446983, rs2446984, rs2446986, |
| 229 SNPs | rs10783287, rs2934998, rs2926806, rs2446999, rs2447001, rs833484, |
| | rs833483, rs833479, rs833478, rs833476, rs10875883, rs10875885, |
| | rs61941141, rs11168779, rs61941142, rs4237853, rs4018508, rs2368701, |
| | rs10875886, rs12228521, rs12227240, rs7489036, rs4433614, rs3825184, |
| | rs4760665, rs11168783, rs11168784, rs11608916, rs10875888, |
| | rs58023472, rs4760661, rs12579249, rs10783292, rs10783293, |
| | rs10875889, rs11168788, rs61941147, rs372743992, rs7299205, |
| | rs10875891, rs10875892, rs11610459, rs11610497, rs11614474, |
| | rs11168790, rs10783295, rs7297958, rs7298330, rs9796008, rs9795742, |
| | rs2293449, rs3759146, rs7134686, rs11168792, rs10875896, rs4237854, |
| | rs11168794, rs12229275, rs61942166, rs61942167, rs1111662, |
| | rs1830192, rs1818996, rs11168795, rs10875897, rs10783297, rs1726435, |
| | rs10747557, rs2131713, rs2030900, rs4018510, rs4760667, rs10875898, |
| | rs11168797, rs4760668, rs2641450, rs61942169, rs2694840, rs2694839, |
| | rs7963018, rs7976473, rs6580696, rs10747558, rs10875900, rs11168798, |
| | rs2463175, rs11168800, rs11168801, rs11168803, rs4526804, rs833820, |
| | rs9783477, rs833821, rs833822, rs9783452, rs1690177, rs2070940, |
| | rs833827, rs833828, rs7313988, rs9645813, rs61942207, rs61942209, |
| | rs833843, rs833841, rs833840, rs833839, rs4760663, rs4760662, |
| | rs1873986, rs10658818, rs201560128, rs2293445, rs1138908, |
| | rs10875910, rs10875911, rs2117029, rs10875912, rs10875913, |
| | rs11168827, rs10875914, rs10875915, rs12580349, rs34436857, |
| | rs11168839, rs7969091, rs7963373, rs10459232, rs11168850, rs6580698, |
| | rs6580699, rs7296288, rs2117028, rs11168854, rs11168857, rs3741621, |
| | rs2279596, rs10747561, rs10747562, rs11168862, rs7958241, rs7958572, |
| | rs7969963, rs7969967, rs10875921, rs7976890, rs9788139, rs1865157, |
| | rs1476137, rs731350, rs9325145, rs11168874, rs10783307, rs35884657, |
| | rs10783308, rs10875925, rs11168882, rs12227610, rs11168885, |
| | rs12229529, rs11168890, rs7954521, rs10875927, rs2114846, |
| | rs11168893, rs11168895, rs10875928, rs12579015, rs58336603, |
| | rs1991427, rs2114845, rs6580702, rs2334931, rs11168902, rs11168903, |
| | rs2334930, rs11168904, rs200142028, rs10875931, rs7954530, |
| | rs7957998, rs2335231, rs1039225, rs1874908, rs12229524, rs11168910, |
| | rs7137978, rs56149019, rs7959497, rs10747563, rs11168917, rs7306084, |
| | rs11168919, rs10875934, rs12309994, rs34022807, rs10875935, |
| | rs34367716, rs68128447, rs10467103, rs10747564, rs10735819, |
| | rs11168930, rs10875938, rs73306251, rs3847766, rs59976042, |
| | rs11168936, rs7307435, rs77996531, rs10875939. |
| CDK4 | rs11172247, rs1678509, rs775322, rs1284464, rs1678542, rs11172254, |
| 12:58141510- | rs10783834, rs2888334, rs10747780, rs2619439, rs1545783, rs4760304, |
| 58149796 | rs812315, rs2306390, rs903067, rs2169987, rs1552842, rs2277324, |
| 227 SNPs | rs1871417, rs10083154, rs703832, rs2307095, rs2619468, rs3899676, |
| | rs7966795, rs1027315, rs774887, rs1082502, rs774888, rs1092472, |
| | rs1082503, rs7134861, rs7304145, rs2640610, rs2640611, rs2640612, |
| | rs7968113, rs7953157, rs11559931, rs7977084, rs7963927, rs1678514, |
| | rs1678515, rs1689604, rs1678520, rs1689601, rs1689600, rs1678522, |
| | rs370834431, rs73123350, rs1689597, rs1689596, rs1678530, rs1678531, |
| | rs1678532, rs1612041, rs11536137, rs1689595, rs1689594, rs1689592, |
| | rs12826708, rs12826985, rs79969666, rs1678511, rs112314469, |
| | rs1678539, rs12302350, rs12302351, rs10876993, rs2928053, rs4760164, |
| | rs4760165, rs10876994, rs1678540, rs55939182, rs61383491, rs2640629, |
| | rs2640631, rs774890, rs774891, rs799575, rs774892, rs2169988, |
| | rs813854, rs1689587, rs7136269, rs10783836, rs7967791, rs2640636, |
| | rs2640637, rs1689586, rs1689585, rs1689584, rs1628552, rs10877001, |
| | rs7311632, rs4760168, rs3825078, rs7979246, rs7952989, rs11172304, |
| | rs10783844, rs7295422, rs10877007, rs7976852, rs10877008, |
| | rs10082911, rs701006, rs799267, rs238516, rs238522, rs1689583, |
| | rs1633359, rs11172315, rs10877011, rs12368653, rs12371356, |
| | rs2069502, rs2072052, rs4646536, rs3782130, rs10877012, rs10877013, |
| | rs10877014, rs10877015, rs10877016, rs11172333, rs10877019, |
| | rs8181644, rs2014886, rs724834, rs56177740, rs4301823, rs4631925, |
| | rs11172342, rs10877020, rs11172343, rs11172344, rs3816896, |
| | rs1875124, rs12582311, rs3825079, rs1021469, rs11172349, rs11172351, |
| | rs871871, rs4760332, rs10877023, rs4760171, rs10877024, rs7489290, |
| | rs10877025, rs10877026, rs10783850, rs10783851, rs10783853, |
| | rs4760335, rs10877028, rs1599750, rs1599751, rs10783854, rs11172362, |
| | rs10783855, rs10783856, rs7298479, rs4760172, rs10877029, |
| | rs10877030, rs1599931, rs59401446, rs12426201, rs10747786, |
| | rs10747787, rs5017863, rs7312623, rs12312955, rs4237844, rs4760339, |
| | rs10444569, rs9645819, rs10877032, rs111169115, rs4760340, |
| | rs11172368, rs12319233, rs11172369, rs2002275, rs10877033, |
| | rs4417327, rs11172372, rs10877034, rs3751331, rs11172375, rs1962047, |
| | rs61935769, rs140185971, rs7963992, rs11172378, rs7954591, |
| | rs7954957, rs4553410, rs4628718, rs4630335, rs4237845, rs7306124, |
| | rs7978588, rs4335583, rs9971803, rs9971722, rs10431506, rs10783857, |
| | rs4583019, rs7298370, rs4760174, rs10877036, rs12308413, rs6581161, |
| | rs4760346, rs4509811, rs3751327, rs3751326, rs7133278, rs7136981, |
| | rs7295748, rs7299249, rs4760347, rs10747789. |
| MDM2 | rs2468424, rs2439756, rs2956529, rs2956528, rs2439759, rs59441419, |
| 12:69201956- | rs7953108, rs2439744, rs2920022, rs7974454, rs2468402, rs1363580, |
| 69239214 | rs4913463, rs2468417, rs2431655, rs751045, rs2468418, rs2259592, |
| 258 SNPs | rs2546509, rs2701090, rs2701088, rs2546527, rs2546526, rs2468428, |
| | rs2701093, rs2546524, rs2261987, rs2546522, rs2910104, rs2910103, |
| | rs2910102, rs2546516, rs2546515, rs2546513, rs2701095, rs2701097, |
| | rs57966403, rs10878858, rs7976871, rs7976876, rs7976687, rs10784723, |
| | rs10878862, rs10878863, rs7301067, rs11177351, rs10748108, |
| | rs10784724, rs10878865, rs111999768, rs3741600, rs11609067, |
| | rs12229810, rs11611360, rs7966848, rs11177362, rs11177365, |
| | rs7956235, rs6581833, rs111945534, rs3741601, rs10878870, |
| | rs10878871, rs11177371, rs10784727, rs11177372, rs4913296, |
| | rs61927838, rs2870819, rs10748111, rs11177378, rs2088577, rs2088578, |
| | rs2120742, rs1144943, rs1144944, rs3930427, rs937282, rs2870820, |
| | rs2279744, rs3730495, rs1196335, rs1196336, rs1690924, rs1196337, |
| | rs1663577, rs2701098, rs1795472, rs3730533, rs3730536, rs1625525, |
| | rs1201644, rs1206251, rs3730556, rs3730558, rs2291857, rs3730581, |
| | rs3730590, rs1663582, rs1795481, rs1695146, rs1690922, rs1695147, |
| | rs3730646, rs1795474, rs73144210, rs11177392, rs3741598, rs1695154, |
| | rs7974293, rs11177397, rs1144949, rs10878874, rs3817605, rs11177399, |
| | rs61926281, rs2904507, rs3853489, rs10878875, rs2589286, rs2044308, |
| | rs2647950, rs2701089, rs1532328, rs7315435, rs3782346, rs1152931, |
| | rs7968202, rs4913472, rs1144954, rs1152933, rs1152934, rs1144959, |
| | rs1144960, rs1144961, rs2120743, rs1565862, rs2642558, rs2454398, |
| | rs2647951, rs2642556, rs2133315, rs2647952, rs1195739, rs1195738, |
| | rs1195817, rs1195816, rs1152939, rs1144962, rs1144963, rs1144964, |
| | rs1144965, rs1195813, rs1195812, rs4363694, rs10878879, rs7315320, |
| | rs12817847, rs10784735, rs35338244, rs7313045, rs12825037, |
| | rs7300126, rs4351896, rs10784736, rs10784737, rs10784738, |
| | rs10784739, rs10878881, rs11177428, rs7959626, rs6581841, rs1908667, |
| | rs7486806, rs9943849, rs34697757, rs7139001, rs11177432, rs1908666, |
| | rs10878883, rs4913481, rs10878884, rs10082715, rs12296712, |
| | rs10082950, rs10878886, rs11514286, rs4913304, rs11533683, |
| | rs1908662, rs11177435, rs1908660, rs2220951, rs10878887, rs10878888, |
| | rs199892067, rs200831866, rs201857027, rs199970546, rs4556632, |
| | rs10878891, rs1908676, rs4913485, rs11177439, rs11177440, rs4315202, |
| | rs7960188, rs55698864, rs2172991, rs2172992, rs2132458, rs11177442, |
| | rs10878895, rs1874429, rs10878896, rs11177445, rs12578667, |
| | rs12581155, rs1908682, rs11177447, rs11177448, rs11177449, |
| | rs2870827, rs7488925, rs10784746, rs11177451, rs12578541, |
| | rs10748116, rs10748117, rs2172989, rs11177453, rs11177454, |
| | rs57996854, rs11613813, rs67362167, rs75844736, rs12296323, |
| | rs10878899, rs11177457, rs8181716, rs11177459, rs11836094, |
| | rs10784749, rs10748118, rs10784750, rs28395241, rs78872601, |
| | rs5009820, rs2132454, rs1908671, rs12825665, rs10878902, |
| | rs113717817, rs10878903, rs10748119, rs1908674, rs7132110, rs871639. |
| BRCA2 | rs431506, rs424118, rs433551, rs112681499, rs77392398, rs204126, |
| 13:32889611- | rs204127, rs457267, rs369666, rs413833, rs467241, rs465023, rs462762, |
| 32973805 | rs456705, rs412226, rs2773962, rs2773963, rs384765, rs1208401, |
| 595 SNPs | rs378850, rs463450, rs462803, rs458324, rs392455, rs455502, rs402881, |
| | rs411126, rs398251, rs388086, rs453109, rs416377, rs407522, rs436227, |
| | rs425029, rs456053, rs120650, rs392655, rs390704, rs463365, rs368140, |
| | rs378205, rs451237, rs371220, rs151297427, rs373981, rs401432, |
| | rs446676, rs421456, rs461603, rs378693, rs389831, rs204568, rs185810, |
| | rs369389, rs407004, rs379261, rs407389, rs407961, rs407412, rs415482, |
| | rs446909, rs423932, rs409496, rs204566, rs7992602, rs389740, |
| | rs389914, rs426510, rs442689, rs451758, rs433669, rs453774, rs434414, |
| | rs438998, rs464935, rs455676, rs457793, rs399197, rs461373, rs434856, |
| | rs387139, rs396150, rs437321, rs407912, rs455428, rs176059, rs430317, |
| | rs2518793, rs2762040, rs799052, rs799054, rs629265, rs379486, |
| | rs9567240, rs4365176, rs9567251, rs2106134, rs12873155, rs66505312, |
| | rs17507316, rs7330056, rs7330812, rs7985410, rs7990912, rs9567260, |
| | rs2518794, rs2762041, rs35876342, rs9567264, rs2285330, rs204571, |
| | rs204572, rs2285331, rs204561, rs9567282, rs9567296, rs9567297, |
| | rs9567301, rs395341, rs558585, rs2427866, rs2427867, rs2476720, |
| | rs537967, rs689401, rs689338, rs539834, rs205003, rs17634824, |
| | rs205004, rs205005, rs205006, rs205008, rs205009, rs205010, |
| | rs17634866, rs9567325, rs9567327, rs205011, rs205012, rs1004075, |
| | rs798972, rs798973, rs798974, rs9533736, rs798975, rs703215, |
| | rs798976, rs703216, rs703217, rs798977, rs798978, rs703218, |
| | rs9533748, rs112618939, rs916732, rs703219, rs703220, rs2520688, |
| | rs2520689, rs2520692, rs2476722, rs1575522, rs798956, rs798957, |
| | rs798958, rs809559, rs798959, rs798960, rs798961, rs2073993, |
| | rs798963, rs2214122, rs2188584, rs2252552, rs881917, rs2520696, |
| | rs2520697, rs1858938, rs1858939, rs2762033, rs2520698, rs2520699, |
| | rs2520700, rs2520701, rs2520702, rs2520703, rs2762034, rs2520704, |
| | rs2762036, rs61347870, rs2520705, rs2520706, rs715129, rs715128, |
| | rs2249149, rs2520708, rs2762037, rs2762038, rs812840, rs2520709, |
| | rs9562553, rs798989, rs798990, rs798991, rs798992, rs798993, |
| | rs798994, rs2073994, rs798982, rs798983, rs798984, rs798985, |
| | rs798986, rs798987, rs798988, rs56079676, rs56289612, rs56186906, |
| | rs55830745, rs9315161, rs8002690, rs7985446, rs6561179, rs9525969, |
| | rs7323213, rs9533886, rs9533903, rs7321831, rs9595159, rs1029398, |
| | rs9567445, rs34197581, rs7992848, rs798967, rs205014, rs2762039, |
| | rs2806623, rs2073996, rs2073997, rs2806628, rs2254299, rs2025415, |
| | rs2520711, rs2806631, rs2520712, rs2806632, rs2806633, rs2806634, |
| | rs2157961, rs2806635, rs2520686, rs2806636, rs2520687, rs2806637, |
| | rs2806638, rs9534010, rs9526028, rs1380945, rs206084, rs7999304, |
| | rs2126043, rs9526033, rs12877839, rs7981590, rs206089, rs206091, |
| | rs1460818, rs206092, rs2078326, rs206093, rs9534076, rs9595279, |
| | rs188207, rs2858212, rs206125, rs206126, rs206127, rs1126250, rs2021, |
| | rs206128, rs762891, rs2013080, rs9567542, rs206132, rs206136, |
| | rs12861790, rs206138, rs206139, rs206140, rs206142, rs206143, |
| | rs206144, rs2032471, rs206101, rs206102, rs206103, rs206104, |
| | rs206105, rs206107, rs12858289, rs206108, rs206109, rs206110, |
| | rs206111, rs206112, rs367234, rs206114, rs206117, rs206119, |
| | rs9562605, rs9567552, rs11571579, rs206122, rs9534174, rs11571613, |
| | rs3752451, rs12869544, rs1963505, rs1029304, rs11571662, rs9567576, |
| | rs206079, rs9567578, rs559067, rs9943876, rs9943890, rs9943888, |
| | rs1460817, rs1460816, rs9567600, rs11571717, rs9534262, rs11571725, |
| | rs9534269, rs206099, rs9534270, rs9534275, rs169546, rs9634672, |
| | rs9567609, rs573014, rs9534318, rs9534323, rs3764791, rs3764792, |
| | rs4942486, rs7328654, rs9534342, rs10870659, rs206146, rs7337574, |
| | rs7337784, rs11571787, rs9567639, rs2238162, rs542551, rs206147, |
| | rs2100785, rs4570704, rs4942505, rs206340, rs9526165, rs7327867, |
| | rs7330025, rs1012130, rs517118, rs1012129, rs1148321, rs1207952, |
| | rs1207953, rs693963, rs1207954, rs2761367, rs472817, rs206319, |
| | rs206320, rs472873, rs471937, rs206322, rs206325, rs206327, rs206329, |
| | rs206330, rs206331, rs206332, rs206333, rs206334, rs169562, rs499966, |
| | rs497255, rs206336, rs206337, rs206339, rs666483, rs2146994, |
| | rs1207944, rs1207945, rs7330459, rs9534494, rs7321117, rs78946385, |
| | rs1207947, rs1207948, rs1576710, rs1081799, rs1081798, rs1088550, |
| | rs1081796, rs703225, rs548369, rs798274, rs703226, rs798273, |
| | rs642604, rs380627, rs408069, rs169600, rs207649, rs207650, rs808880, |
| | rs2010997, rs207642, rs185941, rs798265, rs798266, rs798267, |
| | rs207629, rs207630, rs207631, rs207632, rs207633, rs207637, |
| | rs2858232, rs207643, rs207644, rs207645, rs207646, rs621450, |
| | rs798269, rs535803, rs535783, rs45604, rs392219, rs380916, rs391612, |
| | rs368535, rs394254, rs378429, rs207620, rs207622, rs207623, rs207624, |
| | rs207625, rs207626, rs208434, rs208433, rs208432, rs208431, |
| | rs7998440, rs8000071, rs7991314, rs7984903, rs7328733, rs7328939, |
| | rs10083285, rs208428, rs7335710, rs60875848, rs35817718, rs12323184, |
| | rs7999125, rs718444, rs7332115, rs7332763, rs9591134, rs9591137, |
| | rs208416, rs208417, rs9595850, rs208418, rs11616287, rs208420, |
| | rs9595861, rs9591143, rs9591145, rs797994, rs3742320, rs208430, |
| | rs9595879, rs9595883, rs7981943, rs7327429, rs7337687, rs9595890, |
| | rs7321262, rs422378, rs12429084, rs1324414, rs731414, rs731413, |
| | rs9595893, rs208422, rs28794315, rs9591159, rs9595897, rs28803274, |
| | rs9591162, rs28770476, rs7992980, rs9595901, rs9595905, rs9595908, |
| | rs9591165, rs2021658, rs61947055, rs55645971, rs55880225, rs208426, |
| | rs61947058, rs7998956, rs208410, rs9591180, rs11840502, rs9591185, |
| | rs9595946, rs9595948, rs9595949, rs2032495, rs201740460, rs533730, |
| | rs7983457, rs208411, rs9591189, rs9591191, rs208413, rs208414, |
| | rs2320301, rs61948860, rs9591198, rs408711, rs9595983, rs56194166, |
| | rs2031354, rs7332884, rs9315173, rs9315174, rs7329120, rs7331393, |
| | rs17515873, rs2301388, rs2301389, rs2301390, rs9315175, rs9591219, |
| | rs9591220, rs12428283, rs9596032, rs9596035, rs9591226, rs498372, |
| | rs7335546, rs9591228, rs573022, rs7326809, rs4942791, rs9591230, |
| | rs9596046, rs590558, rs3119702, rs7985656, rs2874281, rs2874282, |
| | rs9596051, rs9596056, rs9596057, rs9596058, rs9596059, rs9596060, |
| | rs513329, rs3783267, rs7317147, rs9591236, rs60088097, rs3135187, |
| | rs3135188, rs6561499, rs6561500, rs3912038, rs687416, rs4942804, |
| | rs7328827, rs3858846, rs3848085, rs590383. |
| RB1 | rs2406697, rs2406698, rs2406699, rs9534912, rs9534913, rs9534914, |
| 13:48877887- | rs9534918, rs9526432, rs9534919, rs9526433, rs9534921, rs61974561, |
| 49056122 | rs9534922, rs9534923, rs4447314, rs9526434, rs9316344, rs7327260, |
| 543 SNPs | rs7328549, rs7327730, rs9534926, rs9534927, rs9567976, rs139762397, |
| | rs7335709, rs9316345, rs6561431, rs6561432, rs7325189, rs7325437, |
| | rs7330130, rs7330472, rs7332304, rs7330913, rs7331285, rs7331900, |
| | rs7338373, rs7337004, rs61973260, rs61973261, rs9534929, rs9567979, |
| | rs9567980, rs1098354, rs4942739, rs1185727, rs17071168, rs7996580, |
| | rs9567986, rs9562800, rs9534931, rs9534932, rs2406709, rs9534933, |
| | rs9595835, rs9595836, rs2148246, rs9567987, rs9595837, rs9534935, |
| | rs59829900, rs56399021, rs4942742, rs9534937, rs7332147, rs9534939, |
| | rs9316346, rs9316347, rs2094192, rs2104480, rs9562802, rs9534943, |
| | rs4942743, rs9534944, rs9534945, rs9534946, rs9534947, rs9534948, |
| | rs7324484, rs113815818, rs4941623, rs9534949, rs9567991, rs9534950, |
| | rs9534951, rs9534952, rs9591135, rs9526443, rs4942747, rs7323969, |
| | rs2148938, rs7990060, rs9316348, rs9316349, rs9567994, rs7981114, |
| | rs7986400, rs1571256, rs7333237, rs7333247, rs7336214, rs9595843, |
| | rs2406797, rs9526445, rs55811249, rs9567999, rs9568000, rs8001243, |
| | rs9526446, rs1410117, rs9526447, rs4941627, rs9568001, rs9316352, |
| | rs9316353, rs9316354, rs9534957, rs9316355, rs9285165, rs9568002, |
| | rs4942751, rs4942752, rs9568003, rs9562806, rs9568004, rs9316356, |
| | rs9316357, rs9316358, rs1927213, rs9316359, rs7326363, rs9568005, |
| | rs7331551, rs7332326, rs9526449, rs9568006, rs9591138, rs9591139, |
| | rs9562808, rs9568008, rs7338844, rs7339370, rs9591140, rs9568009, |
| | rs9568010, rs9534961, rs1327655, rs7317431, rs7318704, rs7322766, |
| | rs9534965, rs9568014, rs9316360, rs9568015, rs9595851, rs9316362, |
| | rs9316363, rs2768789, rs1172549, rs7989898, rs7993691, rs11619475, |
| | rs7981033, rs9526457, rs9534969, rs637831, rs637390, rs1749976, |
| | rs607954, rs595271, rs2765074, rs2765075, rs551034, rs1359861, |
| | rs2765076, rs7981621, rs12875956, rs9534976, rs9534978, rs1815317, |
| | rs7984127, rs7988173, rs7995410, rs4942754, rs9534979, rs9534980, |
| | rs9534981, rs7318104, rs4942755, rs3890027, rs7989671, rs1925747, |
| | rs9534983, rs6561441, rs7329629, rs7994451, rs2182220, rs9534987, |
| | rs9526462, rs7994141, rs17071466, rs9534995, rs7987321, rs4942757, |
| | rs4942758, rs7998964, rs956169, rs2406804, rs4941628, rs7337883, |
| | rs7996316, rs9534996, rs7322987, rs3809328, rs12100135, rs7984554, |
| | rs2243626, rs1886795, rs9526465, rs7984795, rs12877456, rs2248488, |
| | rs2243461, rs12871740, rs9535002, rs2254423, rs34217484, rs55901083, |
| | rs9535007, rs2253520, rs9568029, rs10219833, rs9535011, rs9526470, |
| | rs2406933, rs4600372, rs2252544, rs1981434, rs1981435, rs2854355, |
| | rs2854342, rs3825417, rs2854348, rs2804094, rs9568031, rs198602, |
| | rs7319732, rs9535015, rs9535016, rs2804095, rs9535018, rs4151450, |
| | rs174857, rs573514079, rs9568033, rs9568034, rs9568035, rs171990, |
| | rs198622, rs2854353, rs198619, rs12583516, rs399413, rs2854357, |
| | rs2854362, rs427686, rs9526475, rs171988, rs198561, rs9568036, |
| | rs9562819, rs11839271, rs59128343, rs198610, rs198609, rs198606, |
| | rs12430215, rs1894255, rs471911, rs3020645, rs398158, rs198563, |
| | rs198564, rs9535032, rs425834, rs198568, rs1951775, rs403390, |
| | rs9535033, rs198571, rs198574, rs198575, rs198576, rs198577, |
| | rs198584, rs9568043, rs198590, rs449268, rs367438, rs7325962, |
| | rs9316381, rs9316382, rs943278, rs7326934, rs9535035, rs9316385, |
| | rs6561448, rs2897588, rs9316388, rs943283, rs943284, rs9316393, |
| | rs6561457, rs9316394, rs8001241, rs9316395, rs9316396, rs9316399, |
| | rs9568047, rs8002565, rs6561461, rs7992129, rs1547660, rs9591172, |
| | rs73486867, rs61074776, rs9526479, rs1926597, rs2406935, rs1926595, |
| | rs7996402, rs9316401, rs1536394, rs4942767, rs9591177, rs9591178, |
| | rs7985825, rs7998781, rs9535038, rs7139578, rs7329039, rs7329065, |
| | rs1359688, rs9535039, rs1536395, rs9595929, rs2407093, rs2897587, |
| | rs2104532, rs9535041, rs9634680, rs4635233, rs2407095, rs9535042, |
| | rs9568049, rs9568050, rs9526481, rs8002098, rs9535045, rs9535046, |
| | rs1536396, rs1536397, rs11148110, rs943280, rs1887152, rs7325719, |
| | rs9535049, rs7332322, rs12877288, rs12876717, rs4497565, rs1887154, |
| | rs9316407, rs9568051, rs12855185, rs12859099, rs9595937, rs9562828, |
| | rs7983682, rs9535051, rs9535052, rs9562831, rs9562832, rs1854017, |
| | rs9568056, rs9568058, rs1009719, rs1009718, rs1009717, rs6561469, |
| | rs7990619, rs7990660, rs9568059, rs9568060, rs9568061, rs9568062, |
| | rs9562833, rs1476406, rs2406937, rs4451857, rs9535058, rs9535059, |
| | rs9535060, rs12867213, rs2406938, rs8002035, rs7985015, rs9568064, |
| | rs9568065, rs12864882, rs2209084, rs9595943, rs9591187, rs9595944, |
| | rs7324562, rs12871822, rs9595945, rs12876715, rs11840006, |
| | rs59927903, rs9568066, rs1009529, rs1124925, rs9568068, rs9568069, |
| | rs9568070, rs9568072, rs7332030, rs1980839, rs1980838, rs2146861, |
| | rs9634817, rs12874937, rs7331325, rs2406939, rs9562835, rs9562836, |
| | rs9568077, rs6561474, rs964413, rs9595950, rs7989287, rs1359108, |
| | rs4942769, rs1575465, rs9595955, rs7323160, rs2407246, rs1323556, |
| | rs9595958, rs8001963, rs7318887, rs9285169, rs9742182, rs9316409, |
| | rs9595960, rs9595961, rs7330127, rs7336022, rs12875723, rs7332008, |
| | rs7333434, rs12853985, rs9591194, rs1323553, rs9595966, rs9595967, |
| | rs532700850, rs9568083, rs9568084, rs2146860, rs7324991, rs4608235, |
| | rs1323552, rs4620866, rs9568087, rs1359112, rs1323549, rs1323548, |
| | rs1323547, rs1323546, rs1323545, rs7320290, rs1323544, rs1323543, |
| | rs1323542, rs1323541, rs7327451, rs9535069, rs4110755, rs1853473, |
| | rs986841, rs4942775, rs1323538, rs1323537, rs9595981, rs7325913, |
| | rs1886012, rs7320539, rs4942776, rs7991873, rs7990582, rs7992081, |
| | rs984183, rs984182, rs7994447, rs7994708, rs9526493, rs7337371, |
| | rs7338954, rs7338984, rs9535071, rs9535072, rs9535073, rs7321178, |
| | rs972075, rs7334649, rs962270, rs5001170, rs5001169, rs5001168, |
| | rs4942777, rs1359111, rs1359110, rs1119758, rs1119757, rs4942780, |
| | rs4942781, rs9316410, rs1924089, rs1924090, rs1924091, rs1924092, |
| | rs7324986, rs9595984, rs12862189, rs986801, rs986800, rs990466, |
| | rs767837, rs9535076, rs9535077, rs7327170. |
| ERCC5 | rs673424, rs677179, rs673569, rs613100, rs613097, rs611388, rs629801, |
| 13:103497194- | rs598037, rs595607, rs584504, rs683414, rs681225, rs2442706, |
| 103528345 | rs2442705, rs677594, rs665762, rs9585957, rs9585958, rs9585959, |
| 240 SNPs | rs7334625, rs618562, rs658483, rs2442704, rs626803, rs665416, |
| | rs616831, rs647666, rs659799, rs660236, rs678556, rs584200, rs665479, |
| | rs584710, rs660142, rs639892, rs639862, rs2478931, rs623951, |
| | rs677964, rs623504, rs623429, rs613242, rs613564, rs687386, rs614119, |
| | rs614551, rs616287, rs628033, rs630240, rs630706, rs7988265, |
| | rs9518807, rs896000, rs9514040, rs654454, rs675828, rs9557919, |
| | rs7323315, rs7319093, rs61965998, rs9557924, rs7321796, rs9585976, |
| | rs9585977, rs9582624, rs9557926, rs9557927, rs9585978, rs12859122, |
| | rs7993414, rs8000784, rs1571070, rs9518820, rs9554893, rs9514047, |
| | rs2167091, rs7317303, rs7339102, rs9518824, rs9514049, rs201006552, |
| | rs2893114, rs1375719, rs12855785, rs11069496, rs111675576, |
| | rs9582631, rs9518837, rs9518840, rs9518842, rs2146990, rs60099521, |
| | rs3783234, rs1571068, rs7994575, rs56314598, rs16960594, rs4772504, |
| | rs72653693, rs112179853, rs1408047, rs9557941, rs7334764, rs9585998, |
| | rs2281886, rs4771435, rs9514065, rs7316963, rs1985702, rs3759497, |
| | rs7325496, rs7325708, rs11618723, rs11619749, rs7326943, rs9518854, |
| | rs4150253, rs1998875, rs9554901, rs11069498, rs4150260, rs4150274, |
| | rs4150276, rs4150279, rs4150290, rs3736865, rs4150299, rs3918332, |
| | rs1323698, rs3783236, rs876430, rs751057, rs6491718, rs7318166, |
| | rs9554903, rs9557948, rs9586006, rs9586007, rs7999632, rs9514069, |
| | rs7984853, rs201148764, rs4408399, rs8001540, rs2038710, rs2038708, |
| | rs9300766, rs701545, rs783448, rs701542, rs803961, rs2390807, |
| | rs1864730, rs7994940, rs9514074, rs9518879, rs9300771, rs3916923, |
| | rs9518880, rs59961263, rs9518881, rs9518882, rs9514075, rs9514076, |
| | rs1831287, rs1854521, rs9518883, rs7988961, rs7983085, rs2163587, |
| | rs1025428, rs1025427, rs1019893, rs7985500, rs7322462, rs7323353, |
| | rs7329348, rs7328579, rs1346991, rs766223, rs701555, rs4772514, |
| | rs701559, rs696909, rs696911, rs701561, rs696916, rs9554918, |
| | rs701562, rs11069500, rs783692, rs783693, rs7324320, rs9634534, |
| | rs111734073, rs9634535, rs967472, rs891740, rs1808631, rs1808632, |
| | rs11619346, rs7322528, rs12857732, rs12585665, rs11620536, |
| | rs9554919, rs9554920, rs9557975, rs9557977, rs9554921, rs9554922, |
| | rs9557979, rs12875265, rs2098901, rs9300775, rs929992, rs9557980, |
| | rs9554924, rs2104719, rs12857936, rs3916924, rs9518894, rs9557983, |
| | rs9518897, rs9557984, rs1952590, rs7317756, rs9514082, rs9557986, |
| | rs11069502, rs9300776, rs4142323, rs9557987, rs9586035, rs6491723, |
| | rs6491724, rs4772523, rs2765583, rs696914, rs157380. |
| FOXA1 | rs2415400, rs2006729, rs2006063, rs2006062, rs10150051, rs10139077, |
| 14:38059189- | rs11156952, rs34718937, rs1955950, rs34781514, rs9322980, rs1955949, |
| 38069245 | rs8022848, rs35137324, rs10135206, rs6571812, rs12893226, |
| 258 SNPs | rs17768249, rs17768254, rs56861237, rs10132119, rs7145671, |
| | rs34955525, rs17179057, rs34609612, rs2899860, rs12435038, |
| | rs8008655, rs74845792, rs112195206, rs17179078, rs1955943, |
| | rs7145592, rs7161366, rs7147101, rs1955934, rs4900889, rs10138614, |
| | rs55881111, rs10129947, rs10145135, rs6571814, rs1956427, |
| | rs11156959, rs72674229, rs1011818, rs1989084, rs2208211, rs4444242, |
| | rs1547008, rs7146299, rs11623989, rs7161326, rs12147939, rs12147950, |
| | rs2180609, rs8005136, rs4900945, rs1473715, rs17768343, rs4900958, |
| | rs10133438, rs10133391, rs4898640, rs1159151, rs12884884, |
| | rs10151200, rs7143956, rs4900966, rs4900967, rs4900969, rs4900971, |
| | rs4900972, rs75076266, rs141210824, rs75164522, rs80084476, |
| | rs12232165, rs10144334, rs7146958, rs7153867, rs7159456, rs1956441, |
| | rs1956442, rs7143724, rs1956443, rs11156961, rs11156962, rs1980675, |
| | rs1980676, rs10145473, rs7493775, rs1950826, rs1950827, rs981794, |
| | rs11846416, rs9322981, rs8016138, rs11621659, rs1884798, rs12436923, |
| | rs10149161, rs7160774, rs7161228, rs9322982, rs4901012, rs1884799, |
| | rs1057536, rs10400689, rs10145379, rs968350, rs8019713, rs1956429, |
| | rs7147379, rs1008236, rs11627807, rs11627844, rs11844358, rs1998124, |
| | rs1998125, rs28802081, rs12147245, rs177829, rs457621, rs177835, |
| | rs177848, rs177850, rs177859, rs177861, rs177862, rs177865, rs177867, |
| | rs177869, rs177871, rs177872, rs177875, rs177877, rs177878, rs177882, |
| | rs177885, rs177888, rs11848078, rs177899, rs61988008, rs11849126, |
| | rs61988009, rs1951646, rs10083306, rs4141723, rs1957590, rs12433850, |
| | rs1951647, rs7147915, rs4901110, rs9989212, rs960787, rs12435334, |
| | rs17106954, rs11156965, rs17179337, rs61977077, rs12431417, |
| | rs8010816, rs8022736, rs4901140, rs1957597, rs1535540, rs12879473, |
| | rs2224667, rs1319189, rs7148752, rs966916, rs966918, rs12889959, |
| | rs12890128, rs17106990, rs17768521, rs35834125, rs10151208, |
| | rs10151293, rs8022025, rs6571819, rs7154854, rs28481790, rs11156966, |
| | rs1885713, rs12883286, rs1998425, rs1998426, rs1998427, rs1014992, |
| | rs1014991, rs12895548, rs12894792, rs12436962, rs12436983, |
| | rs1570399, rs1570400, rs1570401, rs1570402, rs1570403, rs1885714, |
| | rs1885715, rs76552998, rs78423492, rs4901157, rs17107021, rs4432183, |
| | rs1957573, rs1957574, rs1957575, rs12437420, rs7141017, rs11845095, |
| | rs11849517, rs728087, rs728088, rs728089, rs11846483, rs12434788, |
| | rs12434817, rs12431986, rs1957578, rs12588751, rs17768594, |
| | rs1957580, rs6571820, rs7144475, rs1040951, rs68016111, rs61977123, |
| | rs12897750, rs12883675, rs61977125, rs7156854, rs7157460, rs8003755, |
| | rs1957582, rs58878623, rs61977137, rs61977138, rs11846354, |
| | rs8013150, rs8016889, rs10872903, rs1957583, rs12888912, rs12432869, |
| | rs4901272, rs12586423, rs8010511, rs4901274, rs1957584, rs17179497, |
| | rs969446, rs7140857, rs8011077, rs12434191. |
| RAD51B | rs8011690, rs1956346, rs7157288, rs11624166, rs4363798, rs10148527, |
| 14:68286496- | rs4902533, rs72725179, rs11846526, rs1958114, rs7140939, rs2877455, |
| 69196935 | rs12880842, rs2064914, rs927220, rs7148322, rs61985135, rs61985136, |
| 246 SNPs | rs10136039, rs12232174, rs911258, rs1108590, rs3784104, rs12889949, |
| | rs11158729, rs7148073, rs7148412, rs3784106, rs12890110, rs12890124, |
| | rs11628890, rs12881502, rs8013078, rs8017304, rs8022860, rs12878858, |
| | rs11626138, rs7146850, rs1956528, rs3784108, rs3784109, rs3825730, |
| | rs4902566, rs28585636, rs12878627, rs4899238, rs9323506, rs7147852, |
| | rs2145422, rs1956526, rs4410007, rs10131654, rs7493547, rs911255, |
| | rs1956523, rs2331727, rs2013413, rs2064911, rs10483807, rs8013968, |
| | rs10144669, rs11158730, rs7149396, rs61986905, rs56399929, |
| | rs2208396, rs7149637, rs12888061, rs11158732, rs61986906, rs1977211, |
| | rs58339869, rs59993918, rs8018477, rs3784113, rs4902574, rs2009721, |
| | rs2877461, rs61986910, rs55749276, rs2025009, rs3784115, rs3784116, |
| | rs3950069, rs61039700, rs8014025, rs8014234, rs11625452, rs55665241, |
| | rs12435046, rs2331696, rs11158736, rs1956537, rs1956536, rs17105561, |
| | rs10146772, rs17105571, rs17105585, rs17105586, rs4545737, |
| | rs12435236, rs4902579, rs746663, rs8010439, rs10873213, rs10131342, |
| | rs4902583, rs7151360, rs2331702, rs4902586, rs12885674, rs1956533, |
| | rs2842322, rs10467820, rs2093202, rs1468279, rs10130683, rs6573837, |
| | rs2107340, rs3784124, rs2074562, rs2074563, rs2074564, rs2097800, |
| | rs10149340, rs10149349, rs2074565, rs5004090, rs2040783, rs12886864, |
| | rs9323512, rs7160842, rs4899244, rs3784130, rs12587231, rs2525510, |
| | rs737387, rs2525511, rs739872, rs757369, rs2078316, rs8015246, |
| | rs2842341, rs1541390, rs1023529, rs1023530, rs576903789, rs9323513, |
| | rs9323514, rs10133625, rs118040061, rs11158749, rs10134446, |
| | rs7140266, rs11158750, rs2842333, rs2525530, rs2679, rs12587232, |
| | rs2525503, rs2525504, rs12586230, rs11158751, rs8016149, rs7153476, |
| | rs6573842, rs7146456, rs11621880, rs8009944, rs10130694, rs10148414, |
| | rs2285883, rs2253168, rs761944, rs2253317, rs2256224, rs8016488, |
| | rs11623751, rs2257022, rs10136315, rs11623593, rs17756404, |
| | rs12878344, rs11845204, rs2236185, rs5028394, rs4362328, rs10151284, |
| | rs4902610, rs17834947, rs12586179, rs12586180, rs71425370, |
| | rs12588477, rs12588461, rs10136390, rs12432197, rs2145154, |
| | rs2145153, rs56388123, rs12890882, rs12893578, rs4643253, |
| | rs12435141, rs116988664, rs116814518, rs8021657, rs10483817, |
| | rs2180611, rs4902627, rs8006682, rs7141529, rs7350738, rs2093101, |
| | rs4902628, rs767127, rs1547011, rs10483820, rs62650186, rs1055840, |
| | rs4902630, rs56144448, rs2145149, rs1884807, rs7145032, rs6573851, |
| | rs6573852, rs1007840, rs1007841, rs8006379, rs8008240, rs12886919, |
| | rs11847289, rs11851586, rs28594092, rs8006663, rs8007140, rs8007722, |
| | rs12897613, rs382179, rs382666, rs56750623, rs454988, rs452870, |
| | rs12895422, rs61984984. |
| AKT1 | rs4077167, rs7155652, rs12101279, rs12101271, rs12101275, rs3809459, |
| 14:105235686- | rs3809458, rs12879330, rs12880651, rs3803316, rs11160812, rs7148772, |
| 105262088 | rs10144804, rs7148960, rs10873548, rs12892016, rs11628289, |
| 259 SNPs | rs10151692, rs10151872, rs10129179, rs7145273, rs6576053, rs8014246, |
| | rs12880146, rs12880486, rs11625777, rs8010873, rs10138223, |
| | rs8003138, rs12431620, rs7146661, rs10147927, rs4983375, rs4983507, |
| | rs8018405, rs4983509, rs4983376, rs4983510, rs4983511, rs4643255, |
| | rs10142723, rs536302651, rs193251778, rs10147047, rs10134910, |
| | rs11850699, rs7494687, rs8023004, rs55792168, rs4983519, rs4075358, |
| | rs4074077, rs8007360, rs12433813, rs55764224, rs9671466, rs6576056, |
| | rs28539404, rs8022211, rs4247027, rs4247028, rs4553558, rs4983520, |
| | rs10137856, rs8004004, rs4983521, rs4983522, rs7145300, rs7153053, |
| | rs4983528, rs12432572, rs10136869, rs3809456, rs4983529, rs4393510, |
| | rs4275790, rs4334222, rs7140154, rs6576058, rs7154981, rs11160817, |
| | rs12588561, rs12882277, rs10144190, rs12888991, rs528760325, |
| | rs12433350, rs4432208, rs12887451, rs35746773, rs72715972, |
| | rs57430375, rs60876857, rs11848612, rs60798007, rs57098433, |
| | rs34272041, rs7142772, rs7141697, rs7149183, rs7160733, rs3803307, |
| | rs4983539, rs4983384, rs4247034, rs4983386, rs7155192, rs4983546, |
| | rs8011461, rs8011779, rs8006580, rs8006310, rs12889167, rs7151424, |
| | rs12587551, rs12590163, rs45607139, rs4983549, rs7158655, |
| | rs66464514, rs4983550, rs2498804, rs2498803, rs2494730, rs2498802, |
| | rs2498801, rs2494731, rs2498800, rs2494732, rs2494733, rs2498797, |
| | rs3001371, rs2494735, rs2498796, rs2498794, rs2494737, rs2494740, |
| | rs2498789, rs2494743, rs2494746, rs2494747, rs2494748, rs2498786, |
| | rs73350342, rs12888969, rs4262882, rs11160819, rs4983557, rs4983558, |
| | rs4983559, rs2498782, rs2498810, rs2498778, rs2498809, rs2498779, |
| | rs2498808, rs66987285, rs67906390, rs11629188, rs8010024, |
| | rs12889174, rs12889498, rs12889599, rs12101186, rs2582519, |
| | rs3001410, rs2919620, rs2582516, rs2582515, rs10138387, rs28562928, |
| | rs2841216, rs3001412, rs3001413, rs2919618, rs79206914, rs2841219, |
| | rs111172815, rs61995982, rs2841220, rs2582565, rs61995986, |
| | rs2841221, rs4983561, rs2841223, rs2841225, rs2841227, rs28427389, |
| | rs2582562, rs2841230, rs28694394, rs11844343, rs2841231, rs2582561, |
| | rs56110037, rs2841233, rs2582558, rs2028416, rs2841235, rs2028415, |
| | rs10145119, rs10145122, rs10145031, rs9788511, rs59949155, |
| | rs2582549, rs894038, rs2251389, rs2582545, rs2289453, rs61996002, |
| | rs61996014, rs1866824, rs1866825, rs3742939, rs45580235, rs7152667, |
| | rs10147348, rs2841245, rs6576063, rs11160824, rs3957307, rs2083540, |
| | rs2841267, rs2819438, rs2582526, rs2841268, rs2841269, rs2841275, |
| | rs2582529, rs2841277, rs2841278, rs2582531, rs2819468, rs2841281, |
| | rs2819469, rs2841283, rs1036713, rs2582533, rs10083490, rs10083496, |
| | rs10083374, rs10149193, rs11160827, rs733130, rs11621410, |
| | rs10137166, rs2819450, rs2582497, rs2582496, rs736066, rs9805957, |
| | rs2819454, rs2582492, rs10136586, rs7159328, rs7159532, rs10143022, |
| | rs10143079. |
| IDH2 | rs3825866, rs28372736, rs10459700, rs10459673, rs10459701, |
| 15:90626277- | rs10459674, rs4932150, rs2043882, rs2043881, rs1910587, rs28754854, |
| 90645736 | rs8034116, rs8033554, rs8038480, rs3853638, rs7183156, rs7178909, |
| 263 SNPs | rs11852306, rs3850996, rs12909376, rs11629802, rs11633304, |
| | rs145225650, rs568230141, rs1106990, rs11856803, rs4932272, |
| | rs10775248, rs10775249, rs11852297, rs2135658, rs2135657, rs2174694, |
| | rs2352051, rs67024503, rs7171721, rs4932154, rs4932155, rs4932276, |
| | rs1317722, rs12903380, rs1876291, rs7496448, rs57047369, rs1876294, |
| | rs1876293, rs1876292, rs950116, rs55951036, rs1317445, rs12593037, |
| | rs9806319, rs6496620, rs2939847, rs2939849, rs2939846, rs2970358, |
| | rs538020019, rs7177165, rs7178604, rs28547012, rs8030346, rs4932278, |
| | rs11853055, rs6496622, rs6496623, rs10459702, rs4932279, rs4932280, |
| | rs34552862, rs9672249, rs9652515, rs9652516, rs28458925, rs28508560, |
| | rs11638845, rs7182369, rs62019188, rs4283211, rs4316732, rs4561444, |
| | rs11632348, rs11073900, rs4932162, rs11637880, rs60760826, |
| | rs61302266, rs55771383, rs12443387, rs56175150, rs56331873, |
| | rs7168371, rs7169036, rs8030429, rs8030465, rs59337025, rs8035004, |
| | rs4932284, rs60462195, rs61215894, rs7163762, rs4932163, rs8032563, |
| | rs8032566, rs28484767, rs4436756, rs62019195, rs9672635, rs7169903, |
| | rs80099646, rs6496632, rs4395050, rs11630423, rs6496634, rs8027425, |
| | rs2970348, rs12908391, rs114035823, rs2970343, rs2943224, rs2970345, |
| | rs8024587, rs2960124, rs8035601, rs2137892, rs2137893, rs2943226, |
| | rs12050478, rs8028247, rs7166826, rs908045, rs2960125, rs2943227, |
| | rs2970353, rs12910458, rs12911052, rs11853144, rs7164245, rs6496635, |
| | rs12441566, rs12438336, rs4932165, rs2352213, rs2352214, rs8023792, |
| | rs10152852, rs10152809, rs8039188, rs4261509, rs59565248, |
| | rs58279224, rs10852127, rs12591576, rs9707882, rs9707883, |
| | rs73473569, rs7172710, rs8042171, rs8042334, rs61403106, rs59448290, |
| | rs28609426, rs12914591, rs11073907, rs11073908, rs11073909, |
| | rs11855301, rs11852420, rs57112180, rs12591277, rs12594445, |
| | rs6496636, rs6496637, rs6496638, rs73473586, rs12906254, rs12439346, |
| | rs12439592, rs7183853, rs4932296, rs34710083, rs8033939, rs10852128, |
| | rs10852129, rs12438592, rs73475867, rs73475870, rs7171150, |
| | rs7172562, rs6496640, rs6496641, rs60209970, rs4932298, rs4932299, |
| | rs883408, rs2352209, rs9806563, rs7178860, rs2352216, rs1877784, |
| | rs59788933, rs7164703, rs2292577, rs11073914, rs1399237, rs6496645, |
| | rs7171735, rs1533325, rs1533326, rs28681010, rs8036117, rs8036241, |
| | rs1543116, rs8041503, rs3826001, rs76619542, rs8030039, rs8025627, |
| | rs2352220, rs2271553, rs8033436, rs2073706, rs2073703, rs4628940, |
| | rs8041338, rs8025875, rs9745131, rs9744242, rs4932306, rs4932307, |
| | rs2063738, rs12900595, rs7173922, rs4932168, rs7497536, rs4932169, |
| | rs4932170, rs8023756, rs8025228, rs12595130, rs12916170, rs12901544, |
| | rs11073920, rs12591585, rs71405691, rs71405692, rs881341, rs881342, |
| | rs4932309, rs4513071, rs4319744, rs4357917, rs11629778, rs34255992, |
| | rs1533327, rs10083710, rs10083711, rs375723134, rs538960691, |
| | rs2048707, rs28714598, rs7161831, rs12912653. |
| ERCC4 | rs179603, rs7184170, rs179606, rs179609, rs7192709, rs12708778, |
| 16:14014014- | rs1573635, rs76508472, rs12930325, rs1547406, rs2866845, rs2866846, |
| 14046202 | rs7198787, rs1029286, rs1029287, rs8054801, rs78103788, rs74454170, |
| 266 SNPs | rs76476936, rs8061168, rs12918144, rs12918319, rs12926143, |
| | rs12447128, rs11864432, rs12444957, rs12448610, rs12445003, |
| | rs12445004, rs7199833, rs12932802, rs7204475, rs9673393, rs7184744, |
| | rs7206756, rs8049127, rs2157072, rs2187678, rs2187679, rs2187680, |
| | rs7190789, rs4291923, rs2227013, rs7189964, rs4781539, rs9925480, |
| | rs9931262, rs12918778, rs34589696, rs2157075, rs2051547, rs12448531, |
| | rs2903404, rs6498468, rs2213302, rs7199244, rs1159166, rs7185370, |
| | rs11861769, rs8061274, rs2157076, rs9302505, rs1029288, rs9921964, |
| | rs8049968, rs12924365, rs12708779, rs12920892, rs1123765, rs8052883, |
| | rs8061984, rs12932396, rs12931108, rs12918580, rs4780544, |
| | rs12919542, rs9936934, rs12922829, rs72779461, rs7194109, |
| | rs10459788, rs12920090, rs6498472, rs8055738, rs8055949, rs8062468, |
| | rs60582851, rs11642741, rs11642795, rs9935515, rs9934964, |
| | rs67796535, rs12600135, rs8062820, rs78773444, rs28522244, |
| | rs4781553, rs4781554, rs7198922, rs7193571, rs2866851, rs725457, |
| | rs4781555, rs1034392, rs79502695, rs16962919, rs16962922, |
| | rs35644981, rs9937737, rs2866854, rs743671, rs12935804, rs963536, |
| | rs9934011, rs16962950, rs61214678, rs57132529, rs58101145, |
| | rs8048012, rs35231659, rs5004317, rs724877, rs7203351, rs9936449, |
| | rs7188652, rs7186773, rs7193899, rs34415747, rs2011296, rs62034058, |
| | rs1424276, rs928974, rs1029290, rs11646379, rs1646314, rs12927127, |
| | rs231128, rs231129, rs231131, rs231127, rs12926155, rs12926156, |
| | rs1987556, rs1646311, rs1794337, rs34089846, rs56381936, rs889822, |
| | rs1862865, rs8045998, rs1862863, rs2113334, rs4780547, rs11647585, |
| | rs8054265, rs567808829, rs10163216, rs10163236, rs10163219, |
| | rs7190291, rs7194972, rs9302510, rs9302511, rs1364363, rs6498482, |
| | rs7184192, rs12444144, rs62035691, rs7204841, rs11644737, rs3888364, |
| | rs12708786, rs7195297, rs7195395, rs7190692, rs9925151, rs12929613, |
| | rs12933628, rs2866859, rs2866860, rs889817, rs753192, rs4078751, |
| | rs9928336, rs2081273, rs11642435, rs7197700, rs9931851, rs4781560, |
| | rs11648436, rs7186212, rs7202291, rs11649492, rs11639593, |
| | rs12926685, rs12921111, rs6498485, rs3136038, rs6498486, rs3136064, |
| | rs3136065, rs3136070, rs2238463, rs3136085, rs957078, rs3136112, |
| | rs3136155, rs3136160, rs3136166, rs3136168, rs3136187, rs3136189, |
| | rs3136202, rs1799799, rs3136218, rs1646332, rs1646331, rs11648736, |
| | rs8060520, rs72781482, rs56960460, rs199545899, rs12922544, |
| | rs11642211, rs8057299, rs4522405, rs1560150, rs1630367, rs1794242, |
| | rs9746645, rs2903447, rs1651191, rs1794244, rs1651190, rs1651189, |
| | rs1651188, rs1794226, rs72783406, rs67174119, rs9936475, rs55995804, |
| | rs1794228, rs1651186, rs3909878, rs55863630, rs1651184, rs9806993, |
| | rs1794230, rs59953078, rs1794232, rs8063309, rs7189421, rs7184976, |
| | rs1794236, rs1630373, rs1629592, rs1794238, rs4781564, rs12920252, |
| | rs12919705, rs9935021, rs12599691, rs8051983, rs11862729, |
| | rs13334256, rs7205210. |
| PALB2 | rs30014, rs39815, rs30012, rs30011, rs40817, rs250579, rs250578, |
| 16:23614488- | rs193937, rs76430970, rs75504766, rs11861636, rs250551, rs250553, |
| 23652631 | rs250558, rs11647292, rs250559, rs540719, rs3096175, rs428438, |
| 249 SNPs | rs437647, rs531805, rs659845, rs10459765, rs369856, rs6497664, |
| | rs11074559, rs66569010, rs11074560, rs36122804, rs11074561, |
| | rs2369013, rs11074562, rs7190131, rs2239951, rs722069, rs9302410, |
| | rs10153059, rs9927961, rs9933261, rs727238, rs12921516, rs2097662, |
| | rs4968014, rs9931842, rs4967958, rs552149672, rs565878282, |
| | rs2040573, rs11074565, rs7405145, rs11642434, rs7199108, rs2106456, |
| | rs9923234, rs2369009, rs11642395, rs11643602, rs4968019, rs2106454, |
| | rs717998, rs9972807, rs152460, rs12447847, rs120962, rs120961, |
| | rs432477, rs460477, rs120963, rs566769, rs3096145, rs570277, |
| | rs249934, rs420259, rs12926135, rs450787, rs249954, rs62032983, |
| | rs459603, rs249855, rs523422, rs249871, rs249870, rs249869, rs249868, |
| | rs34514, rs34513, rs40076, rs35635, rs42873, rs171617, rs35632, |
| | rs26763, rs26762, rs11074570, rs8058610, rs3760108, rs7199034, |
| | rs36047689, rs4968025, rs9940582, rs3899652, rs3852769, rs3852770, |
| | rs9935010, rs9926444, rs11647981, rs11642597, rs4968036, rs9935139, |
| | rs7201192, rs9745523, rs13332138, rs2118217, rs2118215, rs187406, |
| | rs7203684, rs246330, rs11647438, rs7190159, rs780166, rs11643062, |
| | rs780165, rs12918410, rs780164, rs1657542, rs12933387, rs4967960, |
| | rs4967961, rs7191762, rs11646623, rs780169, rs13329786, rs2368594, |
| | rs1657538, rs1657545, rs11649332, rs152041, rs152042, rs109592, |
| | rs194821, rs80950, rs56265975, rs113612782, rs194802, rs194801, |
| | rs194800, rs191788, rs168970, rs187749, rs12918337, rs187748, |
| | rs4277349, rs174217, rs2520009, rs194797, rs34914367, rs13335425, |
| | rs13335481, rs9935512, rs12708651, rs8055310, rs7404693, rs2575389, |
| | rs194788, rs185452, rs181983, rs194789, rs194790, rs11646605, |
| | rs2520013, rs2520014, rs12051208, rs1858873, rs1858874, rs2520016, |
| | rs2520017, rs9923292, rs2520018, rs2520019, rs7196602, rs2575385, |
| | rs2520020, rs2520021, rs2334246, rs194817, rs168974, rs183172, |
| | rs194816, rs194815, rs174219, rs194812, rs194811, rs194810, rs168972, |
| | rs4968042, rs4968041, rs2520026, rs168971, rs7200013, rs194805, |
| | rs11074578, rs9929466, rs12446910, rs2520027, rs2005672, rs187747, |
| | rs168968, rs194793, rs194791, rs60356559, rs35916311, rs4047243, |
| | rs7188028, rs3852779, rs12924979, rs12924854, rs12925342, |
| | rs12708652, rs9927848, rs9927992, rs9927993, rs9933339, rs4081534, |
| | rs7185480, rs7198121, rs194795, rs12708653, rs194796, rs2575390, |
| | rs3900007, rs3900008, rs11645239, rs7185969, rs7184949, rs7185142, |
| | rs7184668, rs12935280, rs3729883, rs2188357, rs2188358, rs2023671, |
| | rs10852255, rs7200394, rs7199385, rs6497687, rs6497688, rs916678, |
| | rs7189218, rs7404078. |
| CYLD | rs4785438, rs7204135, rs4785220, rs12596811, rs7206907, rs8046845, |
| 16:50775961- | rs8047222, rs1558663, rs11640716, rs7198686, rs12924696, rs749986, |
| 50835846 | rs749985, rs7199150, rs4486887, rs6500325, rs9926095, rs6500326, |
| 250 SNPs | rs7186163, rs7186262, rs2066849, rs6500327, rs8050932, rs7203253, |
| | rs1109863, rs2357623, rs7202124, rs2270369, rs2270368, rs2287195, |
| | rs4785223, rs9926569, rs9939349, rs8062727, rs8043770, rs9302752, |
| | rs1420685, rs2004804, rs34746653, rs34550909, rs1981760, rs8063362, |
| | rs9933594, rs12926429, rs4785448, rs11647143, rs7194886, rs34231814, |
| | rs11645448, rs4785224, rs2111235, rs2111234, rs8045009, rs6500328, |
| | rs8057341, rs13339578, rs751271, rs1861758, rs13332952, rs7203691, |
| | rs1077861, rs9921146, rs8056611, rs2357792, rs7205423, rs718226, |
| | rs1548990, rs8060598, rs3785142, rs7342715, rs367939843, |
| | rs372106942, rs554985572, rs12929222, rs536650992, rs200640737, |
| | rs34464167, rs8053457, rs6500329, rs4785451, rs6500331, rs17314341, |
| | rs3785140, rs4027241, rs2111435, rs17314544, rs17223195, rs13337656, |
| | rs4785226, rs2160683, rs1861762, rs9635531, rs4785452, rs4785453, |
| | rs4785227, rs4785454, rs13332720, rs2270396, rs2270395, rs2270394, |
| | rs12922698, rs12932778, rs12445012, rs4785456, rs4785457, rs8062570, |
| | rs2098456, rs11642470, rs1548993, rs62029895, rs62029896, rs2111434, |
| | rs16948899, rs4785229, rs9939940, rs1477174, rs1477173, rs16948930, |
| | rs2160684, rs4785463, rs1548991, rs1001608, rs11643967, rs11649409, |
| | rs8045724, rs7197081, rs7199785, rs7195322, rs8054450, rs6500341, |
| | rs1874499, rs6500343, rs35803377, rs34496721, rs35807785, |
| | rs12927461, rs12927486, rs3135505, rs1390762, rs12929266, rs7199660, |
| | rs7205452, rs8060571, rs11076542, rs16948989, rs60822814, |
| | rs62029939, rs9935279, rs11646619, rs1496645, rs1496644, rs11647703, |
| | rs1874498, rs1874497, rs4082588, rs11644451, rs11643352, rs11648055, |
| | rs9930980, rs9938759, rs2357933, rs35184192, rs4785464, rs8062873, |
| | rs55906843, rs8063149, rs8044305, rs8044493, rs1995066, rs6500345, |
| | rs6500346, rs62032367, rs7194937, rs8053438, rs12446199, rs12448241, |
| | rs1603467, rs1874495, rs1353542, rs4785465, rs1496643, rs1353541, |
| | rs4785466, rs4785467, rs1496642, rs1496641, rs1395603, rs1507046, |
| | rs1507047, rs1507048, rs4785472, rs4785474, rs12596552, rs4785476, |
| | rs4785477, rs4785478, rs7189647, rs729442, rs729443, rs1605377, |
| | rs1605379, rs2357936, rs728868, rs9935368, rs34694866, rs4238818, |
| | rs2357937, rs1553019, rs16949073, rs6416788, rs66502769, rs2357938, |
| | rs9989387, rs6500348, rs75764788, rs12598954, rs1872679, rs7190169, |
| | rs6500351, rs4638569, rs4485368, rs1564981, rs2129247, rs1872678, |
| | rs1528602, rs8059348, rs7203810, rs7203832, rs8052602, rs1948547, |
| | rs9929768, rs10852610, rs2047327, rs2129250, rs2129249, rs2129248, |
| | rs58302873, rs58161685, rs9921820, rs8046199, rs1566047, rs1567150, |
| | rs9927190, rs11076545, rs1976113, rs2171225, rs963231, rs12918168, |
| | rs62028333, rs61539933. |
| ZFHX3 | rs12924937, rs7189059, rs1001493, rs2358801, rs12445443, rs6499577, |
| 16:72816784- | rs12598536, rs11641842, rs4788473, rs7189331, rs11646282, rs1560278, |
| 73093597 | rs7188640, rs7198233, rs7198431, rs7205109, rs12149680, rs12444535, |
| 255 SNPs | rs11648672, rs11648635, rs12708933, rs72803199, rs4788646, |
| | rs8048181, rs8059626, rs11648405, rs201460117, rs292936, rs12923688, |
| | rs2266943, rs7185166, rs8045691, rs8046332, rs9937452, rs8050431, |
| | rs4788668, rs55797313, rs55878167, rs6499590, rs7342782, rs61344899, |
| | rs7500397, rs3923463, rs7404229, rs7197197, rs7198843, rs7197875, |
| | rs7199274, rs11075947, rs7193283, rs7404577, rs11862105, rs11862114, |
| | rs9925261, rs2157787, , rs16971384, rs546212795, |
| | rs564520936, rs6499595, rs4788480, rs1076158, rs7198066, rs4788672, |
| | rs1008275, rs2040509, rs4788678, rs4788680, rs60177356, rs6499600, |
| | rs6499601, rs13336412, rs56252617, rs55709556, rs72795127, |
| | rs63030061, rs11075950, rs876444, rs7190638, rs2106259, rs2106258, |
| | rs554282398, rs2106257, rs2157786, rs756717, rs4788683, rs11075952, |
| | rs11075953, rs12595893, rs9921395, rs1010852, rs8061096, rs12325558, |
| | rs10852516, rs11075954, rs2040508, rs9930445, rs4788684, rs4238967, |
| | rs9922669, rs17681554, rs4788489, rs16971471, rs1858801, rs11640106, |
| | rs4541098, rs11075957, rs13380725, rs2106254, rs58563212, rs7193343, |
| | rs7192350, rs12931021, rs4788687, rs7200212, rs6416747, rs11643592, |
| | rs12445992, rs8056528, rs4788688, rs59288483, rs61078156, |
| | rs55649472, rs58084688, rs719353, rs719354, rs6499606, rs9925403, |
| | rs7199827, rs7185241, rs1548373, rs56274700, rs4788692, rs8057081, |
| | rs4788693, rs4788694, rs9940321, rs4788490, rs11862034, rs728646, |
| | rs61164185, rs11641701, rs60602157, rs8049936, rs4788697, rs9930504, |
| | rs72626185, rs7498798, rs6416749, rs1183965, rs1183964, rs9940161, |
| | rs11075962, rs11640435, rs4344770, rs28539433, rs11075964, |
| | rs4788700, rs56699618, rs59413571, rs9938769, rs8045914, rs8045933, |
| | rs73591197, rs74028030, rs4278773, rs4482318, rs4281746, rs4544265, |
| | rs7206167, rs80297838, rs7185870, rs8054288, rs535851046, rs7186458, |
| | rs10852517, rs9928752, rs7187836, rs7187519, rs13329908, rs1074564, |
| | rs34954088, rs9940468, rs12149392, rs12149930, rs12149399, |
| | rs12149931, rs4307972, rs35744751, rs12446778, rs12446805, |
| | rs12446752, rs12446786, rs12444072, rs11075966, rs11075967, |
| | rs4788491, rs4788492, rs4788703, rs12051003, rs9922206, rs62052404, |
| | rs4268781, rs6499616, rs4261572, rs11863839, rs7192296, rs4451977, |
| | rs4318234, rs4405578, rs7190071, rs4788705, rs4788706, rs11862861, |
| | rs11866596, rs4788495, rs11075970, rs11075971, rs59029059, |
| | rs59066461, rs59662278, rs12922660, rs9936439, rs7202562, rs4503824, |
| | rs9921088, rs11639665, rs62052437, rs11075972, rs11641092, |
| | rs8055289, rs11645441, rs11645667, rs28715831, rs7359443, rs6499618, |
| | rs8062728, rs9921381, rs7193833, rs9939492, rs9922122, rs59195934, |
| | rs7203458, rs7498310, rs8054577, rs74249780, rs4129177, rs111067768, |
| | rs28723625, rs28742971, rs28698707, rs62052454, rs34934522, |
| | rs73603314, rs55639728. |
| FANCA | rs7199850, rs11862848, rs397564, rs464349, rs464274, rs2377054, |
| 16:89803957- | rs2889542, rs9708822, rs4479222, rs4448938, rs7184767, rs154658, |
| 89883065 | rs8048760, rs164737, rs154659, rs154660, rs77717194, rs407984, |
| 221 SNPs | rs8058428, rs8057288, rs391773, rs8064126, rs56164821, rs62068703, |
| | rs664020, rs455344, rs8044136, rs35078537, rs409170, rs7404028, |
| | rs258341, rs9673694, rs3794633, rs908951, rs2250598, rs2434858, |
| | rs420332, rs4785580, rs4785581, rs1657380, rs386458147, rs2460448, |
| | rs164749, rs352939, rs460879, rs62068735, rs466598, rs258337, |
| | rs258336, rs11647958, rs258335, rs7203947, rs76762453, rs7188458, |
| | rs11646741, rs13332439, rs16965913, rs2377058, rs12445808, |
| | rs2115401, rs12443954, rs12446791, rs68092778, rs12924138, |
| | rs2075880, rs164744, rs164742, rs2115400, rs4785587, rs1110331, |
| | rs59660050, rs12149952, rs57119673, rs7204478, rs4785591, rs4785592, |
| | rs4785711, rs4785712, rs4785713, rs3803689, rs4785715, rs11355220, |
| | rs2003522, rs12445480, rs12445695, rs12923751, rs12922302, |
| | rs1800345, rs2377043, rs9926296, rs3743860, rs7205519, rs7203511, |
| | rs7206120, rs7206308, rs7205604, rs7206721, rs7205993, rs7195752, |
| | rs7201028, rs12709093, rs3819567, rs3819568, rs2238526, rs4785720, |
| | rs2376885, rs9806894, rs2159113, rs2376880, rs7498439, rs11860203, |
| | rs1010356, rs4785595, rs1800340, rs1007931, rs3785276, rs3785275, |
| | rs1006548, rs1006547, rs2016571, rs7198982, rs2239360, rs8054489, |
| | rs60828994, rs2238529, rs12709095, rs1968109, rs1476761, rs6500447, |
| | rs6500448, rs7200111, rs11076625, rs34665267, rs8044210, rs1057042, |
| | rs8049660, rs1800330, rs6500452, rs3856, rs2159227, rs8051231, |
| | rs12709096, rs9933901, rs4785722, rs8051915, rs9927381, rs9927395, |
| | rs4313809, rs11859183, rs11076631, rs9927089, rs78004870, |
| | rs72631431, rs4785723, rs7185897, rs11864372, rs4785727, rs1558183, |
| | rs9928396, rs1079558, rs11866688, rs75907499, rs4785731, rs57027421, |
| | rs11643251, rs8060934, rs6500459, rs6500460, rs7200842, rs55911767, |
| | rs1109334, rs9939914, rs55703708, rs28630471, rs538986066, |
| | rs557599094, rs149602789, rs11648301, rs11648648, rs151302653, |
| | rs28376048, rs550128311, rs553894401, rs573262348, rs534004067, |
| | rs558546384, rs112818457, rs575035566, rs542060861, rs560706700, |
| | rs527408418, rs552336012, rs564149910, rs531382163, rs549793936, |
| | rs568277185, rs7190567, rs72813442, rs72813445, rs3212349, |
| | rs7191759, rs7191944, rs60723967, rs4586434, rs4395073, rs56378598, |
| | rs4785741, rs4785745, rs7205758, rs7184956, rs4785749, rs62052219, |
| | rs561747125, rs559926519, rs3803687, rs3803686, rs62052238, |
| | rs8051733, rs62052243, rs8063761, rs13330431. |
| TP53 | rs11868624, rs62059238, rs8071870, rs28792688, rs62061162, |
| 17:7565097- | rs7219773, rs34140799, rs12948090, rs4796414, rs2269762, rs11653257, |
| 7590856 | rs12945656, rs9904883, rs968580, rs8075287, rs7214490, rs7216864, |
| 384 SNPs | rs62061168, rs62061169, rs9901523, rs74618501, rs56206519, |
| | rs8071437, rs55964017, rs9900153, rs11078674, rs3752031, rs11652328, |
| | rs12946520, rs34280588, rs4151120, rs4151121, rs2302767, rs12451961, |
| | rs67328311, rs12943744, rs12600861, rs12600863, rs2302762, |
| | rs3867593, rs35490807, rs55749333, rs12942267, rs60488855, |
| | rs4796307, rs55894190, rs4796423, rs8076791, rs8081271, rs8065577, |
| | rs11078680, rs9901161, rs6503025, rs3722, rs8074586, rs4354991, |
| | rs4796308, rs11651206, rs11650977, rs2871327, rs2111437, rs3867596, |
| | rs9906630, rs11652829, rs4627421, rs7217258, rs11078682, rs7225954, |
| | rs4407107, rs56226499, rs9303218, rs9890920, rs4632183, rs7225446, |
| | rs2277638, rs12936464, rs8075218, rs11658168, rs7215922, rs10153276, |
| | rs11078685, rs3826440, rs9889368, rs2269459, rs7213878, rs2269460, |
| | rs35868540, rs9901643, rs9901892, rs58868571, rs9675122, rs58742827, |
| | rs4468689, rs2111438, rs11078690, rs11653815, rs11078691, |
| | rs11078692, rs9908275, rs11656734, rs9916183, rs9630708, rs7213231, |
| | rs11658305, rs35061142, rs6503033, rs6503036, rs9907045, rs11078693, |
| | rs11870720, rs8078222, rs8073584, rs8075280, rs11654632, rs11654638, |
| | rs8073177, rs9902027, rs34445439, rs4968216, rs4968221, rs9912001, |
| | rs28672997, rs9892297, rs11656013, rs9900162, rs4968200, rs77294902, |
| | rs12940684, rs4511593, rs4968210, rs12937543, rs11078696, rs8068222, |
| | rs12939472, rs10438740, rs4602096, rs4968213, rs28625968, rs4968214, |
| | rs1142832, rs9897307, rs2270341, rs12942088, rs8066665, rs7211847, |
| | rs858527, rs858526, rs3933469, rs1641550, rs1642772, rs4968190, |
| | rs2270339, rs858524, rs858523, rs1641546, rs888110, rs34416693, |
| | rs858522, rs2541012, rs858521, rs858518, rs727428, rs1642795, |
| | rs1642796, rs1642797, rs12450254, rs11078702, rs1642798, rs1642800, |
| | rs1641543, rs1642802, rs1641542, rs12949514, rs1614027, rs1642803, |
| | rs1642804, rs1641541, rs1642809, rs1641539, rs1641538, rs1641537, |
| | rs1641536, rs1641535, rs2543554, rs1641530, rs1641529, rs1641528, |
| | rs62059859, rs2543555, rs1642811, rs1641525, rs2955613, rs2955612, |
| | rs2955611, rs2955610, rs1642812, rs1641524, rs1641523, rs1641522, |
| | rs1641521, rs1641520, rs1641519, rs1619016, rs2541010, rs1641518, |
| | rs1641517, rs1641516, rs1641515, rs12936842, rs1624085, rs1642764, |
| | rs1641510, rs8073498, rs9893249, rs12949853, rs4968204, rs1614984, |
| | rs1794284, rs12602273, rs2078486, rs11652704, rs35175270, |
| | rs17881035, rs17885845, rs17882227, rs2287497, rs4559962, rs8076771, |
| | rs35922565, rs12939910, rs11659090, rs12949541, rs12941981, |
| | rs56371490, rs9907011, rs62062589, rs9906502, rs67022015, |
| | rs11870307, rs60370790, rs34873228, rs4246412, rs3744259, rs3744258, |
| | rs62062594, rs307627, rs72829783, rs7213942, rs12451505, rs2543539, |
| | rs2642158, rs2543540, rs839721, rs12938680, rs62062620, rs7225101, |
| | rs11656201, rs7212989, rs9889453, rs34001984, rs9902382, rs9903543, |
| | rs8066124, rs11653849, rs11655108, rs11658349, rs9905906, rs8070826, |
| | rs2309810, rs4791329, rs9905612, rs11658635, rs12942745, rs1007448, |
| | rs4233018, rs1565817, rs12949280, rs4791834, rs8066334, rs6503062, |
| | rs9905830, rs9912616, rs9907759, rs872345, rs9916791, rs1106826, |
| | rs2279621, rs4791806, rs7406118, rs57828851, rs10852892, rs12603355, |
| | rs34121753, rs12945870, rs7208787, rs4130668, rs4791840, rs2270517, |
| | rs3736305, rs55809496, rs1565816, rs4290529, rs8070084, rs8074085, |
| | rs57885072, rs16957013, rs16957022, rs60450152, rs4265881, |
| | rs8078725, rs11078710, rs12937245, rs7223364, rs7406902, rs12450454, |
| | rs11078713, rs9889962, rs62062524, rs7222046, rs11078716, |
| | rs11654506, rs66593967, rs12451077, rs4444395, rs10852894, |
| | rs4396585, rs8070212, rs9908139, rs58442588, rs9898955, rs9911390, |
| | rs11656822, rs9899694, rs4239114, rs4239115, rs73235718, rs4791987, |
| | rs7224754, rs11078717, rs11078718, rs11652681, rs11650894, |
| | rs35395589, rs4300713, rs12950912, rs111793121, rs12601767, |
| | rs7207022, rs7209540, rs7207775, rs7209704, rs4791993, rs4239116, |
| | rs4462664, rs4473260, rs11654985, rs4791409, rs4791410, rs7226300, |
| | rs35195686, rs7211792, rs4792002, rs12601100, rs4239118, rs11654421, |
| | rs11650268, rs4491592, rs4239119, rs4239120, rs7212233, rs11655693, |
| | rs7225505, rs62062546, rs7211456, rs9894662, rs12951228, rs11078720, |
| | rs11650003, rs12185224, rs4792020, rs11078721, rs4791423, |
| | rs34212807, rs62062552. |
| CDK12 | rs60083830, rs634365, rs588193, rs587343, rs679995, rs585961, |
| 17:37617764- | rs677888, rs801420, rs801419, rs8079355, rs8066704, rs8072297, |
| 37721160 | rs7223331, rs9944411, rs7220650, rs8079560, rs2338800, rs9903363, |
| 243 SNPs | rs9909064, rs6503502, rs8069451, rs9894586, rs7214151, rs9892055, |
| | rs7208274, rs6503503, rs12935933, rs8065879, rs8076494, rs11869259, |
| | rs11869109, rs7223438, rs6503507, rs68178216, rs35546703, rs9898773, |
| | rs4511574, rs73293051, rs4795356, rs11078898, rs13341996, rs9896913, |
| | rs7210488, rs9892675, rs57399933, rs10491128, rs34432983, |
| | rs35322295, rs12600751, rs2338796, rs35285898, rs9905432, rs4795357, |
| | rs35667594, rs12938268, rs4795359, rs4795360, rs4795361, rs12452509, |
| | rs11650081, rs7501488, rs11870713, rs7405667, rs36064019, |
| | rs12150493, rs11868133, rs4410117, rs11651089, rs10445305, |
| | rs12452880, rs10445306, rs34363096, rs11867312, rs11659010, |
| | rs11654153, rs4794809, rs12947620, rs6503515, rs7212715, rs4795369, |
| | rs12943928, rs55722796, rs35112839, rs11078900, rs4390625, |
| | rs35497503, rs12950666, rs62075056, rs7219294, rs7223896, |
| | rs11078901, rs11657899, rs12947600, rs11078902, rs11078903, |
| | rs12453397, rs57448077, rs4286140, rs11650776, rs4462633, |
| | rs12948906, rs11870631, rs12452682, rs34957982, rs12942360, |
| | rs12453447, rs11489942, rs6503517, rs11868029, rs11868030, |
| | rs11078904, rs62077463, rs12940193, rs72827112, rs2018026, |
| | rs6503518, rs7224067, rs11078905, rs11078906, rs11078907, rs7225096, |
| | rs12936996, rs4795377, rs12952384, rs7503705, rs4794813, rs11078910, |
| | rs11078912, rs4795378, rs8067884, rs7406928, rs8065963, rs55660616, |
| | rs12450559, rs4239222, rs4794814, rs7503069, rs11655531, rs10852933, |
| | rs4795381, rs4795382, rs11654853, rs11654018, rs7219909, rs6503519, |
| | rs35335692, rs11078915, rs4795384, rs4357971, rs7503195, rs8075737, |
| | rs412430, rs1619021, rs12453198, rs12951665, rs9903269, rs12948096, |
| | rs11654954, rs4795386, rs74890597, rs4795388, rs12453682, rs1874228, |
| | rs33953566, rs4795390, rs879606, rs10852934, rs3751903, rs1495099, |
| | rs2271309, rs11651497, rs907094, rs3764353, rs3764352, rs3817160, |
| | rs4794817, rs9972882, rs881844, rs11869286, rs11658786, rs2271308, |
| | rs407307, rs2247862, rs903503, rs903502, rs2934956, rs1565922, |
| | rs10558975, rs1565920, rs1495102, rs1495101, rs1495100, rs2934953, |
| | rs2934952, rs2941505, rs2952152, rs2941506, rs2934951, rs907088, |
| | rs907089, rs907090, rs9675194, rs2313171, rs732083, rs12150298, |
| | rs12150603, rs8077172, rs8078228, rs11078919, rs1476278, rs9303274, |
| | rs12940986, rs2517957, rs2517958, rs903501, rs2517953, rs2517954, |
| | rs2517955, rs2517956, rs2517959, rs2517960, rs2904765, rs2904766, |
| | rs55717377, rs2904768, rs2643194, rs2517951, rs2643195, rs2934971, |
| | rs1565923, rs2952155, rs1810132, rs4252627, rs2934967, rs2952156, |
| | rs2952157, rs11653998, rs903506, rs4795393, rs2941519, rs9747973, |
| | rs2941522, rs12946510. |
| BRCA1 | rs9902963, rs692180, rs4349197, rs2593594, rs692198, rs709629, |
| 17:41196312- | rs323495, rs323494, rs225583, rs62079763, rs2667933, rs8067341, |
| 41277500 | rs691801, rs8081535, rs12451424, rs28678167, rs12950717, rs2298862, |
| 215 SNPs | rs2298861, rs12951869, rs9911630, rs11654452, rs4793189, rs11657053, |
| | rs7223952, rs11659028, rs8176323, rs8176322, rs8176314, rs4793190, |
| | rs8176296, rs4793191, rs4793192, rs8176290, rs7212284, rs8176289, |
| | rs372544924, rs8176282, rs8176279, rs4793193, rs35330014, rs8176268, |
| | rs3092994, rs3785546, rs4793194, rs8176234, rs8176233, rs8176231, |
| | rs8176220, rs273900734, rs2236762, rs8176198, rs8176193, rs3950989, |
| | rs8176171, rs8176161, rs8176160, rs2070834, rs799916, rs8176145, |
| | rs8176140, rs35908185, rs8176126, rs55737636, rs375673256, |
| | rs8176109, rs8176103, rs8176098, rs8176092, rs8176082, rs3765640, |
| | rs8176077, rs799906, rs11655505, rs799908, rs4793204, rs33925201, |
| | rs11653069, rs11651905, rs2175957, rs1554063, rs33988650, |
| | rs11656097, rs11658754, rs7223062, rs33968979, rs12951574, |
| | rs12952790, rs12950607, rs11079056, rs12936816, rs33926631, |
| | rs2037076, rs12937015, rs4340367, rs4792981, rs35521261, rs2137990, |
| | rs11652254, rs33946455, rs2271574, rs33994002, rs33920795, |
| | rs33918839, rs11653231, rs1399323, rs11655049, rs4793216, |
| | rs36062488, rs8070085, rs8074136, rs33911743, rs34234065, |
| | rs34142696, rs17527802, rs17527933, rs71379212, rs2356310, |
| | rs1565163, rs1807433, rs71367985, rs111274735, rs3744244, |
| | rs35960318, rs78845268, rs34093730, rs71379217, rs111241581, |
| | rs35332877, rs75566810, rs35312672, rs75467880, rs17600502, |
| | rs77940339, rs17600553, rs2356308, rs1137525, rs369484418, |
| | rs62079626, rs62079627, rs62079628, rs62079646, rs62079647, |
| | rs62079648, rs62079649, rs62079650, rs62079651, rs62079652, |
| | rs62079653, rs62079654, rs62079655, rs140964974, rs138298259, |
| | rs2005942, rs1574328, rs111535713, rs74252763, rs34053645, |
| | rs2736686, rs4793226, rs35788301, rs36035445, rs35993618, rs4356517, |
| | rs2343819, rs36114164, rs34576341, rs4793227, rs71190320, rs4346244, |
| | rs4600503, rs9675035, rs9675026, rs4520874, rs71379220, rs12951008, |
| | rs4793229, rs11649954, rs35956818, rs9646416, rs4584865, rs12940133, |
| | rs9646413, rs36042146, rs11651341, rs34572725, rs9897425, rs4534897, |
| | rs1842144, rs1842145, rs4793233, rs4793234, rs4793236, rs9904639, |
| | rs4792988, rs4792989, rs60958888, rs4793241, rs528193640, |
| | rs74735586, rs4793243, rs74847178, rs11657004, rs9912203, |
| | rs34474989, rs199735290, rs4793247, rs4792990, rs880690, rs9890479, |
| | rs12450913, rs4262987, rs4426385, rs1871338, rs753396. |
| SPOP | rs56366200, rs7212890, rs2197159, rs7208925, rs58988973, rs2233669, |
| 17:47676246- | rs935129, rs2233659, rs116953353, rs71379362, rs7222591, rs7209261, |
| 47755596 | rs7216547, rs4477768, rs9912738, rs7207617, rs8075138, rs8075428, |
| 226 SNPs | rs8079832, rs2165703, rs2119930, rs2119929, rs2584686, rs9913079, |
| | rs62077704, rs9889680, rs34312354, rs2671661, rs34686525, rs2671662, |
| | rs2584681, rs2671663, rs2584680, rs35790065, rs12936017, rs2671664, |
| | rs1867088, rs2671666, rs1814451, rs2671636, rs2671629, rs60276708, |
| | rs2165705, rs2584670, rs2584669, rs11079871, rs1319888, rs1316794, |
| | rs7216476, rs7215916, rs12941629, rs1899463, rs870419, rs870420, |
| | rs870421, rs2412102, rs871046, rs2671626, rs12936768, rs12449692, |
| | rs2671624, rs9894185, rs2671623, rs28663220, rs2001868, rs2584667, |
| | rs872638, rs1814452, rs885370, rs2671689, rs585916, rs1035050, |
| | rs2671687, rs2671686, rs2671685, rs2671684, rs489484, rs2537710, |
| | rs2671682, rs603769, rs11466117, rs4239164, rs2584653, rs9908234, |
| | rs600120, rs2584685, rs563205, rs565042, rs601910, rs3785931, |
| | rs614455, rs11466129, rs2072444, rs627905, rs657770, rs2584674, |
| | rs613495, rs534561, rs4257247, rs9914609, rs2671640, rs2458669, |
| | rs11653695, rs2537711, rs8081715, rs2537712, rs2671644, rs2671645, |
| | rs2671646, rs2671648, rs7217988, rs2671650, rs7217331, rs201097321, |
| | rs2537719, rs2537720, rs2458666, rs2430078, rs2537721, rs2458665, |
| | rs2537723, rs2671673, rs2537724, rs11649869, rs11868439, rs2537727, |
| | rs2041598, rs2041599, rs11653251, rs741074, rs55719413, rs11871859, |
| | rs2537728, rs11651407, rs759019, rs9807031, rs35198299, rs574750728, |
| | rs60307484, rs6504616, rs57267712, rs9893555, rs111474967, |
| | rs7224097, rs8067995, rs8066885, rs9900258, rs2676791, rs2537729, |
| | rs7209457, rs8079756, rs55988045, rs57066600, rs6504617, rs6504618, |
| | rs11079873, rs7219984, rs7221354, rs3815474, rs2537702, rs62079290, |
| | rs8072037, rs6504619, rs2008954, rs6504620, rs12451482, rs6504621, |
| | rs7210870, rs7211112, rs57508160, rs11870879, rs2044165, rs4997224, |
| | rs55817237, rs77467424, rs12601880, rs2017835, rs2304916, |
| | rs12449950, rs2120106, rs59917700, rs12601082, rs12949906, |
| | rs8080075, rs11079876, rs7211603, rs4793620, rs4793621, rs8081972, |
| | rs16948378, rs755736, rs12449890, rs4794066, rs1105547, rs10853112, |
| | rs8072700, rs60157068, rs4794068, rs62076793, rs35726347, rs271665, |
| | rs8064789, rs8082491, rs7218139, rs73337060, rs59946755, rs56678296, |
| | rs55733038, rs55901362, rs8079058, rs8080832, rs166613, rs73987397, |
| | rs8071305, rs12936839, rs8082024, rs73987401, rs60586860, rs271639, |
| | rs8077855, rs73337077, rs271641, rs4393614, rs7217156, rs8081882, |
| | rs8069785. |
| RNF43 | rs4793934, rs1075009, rs9908511, rs8071251, rs2052083, rs2052084, |
| 17:56429861- | rs8081942, rs8081708, rs3863503, rs1987665, rs3760172, rs57078284, |
| 56494956 | rs59179740, rs2052087, rs9894859, rs2109248, rs9896621, rs9904720, |
| 186 SNPs | rs887398, rs2191242, rs9902044, rs9889505, rs917606, rs113364181, |
| | rs9910092, rs9901765, rs11079340, rs10853004, rs12602498, rs3785496, |
| | rs9892223, rs34409274, rs8077426, rs68117086, rs3213698, rs3826300, |
| | rs3744105, rs7225148, rs62083740, rs2240263, rs8178290, rs7219860, |
| | rs7226197, rs8178307, rs7208924, rs7209537, rs11868650, rs7218620, |
| | rs7222295, rs8178406, rs8178407, rs8079006, rs11649901, rs2333227, |
| | rs2243828, rs2107545, rs4401102, rs12953025, rs10853005, rs12944679, |
| | rs16942914, rs9908667, rs2240261, rs11652685, rs11652816, |
| | rs36057095, rs2057807, rs12600886, rs2072144, rs2680706, rs2526378, |
| | rs2526377, rs2632527, rs1985749, rs12936976, rs2680690, rs1859401, |
| | rs757485, rs12938538, rs7207531, rs56058331, rs2526376, rs7222143, |
| | rs12452186, rs2680700, rs2680699, rs2680698, rs2632513, rs2680688, |
| | rs4793583, rs1811202, rs12601867, rs2680708, rs8070135, rs1990008, |
| | rs9788975, rs6503859, rs8067183, rs7211294, rs9904993, rs9889419, |
| | rs723991, rs58647683, rs9303398, rs1476596, rs7221205, rs2526370, |
| | rs10853006, rs2429369, rs72628392, rs7207286, rs2531725, rs2531726, |
| | rs2632508, rs717828, rs2531729, rs11656888, rs2531732, rs6503864, |
| | rs6503865, rs2531734, rs7221464, rs8065388, rs2270097, rs2270098, |
| | rs9901114, rs11650710, rs72628393, rs11650105, rs3809709, rs4793950, |
| | rs3744109, rs11079354, rs12449365, rs8066773, rs2079703, rs11870671, |
| | rs9901693, rs9903173, rs3744111, rs4793952, rs11869828, rs12939703, |
| | rs12453049, rs2009948, rs12942018, rs6503866, rs12453459, rs4793954, |
| | rs9909763, rs9889631, rs2333332, rs8081906, rs6503869, rs9902820, |
| | rs2013182, rs1974586, rs7226008, rs1267545, rs3803751, rs9893027, |
| | rs8068135, rs1267542, rs11658998, rs8075108, rs8071916, rs9907940, |
| | rs7218778, rs8072873, rs7213415, rs302840, rs9911609, rs7209650, |
| | rs391370, rs417005, rs7210714, rs437732, rs7209498, rs16943134, |
| | rs302854, rs302851, rs302850, rs302849, rs302847, rs302846, |
| | rs8076021. |
| RAD51C | rs302848, rs302859, rs302860, rs6503872, rs302861, rs9898626, |
| 17:56769934- | rs302863, rs2611781, rs368243, rs9904996, rs9900405, rs2611782, |
| 56811703 | rs575403436, rs16943147, rs79155614, rs113927892, rs302842, |
| 172 SNPs | rs7217890, rs2567912, rs11651859, rs1631237, rs2567911, rs199890493, |
| | rs540939868, rs191825334, rs654778, rs372648, rs4340382, rs11655607, |
| | rs2643124, rs7209327, rs9890924, rs304264, rs304263, rs302870, |
| | rs9909635, rs9899893, rs7221142, rs302878, rs302877, rs302874, |
| | rs302873, rs9916423, rs12938772, rs686425, rs405684, rs7208872, |
| | rs8071942, rs6503874, rs2567905, rs304280, rs73996262, rs28363312, |
| | rs2567894, rs9895162, rs76708174, rs28363318, rs304267, rs304268, |
| | rs16943186, rs304269, rs304270, rs17222733, rs9797290, rs77430462, |
| | rs78253932, rs62081324, rs9904234, rs172565, rs304262, rs73993803, |
| | rs7342862, rs8075786, rs7222789, rs304292, rs8074787, rs16943200, |
| | rs304297, rs170024, rs12450766, rs304296, rs8081074, rs304299, |
| | rs304298, rs6503877, rs9915935, rs12952429, rs7211439, rs9303401, |
| | rs11653599, rs7214266, rs999431, rs7213035, rs1494977, rs7212271, |
| | rs11079356, rs9904879, rs8067571, rs8068677, rs11871453, rs2029792, |
| | rs9904677, rs34102441, rs6503878, rs6503879, rs1390006, rs4793593, |
| | rs7217923, rs4482353, rs12602346, rs9897326, rs74961279, rs77326197, |
| | rs11870601, rs28605629, rs12952832, rs7209253, rs1018051, rs1472288, |
| | rs6503881, rs62083375, rs7342903, rs6503882, rs7342841, rs7223955, |
| | rs6503883, rs35277036, rs113815236, rs7214805, rs12940681, |
| | rs4453568, rs4076229, rs8080281, rs35082135, rs6503884, rs7215808, |
| | rs7219856, rs11079365, rs9902994, rs9907499, rs4337355, rs12944948, |
| | rs9911578, rs10401050, rs12940492, rs12938821, rs9901181, rs4636974, |
| | rs11659035, rs4398153, rs11650809, rs66526343, rs9892062, rs9894274, |
| | rs9911753, rs11652761, rs9901655, rs7216680, rs1588322, rs6503887, |
| | rs10853008, rs66954617, rs9894064, rs112012480, rs1971659, |
| | rs12232477, rs7216698, rs7221274, rs9900413, rs28709536, rs11079367, |
| | rs8071280. |
| AKT2 | rs234299, rs55798334, rs7249286, rs170900, rs163213, rs3000956, |
| 19:40736224- | rs8108793, rs337779, rs337799, rs337800, rs337801, rs186815, |
| 40791443 | rs186816, rs13344062, rs106082, rs2569656, rs2569652, rs2970098, |
| 229 SNPs | rs2569651, rs2569650, rs2767617, rs2569646, rs56385001, rs577514332, |
| | rs1836008, rs10420328, rs10402038, rs7257852, rs7246059, rs28890790, |
| | rs58003479, rs16974093, rs8103294, rs28629274, rs10401980, |
| | rs2163847, rs10406471, rs10413414, rs10415437, rs8110579, rs634520, |
| | rs8106007, rs533318, rs11881374, rs12985743, rs147604049, rs569233, |
| | rs596708, rs521783, rs598949, rs546453, rs1654569, rs610549, |
| | rs1654620, rs471793, rs656650, rs658295, rs1612768, rs557319, |
| | rs557320, rs558154, rs687136, rs687155, rs476922, rs594846, rs508531, |
| | rs509354, rs73042639, rs1654610, rs2356227, rs535840, rs590025, |
| | rs34594459, rs539198, rs1622683, rs592417, rs591986, rs591445, |
| | rs533015, rs530366, rs339512, rs339513, rs339514, rs339515, rs339516, |
| | rs339517, rs339518, rs647909, rs189281, rs339522, rs339523, rs339524, |
| | rs339525, rs339526, rs10402740, rs73042673, rs4530264, rs7258548, |
| | rs73042675, rs9749056, rs60568017, rs2356229, rs892117, rs34894858, |
| | rs3746006, rs55710849, rs79666308, rs7247201, rs12609014, rs4802070, |
| | rs12975651, rs7260249, rs4239504, rs934426, rs4490099, rs8113426, |
| | rs10853739, rs79746268, rs2304189, rs4803320, rs3730051, rs8100018, |
| | rs8104727, rs4802071, rs35562135, rs3730050, rs12460555, rs4558508, |
| | rs748236, rs10405207, rs969531, rs7260517, rs7250897, rs4332845, |
| | rs7259541, rs8102171, rs12459567, rs10416620, rs7254617, rs34716810, |
| | rs62107593, rs10419465, rs11673367, rs9304588, rs58502633, |
| | rs10426842, rs60025005, rs12974529, rs2368574, rs56655668, |
| | rs2368575, rs4239506, rs4803324, rs4803325, rs7508604, rs4479364, |
| | rs4524046, rs55710016, rs4257318, rs4337402, rs16974196, rs6508936, |
| | rs11668242, rs7255581, rs59263025, rs4803327, rs7257972, rs12151335, |
| | rs3745195, rs4802073, rs8102720, rs4802074, rs8104162, rs4502340, |
| | rs117356447, rs7249146, rs10414431, rs8110888, rs12460615, |
| | rs7260106, rs8106335, rs4490097, rs73046335, rs7507651, rs4803328, |
| | rs4802076, rs11083554, rs11672825, rs11083555, rs11670295, |
| | rs4803330, rs2278227, rs150385742, rs10407266, rs268666, rs268664, |
| | rs6508939, rs268662, rs268686, rs166539, rs4622626, rs67177566, |
| | rs7258277, rs268676, rs34033973, rs2007602, rs7248033, rs185593, |
| | rs268688, rs10418696, rs10421779, rs2169275, rs7257546, rs9749496, |
| | rs268693, rs57796734, rs28608147, rs12981511, rs4802078, rs2613843, |
| | rs10411033, rs12972755, rs7245732, rs4803342, rs4803343, rs4803344, |
| | rs12462773, rs10401458, rs10407864. |
| ERCC2 | rs10421247, rs12460975, rs2109065, rs2159324, rs10421129, |
| 19:45853095- | rs11083765, rs11667197, rs11671390, rs73034856, rs3848526, |
| 45874176 | rs11673520, rs631920, rs62118495, rs8100000, rs8102309, rs8102230, |
| 225 SNPs | rs56413312, rs10406257, rs632235, rs620807, rs8105802, rs8105818, |
| | rs606757, rs582747, rs595290, rs2627641, rs597668, rs4803806, |
| | rs598183, rs7248787, rs10415550, rs10415983, rs10416346, rs10417555, |
| | rs10405928, rs12461138, rs12461144, rs346751, rs8109472, rs346750, |
| | rs346747, rs346746, rs35569326, rs67915086, rs346745, rs368254, |
| | rs346741, rs346742, rs12981145, rs12976518, rs12985921, rs8110992, |
| | rs8111118, rs6509202, rs6509203, rs3810365, rs3826910, rs6509204, |
| | rs12981324, rs8105501, rs10445572, rs345409, rs386569, rs441728, |
| | rs11883302, rs8106469, rs344810, rs12460848, rs11083768, rs11666411, |
| | rs344809, rs344807, rs344806, rs2098088, rs11672894, rs344802, |
| | rs11667235, rs344801, rs8111989, rs2377326, rs7260463, rs7260359, |
| | rs10402747, rs1967310, rs1967311, rs12984728, rs10411862, |
| | rs12983935, rs7251748, rs12976956, rs344819, rs6509208, rs344815, |
| | rs344791, rs344790, rs123187, rs12973246, rs12973496, rs1793978, |
| | rs7252037, rs9304647, rs10404025, rs10404465, rs454421, rs12462541, |
| | rs10416849, rs55990241, rs74915, rs12609631, rs10853773, rs238418, |
| | rs238416, rs238415, rs238414, rs106433, rs238411, rs50871, rs171140, |
| | rs238403, rs238404, rs238405, rs238407, rs3916804, rs8109829, |
| | rs1618536, rs1799783, rs3810366, rs2097215, rs11878644, rs112536753, |
| | rs7252567, rs10422489, rs8103601, rs4544343, rs8101662, rs61177445, |
| | rs35544084, rs2017104, rs28670042, rs11673653, rs10401973, |
| | rs10402584, rs4572514, rs7507578, rs4803816, rs959457, rs2336218, |
| | rs4803817, rs10412761, rs1005165, rs8113779, rs3212980, rs3212971, |
| | rs3212969, rs3212964, rs3212955, rs58106854, rs61390465, rs7259762, |
| | rs7247671, rs10408989, rs28478677, rs35537738, rs4803823, rs4802257, |
| | rs12610932, rs12610945, rs10405048, rs6509213, rs7251807, rs8109532, |
| | rs7250092, rs10415949, rs28503834, rs8106228, rs10413123, |
| | rs34364691, rs10402243, rs3760845, rs2282696, rs11883164, rs7259071, |
| | rs8106229, rs8106233, rs10417603, rs7256242, rs8103655, rs6509214, |
| | rs7245548, rs3038784, rs7254043, rs6509216, rs7250901, rs57125828, |
| | rs34013829, rs11879629, rs11882534, rs4511641, rs35025952, |
| | rs1573606, rs10421736, rs8110916, rs4625825, rs758761, rs4802259, |
| | rs12609861, rs12609862, rs8099878, rs12978806, rs2239378, rs2301284, |
| | rs11880111, rs62109648, rs12971437, rs16979873, rs8111589, |
| | rs10439108, rs7251522, rs12971754, rs28506127, rs12978460, |
| | rs12979939, rs11672063, rs8112944, rs8108463. |
| ERCC1 | rs377993, rs11673516, rs1264226, rs1264229, rs6509222, rs6509223, |
| 19:45910591- | rs7256951, rs1079273, rs1079272, rs1079271, rs1079270, rs7258777, |
| 45982086 | rs7259298, rs7253109, rs10409728, rs2334252, rs10406701, rs10414464, |
| 71 SNPs | rs10411821, rs10405576, rs56038031, rs10415062, rs59063421, |
| | rs7252658, rs2334253, rs2060245, rs8102440, rs12386125, rs10853775, |
| | rs10775545, rs6509226, rs6509227, rs147260403, rs10426693, |
| | rs35816837, rs10420309, rs58754078, rs12463090, rs4803839, |
| | rs4802263, rs4802264, rs28433019, rs28473282, rs7251629, rs12982191, |
| | rs8106394, rs6509230, rs62111720, rs62111721, rs55914970, |
| | rs10408179, rs10406431, rs10407429, rs7507893, rs7507912, rs6509231, |
| | rs8108269, rs10415769, rs10421206, rs10420680, rs10421259, |
| | rs10421566, rs10422601, rs7258890, rs71352280, rs55698417, |
| | rs58069157, rs55864746, rs35845603, rs10411648, rs2238689. |
| MYCN | rs7605046, rs10929387, rs2112100, rs56243489, rs200886610, |
| 2:16080686- | rs58084337, rs74892825, rs72784916, rs60082005, rs2380674, |
| 16087129 | rs2380675, rs3954898, rs7587082, rs10929390, rs10166110, rs10189998, |
| 261 SNPs | rs4668990, rs7604003, rs9678924, rs7569101, rs61226177, rs7583876, |
| | rs4668993, rs7586898, rs13405989, rs62115371, rs7595922, rs11900348, |
| | rs7582501, rs10165926, rs4668469, rs13382914, rs13397723, |
| | rs55705810, rs35962638, rs56003245, rs61557349, rs2380701, |
| | rs2380702, rs7591594, rs12613661, rs12613665, rs12613688, rs2380703, |
| | rs4668470, rs4668471, rs4669005, rs12993108, rs12615803, rs12612477, |
| | rs2380705, rs2380706, rs12613420, rs12619745, rs10495645, |
| | rs10495646, rs11691204, rs13015937, rs57542943, rs56848490, |
| | rs2890493, rs4669006, rs10929394, rs7563259, rs7602146, rs10929395, |
| | rs6725714, rs6754791, rs6726202, rs6726347, rs2380684, rs6740046, |
| | rs2890496, rs4668473, rs58006036, rs7602028, rs35740324, rs4474853, |
| | rs2559064, rs12611818, rs2693012, rs2559069, rs2693013, rs2254328, |
| | rs2559070, rs1820599, rs2693017, rs2693006, rs7594782, rs2693005, |
| | rs2462780, rs9751485, rs2559052, rs2559076, rs2559075, rs2559074, |
| | rs2559073, rs1974997, rs934050, rs13009427, rs11883561, rs11691519, |
| | rs10495648, rs1863803, rs1863804, rs10210656, rs12692281, |
| | rs12692283, rs12986946, rs10173329, rs10929400, rs57982553, |
| | rs9653226, rs3755135, rs79223268, rs10205809, rs55912884, |
| | rs12619709, rs4669018, rs7577066, rs35447596, rs36022555, |
| | rs12472968, rs12474047, rs6431741, rs6431742, rs4669019, rs2380711, |
| | rs12621288, rs6750986, rs1344662, rs6707485, rs1344660, rs1344661, |
| | rs6716344, rs12995948, rs13019419, rs6755683, rs6741005, rs10803676, |
| | rs11685620, rs11903230, rs71447244, rs6751458, rs6727137, rs1863806, |
| | rs7588144, rs67929574, rs10929405, rs11681121, rs9287666, rs6709176, |
| | rs11677992, rs55976064, rs56396163, rs55741908, rs56073739, |
| | rs13399600, rs13399605, rs13402547, rs55761643, rs13405836, |
| | rs6748658, rs6707051, rs10172827, rs1365966, rs1365967, rs1365968, |
| | rs61052094, rs10207969, rs55738104, rs11688817, rs7575773, |
| | rs34745530, rs16863197, rs6735335, rs35969199, rs934053, rs6744354, |
| | rs13415019, rs6716719, rs6760769, rs11896089, rs4669032, rs6431753, |
| | rs7579022, rs10186238, rs13405617, rs7609269, rs7609375, rs7607705, |
| | rs7605722, rs6719864, rs6704613, rs13427501, rs7600440, rs4499395, |
| | rs6760942, rs1427682, rs28774402, rs28725395, rs4073262, rs4073263, |
| | rs28668345, rs34080837, rs28658576, rs28565173, rs28469569, |
| | rs28592961, rs34073093, rs28624646, rs28704916, rs28704654, |
| | rs28504413, rs28413818, rs35778097, rs28685264, rs28541983, |
| | rs28697118, rs28754708, rs3924087, rs28620529, rs28722647, |
| | rs28716566, rs28406284, rs28472365, rs28689489, rs3924151, |
| | rs3924150, rs4669037, rs28403285, rs4668481, rs28672374, rs56931028, |
| | rs114203506, rs12467165, rs12471506, rs28582644, rs4669039, |
| | rs7566405, rs28368975, rs28613316, rs4668482, rs4668483, rs28464632, |
| | rs28416554, rs7604071, rs28430871, rs28680411, rs56080621, |
| | rs55945930, rs62118716, rs62118717, rs60772479, rs28549395, |
| | rs61728643, rs61725044, rs375154350, rs138599929, rs141498886, |
| | rs36189325. |
| MSH2 | rs61371951, rs1021610, rs1021609, rs10191588, rs74453940, rs2132923, |
| 2:47630108- | rs904166, rs13401365, rs7592268, rs6733907, rs11899692, rs11894063, |
| 47789450 | rs12712990, rs35265088, rs12712991, rs1995110, rs6704539, rs4953480, |
| 331 SNPs | rs12620038, rs10209637, rs4953481, rs12468717, rs12478003, |
| | rs10172689, rs10186190, rs10209762, rs10210258, rs2036164, |
| | rs9752054, rs9798303, rs183185556, rs150024302, rs931639, |
| | rs11894768, rs11900317, rs6751203, rs6738381, rs6738753, rs17036412, |
| | rs10190217, rs11896275, rs6755555, rs6727256, rs13426634, |
| | rs11125130, rs2055224, rs2055225, rs2055226, rs12614265, rs904159, |
| | rs11125132, rs7565161, rs967436, rs7355664, rs6544973, rs6544974, |
| | rs6544975, rs7568590, rs7576935, rs1979774, rs904161, rs904162, |
| | rs11898296, rs12988246, rs67898779, rs11894871, rs7599772, rs904165, |
| | rs904164, rs17558904, rs17499838, rs62138411, rs56168461, |
| | rs57259666, rs56146386, rs4471930, rs4290703, rs4401255, rs4395290, |
| | rs4374418, rs12463530, rs2036161, rs57409646, rs55656943, |
| | rs10178343, rs4953492, rs4953493, rs10200739, rs10188537, rs7604511, |
| | rs10196777, rs10172899, rs4996081, rs6751832, rs6712843, rs10439428, |
| | rs55941733, rs112874727, rs10202878, rs6726678, rs7564838, |
| | rs4399763, rs4614976, rs113586794, rs10185866, rs10173799, |
| | rs10197484, rs10195858, rs7584256, rs3924917, rs113967029, |
| | rs6757310, rs1863333, rs3815865, rs10179950, rs2162123, rs2162122, |
| | rs11889077, rs2347794, rs3771272, rs3771273, rs11125137, rs11125138, |
| | rs3771274, rs7588420, rs6726691, rs6726832, rs7607076, rs7607312, |
| | rs1981928, rs7602094, rs6739590, rs2347795, rs4596025, rs3771275, |
| | rs12712993, rs10194410, rs147078654, rs6544989, rs3771276, |
| | rs6707467, rs2162121, rs6758327, rs6724382, rs6724876, rs6757035, |
| | rs6729015, rs6544990, rs7586575, rs6711675, rs3771279, rs6748435, |
| | rs3771281, rs10179452, rs7565513, rs3732182, rs3732183, rs6754473, |
| | rs7596629, rs570806250, rs3764959, rs3764960, rs10175959, rs3821227, |
| | rs2059520, rs4583514, rs11886591, rs11694689, rs11684737, |
| | rs11684661, rs10188090, rs6544992, rs10209369, rs6759990, |
| | rs12991125, rs10206575, rs7599880, rs10175972, rs7585174, |
| | rs12712994, rs13031342, rs13031209, rs11901645, rs11891189, |
| | rs6746426, rs6718364, rs876937, rs879722, rs9784092, rs7587143, |
| | rs6744097, rs11125142, rs10495945, rs11896864, rs11675417, |
| | rs6751501, rs6722911, rs7606984, rs17502941, rs34713419, rs6544994, |
| | rs748780, rs7572456, rs6749075, rs13420102, rs6544995, rs6736039, |
| | rs12476727, rs1425610, rs1882446, rs1559713, rs1425613, rs4953503, |
| | rs1065650, rs7594890, rs7594899, rs6712595, rs7568787, rs7609322, |
| | rs6712789, rs6720323, rs11686238, rs113070032, rs4952891, rs2114023, |
| | rs11125145, rs4953509, rs4953510, rs4521097, rs10153687, rs56304087, |
| | rs1425612, rs10169431, rs6756180, rs10166990, rs7587517, rs13422917, |
| | rs4953513, rs6742817, rs6758037, rs1425611, rs2348096, rs11694783, |
| | rs17036703, rs752836, rs6754404, rs4953514, rs10198305, rs6705670, |
| | rs6709064, rs1863336, rs4953516, rs13403625, rs10182660, rs7566192, |
| | rs7605412, rs11681403, rs11682666, rs6716883, rs6544997, rs6723656, |
| | rs11125147, rs10172010, rs4953517, rs6705888, rs10199968, rs6705302, |
| | rs10153534, rs10166155, rs55934410, rs34897631, rs1317855, |
| | rs1317808, rs11892597, rs7559733, rs4952894, rs7563740, rs58310154, |
| | rs34472798, rs1922042, rs6716984, rs34682119, rs2881829, rs34662280, |
| | rs4266057, rs35141167, rs72815875, rs13391328, rs12712995, |
| | rs12712996, rs7596451, rs1574378, rs1574380, rs2348097, rs11685572, |
| | rs13426166, rs13413097, rs17570215, rs17570243, rs11125149, |
| | rs35102838, rs6712934, rs17036800, rs2969775, rs6722724, rs6729891, |
| | rs2677266, rs2944780, rs2969774, rs2952372, rs2677268, rs2952373, |
| | rs2952374, rs12479228, rs2952376, rs9309149, rs11688709, rs7603494, |
| | rs3844582, rs9789342, rs9789343, rs2348238, rs797703, rs797699, |
| | rs797683, rs810016, rs330795, rs330789, rs2969769, rs406334, |
| | rs7575752. |
| MSH6 | rs6723417, rs2348239, rs2348240, rs4358172, rs2881861, rs2881862, |
| 2:47922669- | rs4358173, rs4325805, rs4588237, rs66735853, rs61637835, rs4953522, |
| 48037240 | rs797702, rs797701, rs797700, rs797695, rs797694, rs797693, rs797692, |
| 87 SNPs | rs7594412, rs797691, rs797690, rs797689, rs797687, rs797686, |
| | rs797685, rs6545003, rs330794, rs229968, rs441327, rs3136233, |
| | rs3136302, rs1800934, rs3136329, rs2020911, rs3136354, rs7562048, |
| | rs330787, rs2651766, rs56101865, rs960106, rs12712997, rs10182633, |
| | rs874869, rs6742037, rs72811519, rs12620679, rs28625843, |
| | rs115477629, rs13008929, rs6545005, rs12105520, rs4952897, |
| | rs4583515, rs1962478, rs13382470, rs10171476, rs62141260, |
| | rs67890335, rs58112900, rs10168281, rs13392488, rs6716700, |
| | rs6720883, rs6545008, rs4254549, rs4277571, rs11695102, rs12620652, |
| | rs1483221, rs4952901, rs7600005, rs62141276, rs976416, rs1483219, |
| | rs7570621, rs6740753, rs7558634, rs11125159, rs4953538, rs4952902, |
| | rs4953541, rs10205889, rs7562885, rs13422058, rs11896243, rs1119024. |
| ERCC3 | rs2276582, rs880436, rs72846676, rs17014851, rs2071268, rs72481904, |
| 2:128014866- | rs75308426, rs66513437, rs744887, rs3768860, rs3768859, rs3820757, |
| 128051752 | rs752780, rs753430, rs753429, rs7562227, rs2164871, rs17014938, |
| 248 SNPs | rs6708935, rs10929006, rs6709706, rs6706041, rs6709337, rs4662705, |
| | rs4663094, rs6718147, rs55678466, rs11695424, rs11678252, |
| | rs11695710, rs13403184, rs13388170, rs12617750, rs13019394, |
| | rs746979, rs2404527, rs734115, rs873600, rs749008, rs755639, |
| | rs754107, rs6431219, rs11679418, rs730471, rs730294, rs13032148, |
| | rs10189896, rs6759813, rs13416037, rs11693218, rs7602810, rs3856378, |
| | rs7601252, rs7560621, rs11675682, rs10929008, rs10167189, rs6431221, |
| | rs58682665, rs719054, rs13025717, rs13025765, rs12618593, |
| | rs12617835, rs6740232, rs4663101, rs4663102, rs4663103, rs4663105, |
| | rs6733839, rs730482, rs744373, rs1866235, rs1469980, rs6759089, |
| | rs6759460, rs2118506, rs1875803, rs4663106, rs10208701, rs10208635, |
| | rs2118507, rs10208217, rs6431224, rs6722345, rs6722347, rs2896885, |
| | rs7566639, rs56401631, rs76271076, rs6706970, rs6710449, rs6720233, |
| | rs11681085, rs4663107, rs11682330, rs4663110, rs4663111, rs1875801, |
| | rs1875800, rs7587461, rs7587554, rs6751149, rs905657, rs6431231, |
| | rs2077309, rs7576123, rs7605733, rs4663113, rs4662707, rs4662708, |
| | rs34689155, rs7564541, rs4413109, rs11694404, rs59128670, |
| | rs58255944, rs17887116, rs13035268, rs13015329, rs7574851, |
| | rs7600500, rs13010572, rs35577992, rs12468441, rs2404535, |
| | rs59318157, rs885276, rs885275, rs2089109, rs12476709, rs12477559, |
| | rs12477653, rs13001179, rs13001184, rs13006847, rs67996371, |
| | rs4273176, rs59038595, rs7585198, rs2896888, rs4580338, rs4662710, |
| | rs9636236, rs1566822, rs3902960, rs4662711, rs201535044, rs4662714, |
| | rs6712551, rs35328149, rs1604817, rs10209678, rs12623948, |
| | rs58475809, rs10928761, rs2174270, rs6430935, rs12465955, rs4662717, |
| | rs2276583, rs1566823, rs4662575, rs11886231, rs4150496, rs4150495, |
| | rs4150477, rs4150454, rs4150441, rs3768866, rs55998967, rs4150407, |
| | rs528914855, rs12463451, rs62157549, rs6731176, rs6761447, |
| | rs9636237, rs12622436, rs11890187, rs6732279, rs17015199, rs4662722, |
| | rs4662580, rs62157555, rs7568261, rs12479003, rs34832797, |
| | rs12991062, rs12477214, rs7589451, rs10803585, rs2090574, |
| | rs12052483, rs9789510, rs2896980, rs4662725, rs12989081, rs79789049, |
| | rs12991768, rs13029237, rs62160261, rs6761391, rs7580658, |
| | rs61185143, rs6753288, rs4662726, rs2069901, rs1799809, rs1158867, |
| | rs2069910, rs2069912, rs2069914, rs2069915, rs2069920, rs2069921, |
| | rs973760, rs971207, rs1518759, rs2069924, rs2069931, rs2069933, |
| | rs1401296, rs6728362, rs6710535, rs13001648, rs12468304, rs1967425, |
| | rs6754772, rs6754999, rs6755028, rs1473623, rs6749002, rs12478656, |
| | rs12476767, rs12463538, rs13408910, rs12151780, rs13004942, |
| | rs62160290, rs12464295, rs34039606, rs4662732, rs334135, rs11694581, |
| | rs3732207, rs12475191, rs12476558. |
| NFE2L2 | rs12465125, rs12465162, rs13012236, rs4894234, rs6755239, rs6755240, |
| 2:178092323- | rs185189885, rs62173644, rs62173645, rs4894236, rs6712636, |
| 178257425 | rs16864877, rs1529093, rs1529098, rs6706601, rs10193787, rs7588467, |
| 243 SNPs | rs1529096, rs62173650, rs9789353, rs11674536, rs62173652, |
| | rs57778825, rs11676048, rs12693110, rs17842283, rs10176401, |
| | rs6705298, rs12105278, rs1358622, rs1406571, rs1406572, rs1534681, |
| | rs1554872, rs1529089, rs10497466, rs4894238, rs1529091, rs4893926, |
| | rs7578265, rs7592485, rs7578683, rs6433639, rs2056776, rs1529094, |
| | rs10166245, rs1373501, rs1373503, rs7578493, rs4894241, rs6433640, |
| | rs10930776, rs1444408, rs1444409, rs1444410, rs1348544, rs12467538, |
| | rs1444415, rs2364107, rs10930778, rs12693111, rs4368367, rs6433641, |
| | rs1822692, rs1373499, rs1373500, rs6433642, rs10192991, rs4894243, |
| | rs6736906, rs6708177, rs7598472, rs1444414, rs4894246, rs981584, |
| | rs7582291, rs7574502, rs10197258, rs6706797, rs13417670, rs7559961, |
| | rs2364712, rs2588873, rs13005039, rs13005606, rs2706129, rs2706131, |
| | rs2706133, rs2588888, rs3113786, rs10930780, rs7368646, rs3109083, |
| | rs2588875, rs2247763, rs2247754, rs2247446, rs2706112, rs2706110, |
| | rs2364717, rs6726395, rs10803905, rs4243387, rs2001297, rs13001694, |
| | rs13028464, rs13005431, rs12471433, rs12185576, rs2364722, |
| | rs2364723, rs34009885, rs2886161, rs7564947, rs35652124, rs4893819, |
| | rs11901211, rs2364724, rs2364725, rs2886162, rs6433657, rs7583487, |
| | rs7557529, rs7598494, rs12476773, rs2001530, rs2364729, rs2364730, |
| | rs35955110, rs4132130, rs4132129, rs6716391, rs1986691, rs3731801, |
| | rs1806686, rs2001349, rs6433658, rs6713305, rs2364731, rs13022567, |
| | rs12474344, rs2602533, rs12990594, rs13025552, rs11688684, |
| | rs2069043, rs12476383, rs13030571, rs146141734, rs78677544, |
| | rs35635511, rs200573114, rs112923128, rs1453373, rs2034678, |
| | rs12620035, rs1901826, rs1901825, rs1901824, rs10176632, rs1453371, |
| | rs4893931, rs4893821, rs4893935, rs4893936, rs10170546, rs7573763, |
| | rs4893938, rs7591807, rs12104588, rs3813259, rs75750814, rs4893941, |
| | rs13027959, rs7586051, rs11893448, rs2060929, rs12693120, |
| | rs12996447, rs4243388, rs4893822, rs4893823, rs4893944, rs4893825, |
| | rs4893950, rs952746, rs4893951, rs7587604, rs12616443, rs3902850, |
| | rs2121430, rs7565022, rs10803909, rs1979058, rs538506852, rs1453370, |
| | rs9789582, rs2084233, rs10170667, rs10194648, rs13000048, rs6742951, |
| | rs6433670, rs1348849, rs1348848, rs12469386, rs1374437, rs2197611, |
| | rs10497503, rs2166558, rs6710575, rs2364854, rs4893957, rs7604673, |
| | rs13004955, rs4893959, rs7606235, rs4893961, rs1348850, rs4893962, |
| | rs1975543, rs10165142, rs7571386, rs10199016, rs546331412, |
| | rs10198869, rs3927978, rs10164417, rs10930798, rs4893964, |
| | rs12471741, rs12463893, rs11896145, rs12622298, rs6761593, |
| | rs7559521, rs714598, rs13027299, rs2166554, rs11689550, rs11684320, |
| | rs13013752, rs12991272. |
| ASXL1 | rs6061195, rs4911553, rs6061197, rs6061198, rs2281364, rs6061203, |
| 20:30946155- | rs4589835, rs2377416, rs6058541, rs6061206, rs67703205, rs6119771, |
| 31027122 | rs6142632, rs3746604, rs6121381, rs733320, rs3787371, rs3787372, |
| 253 SNPs | rs6087835, rs11905172, rs6141636, rs6089216, rs6058556, rs3803989, |
| | rs3803990, rs6119776, rs6141639, rs6119777, rs6087839, rs6119778, |
| | rs6119784, rs3818219, rs1884866, rs1884865, rs6119786, rs6141277, |
| | rs6087845, rs6119792, rs113844904, rs2377472, rs6087848, rs6058597, |
| | rs4911087, rs4911208, rs4243971, rs6119230, rs2377473, rs6141651, |
| | rs7360540, rs6058603, rs6141652, rs6119802, rs4413223, rs4911088, |
| | rs62208761, rs1984792, rs6087862, rs6141659, rs35290636, rs3787373, |
| | rs56930149, rs6141282, rs3787374, rs6141662, rs6141285, rs6141666, |
| | rs6141668, rs6141670, rs2145245, rs6141671, rs4911216, rs4911217, |
| | rs56012551, rs56366536, rs13043343, rs6141289, rs6141675, rs2024952, |
| | rs3746606, rs933004, rs1028563, rs1022685, rs2027030, rs6141680, |
| | rs6087884, rs966979, rs6141685, rs6141687, rs1412997, rs1412998, |
| | rs2208131, rs11699120, rs6087887, rs6141689, rs2424876, rs6141294, |
| | rs2295454, rs6141295, rs9798511, rs4911223, rs78610212, rs6141297, |
| | rs2889646, rs6057567, rs2424880, rs2424881, rs6141695, rs6141696, |
| | rs6141697, rs6141299, rs6058669, rs3861818, rs8126139, rs4575791, |
| | rs2424882, rs8124037, rs2424883, rs6141302, rs2424884, rs2424885, |
| | rs2424886, rs13043838, rs201248496, rs6141304, rs55950726, |
| | rs55686374, rs55935733, rs6141706, rs6141707, rs62206927, rs6141714, |
| | rs6141306, rs6141307, rs369632590, rs6141308, rs6141309, rs17270685, |
| | rs6141716, rs4911229, rs2281542, rs3818190, rs4911093, rs6141720, |
| | rs1885292, rs1737896, rs13038256, rs13043144, rs6141721, rs1040751, |
| | rs1040752, rs4911232, rs1467479, rs3746612, rs6141310, rs6141723, |
| | rs2236155, rs6141311, rs1742981, rs1737890, rs1555132, rs2424888, |
| | rs1555133, rs6119248, rs17356521, rs1737893, rs34310097, rs2143534, |
| | rs4911234, rs1742983, rs12480462, rs293534, rs2247837, rs56377832, |
| | rs74651974, rs293536, rs13044256, rs6141734, rs293543, rs293544, |
| | rs4911235, rs293549, rs293550, rs293553, rs293554, rs293558, |
| | rs293559, rs293561, rs875519, rs6141314, rs296472, rs67918653, |
| | rs910889, rs6119879, rs6119880, rs6141741, rs6141743, rs159058, |
| | rs3746614, rs6141745, rs910890, rs733852, rs1868385, rs910891, |
| | rs12625036, rs879829, rs6141746, rs4911097, rs4911237, rs4911238, |
| | rs4911241, rs6119897, rs911529, rs7268588, rs6141752, rs6141753, |
| | rs6119904, rs1535374, rs1535375, rs6141755, rs10875486, rs911527, |
| | rs7267720, rs6119909, rs6058746, rs34627022, rs1555445, rs6141759, |
| | rs4911102, rs6087396, rs7266401, rs7266642, rs6057602, rs6058753, |
| | rs6087398, rs6087945, rs2005269, rs2889678, rs6058755, rs59609538, |
| | rs6087951, rs456798, rs6087953, rs59107435, rs463402, rs4911248, |
| | rs4911249, rs4911250, rs6087956, rs750396, rs4911104, rs6141766, |
| | rs6141767, rs6141328. |
| AURKA | rs6024649, rs6014619, rs4811657, rs6127659, rs4811659, rs6024653, |
| 20:54944445- | rs6024654, rs6024655, rs6099024, rs6069630, rs2870717, rs2870718, |
| 54967393 | rs6024656, rs2870719, rs6024661, rs4811661, rs11905467, rs6024668, |
| 258 SNPs | rs6069633, rs6069634, rs981872, rs6092262, rs6099030, rs6099031, |
| | rs6064348, rs6024672, rs4811664, rs6014629, rs6014630, rs6069638, |
| | rs1926078, rs1926080, rs6024680, rs6024681, rs6024682, rs6024685, |
| | rs6069639, rs6069640, rs6069641, rs6064350, rs1326010, rs1326009, |
| | rs1326008, rs6024688, rs6092267, rs6024692, rs2870720, rs4303767, |
| | rs6069643, rs8118536, rs6024700, rs6099036, rs3920612, rs2209595, |
| | rs957505, rs1590554, rs984257, rs6099038, rs6069648, rs10485804, |
| | rs6024717, rs6123559, rs6127688, rs4811684, rs6024725, rs6069669, |
| | rs11086538, rs4627642, rs6014645, rs6092275, rs6014646, rs16979603, |
| | rs6014647, rs6024731, rs6014648, rs6123567, rs6092276, rs6127698, |
| | rs6099056, rs6099057, rs6099058, rs6127700, rs6127701, rs6099060, |
| | rs6069674, rs1926065, rs12106274, rs6064361, rs1409224, rs1326022, |
| | rs1326021, rs6069681, rs6064362, rs6099065, rs2104474, rs2094183, |
| | rs115828762, rs62645516, rs201223517, rs116769050, rs202100873, |
| | rs200500234, rs202039313, rs2426605, rs6014678, rs6123579, |
| | rs6064375, rs6014682, rs6024784, rs1926071, rs2148218, rs2182358, |
| | rs6024787, rs6014684, rs6014685, rs11698420, rs2209593, rs6014688, |
| | rs6024802, rs6014691, rs6099092, rs7271106, rs988166, rs988167, |
| | rs6024807, rs6024810, rs6014699, rs6024813, rs6024817, rs6024818, |
| | rs6014704, rs6024820, rs6099108, rs6024821, rs6014706, rs67718983, |
| | rs6024830, rs6024831, rs6014708, rs6024836, rs370429433, rs8117896, |
| | rs6024840, rs911160, rs2180691, rs28403621, rs6069724, rs6069726, |
| | rs6069730, rs6014718, rs6024860, rs911163, rs6127748, rs3970972, |
| | rs6014730, rs3787390, rs6024879, rs6069753, rs442481, rs376900, |
| | rs1884910, rs6024888, rs386274, rs3787393, rs2870739, rs2870740, |
| | rs2870741, rs4811695, rs6099152, rs6099153, rs6099154, rs6099155, |
| | rs6099156, rs6014735, rs1570044, rs4514943, rs61622546, rs6127756, |
| | rs6024893, rs35636253, rs6024894, rs4811698, rs4811699, rs4811700, |
| | rs4811701, rs4811702, rs6064397, rs3761208, rs3761209, rs6014736, |
| | rs6099159, rs6099160, rs6014738, rs3761210, rs3761211, rs6024897, |
| | rs6024898, rs6024900, rs6024901, rs6099161, rs6123595, rs56104586, |
| | rs6092320, rs6014749, rs4811708, rs8125425, rs62210663, rs732281, |
| | rs4810021, rs6064404, rs4811709, rs6014753, rs2144609, rs3827108, |
| | rs6024913, rs6092324, rs6069767, rs2056, rs4810024, rs4810025, |
| | rs6099184, rs6024927, rs28620942, rs6069770, rs6099186, rs6099187, |
| | rs932530, rs6127783, rs6127784, rs6127786, rs6064411, rs6099191, |
| | rs6099193, rs6099194, rs73614317, rs6099195, rs6099196, rs6099197, |
| | rs6123604, rs11086550, rs6092328, rs869261, rs1853485, rs6092329, |
| | rs6127788, rs1884424, rs4811713, rs6099199, rs6099200, rs35671940, |
| | rs4811715, rs8124927, rs6127792, rs6014763, rs7267900, rs6064422, |
| | rs6024978, rs1886052. |
| GNAS | rs218465, rs218466, rs218468, rs218469, rs218472, rs218475, rs218476, |
| 20:57414773- | rs218477, rs218478, rs6026436, rs6100156, rs6100158, rs6123818, |
| 57486247 | rs6123819, rs6128398, rs2182249, rs2031538, rs6100163, rs6026444, |
| 244 SNPs | rs6100164, rs375018050, rs6123822, rs6100166, rs7362703, rs62203764, |
| | rs6015340, rs6128405, rs6092684, rs6128408, rs568880773, rs6100172, |
| | rs736144, rs1886839, rs745405, rs2426776, rs6026470, rs2426777, |
| | rs12480759, rs2426778, rs2246228, rs6070563, rs62203770, rs369192, |
| | rs435208, rs412181, rs6070566, rs6128410, rs2426780, rs2426782, |
| | rs446021, rs2182246, rs6015349, rs6026478, rs6026479, rs6070567, |
| | rs436778, rs2182247, rs6128412, rs6128413, rs6064697, rs6070581, |
| | rs6026486, rs2865713, rs6026504, rs6070594, rs6064699, rs4484086, |
| | rs6128421, rs4640451, rs116731793, rs2180796, rs11696939, rs927240, |
| | rs1022697, rs13038360, rs2865714, rs6026511, rs6015366, rs6070598, |
| | rs6026515, rs6064704, rs6100212, rs6513412, rs6070603, rs6128427, |
| | rs6064707, rs6100227, rs2180797, rs7267516, rs13041735, rs6026535, |
| | rs6064709, rs748951, rs6015379, rs6026536, rs911300, rs736266, |
| | rs911301, rs911302, rs911303, rs733773, rs733772, rs4810144, |
| | rs3746709, rs6026544, rs6070625, rs6026545, rs4810145, rs4810146, |
| | rs4812034, rs4812035, rs3761263, rs1800905, rs3787497, rs6123832, |
| | rs6026557, rs6026567, rs968519, rs2180696, rs6026574, rs4481060, |
| | rs6026576, rs4810149, rs4812042, rs6026578, rs6026580, rs8121252, |
| | rs8114689, rs6128461, rs6026591, rs6026592, rs3730168, rs6015400, |
| | rs234625, rs234621, rs234618, rs6070651, rs151331, rs234617, rs47223, |
| | rs234615, rs7353214, rs2114753, rs2114754, rs2114755, rs6070669, |
| | rs1559973, rs6026629, rs56846108, rs235196, rs6064731, rs467040, |
| | rs465047, rs12479678, rs151362, rs236727, rs163780, rs163781, |
| | rs149265, rs448943, rs417847, rs163786, rs163788, rs235200, rs189356, |
| | rs163792, rs163793, rs163794, rs2021793, rs163796, rs163797, |
| | rs163798, rs163799, rs151343, rs108727, rs151344, rs24048, rs151336, |
| | rs151346, rs151347, rs151348, rs151349, rs151350, rs6026647, |
| | rs6123845, rs2162593, rs4810150, rs200505206, rs6064738, rs6026656, |
| | rs6092730, rs1028693, rs1023654, rs6015425, rs6026658, rs6015426, |
| | rs6070709, rs6100323, rs6070711, rs6064740, rs4812052, rs2162594, |
| | rs6070712, rs6026669, rs6026670, rs6070714, rs11086663, rs6064742, |
| | rs2146666, rs6026673, rs4812053, rs2865724, rs4810151, rs2023511, |
| | rs6026674, rs6026675, rs6026676, rs6026677, rs6015430, rs6015431, |
| | rs6070715, rs6026681, rs6026684, rs6070719, rs6070720, rs6026686, |
| | rs6026689, rs6123849, rs4812055, rs2903072, rs6026691, rs4449199, |
| | rs6015433, rs6026693, rs4337535, rs890633, rs2865730, rs6015434, |
| | rs6015435, rs6026695, rs2865731, rs6070722, rs6064745, rs200664963. |
| RUNX1 | rs11700756, rs2268276, rs2268277, rs2268278, rs2834643, rs2834644, |
| 21:36160098- | rs2253313, rs2253319, rs762164, rs4817695, rs2154450, rs2268285, |
| 37376965 | rs188695559, rs2409534, rs2409536, rs60855390, rs2252585, |
| 229 SNPs | rs79908548, rs9975428, rs2834693, rs2834699, rs8127732, rs2834701, |
| | rs11088300, rs9974986, rs2834703, rs2834705, rs62216442, rs990116, |
| | rs28391463, rs58709766, rs2242888, rs2236431, rs7277157, rs8133634, |
| | rs7283025, rs2834741, rs928282, rs8132078, rs1003473, rs1571696, |
| | rs2834748, rs2031088, rs2834756, rs2834757, rs2834758, rs2834767, |
| | rs1883063, rs2834776, rs2247822, rs2094869, rs12483078, rs2242903, |
| | rs2834782, rs2834785, rs8131196, rs2834793, rs760053, rs2007324, |
| | rs2834796, rs2834797, rs9975836, rs2834805, rs2017243, rs76629697, |
| | rs2834817, rs4816504, rs12483023, rs7282507, rs56059277, rs2834839, |
| | rs2834842, rs2834845, rs2834851, rs2065296, rs8133443, rs2834857, |
| | rs2075901, rs2834877, rs2834884, rs10427505, rs10211916, rs7282359, |
| | rs11701367, rs2834886, rs7277594, rs56015451, rs2142049, rs2178817, |
| | rs34385074, rs9975485, rs13048915, rs13049777, rs2834896, |
| | rs56007971, rs2834904, rs2409620, rs35640344, rs28460413, rs2834907, |
| | rs2834909, rs2409621, rs2834913, rs12152004, rs2834916, rs13046151, |
| | rs6517292, rs62218300, rs13052531, rs2742148, rs2742147, rs2742146, |
| | rs8131707, rs8132050, rs57824579, rs2834928, rs760137, rs2834931, |
| | rs76442119, rs11701941, rs2242734, rs2077323, rs2205109, rs12483567, |
| | rs66997518, rs9983096, rs2242747, rs1888461, rs2242748, rs1003261, |
| | rs2834953, rs1003262, rs2834956, rs440544, rs440854, rs387136, |
| | rs1734895, rs1735222, rs1734891, rs429287, rs990558, rs9984503, |
| | rs928286, rs28496528, rs9984332, rs2205030, rs2834985, rs2834997, |
| | rs2835001, rs2835010, rs2835019, rs12482734, rs1984014, rs1888457, |
| | rs1014593, rs4817734, rs8133254, rs2222957, rs4817736, rs2835049, |
| | rs2835057, rs1734523, rs1235277, rs1734505, rs1734501, rs909446, |
| | rs7364091, rs2835075, rs2026257, rs7275822, rs9980935, rs12483340, |
| | rs11701127, rs2835084, rs8130799, rs7282866, rs1014803, rs2835108, |
| | rs2255731, rs2242784, rs2835115, rs2835117, rs12627212, rs2835121, |
| | rs2835122, rs2835126, rs1011820, rs2835134, rs13047820, rs427899, |
| | rs407611, rs9983707, rs8132625, rs7282075, rs9977418, rs7277535, |
| | rs7278529, rs119491, rs2250427, rs2835169, rs2835170, rs2835171, |
| | rs1467369, rs2835172, rs2835173, rs1467368, rs1467367, rs1467366, |
| | rs9974250, rs8129662, rs2835174, rs6517319, rs8134354, rs10775654, |
| | rs10775655, rs2835182, rs1573467, rs2850052, rs2845726, rs67801135, |
| | rs2212912, rs2243634, rs2243635, rs2850067, rs2409738, rs2256001, |
| | rs2248898, rs1811074, rs12233319. |
| ERG | rs7280665, rs2073359, rs2836384, rs58796403, rs59249418, rs77000871, |
| TMPRSS2 | rs7279458, rs2836399, rs2836402, rs2836409, rs8127366, rs2410035, |
| 21:39739182- | rs1888471, rs1888472, rs1475877, rs2836482, rs62219614, rs7280823, |
| 42880085 | rs4318465, rs2898354, rs2836504, rs3787917, rs2836507, rs2836508, |
| 606 SNPs | rs113956148, rs16996463, rs2212601, rs56080396, rs12482179, |
| | rs9978269, rs2836532, rs2836542, rs9789875, rs2244890, rs2836546, |
| | rs2070526, rs2836554, rs460574, rs460695, rs462507, rs462588, |
| | rs464519, rs61085980, rs2212606, rs2836621, rs8134419, rs8134675, |
| | rs8126579, rs2836635, rs9974911, rs2836650, rs9976432, rs9976385, |
| | rs10048862, rs8134461, rs2252185, rs2836667, rs2410052, rs13052279, |
| | rs9636946, rs2836669, rs2836671, rs2836672, rs1209916, rs1209921, |
| | rs1209922, rs1209940, rs1209943, rs1041781, rs459411, rs6517481, |
| | rs2836686, rs2836687, rs12482470, rs2836693, rs998855, rs762386, |
| | rs1123350, rs7276587, rs2836696, rs57660260, rs1534824, rs1534826, |
| | rs2836705, rs2836708, rs7283959, rs468107, rs468456, rs468529, |
| | rs464074, rs9680142, rs2836719, rs2836735, rs8126904, rs28514570, |
| | rs2836754, rs11701004, rs11701117, rs2836755, rs7279192, rs2256553, |
| | rs8134031, rs13051086, rs2410069, rs1006892, rs9975660, rs2836767, |
| | rs2836770, rs2251210, rs2012898, rs2836782, rs2836783, rs2836786, |
| | rs8130031, rs2836790, rs2836791, rs9979186, rs2836795, rs2836803, |
| | rs8133084, rs4414759, rs2836820, rs2836821, rs11910233, rs2836827, |
| | rs9982978, rs2836836, rs8130903, rs4816610, rs2836842, rs6517502, |
| | rs8133785, rs8127295, rs2836848, rs2242932, rs2836853, rs9980540, |
| | rs914155, rs2836859, rs2836860, rs2410099, rs4279007, rs4266093, |
| | rs8128799, rs2836865, rs119244, rs378999, rs417222, rs450275, |
| | rs56827822, rs2735298, rs8128521, rs369906, rs13046927, rs28756733, |
| | rs13048321, rs9982724, rs2242944, rs378690, rs7275673, rs1554931, |
| | rs2063970, rs450717, rs2242947, rs438649, rs7510385, rs1013128, |
| | rs2836899, rs2410101, rs34912726, rs2836904, rs4817991, rs2836916, |
| | rs2222995, rs11700813, rs11700449, rs1888489, rs442972, rs377098, |
| | rs7282604, rs8131132, rs2142111, rs2150411, rs8130320, rs2836945, |
| | rs2836948, rs2836949, rs2836959, rs7276774, rs2836966, rs1011413, |
| | rs12483559, rs34436313, rs8127986, rs2836980, rs2836981, rs8129595, |
| | rs7281853, rs4818018, rs4818019, rs11088472, rs2410123, rs7280326, |
| | rs2836985, rs9636957, rs2735307, rs1474490, rs9305674, rs2837007, |
| | rs8129564, rs9983435, rs9983404, rs13050837, rs2049823, rs9981594, |
| | rs12482418, rs11088478, rs2837046, rs2026268, rs9983297, rs2837047, |
| | rs7278885, rs13050145, rs3787927, rs8128795, rs2837049, rs7277107, |
| | rs9976202, rs9976208, rs4816629, rs4818040, rs2837053, rs1524929, |
| | rs13046966, rs11700977, rs588903, rs656618, rs670316, rs4372991, |
| | rs538329, rs599377, rs647448, rs58579319, rs1734932, rs550569, |
| | rs667432, rs679857, rs490561, rs497933, rs498027, rs1534080, |
| | rs8132770, rs2239386, rs4818069, rs2837113, rs4818070, rs12482941, |
| | rs12483089, rs2210266, rs4816641, rs9979149, rs2837123, rs2837124, |
| | rs2837125, rs8134049, rs2837126, rs9976947, rs9983659, rs6517566, |
| | rs2837213, rs1571712, rs2837224, rs1984023, rs2410183, rs2898387, |
| | rs381716, rs2837269, rs2299774, rs2299775, rs35716588, rs2251552, |
| | rs2299798, rs2037918, rs2037919, rs2252048, rs4818101, rs2837306, |
| | rs55651028, rs11088500, rs2837315, rs720566, rs7283765, rs2837327, |
| | rs11088502, rs59901545, rs60596029, rs62236605, rs2837343, |
| | rs2837345, rs7281341, rs6517581, rs9980452, rs2009920, rs2091886, |
| | rs926084, rs2837369, rs2837381, rs4816672, rs2837393, rs2249498, |
| | rs2178840, rs12626218, rs2837408, rs2837416, rs401936, rs7282525, |
| | rs28399843, rs403280, rs741801, rs9975693, rs2297266, rs1554941, |
| | rs2837469, rs9981851, rs7279225, rs7278294, rs66861713, rs565883236, |
| | rs67569529, rs726106, rs11702340, rs2837482, rs2837483, rs8128592, |
| | rs2837485, rs2837486, rs2837488, rs2837489, rs2014678, rs2837493, |
| | rs2178851, rs2837495, rs4818120, rs9984703, rs2837501, rs13053013, |
| | rs4375957, rs8131481, rs13052377, rs1997551, rs7282686, rs1554939, |
| | rs1984024, rs2837509, rs405531, rs9982499, rs9982501, rs413465, |
| | rs364721, rs448051, rs437083, rs424372, rs2837520, rs2837524, |
| | rs2837525, rs571455932, rs9305695, rs2837532, rs4818122, rs2837534, |
| | rs8133850, rs8130487, rs7280961, rs6517587, rs2837546, rs9975585, |
| | rs1882759, rs1882760, rs4816681, rs4818127, rs1571717, rs8134631, |
| | rs9983208, rs2837574, rs8132944, rs7410084, rs2837608, rs2205083, |
| | rs2837624, rs6517592, rs9985057, rs2837637, rs4818135, rs35955958, |
| | rs9977267, rs8134455, rs553907861, rs2178844, rs2056845, rs2837669, |
| | rs1882782, rs11700548, rs72613624, rs11088520, rs10211949, |
| | rs2837701, rs13046409, rs9983654, rs9983411, rs4816689, rs2837704, |
| | rs4818142, rs4818143, rs56173150, rs2150419, rs2150420, rs11088521, |
| | rs11088522, rs11088523, rs2837707, rs9984812, rs2837710, rs2837711, |
| | rs9981739, rs9976820, rs2410255, rs9983033, rs9977467, rs2178841, |
| | rs914163, rs4346468, rs465127, rs926085, rs2837730, rs455473, |
| | rs2837736, rs456051, rs461094, rs1072194, rs2205136, rs8134874, |
| | rs2837747, rs2837750, rs34259020, rs9977890, rs67533908, rs9984989, |
| | rs2205137, rs909184, rs9984489, rs8133820, rs8133824, rs8130081, |
| | rs2410261, rs2245115, rs2410265, rs2410266, rs12482259, rs12482304, |
| | rs62223649, rs987897, rs1882785, rs1812234, rs2837763, rs932245, |
| | rs926088, rs8132096, rs7279323, rs6517605, rs8126774, rs9984320, |
| | rs2142127, rs9983505, rs741910, rs2003642, rs741912, rs1022426, |
| | rs2142129, rs2178848, rs11088526, rs7276854, rs7281715, rs7281585, |
| | rs9976172, rs9976262, rs9976496, rs9979024, rs7283122, rs61513664, |
| | rs569911079, rs56122090, rs7279397, rs10854405, rs12482503, |
| | rs2837814, rs2837815, rs2837818, rs2837823, rs5006701, rs2091899, |
| | rs2837828, rs9647188, rs35480596, rs28654069, rs6517625, rs8131683, |
| | rs980274, rs8133945, rs4143757, rs28655258, rs60319342, rs2837858, |
| | rs1012852, rs9636977, rs9305711, rs8132824, rs928299, rs9977379, |
| | rs7279115, rs8127007, rs1888514, rs112775759, rs7275921, rs9975772, |
| | rs2837875, rs2026276, rs7275852, rs7276000, rs7283882, rs1041447, |
| | rs6517642, rs2837884, rs2065319, rs8134414, rs2898433, rs2410352, |
| | rs1467754, rs7275291, rs7280718, rs9980324, rs748562, rs748563, |
| | rs2837904, rs8129321, rs2837906, rs914165, rs2011706, rs2837913, |
| | rs4818192, rs2837918, rs9305718, rs7277376, rs60049918, rs57955932, |
| | rs12053704, rs10211943, rs744857, rs744859, rs4816708, rs8130642, |
| | rs2837942, rs2837954, rs1029009, rs1029013, rs75965935, rs8131452, |
| | rs2837961, rs2297271, rs11700578, rs11700599, rs11701114, |
| | rs67757006, rs914176, rs6517655, rs8134160, rs2776344, rs13049769, |
| | rs201197092, rs4818231, rs12627105, rs457870, rs468672, rs468105, |
| | rs468207, rs469083, rs468440, rs468747, rs1638369, rs469073, |
| | rs469270, rs364199, rs467639, rs8133682, rs6517666, rs6517668, |
| | rs13050773, rs3737400, rs468161, rs468972, rs462321, rs2104810, |
| | rs383510, rs422471, rs391099, rs430915, rs402303. |
| FANCD2 | rs12490900, rs11717031, rs9846665, rs9827315, rs9879307, rs7636337, |
| 3:10068098- | rs7613920, rs9834636, rs9838916, rs9835131, rs9835132, rs9835288, |
| 10143614 | rs1061154, rs11712124, rs7612722, rs7622954, rs12495401, rs12495429, |
| 249 SNPs | rs11131194, rs13061591, rs3856908, rs34022591, rs203562, rs34082649, |
| | rs192495, rs13073918, rs452610, rs429011, rs1687742, rs1695432, |
| | rs1166374, rs1166375, rs279591, rs279592, rs279593, rs183957, |
| | rs279595, rs279596, rs375024, rs462378, rs708570, rs465492, rs386677, |
| | rs279557, rs279556, rs71623082, rs279554, rs1687739, rs1642283, |
| | rs6443271, rs33970214, rs783493, rs394429, rs395042, rs279590, |
| | rs454607, rs462474, rs279589, rs279588, rs279587, rs279586, rs279585, |
| | rs279584, rs279582, rs279581, rs279580, rs279578, rs279577, rs279576, |
| | rs279575, rs432815, rs432813, rs172155, rs188090, rs279574, rs279573, |
| | rs279571, rs279570, rs279569, rs279566, rs172154, rs693802, rs783494, |
| | rs376511, rs372559, rs2616568, rs6776272, rs7627880, rs279547, |
| | rs3894571, rs55862344, rs13059144, rs66559400, rs13076489, |
| | rs68153882, rs113940455, rs35056669, rs7636817, rs3732966, |
| | rs7633769, rs34779351, rs9812006, rs13100734, rs13077641, |
| | rs13074282, rs68080705, rs17050674, rs17050675, rs3755782, |
| | rs2287438, rs13099136, rs6763366, rs13075308, rs7614810, rs7615088, |
| | rs6807326, rs17050699, rs61429272, rs9312044, rs2886396, rs2030564, |
| | rs9757159, rs7618628, rs6796419, rs17050701, rs55840655, rs56387520, |
| | rs9867091, rs452792, rs2272122, rs67301745, rs55808392, rs8411, |
| | rs35293461, rs13433680, rs6792811, rs6771697, rs9857329, rs779803, |
| | rs779807, rs779811, rs2600005, rs1703153, rs149915954, rs1678595, |
| | rs166538, rs462879, rs457414, rs458952, rs11465853, rs73026506, |
| | rs708030, rs708031, rs2619508, rs1619378, rs1681663, rs55806638, |
| | rs17610830, rs3864021, rs1177614, rs155265, rs155266, rs155583, |
| | rs3732957, rs155584, rs696040, rs697919, rs696356, rs696319, |
| | rs696041, rs263410, rs263411, rs263413, rs6442158, rs6442159, |
| | rs9877206, rs873405, rs358849, rs17815972, rs708033, rs7373858, |
| | rs459864, rs56111270, rs461602, rs459483, rs6442161, rs2302862, |
| | rs2302861, rs6804954, rs6780464, rs55890954, rs3895947, rs11465911, |
| | rs2302859, rs779910, rs4684669, rs813591, rs1642736, rs3844284, |
| | rs467190, rs779905, rs2675489, rs1678598, rs779903, rs779902, |
| | rs779901, rs6796426, rs4684671, rs4684672, rs776516, rs776514, |
| | rs11717636, rs11926277, rs35276047, rs11717920, rs11706450, |
| | rs713016, rs11711184, rs709634, rs11928386, rs34355838, rs67266168, |
| | rs11465932, rs1678586, rs242723, rs242722, rs242721, rs2270454, |
| | rs2241307, rs2241308, rs2241310, rs1642974, rs1642975, rs151759, |
| | rs164938, rs242730, rs2075355, rs2544005, rs582868, rs2544008, |
| | rs26802, rs10490815, rs1678599, rs1629816, rs57221010, rs2578476, |
| | rs9683165, rs9681908, rs12630739. |
| MLH1 | rs9849965, rs141822432, rs13072940, rs931913, rs9882879, rs13080204, |
| 3:37034823- | rs4678552, rs9876421, rs7646422, rs7646626, rs11707149, rs7636849, |
| 37107380 | rs4678908, rs925134, rs925135, rs4678554, rs9834970, rs906482, |
| 260 SNPs | rs906483, rs906484, rs1553656, rs1532964, rs1472864, rs1532965, |
| | rs1532966, rs1006834, rs1507882, rs4072458, rs4624519, rs9917659, |
| | rs35784452, rs6550435, rs576390306, rs906481, rs199501093, |
| | rs11129735, rs7652637, rs3732386, rs3732385, rs3732384, rs9811916, |
| | rs4678909, rs4234258, rs4476463, rs4392371, rs9821223, rs6771655, |
| | rs9882911, rs4678910, rs4678911, rs9819304, rs9985296, rs11712098, |
| | rs4441609, rs17195098, rs17807744, rs9883001, rs4678912, rs11706780, |
| | rs4327336, rs7619089, rs4642070, rs11129738, rs57251184, rs12637747, |
| | rs9311139, rs28522146, rs68185107, rs9859093, rs17035672, rs6550437, |
| | rs6808230, rs6775274, rs9881419, rs12488181, rs7649846, rs4510338, |
| | rs4296548, rs4591457, rs6764968, rs6765139, rs6550438, rs9810132, |
| | rs6769594, rs4497988, rs12629833, rs4404400, rs4340652, rs67702069, |
| | rs7633292, rs7613678, rs12107029, rs11129739, rs9830322, rs9830379, |
| | rs9882814, rs11918190, rs11921747, rs11129740, rs4583586, rs6794134, |
| | rs6765624, rs72861201, rs9862188, rs6776162, rs6550439, rs10222527, |
| | rs6783176, rs9832646, rs34707335, rs717734, rs717733, rs6793902, |
| | rs11129741, rs6781803, rs11129742, rs4508725, rs4423718, rs4328757, |
| | rs6550440, rs6550441, rs78036468, rs6550442, rs7643366, rs11708164, |
| | rs7611656, rs4678914, rs6805239, rs9819348, rs6779665, rs9867455, |
| | rs9813869, rs7624498, rs6769400, rs6772646, rs28576724, rs7622114, |
| | rs13314421, rs7628483, rs6806158, rs6777094, rs4535177, rs11706926, |
| | rs6777217, rs56231935, rs7619689, rs7619703, rs9857252, rs6776697, |
| | rs2276808, rs2276807, rs73826924, rs6550443, rs7628085, rs7649344, |
| | rs7651903, rs9847384, rs9883604, rs7632760, rs4678922, rs6769734, |
| | rs9831084, rs6789043, rs4647218, rs3774341, rs4234259, rs1558528, |
| | rs4647277, rs2286939, rs3774339, rs3774338, rs6550445, rs6550446, |
| | rs9819025, rs997588, rs6772548, rs748766, rs9876116, rs9822082, |
| | rs2241031, rs4678931, rs749072, rs3774327, rs12489874, rs7639375, |
| | rs2302503, rs6550448, rs2302504, rs6806487, rs6550450, rs1990492, |
| | rs17204675, rs7616160, rs6800554, rs2302507, rs9859086, rs7643836, |
| | rs6806252, rs9861967, rs6780799, rs1500794, rs3733030, rs3733029, |
| | rs59594366, rs61493618, rs11717310, rs9832138, rs2885608, rs6775008, |
| | rs4678938, rs6809297, rs6550456, rs6550457, rs7640224, rs4678940, |
| | rs7630221, rs9839933, rs4678942, rs9869432, rs4678943, rs9823120, |
| | rs9823617, rs2362824, rs7638620, rs6786857, rs1392749, rs7634972, |
| | rs138124594, rs7637994, rs9755599, rs11920928, rs4678944, rs9854641, |
| | rs9854956, rs963078, rs11717564, rs9852386, rs6773752, rs6786906, |
| | rs13098279, rs7631605, rs6801864, rs7632108, rs9876275, rs9839105, |
| | rs7628444, rs9874437, rs7639607, rs6809976, rs2062587, rs6770520, |
| | rs11921099, rs4678562, rs4678953, rs6800842, rs35953631, rs9810522, |
| | rs6778387, rs7637545, rs1533321. |
| CTNNB1 | rs6785706, rs9868157, rs11719036, rs6792760, rs7626598, rs7626599, |
| 3:41236328- | rs9809224, rs11129891, rs11922967, rs11915349, rs9814078, rs4569635, |
| 41301587 | rs4435612, rs4337622, rs9311256, rs7630628, rs2217080, rs6776728, |
| 231 SNPs | rs9877619, rs9822113, rs9311258, rs2371348, rs2371349, rs2371350, |
| | rs9311259, rs9832585, rs9311260, rs6802872, rs9827812, rs6599135, |
| | rs411259, rs441655, rs365510, rs3886317, rs415039, rs398638, |
| | rs400952, rs453509, rs451553, rs388364, rs382863, rs444419, |
| | rs12715362, rs418060, rs408499, rs9284874, rs380285, rs442683, |
| | rs442675, rs442045, rs9311261, rs9311262, rs7629605, rs394831, |
| | rs417265, rs9863665, rs387157, rs391244, rs405651, rs6599141, |
| | rs404477, rs12488693, rs437763, rs441140, rs429988, rs390910, |
| | rs370243, rs387329, rs386723, rs448359, rs376362, rs416486, rs416183, |
| | rs395349, rs384934, rs384260, rs374289, rs442024, rs391459, rs436705, |
| | rs107236, rs442115, rs412476, rs431867, rs431052, rs430848, rs436448, |
| | rs383911, rs422623, rs370387, rs406589, rs417052, rs384097, rs447911, |
| | rs401680, rs385905, rs444561, rs430727, rs430577, rs367027, rs450615, |
| | rs447408, rs411124, rs440719, rs428903, rs87938, rs428510, rs378967, |
| | rs386132, rs398993, rs423170, rs446717, rs440640, rs436792, rs389264, |
| | rs108606, rs1984856, rs1156916, rs9820407, rs9878224, rs12485899, |
| | rs4437116, rs6762550, rs7609599, rs6599143, rs4974070, rs378422, |
| | rs7621892, rs417183, rs4419359, rs414272, rs369145, rs374668, |
| | rs375583, rs454690, rs9832204, rs406664, rs1722850, rs3953307, |
| | rs2059601, rs1625743, rs9821710, rs1722848, rs4973924, rs4973925, |
| | rs4974074, rs9872276, rs1722846, rs9859392, rs9870255, rs3864004, |
| | rs4533622, rs2371452, rs3915129, rs6776881, rs2140090, rs9813198, |
| | rs4973927, rs34565533, rs1798802, rs13072632, rs1880481, rs11564450, |
| | rs11564452, rs9311265, rs9883073, rs9883839, rs1880480, rs9849022, |
| | rs1608726, rs4973930, rs6599147, rs76376852, rs9853680, rs4973933, |
| | rs9869660, rs7637185, rs9878048, rs4973934, rs4973875, rs7630526, |
| | rs6768531, rs9311268, rs6775334, rs7610589, rs6599150, rs1532015, |
| | rs9857557, rs9857720, rs6790569, rs1352272, rs9311270, rs9311271, |
| | rs9311272, rs729093, rs4973942, rs17030371, rs10049320, rs58977456, |
| | rs7637424, rs6809724, rs1386601, rs9835690, rs7618346, rs4973943, |
| | rs6786191, rs6800030, rs9311274, rs9813781, rs1586457, rs9838735, |
| | rs9824480, rs2371493, rs9826206, rs9831275, rs9812079, rs9827610, |
| | rs58920852, rs6788792, rs4973949, rs9841849, rs7621118, rs10446425, |
| | rs1495691, rs9834718, rs9852344, rs112317105, rs9858040, |
| | rs147552237, rs7374528, rs7373832. |
| FOXP1 | rs34603408, rs7611254, rs2140132, rs12630880, rs9845197, rs62257217, |
| 3:71003844- | rs8179901, rs6780338, rs73118209, rs2140131, rs17008114, rs7615404, |
| 71633140 | rs73118214, rs1288987, rs7616330, rs1288973, rs1288974, rs377165288, |
| 272 SNPs | rs1288981, rs56237264, rs74623476, rs2686268, rs17008204, rs831435, |
| | rs6549377, rs831440, rs831444, rs3843363, rs831447, rs844245, |
| | rs9844845, rs831450, rs831452, rs13079951, rs861251, rs6802640, |
| | rs2687759, rs2593851, rs2686270, rs115756824, rs2121783, rs2687760, |
| | rs6772367, rs2593852, rs2687761, rs2593853, rs2687762, rs2593854, |
| | rs9850599, rs2686271, rs1474273, rs34925588, rs13088128, rs4677532, |
| | rs1568555, rs2321499, rs61593374, rs9877741, rs61651183, rs58874807, |
| | rs17008267, rs4677551, rs1522177, rs6800929, rs10865657, rs12633797, |
| | rs7647909, rs7653672, rs7632470, rs17699304, rs11923661, rs6790679, |
| | rs1402994, rs1522178, rs12054400, rs67178525, rs2321500, rs2321501, |
| | rs4334611, rs9826573, rs7650867, rs62256868, rs9840529, rs34347778, |
| | rs1522169, rs10511014, rs2166780, rs10084728, rs4677584, rs9845218, |
| | rs12330648, rs2321506, rs73106142, rs73106143, rs35390588, |
| | rs2056467, rs4677029, rs4677030, rs4677595, rs9830657, rs9844174, |
| | rs9827137, rs9827138, rs9310211, rs7430246, rs7429997, rs9825176, |
| | rs7645130, rs9837880, rs9866910, rs878118, rs2293138, rs2037474, |
| | rs7630538, rs10865658, rs6786408, rs17654359, rs9819895, rs6549383, |
| | rs4677600, rs880543, rs1288814, rs17008389, rs2568844, rs17008402, |
| | rs17008403, rs1148749, rs2568838, rs952814, rs2568833, rs1522174, |
| | rs557869288, rs73839471, rs200764586, rs2704804, rs2280518, |
| | rs2280519, rs6784514, rs2568847, rs2704800, rs11128211, rs1612181, |
| | rs1288693, rs1288694, rs1288695, rs1288696, rs1295495, rs2321695, |
| | rs4613421, rs2704806, rs1288821, rs1288822, rs1288823, rs1288824, |
| | rs1288825, rs1653969, rs9861531, rs9840242, rs12488742, rs59493678, |
| | rs1653974, rs4677035, rs13075274, rs17043920, rs4677602, rs4677604, |
| | rs11710517, rs11128212, rs11128213, rs56393771, rs4677605, |
| | rs6801488, rs6809994, rs6764264, rs6789892, rs6804794, rs4677606, |
| | rs4677607, rs4677608, rs13092790, rs6771515, rs35131918, rs1288701, |
| | rs11713665, rs6799205, rs13064984, rs36047018, rs4677609, rs4677610, |
| | rs60905580, rs60380279, rs9854559, rs9859586, rs13083901, |
| | rs78101305, rs62245965, rs62245966, rs59996336, rs9865527, |
| | rs1997479, rs3732734, rs17717717, rs4677611, rs13072512, rs4452296, |
| | rs831078, rs831082, rs9815974, rs10865659, rs11720193, rs11711425, |
| | rs13067084, rs9840310, rs13099575, rs7633837, rs7640164, rs7626396, |
| | rs10212422, rs7647190, rs7647195, rs11714097, rs937839, rs2321694, |
| | rs11917612, rs11928310, rs7640269, rs6549389, rs55705945, |
| | rs56238933, rs6771130, rs6772376, rs12492180, rs2135548, rs1911152, |
| | rs9853081, rs11715828, rs6798507, rs17718783, rs17718831, rs6779041, |
| | rs1079409, rs1979843, rs13059691, rs830605, rs6785868, rs6773658, |
| | rs9834492, rs13316357, rs9879910, rs878173, rs9828619, rs2322031, |
| | rs830659, rs830660, rs830661, rs830652, rs830655, rs830525, rs704249, |
| | rs704250, rs704251, rs704253, rs704254, rs1447895, rs1717281, |
| | rs1295372, rs861231, rs862689, rs830650, rs2568918, rs862690, |
| | rs2684380. |
| RYBP | rs4588330, rs4073296, rs4074146, rs62245242, rs7642040, rs4677108, |
| 3:72420976- | rs34021333, rs7651697, rs34602864, rs9820358, rs9839863, rs4677109, |
| 72496069 | rs9917816, rs9917700, rs13073324, rs6767307, rs9758166, rs9872574, |
| 240 SNPs | rs9881127, rs4386458, rs9790178, rs28669100, rs7646140, rs9822424, |
| | rs13325039, rs62245246, rs4677114, rs4677115, rs6763805, rs9862813, |
| | rs4676865, rs4676866, rs13324045, rs4677117, rs11128254, rs12632137, |
| | rs12632171, rs11128255, rs7432379, rs12632003, rs4282028, rs4078475, |
| | rs6791309, rs12634231, rs4677118, rs4677119, rs4525828, rs4677120, |
| | rs9862489, rs9862646, rs56139541, rs11920898, rs9841246, rs9875432, |
| | rs57416770, rs4677126, rs4677127, rs9878609, rs11128256, rs9818600, |
| | rs9818601, rs9819521, rs6549448, rs6549449, rs9821750, rs4621297, |
| | rs4464415, rs4234161, rs9815397, rs9838862, rs4677129, rs4398399, |
| | rs4677132, rs59299939, rs35333734, rs13085929, rs4677133, rs9858942, |
| | rs4677134, rs62244324, rs6771836, rs11128261, rs6783293, rs34985305, |
| | rs6549450, rs4076388, rs4677135, rs9855548, rs11710566, rs11128262, |
| | rs67166817, rs4677137, rs9847707, rs6549451, rs4677140, rs4128691, |
| | rs61065474, rs11715508, rs62245267, rs61139766, rs4677142, |
| | rs13326397, rs9682388, rs11714865, rs4677143, rs62245272, |
| | rs11707495, rs12631764, rs9820851, rs4075816, rs7349566, rs4303823, |
| | rs62245299, rs28436913, rs11918732, rs4677147, rs112641129, |
| | rs9869961, rs11128263, rs11128264, rs4677148, rs9832740, rs9815886, |
| | rs4677150, rs4677151, rs9815599, rs56251132, rs9821691, rs9809116, |
| | rs9870681, rs9832502, rs9832668, rs4676872, rs4676873, rs6792408, |
| | rs6787689, rs9841241, rs6792087, rs7639407, rs144588727, rs6781590, |
| | rs9840391, rs11921972, rs7652333, rs12631820, rs7628338, rs753319, |
| | rs6549455, rs4676875, rs2028240, rs2118593, rs57357027, rs920289, |
| | rs2118592, rs11128267, rs9870697, rs9813551, rs9876315, rs28619449, |
| | rs11128268, rs11128269, rs11128270, rs13072744, rs6796880, |
| | rs6549457, rs6790808, rs6806741, rs10049211, rs3943699, rs11128273, |
| | rs11128274, rs11128275, rs934841, rs934840, rs893468, rs2164920, |
| | rs7631852, rs4677167, rs7613906, rs4676876, rs9873309, rs55756235, |
| | rs2043812, rs2043813, rs2043814, rs2043815, rs2043817, rs2043818, |
| | rs4677169, rs9845371, rs9881547, rs7644146, rs4677172, rs4677173, |
| | rs6794917, rs1898634, rs151295697, rs11926839, rs7645683, rs7648311, |
| | rs13077631, rs9849164, rs7653090, rs4380357, rs4367006, rs6549465, |
| | rs11705764, rs9819039, rs11128281, rs13099729, rs4234162, rs6784906, |
| | rs7643429, rs4677191, rs17010000, rs7621533, rs983106, rs1873351, |
| | rs1994626, rs4676882, rs13091510, rs13091668, rs2130665, rs923575, |
| | rs4322944, rs4677198, rs4676884, rs902421, rs2029505, rs2017253, |
| | rs902419, rs1982842, rs7647258, rs9823664, rs1024008, rs4677201, |
| | rs201650413, rs6769710, rs12714789, rs28616266. |
| PIK3CB | rs10935281, rs56236670, rs774003, rs66644444, rs67037472, rs6763380, |
| 3:138372860- | rs1720836, rs1679175, rs1679165, rs11709290, rs9848938, rs1679167, |
| 138553780 | rs3813264, rs1618357, rs1679169, rs2086351, rs4678264, rs1720833, |
| 230 SNPs | rs1479930, rs1600511, rs4678266, rs1625751, rs774011, rs9855306, |
| | rs7653815, rs7627685, rs6439809, rs35471402, rs9822037, rs13101081, |
| | rs9827256, rs3884240, rs7625326, rs9848718, rs3884241, rs7645920, |
| | rs7626226, rs9853998, rs13098789, rs60513493, rs56071208, rs2881793, |
| | rs6771237, rs6439810, rs7643973, rs1672927, rs612471, rs611134, |
| | rs537210, rs609797, rs814022, rs542010, rs564800, rs582386, |
| | rs1088055, rs629884, rs550507, rs620035, rs773445, rs1082034, |
| | rs1082037, rs4432674, rs1672963, rs1673593, rs1673594, rs1379257, |
| | rs497334, rs1672932, rs936316, rs11717771, rs773450, rs559313, |
| | rs587331, rs586946, rs10935287, rs1673622, rs12490633, rs12497070, |
| | rs12487652, rs773444, rs12636910, rs529615, rs634374, rs664928, |
| | rs2607795, rs2348146, rs587005, rs503922, rs773455, rs773456, |
| | rs111398337, rs75275561, rs67141870, rs1585188, rs654528, rs1672931, |
| | rs642258, rs1672928, rs1672936, rs1673612, rs1672933, rs361077, |
| | rs361076, rs361075, rs12636246, rs470678, rs537894, rs600590, |
| | rs2607796, rs13433948, rs550768, rs618457, rs1068685, rs470359, |
| | rs635592, rs361089, rs2595928, rs603723, rs361087, rs361088, |
| | rs361091, rs361093, rs361094, rs361096, rs2595932, rs1663556, |
| | rs7615854, rs541131213, rs61508609, rs361064, rs524164, rs493152, |
| | rs361056, rs361079, rs361080, rs361081, rs361082, rs393853, |
| | rs12631204, rs201997168, rs6148090, rs439720, rs191713, rs361084, |
| | rs191714, rs4305444, rs497900, rs385254, rs361060, rs244381, |
| | rs361058, rs361059, rs191775, rs9863481, rs558905, rs564455, |
| | rs473388, rs491371, rs647972, rs35926694, rs500687, rs540907, |
| | rs398065, rs772612, rs482334, rs476800, rs361072, rs361069, rs361071, |
| | rs361067, rs168633, rs388649, rs367058, rs4894351, rs409507, |
| | rs404110, rs9822714, rs9834963, rs6801207, rs11713049, rs7612106, |
| | rs13082808, rs6780989, rs6784610, rs9647336, rs9647337, rs6778874, |
| | rs10935288, rs17484361, rs4894359, rs9873179, rs7613579, rs9813576, |
| | rs12631035, rs11708762, rs7646689, rs7628172, rs7650967, rs10804659, |
| | rs11706016, rs13061496, rs6776833, rs9871842, rs9882957, rs35514824, |
| | rs11706291, rs727191, rs9850990, rs6439828, rs13060060, rs7641916, |
| | rs56799713, rs2625077, rs191709, rs360706, rs360704, rs360703, |
| | rs360702, rs11919100, rs9863639, rs9849242, rs9990283, rs9810032, |
| | rs4894374, rs55793930, rs13082612, rs9831078, rs4364192, rs9843765, |
| | rs9824598. |
| ATR | rs7645491, rs11713316, rs11711841, rs13070171, rs571086737, |
| 3:142168077- | rs13081882, rs7613314, rs13068506, rs7627347, rs11710559, rs7625539, |
| 142297668 | rs11711821, rs13093191, rs4386518, rs7621485, rs7621757, rs6791161, |
| 251 SNPs | rs11715327, rs7645657, rs6440074, rs6440075, rs13083432, rs13077902, |
| | rs10428215, rs10935459, rs2171035, rs1825522, rs4434160, rs1552340, |
| | rs6440077, rs13067197, rs9289648, rs2862746, rs77600880, rs6800695, |
| | rs13080394, rs13087972, rs1485393, rs6440078, rs2862745, rs12632726, |
| | rs7641492, rs3816805, rs10935462, rs6802631, rs6769786, rs7627694, |
| | rs2862744, rs9859834, rs2862743, rs6440082, rs7643377, rs7623785, |
| | rs62276415, rs13061823, rs7613664, rs953135, rs2306699, rs7652687, |
| | rs9814159, rs13082648, rs9836038, rs9867210, rs28708455, rs9826786, |
| | rs4441672, rs9835160, rs6772400, rs11925693, rs9829125, rs9847364, |
| | rs10935463, rs13075259, rs9828178, rs9816736, rs13099184, rs9820770, |
| | rs9855140, rs4582075, rs4318566, rs140512510, rs6440084, |
| | rs138975449, rs4558780, rs3922730, rs6775718, rs7630115, rs13092593, |
| | rs4273389, rs73864545, rs201323437, rs4256152, rs11916455, |
| | rs3923464, rs13098343, rs9876254, rs6440085, rs6791816, rs9855919, |
| | rs13066441, rs11708470, rs7630130, rs9808914, rs9808943, rs13085247, |
| | rs13085261, rs7651071, rs7623524, rs6440087, rs9683085, rs12629713, |
| | rs7647573, rs13065800, rs7636909, rs13063075, rs13085998, |
| | rs13067795, rs13065075, rs6440088, rs10935465, rs9869842, |
| | rs13070239, rs13092234, rs7641914, rs9990092, rs6440089, rs1129391, |
| | rs6776402, rs11923612, rs12639363, rs13068397, rs6782400, rs6768093, |
| | rs13072346, rs13072355, rs6792259, rs9825207, rs7641330, rs13089219, |
| | rs12638158, rs9879995, rs12634255, rs9846960, rs6770155, rs6440090, |
| | rs6799752, rs7610557, rs6440091, rs6440092, rs13069307, rs6440093, |
| | rs6440094, rs6414350, rs13086864, rs7651542, rs11718899, rs13093304, |
| | rs13093869, rs7635052, rs7428496, rs7432464, rs9824176, rs4413348, |
| | rs9856302, rs2049325, rs2049326, rs75274088, rs9866958, rs7609842, |
| | rs9862919, rs9824893, rs9882763, rs34392719, rs2030613, rs2177396, |
| | rs2140432, rs6775813, rs1916639, rs1983434, rs2354860, rs13078890, |
| | rs1357530, rs1357529, rs6802774, rs7646880, rs13074172, rs7631313, |
| | rs6440098, rs7615316, rs6440099, rs6784089, rs34372236, rs2354858, |
| | rs13079246, rs9832606, rs2883380, rs17623267, rs16852486, rs6808205, |
| | rs6808302, rs6784361, rs13069113, rs4683690, rs12695768, rs13080785, |
| | rs13058771, rs13100985, rs3736561, rs3736560, rs940212, rs940211, |
| | rs6798232, rs2354175, rs11917460, rs7430429, rs4683691, rs11928203, |
| | rs4594656, rs13074153, rs2140437, rs7648354, rs75149239, rs62278480, |
| | rs13099848, rs4683427, rs13091247, rs6797492, rs7650047, rs3773504, |
| | rs6440104, rs7623762, rs28571594, rs9849265, rs7629249, rs60486171, |
| | rs953239, rs4539980, rs35467195, rs7626908, rs6784998, rs6440107, |
| | rs9820721, rs6790959, rs7644704, rs56775607, rs7628728, rs9823836, |
| | rs35862982, rs11915805, rs9754885, rs66897945. |
| PIK3CA | rs59209782, rs4607154, rs9882690, rs61796628, rs9810920, rs10804875, |
| 3:178865902- | rs4362766, rs4245916, rs61796632, rs9862902, rs56182354, rs61796652, |
| 178957881 | rs4328857, rs6794243, rs9841497, rs4583702, rs6797712, rs6800994, |
| 242 SNPs | rs4955799, rs4955798, rs9871126, rs6761999, rs4955797, rs9815772, |
| | rs9816837, rs4254707, rs6787571, rs9836168, rs61798163, rs68090520, |
| | rs56057195, rs4955795, rs79005236, rs11921986, rs4504197, rs6769215, |
| | rs6767991, rs9855743, rs4245917, rs4256180, rs58825377, rs59402446, |
| | rs11708890, rs6808065, rs4955808, rs4955794, rs72622564, rs9861291, |
| | rs10936990, rs13060555, rs61798209, rs55783691, rs9681615, |
| | rs9841670, rs4854965, rs7611081, rs6807293, rs2699888, rs55676624, |
| | rs11919383, rs7621329, rs7644648, rs67562832, rs6443624, rs7646409, |
| | rs9841051, rs2677760, rs7650809, rs7614305, rs870995, rs3729674, |
| | rs9865448, rs2699895, rs3729676, rs2699896, rs3729679, rs6803219, |
| | rs3975513, rs1970901, rs12488074, rs12494623, rs1568205, rs6443625, |
| | rs2699890, rs6769154, rs6769162, rs6782557, rs199659487, rs6798817, |
| | rs6786114, rs6443628, rs9829903, rs62408837, rs9813190, rs9850621, |
| | rs6443629, rs1180368, rs9850477, rs1968207, rs1968208, rs1477189, |
| | rs1477190, rs10936995, rs7617552, rs7642066, rs56295844, rs10804876, |
| | rs10804877, rs10804878, rs11719746, rs12633678, rs12633724, |
| | rs56400023, rs62410370, rs62410371, rs62410372, rs7429685, |
| | rs62410373, rs62410374, rs6773405, rs2864412, rs6443632, rs7631737, |
| | rs7609570, rs1881920, rs7643916, rs10936997, rs62410377, rs9830427, |
| | rs9830432, rs7641524, rs11920947, rs9841199, rs73188923, rs6781173, |
| | rs1861803, rs1861804, rs13101105, rs9872690, rs6769913, rs6805615, |
| | rs11714155, rs6443636, rs6784734, rs4488828, rs6809160, rs7611212, |
| | rs12494080, rs12497474, rs55661900, rs55815422, rs11919371, |
| | rs6443638, rs6443639, rs6443640, rs6807655, rs6807729, rs6772058, |
| | rs6807672, rs6807823, rs6762743, rs9290676, rs9968149, rs9816053, |
| | rs7627922, rs7628077, rs6443641, rs12487677, rs13069902, rs35598915, |
| | rs9879306, rs9290677, rs9290678, rs7626206, rs9834223, rs9290679, |
| | rs9283645, rs8179984, rs2111537, rs13323081, rs12634043, rs13082816, |
| | rs13084215, rs7636710, rs7625091, rs16848314, rs9837033, rs11716462, |
| | rs13076278, rs6794192, rs11714246, rs13076106, rs34162409, |
| | rs35519412, rs11715130, rs7642257, rs6786409, rs9810088, rs6781837, |
| | rs6795997, rs55916170, rs9846848, rs9867380, rs6792113, rs9823440, |
| | rs58111121, rs6762399, rs6781016, rs6804758, rs9822116, rs7620017, |
| | rs6443643, rs2111534, rs9865666, rs6784871, rs6443644, rs3976523, |
| | rs7427053, rs10049286, rs3853153, rs2287209, rs6443645, rs3961211, |
| | rs13074206, rs6443646, rs7651451, rs3853149, rs6443647, rs2287212, |
| | rs374211124, rs12488106, rs6803314, rs6443648, rs6443649, rs7618348, |
| | rs6443650, rs6443651, rs3806682, rs9844549, rs13327561, rs2024497, |
| | rs7625375. |
| FBXW7 | rs111941044, rs11944838, rs10030518, rs10030896, rs6535839, |
| 4:153242410- | rs1986702, rs6833616, rs13103998, rs34349153, rs6535840, rs6535841, |
| 153457253 | rs6836811, rs6837375, rs13126953, rs13132227, rs2406625, rs1806636, |
| 256 SNPs | rs1806637, rs7675252, rs4561915, rs6843947, rs13143496, rs35752631, |
| | rs13130168, rs35111904, rs34205115, rs6823091, rs6833264, rs2897478, |
| | rs1962001, rs11099843, rs6827083, rs7671283, rs28561646, rs6848307, |
| | rs56780740, rs10023167, rs1986740, rs34174274, rs6535843, |
| | rs11721471, rs9990966, rs9993769, rs6849420, rs2897464, rs2001705, |
| | rs2001706, rs72736219, rs4586908, rs13121739, rs28605432, |
| | rs11938570, rs11934476, rs72736223, rs2406602, rs16998856, |
| | rs13107216, rs6858503, rs62319036, rs17361412, rs35225072, |
| | rs13112628, rs17276680, rs4588438, rs11732677, rs6858062, rs2897462, |
| | rs13128788, rs11099844, rs10050011, rs7664575, rs13109486, |
| | rs4461503, rs13111373, rs2003376, rs6856727, rs6535844, rs7690973, |
| | rs6535845, rs17028730, rs3749541, rs61327034, rs6856075, rs6830649, |
| | rs72736262, rs56125937, rs6810751, rs6810757, rs6836275, rs2406624, |
| | rs17028751, rs4554046, rs57575846, rs17028759, rs77061243, |
| | rs55820787, rs6838430, rs12502675, rs76884480, rs10033601, |
| | rs7685296, rs6535847, rs2676330, rs2676329, rs2203644, rs10023172, |
| | rs11931711, rs2714805, rs7673175, rs4696318, rs2676344, rs2255137, |
| | rs2406600, rs2676343, rs1516822, rs2292743, rs4696321, rs2035535, |
| | rs964475, rs57199846, rs2676337, rs6852336, rs2406375, rs4146039, |
| | rs10031972, rs2628570, rs1516820, rs1356300, rs7660590, rs28587268, |
| | rs10021011, rs9991574, rs718089, rs17028932, rs1961679, rs6535851, |
| | rs6535852, rs28434212, rs1516819, rs55885666, rs17028954, rs9307893, |
| | rs34234777, rs59367428, rs6816962, rs1484879, rs10005169, rs6832998, |
| | rs6810720, rs4696324, rs2054757, rs10023327, rs10011174, rs1545904, |
| | rs13103278, rs13435508, rs13435513, rs13434765, rs4696325, |
| | rs77265360, rs112018465, rs56186764, rs6827614, rs2054759, |
| | rs4533752, rs12650423, rs17029013, rs17029015, rs12647428, |
| | rs6818473, rs35215853, rs6818312, rs6819331, rs4696135, rs4696326, |
| | rs7686797, rs7669833, rs6827821, rs4696327, rs7659677, rs10014477, |
| | rs10014554, rs4696136, rs4696137, rs4696138, rs6837269, rs6837634, |
| | rs10020796, rs10011838, rs6813195, rs67894428, rs71620214, |
| | rs7695877, rs13149950, rs28689703, rs6825224, rs12649224, |
| | rs28419333, rs28549186, rs10032212, rs11737220, rs4696142, |
| | rs4505804, rs12650966, rs3935569, rs4443258, rs12641337, rs12650650, |
| | rs10031635, rs10034000, rs7700135, rs7678110, rs6535857, rs4508863, |
| | rs13149145, rs6814401, rs4292315, rs12644557, rs6841692, rs28858132, |
| | rs9996077, rs34376792, rs6841238, rs4569733, rs4437231, rs6857876, |
| | rs4513549, rs4235224, rs4613551, rs4696343, rs4696152, rs7656493, |
| | rs57191027, rs35150031, rs6535870, rs10857266, rs4235225, rs4128306, |
| | rs6848532, rs6848604, rs6825975, rs4696345, rs34073454, rs56369146, |
| | rs9759686, rs142380969, rs4350998, rs6826890, rs59255119, |
| | rs60707972, rs60216726, rs6841526, rs6841369, rs6841876, rs11938938, |
| | rs4077297. |
| PIK3R1 | rs4976030, rs282295, rs282296, rs282297, rs282298, rs10038106, |
| 5:67511548- | rs249480, rs157446, rs249478, rs249477, rs249475, rs282300, |
| 67597649 | rs2372405, rs451757, rs281465, rs32949, rs4976031, rs12697058, |
| 242 SNPs | rs55853207, rs60817000, rs281445, rs58377381, rs12186643, rs6874150, |
| | rs572577, rs2253797, rs465531, rs17237286, rs17309601, rs2888306, |
| | rs5020565, rs2372407, rs59188743, rs1455024, rs17237377, rs12655278, |
| | rs12657914, rs1455025, rs10515067, rs12652064, rs6449946, rs1349966, |
| | rs1349967, rs1349968, rs9332463, rs17309796, rs35255715, rs17316202, |
| | rs35268056, rs10940152, rs6449947, rs59724266, rs56739889, |
| | rs34049639, rs58028804, rs34619487, rs1584959, rs1584960, |
| | rs13188277, rs10940153, rs10461538, rs1120968, rs6867176, |
| | rs113008178, rs10461400, rs1869667, rs2372410, rs13176053, |
| | rs13176511, rs13176222, rs13176523, rs993862, rs993863, rs61128998, |
| | rs34487182, rs36078337, rs966392, rs966393, rs9291924, rs1869668, |
| | rs1869669, rs7727769, rs9688196, rs12659583, rs12655367, rs12659629, |
| | rs13168823, rs897317, rs897318, rs3886182, rs2372412, rs7731423, |
| | rs34011043, rs2372413, rs2888307, rs2372414, rs4404637, rs13169206, |
| | rs2124183, rs17237907, rs13162357, rs35855982, rs35799167, |
| | rs7719938, rs7720262, rs2198647, rs2198646, rs2198645, rs6892163, |
| | rs12660017, rs960383, rs6889193, rs7715780, rs6882730, rs921792, |
| | rs11742365, rs73126520, rs6449956, rs6449957, rs7734515, rs7703736, |
| | rs6886884, rs7714383, rs4016108, rs251397, rs251398, rs173701, |
| | rs40419, rs831227, rs706714, rs13173003, rs13156223, rs10515070, |
| | rs7713645, rs7707370, rs7709243, rs12652661, rs4122269, rs1823023, |
| | rs251399, rs34303, rs831121, rs1946207, rs1946208, rs10940160, |
| | rs2161120, rs1819987, rs2302975, rs6890202, rs6894871, rs6876003, |
| | rs1010793, rs6861401, rs831124, rs34305, rs1445760, rs6873192, |
| | rs9291928, rs830557, rs10058850, rs256508, rs569778802, rs537658360, |
| | rs549516143, rs567908774, rs535398992, rs200592295, rs10051886, |
| | rs34289, rs6859287, rs1466666, rs34299, rs34297, rs34294, rs256497, |
| | rs256496, rs194377, rs256499, rs256500, rs256501, rs256503, rs256498, |
| | rs393552, rs370285, rs7703560, rs7719763, rs716813, rs7707989, |
| | rs10038770, rs10038774, rs6865369, rs7721613, rs12234004, rs4976033, |
| | rs853813, rs853812, rs853811, rs42904, rs853810, rs853809, rs706721, |
| | rs706722, rs42905, rs853807, rs853806, rs853805, rs861230, rs853803, |
| | rs706723, rs853801, rs853800, rs853799, rs853798, rs853797, rs853796, |
| | rs853795, rs706724, rs853794, rs11749527, rs6896001, rs1896628, |
| | rs1896627, rs11744964, rs6449962, rs6858907, rs10515087, rs16897882, |
| | rs56103849, rs12697066, rs13165012, rs13165017, rs7722245, |
| | rs56049648, rs767046, rs11738020, rs1423233, rs7444492, rs11747393, |
| | rs9291930, rs11750641, rs7719353, rs6449963. |
| CHD1 | rs162674, rs162676, rs456446, rs2547958, rs720859, rs1508791, |
| 5:98190908- | rs2549200, rs2591443, rs160718, rs2545678, rs159161, rs2591445, |
| 98262240 | rs316512, rs2591446, rs316511, rs2591447, rs2662250, rs160710, |
| 251 SNPs | rs184143, rs160711, rs408005, rs456470, rs2547976, rs457260, |
| | rs294031, rs2545685, rs382202, rs460295, rs461548, rs461258, |
| | rs465003, rs2545683, rs2547975, rs2662247, rs2545682, rs455749, |
| | rs2591450, rs456367, rs2547974, rs465427, rs444672, rs403888, |
| | rs408400, rs439509, rs459174, rs2662244, rs2545670, rs2547957, |
| | rs2545692, rs2547961, rs981566, rs981567, rs1967318, rs992591, |
| | rs992592, rs2545671, rs2547963, rs2662271, rs2545672, rs2545689, |
| | rs2662269, rs2545690, rs2547969, rs2547970, rs2545673, rs1911904, |
| | rs2662264, rs2545674, rs1979980, rs2249797, rs1508793, rs1818348, |
| | rs2545730, rs1002859, rs2545676, rs1002952, rs2250807, rs331550, |
| | rs331551, rs331552, rs970079, rs2662258, rs2035230, rs331547, |
| | rs331917, rs750632, rs331930, rs692759, rs10069735, rs466442, |
| | rs459774, rs34487, rs7718898, rs327786, rs161955, rs6860504, |
| | rs161937, rs327807, rs467728, rs34497, rs34496, rs326493, rs162341, |
| | rs326491, rs34491, rs34490, rs34489, rs161934, rs161935, rs161936, |
| | rs109843, rs363929, rs2927646, rs10045959, rs10037147, rs113308514, |
| | rs112956762, rs111357081, rs112047336, rs112042355, rs75001231, |
| | rs2927647, rs2927648, rs79191251, rs116183885, rs115121903, |
| | rs78254063, rs2455423, rs559574, rs443726, rs393697, rs407562, |
| | rs395668, rs451141, rs366000, rs449204, rs378089, rs422127, |
| | rs113423144, rs434039, rs370529, rs380156, rs145603822, rs113868271, |
| | rs112393710, rs111871078, rs111566434, rs78721487, rs2937709, |
| | rs2927654, rs1093794, rs709390, rs366353, rs377731, rs539024, |
| | rs416750, rs819232, rs1472622, rs10040066, rs4565239, rs13155749, |
| | rs4703049, rs13185489, rs10050423, rs1501911, rs4703052, rs4703340, |
| | rs11950452, rs13179812, rs1393210, rs11242567, rs4128275, rs4448016, |
| | rs6885458, rs11750764, rs12659212, rs12659251, rs10054188, |
| | rs57544561, rs59910509, rs6890939, rs55946434, rs56161473, |
| | rs11242579, rs7717324, rs7737983, rs6894215, rs11742975, rs61110940, |
| | rs7730642, rs7700837, rs7735294, rs7714788, rs6866426, rs4329038, |
| | rs6596673, rs61198176, rs2369574, rs6596679, rs6596680, rs6596681, |
| | rs7712453, rs10060754, rs2121668, rs10056306, rs10064016, rs2121667, |
| | rs2121666, rs6596689, rs7703628, rs3866454, rs4035624, rs4597999, |
| | rs10477871, rs7724878, rs6596702, rs10067087, rs1374660, rs57154911, |
| | rs6880487, rs11242610, rs10073913, rs1447379, rs1447378, rs7708331, |
| | rs7730656, rs17166691, rs10038344, rs28684518, rs10515287, |
| | rs6883882, rs12054753, rs10464031, rs10900893, rs3886512, |
| | rs10464032, rs1073555, rs726172, rs1447381, rs1447382, rs9327984, |
| | rs11744104, rs11740051, rs10060140, rs10059925, rs58798069, |
| | rs6886794, rs6875635, rs1374662, rs7727262, rs1447384. |
| APC | rs338850, rs338851, rs338852, rs338853, rs11749928, rs12655349, |
| 5:112043195- | rs58871758, rs11951455, rs58773763, rs56317607, rs189296, rs163452, |
| 112181936 | rs163453, rs338859, rs56963388, rs163446, rs137979521, rs338870, |
| 248 SNPs | rs7442992, rs4269327, rs11747636, rs10072379, rs7378714, rs7703562, |
| | rs13176097, rs7704906, rs6594613, rs4455608, rs7711347, rs10078416, |
| | rs10065454, rs66626928, rs35187114, rs4336402, rs13171792, |
| | rs9790990, rs7380387, rs7381078, rs4958066, rs6897331, rs7445994, |
| | rs6594636, rs6594638, rs6891162, rs6869830, rs7449112, rs11750687, |
| | rs6594639, rs13162066, rs11746594, rs7446319, rs4705700, rs4705574, |
| | rs4705490, rs1372217, rs989683, rs7380381, rs964417, rs7709207, |
| | rs12522678, rs12517656, rs1441373, rs1441374, rs13182407, rs748628, |
| | rs13160012, rs114648282, rs34603702, rs11956702, rs6860333, |
| | rs4705739, rs4705740, rs10062652, rs4705742, rs6895205, rs6895116, |
| | rs1441370, rs4705772, rs3919709, rs4705776, rs2044030, rs17134854, |
| | rs11749632, rs11241178, rs35935095, rs17285505, rs10463641, |
| | rs10463642, rs971517, rs4705815, rs4705816, rs12719151, rs2900066, |
| | rs6594643, rs6594644, rs12513756, rs12513804, rs1441375, rs6862050, |
| | rs6894351, rs1661036, rs1465910, rs1661035, rs6864688, rs9326862, |
| | rs10076641, rs11241182, rs6898133, rs3846716, rs10075281, |
| | rs10075323, rs10075377, rs861674, rs2020383, rs28373740, rs11954856, |
| | rs4099181, rs11241184, rs6867243, rs12659119, rs1816769, rs62364017, |
| | rs35130225, rs2431242, rs2952615, rs2545165, rs2545164, rs464708, |
| | rs501250, rs2545162, rs2546109, rs187075, rs544243, rs569940, |
| | rs2909958, rs2909786, rs2909787, rs411356, rs1966477, rs1966476, |
| | rs433429, rs386830, rs448162, rs565453, rs565603, rs497844, rs395858, |
| | rs373059, rs454968, rs374613, rs429427, rs712668, rs431287, rs372492, |
| | rs351770, rs351769, rs351767, rs495794, rs463229, rs410701, rs460301, |
| | rs382260, rs448613, rs712671, rs514763, rs456531, rs419632, |
| | rs35726351, rs67293732, rs1093611, rs1093677, rs818797, rs818430, |
| | rs864682, rs818428, rs712662, rs712664, rs57721139, rs461424, |
| | rs462692, rs465454, rs461866, rs469663, rs387577, rs151978, rs151977, |
| | rs153551, rs153550, rs151976, rs153548, rs153547, rs153546, rs153545, |
| | rs818783, rs818782, rs469827, rs469727, rs698405, rs7710836, |
| | rs468751, rs469760, rs469886, rs468610, rs193527, rs153564, rs173466, |
| | rs149192, rs153563, rs153561, rs153559, rs149190, rs153558, rs151980, |
| | rs151979, rs469523, rs17135222, rs1624259, rs33566, rs33560, rs33558, |
| | rs33557, rs33554, rs33552, rs33551, rs33549, rs33545, rs33544, |
| | rs62366969, rs13164167, rs7733863, rs2112454, rs9784647, rs4705529, |
| | rs373005431, rs4705744, rs12654474, rs4334876, rs13171553, |
| | rs13171773, rs13176362, rs13177101, rs17312754, rs13183347, |
| | rs4705531, rs4705745, rs959949, rs13159449, rs7701781. |
| FANCE | rs9394288, rs9469975, rs1929847, rs9296144, rs10080361, rs4713845, |
| 6:35420138- | rs1888825, rs9462065, rs975496, rs2267657, rs2267658, rs3777742, |
| 35434880 | rs3777743, rs4713847, rs62403575, rs10947540, rs6902523, rs6457812, |
| 186 SNPs | rs10484578, rs2284195, rs9394289, rs7772212, rs2267662, rs2267663, |
| | rs9469979, rs965272, rs1894651, rs991145, rs4711414, rs9380500, |
| | rs9469982, rs7769906, rs1888823, rs1888824, rs9380501, rs10807150, |
| | rs6907324, rs6913037, rs9469985, rs6938946, rs2183834, rs13197551, |
| | rs6920432, rs9469998, rs12211943, rs11571504, rs111244160, |
| | rs4713853, rs6901410, rs6902123, rs9470001, rs9658083, rs9296148, |
| | rs7739752, rs6919334, rs10080411, rs2284196, rs7754871, rs9470007, |
| | rs374398211, rs6922548, rs7763692, rs3777744, rs9658100, rs73413718, |
| | rs6457816, rs1040436, rs1883322, rs2267666, rs7751481, rs2267667, |
| | rs2038068, rs67056409, rs2299871, rs2395623, rs28675889, rs9462083, |
| | rs9462084, rs2206030, rs9462085, rs12212952, rs2894401, rs9394297, |
| | rs3800378, rs4713864, rs10214770, rs7758978, rs6907678, rs9470029, |
| | rs10947548, rs11755391, rs2395626, rs7757405, rs4713868, rs4711416, |
| | rs7739139, rs7739148, rs12525930, rs11962335, rs41270072, rs2076170, |
| | rs1016146, rs760782, rs9394299, rs9462091, rs7740758, rs9366886, |
| | rs7757174, rs7744287, rs7738710, rs12524582, rs7755016, rs7769417, |
| | rs2395628, rs2075611, rs2064319, rs7752728, rs2273000, rs2273001, |
| | rs2273003, rs1540910, rs2894402, rs4267954, rs2395629, rs4711420, |
| | rs12210757, rs7766243, rs12214540, rs6935659, rs55802301, rs9368873, |
| | rs112232059, rs9394303, rs6457828, rs10947554, rs2103681, rs7743152, |
| | rs2395630, rs4713884, rs7741804, rs2894403, rs2395631, rs4713888, |
| | rs11751447, rs9296156, rs3807049, rs9462094, rs9368874, rs9368875, |
| | rs9394304, rs74292921, rs9348978, rs9800506, rs6931456, rs5017617, |
| | rs6931945, rs1984116, rs1984117, rs10947557, rs6923449, rs6911901, |
| | rs6911621, rs6910300, rs9296157, rs7757037, rs3798345, rs9296158, |
| | rs2395634, rs3777747, rs9368878, rs10947562, rs3798347, rs1591365, |
| | rs7751598, rs1360780, rs6902124, rs9394307, rs6912833, rs4713903, |
| | rs9394309, rs4713904, rs6929523, rs4711425, rs4713905, rs9380525, |
| | rs9368881. |
| CDK6 | rs4727274, rs38796, rs39744, rs38798, rs38799, rs38800, rs38801, |
| 7:92234235- | rs38802, rs6977036, rs4729040, rs10267476, rs79068780, rs11978531, |
| 92465908 | rs6963580, rs6465358, rs6962245, rs28702411, rs1053204, rs10232215, |
| 267 SNPs | rs13229255, rs60270139, rs11763017, rs4727276, rs1468867, rs7791858, |
| | rs2111200, rs38808, rs6944715, rs112368668, rs6974429, rs79325318, |
| | rs2058015, rs113639632, rs2190507, rs4727279, rs10229615, |
| | rs73404427, rs6942679, rs4141273, rs34904849, rs6948824, rs57881351, |
| | rs10276838, rs10278093, rs732330, rs6955925, rs28495511, rs42042, |
| | rs42235, rs42045, rs191777, rs10269774, rs2040494, rs2237572, |
| | rs2282978, rs3731343, rs10225965, rs2282982, rs56201652, rs2282983, |
| | rs6954290, rs11773884, rs10264916, rs60814640, rs17164683, |
| | rs201539842, rs201960076, rs56353205, rs4727280, rs2301556, |
| | rs9640606, rs6956477, rs3757823, rs6977712, rs6973871, rs10249013, |
| | rs10278890, rs12670783, rs2237573, rs10243384, rs11533994, |
| | rs10267477, rs1972508, rs11981129, rs2079147, rs10254840, |
| | rs11972740, rs73404497, rs10488521, rs73406758, rs28691637, |
| | rs10234981, rs3802078, rs10228493, rs1008601, rs3802076, rs17164772, |
| | rs1014768, rs2374593, rs1541380, rs1076218, rs3802073, rs3802072, |
| | rs3731299, rs11977300, rs1005346, rs3731288, rs6975474, rs6976387, |
| | rs3731276, rs2106930, rs992519, rs3731267, rs929244, rs12672969, |
| | rs60667771, rs57337700, rs10230506, rs4285416, rs4380868, |
| | rs58634836, rs1858919, rs4605988, rs10243167, rs10953073, rs2023699, |
| | rs11760222, rs11983700, rs58384114, rs10953075, rs10953076, |
| | rs2157924, rs1012659, rs2106514, rs9785033, rs9785040, rs10428948, |
| | rs28758759, rs28568867, rs2888889, rs12666771, rs17164857, |
| | rs2188513, rs10953077, rs10240452, rs10240724, rs6948945, rs6949263, |
| | rs2374611, rs7776536, rs6944020, rs200373861, rs187361378, |
| | rs143409753, rs10252453, rs2106515, rs2106516, rs2157925, rs6465366, |
| | rs6465367, rs2374613, rs1858920, rs7794550, rs7794679, rs12333413, |
| | rs10230387, rs10230415, rs10276763, rs10277139, rs2051708, |
| | rs6972817, rs17164878, rs10255859, rs10271579, rs6978432, |
| | rs11980985, rs6950503, rs6950737, rs6961866, rs10216151, rs6955627, |
| | rs9886048, rs10224528, rs11977873, rs11971809, rs11971827, |
| | rs11971884, rs1548400, rs10953078, rs10953079, rs149967693, |
| | rs10155961, rs10249104, rs10257188, rs17164892, rs2888890, |
| | rs66824728, rs12538862, rs1468148, rs6965615, rs60961479, |
| | rs13225760, rs10488523, rs2157923, rs4729053, rs2374614, rs11974738, |
| | rs10953080, rs2097670, rs73712954, rs35503251, rs7808280, rs4727283, |
| | rs4443589, rs1003827, rs7795033, rs12532769, rs1544343, rs7790945, |
| | rs1013803, rs59730932, rs56742403, rs7780297, rs7782902, rs4729056, |
| | rs17164959, rs55853091, rs17164961, rs4729057, rs7801759, rs6949790, |
| | rs6953868, rs12667084, rs9767816, rs9768802, rs73214410, rs10244152, |
| | rs7803519, rs7807666, rs6958719, rs12672660, rs6948574, rs6973703, |
| | rs7806426, rs4729059, rs719478, rs10236337, rs11976485, rs11976540, |
| | rs4729060, rs7801082, rs12704648, rs13224092, rs11979483, rs6979077, |
| | rs12671937, rs10953083, rs6967490, rs10215082, rs10259658, |
| | rs2188177, rs2106163, rs12534506, rs28813128, rs13310155, |
| | rs28882214, rs35535365. |
| MET | rs28485381, rs717957, rs28495552, rs12671606, rs28557111, rs6980387, |
| 7:116312444- | rs12530912, rs201110585, rs12531767, rs6954077, rs6953982, |
| 116438440 | rs10282556, rs10228178, rs2402080, rs2191500, rs3779511, rs11983864, |
| 246 SNPs | rs11983865, rs11980719, rs12668473, rs28587043, rs10271007, |
| | rs4730743, rs6466579, rs3919515, rs17588172, rs17516287, rs6466580, |
| | rs10261304, rs7811851, rs7795510, rs2109514, rs55883210, rs6466581, |
| | rs6975442, rs926198, rs917664, rs7778733, rs926200, rs3779512, |
| | rs9649394, rs3807986, rs3807989, rs3801995, rs3815412, rs11773845, |
| | rs9886216, rs3757732, rs3757733, rs7804372, rs7789117, rs729949, |
| | rs3807990, rs3807992, rs3807994, rs6466588, rs10953822, rs1997571, |
| | rs1997572, rs11769163, rs2052106, rs10270072, rs7801180, rs12706096, |
| | rs2109517, rs2191502, rs2056865, rs13246051, rs6466590, rs6955302, |
| | rs6978354, rs1028265, rs1989795, rs13238514, rs10277113, rs437, |
| | rs1858830, rs62469051, rs10271561, rs714180, rs763538, rs4330631, |
| | rs38841, rs38842, rs38845, rs39748, rs58131984, rs2299433, rs2299434, |
| | rs38848, rs7798983, rs7785933, rs38849, rs4566974, rs2237708, |
| | rs39749, rs10223961, rs2237709, rs2237710, rs17138937, rs2299436, |
| | rs2299437, rs2299438, rs10248537, rs38851, rs10246585, rs10234854, |
| | rs10243024, rs38852, rs2237714, rs38853, rs38854, rs1476454, rs38855, |
| | rs38856, rs38857, rs38858, rs38859, rs10215153, rs2237717, rs2299439, |
| | rs11770163, rs6947629, rs10435378, rs6978135, rs193686, rs193688, |
| | rs183642, rs6951311, rs168686, rs41735, rs41741, rs16945, rs41743, |
| | rs42336, rs41746, rs41747, rs187437, rs41748, rs56355906, rs41750, |
| | rs41751, rs41752, rs41754, rs41756, rs41757, rs41758, rs42383, rs41759, |
| | rs41760, rs41763, rs41764, rs41765, rs41768, rs28167, rs41769, rs41770, |
| | rs28896, rs41773, rs42337, rs59498189, rs11766819, rs41774, rs41775, |
| | rs41776, rs41777, rs35099490, rs41778, rs1356, rs41779, rs41780, |
| | rs41781, rs41783, rs41784, rs41785, rs438, rs41786, rs41787, rs42375, |
| | rs41789, rs41790, rs41791, rs41793, rs41794, rs34630907, rs42338, |
| | rs11977452, rs12706108, rs13224082, rs12537143, rs9791495, |
| | rs12533755, rs10953829, rs2237720, rs9791721, rs7800249, rs56403542, |
| | rs12533164, rs12669383, rs12706110, rs10953830, rs964449, |
| | rs12674017, rs10487354, rs2301649, rs60899920, rs13239000, |
| | rs12670056, rs12672008, rs10487355, rs12706115, rs12670264, |
| | rs2074025, rs2214168, rs7778381, rs12706120, rs12706121, rs12665952, |
| | rs6947485, rs12665999, rs12666016, rs9649395, rs9649396, rs9649397, |
| | rs12706122, rs7803820, rs7804499, rs7804651, rs7807976, rs7808129, |
| | rs13229091, rs12532926, rs12668426, rs10953831, rs7801824, |
| | rs7802605, rs7782376, rs10244937, rs10953832, rs117106385, |
| | rs118039691, rs116351612, rs201922278, rs55840193, rs11771917, |
| | rs35057858. |
| BRAF | rs12703977, rs6963892, rs6948786, rs6964876, rs6965362, rs2252397, |
| 7:140419127- | rs6943131, rs6965644, rs6963099, rs4726889, rs870217, rs870216, |
| 140624564 | rs7806065, rs10254654, rs10254777, rs6951170, rs10275801, |
| 241 SNPs | rs10254745, rs4458784, rs10952729, rs10952730, rs10952731, |
| | rs10808050, rs12670146, rs4236489, rs6965765, rs1109939, rs10952732, |
| | rs7800558, rs34608876, rs2293176, rs1879901, rs12540766, rs60750216, |
| | rs4725750, rs4342501, rs11763409, rs12704002, rs11975074, rs766690, |
| | rs1681794, rs1681795, rs1639945, rs1639948, rs667416, rs526070, |
| | rs668402, rs528768, rs668466, rs60901407, rs530660, rs669797, |
| | rs555516, rs684246, rs685182, rs2109925, rs10952751, rs2159796, |
| | rs1681786, rs568961, rs590083, rs587373, rs58590331, rs518206, |
| | rs2536955, rs625960, rs674866, rs1639944, rs1860841, rs269224, |
| | rs269222, rs269221, rs269220, rs269219, rs269218, rs269216, rs553597, |
| | rs2536956, rs2429996, rs269235, rs17621539, rs9691566, rs11767550, |
| | rs10952779, rs7787017, rs1681777, rs17621746, rs17621775, |
| | rs10458275, rs10279057, rs269232, rs67718311, rs2109927, rs269227, |
| | rs269263, rs269266, rs2968555, rs2968548, rs2364399, rs7791391, |
| | rs10282499, rs7801086, rs6464037, rs11768253, rs11769042, rs7357151, |
| | rs202148772, rs6943643, rs6961984, rs6947491, rs6953658, rs1267637, |
| | rs1267641, rs1267645, rs1267647, rs1267632, rs1267634, rs986050, |
| | rs1267635, rs6964724, rs2109738, rs1267648, rs4140846, rs1267612, |
| | rs1267615, rs1267616, rs2535948, rs2706775, rs10234502, rs1267608, |
| | rs1267624, rs1267625, rs2535946, rs2706774, rs6947950, rs1267602, |
| | rs1267603, rs1267621, rs1267618, rs6957490, rs10281508, rs7798324, |
| | rs10272940, rs10261732, rs6950312, rs6950946, rs62484301, |
| | rs56797136, rs57248447, rs6944039, rs6948703, rs10258390, rs6954652, |
| | rs10251380, rs10251793, rs10255130, rs62485365, rs11981790, |
| | rs17161762, rs17161763, rs2109307, rs6966197, rs6952095, rs6965532, |
| | rs7800472, rs7793758, rs6957578, rs7776491, rs6952601, rs10255711, |
| | rs9648715, rs9648716, rs10240403, rs28529157, rs10263448, rs7784523, |
| | rs964942, rs765373, rs6464157, rs6464159, rs61640737, rs11974112, |
| | rs557088504, rs801093, rs801098, rs10250563, rs4725437, rs10265001, |
| | rs13236223, rs10268118, rs3864527, rs3852286, rs28618237, |
| | rs12703204, rs10255167, rs12538296, rs7810541, rs9886312, |
| | rs11769174, rs6464278, rs6960603, rs536809, rs498933, rs6944492, |
| | rs553046, rs491554, rs4726263, rs801157, rs801158, rs511001, |
| | rs534348, rs7806026, rs11767331, rs560981, rs722219, rs7778658, |
| | rs970599, rs9640323, rs6962233, rs55949610, rs6967482, rs7805034, |
| | rs7810146, rs11762357, rs6464330, rs1860752, rs56205854, rs10272360, |
| | rs887618, rs10215085, rs1005516, rs17161916, rs10252472, rs11979322, |
| | rs76670977, rs17161924, rs17161926, rs10952447, rs11974746, |
| | rs10242365, rs2270214. |
| CUL1 | rs2717795, rs2717799, rs2717800, rs2538481, rs2160007, rs2253367, |
| 7:148395006- | rs2538478, rs2538477, rs2717804, rs2538476, rs2717805, rs2717806, |
| 148498128 | rs2717807, rs28469775, rs2430227, rs13234330, rs2538473, rs2538470, |
| 257 SNPs | rs2538469, rs2717817, rs2538468, rs11769463, rs1962815, rs200824136, |
| | rs201787709, rs13309779, rs6957883, rs1971691, rs10236063, |
| | rs10280054, rs6968286, rs6964254, rs6971115, rs12704035, rs6464910, |
| | rs6464911, rs6464912, rs4296961, rs7807268, rs55833616, rs2373399, |
| | rs4598173, rs12704037, rs10231800, rs6978741, rs11767479, |
| | rs17171051, rs6948227, rs6974791, rs2888563, rs10233151, rs10259646, |
| | rs7794872, rs36028645, rs4726986, rs12704039, rs10952763, |
| | rs11971205, rs7806010, rs735788, rs2018883, rs10952764, rs10952765, |
| | rs4263667, rs876201, rs4726987, rs6955192, rs6959357, rs6464915, |
| | rs11979232, rs6965357, rs6962115, rs6942631, rs10952766, rs11980746, |
| | rs7782036, rs1015665, rs1024576, rs1024577, rs9648855, rs2190640, |
| | rs2190641, rs2190642, rs6955951, rs6974233, rs6960641, rs10267756, |
| | rs10267894, rs4726988, rs12539045, rs12534012, rs7793987, |
| | rs10229494, rs11763142, rs12669487, rs13240534, rs36059807, |
| | rs377024367, rs11763772, rs13235454, rs10259040, rs6464916, |
| | rs6963551, rs2041074, rs3887126, rs6464918, rs6973481, rs10952773, |
| | rs6952285, rs7812293, rs7789186, rs7781783, rs10225019, rs10262931, |
| | rs6464919, rs6966980, rs4577876, rs6971248, rs10257756, rs6464921, |
| | rs7791723, rs7790647, rs243547, rs243545, rs243544, rs243542, |
| | rs243539, rs243537, rs243536, rs243535, rs243531, rs243529, rs243528, |
| | rs243527, rs243526, rs243525, rs243524, rs243522, rs243519, rs243517, |
| | rs243516, rs243515, rs243514, rs243512, rs243509, rs243507, rs243505, |
| | rs243504, rs243503, rs243502, rs243501, rs243495, rs243494, rs243493, |
| | rs243492, rs243491, rs7810457, rs10255402, rs10278305, rs243482, |
| | rs243481, rs243478, rs11773084, rs11768249, rs3823635, rs3779034, |
| | rs740951, rs2373414, rs3779035, rs6977453, rs758880, rs1558419, |
| | rs10241408, rs2072406, rs933436, rs2007404, rs3779036, rs1019218, |
| | rs7802887, rs9987055, rs10255478, rs10225300, rs887571, rs887570, |
| | rs887569, rs734004, rs734003, rs734002, rs2072407, rs2268036, |
| | rs6464926, rs10245290, rs7810925, rs10952780, rs10952782, |
| | rs10952783, rs12670401, rs13222820, rs71532745, rs34003774, |
| | rs6958755, rs2177567, rs6973259, rs67648693, rs62505405, rs7790642, |
| | rs1357751, rs62505409, rs58371016, rs1676901, rs55692710, |
| | rs12531078, rs6955988, rs6979674, rs6980019, rs7792965, rs6957502, |
| | rs9691477, rs7801276, rs6950380, rs6950521, rs9986856, rs2035217, |
| | rs62505448, rs10243744, rs9690406, rs12667281, rs9691157, rs4302762, |
| | rs2035216, rs9648865, rs13226537, rs146053185, rs117649394, |
| | rs6464927, rs9648866, rs10258164, rs2086087, rs7804977, rs7805123, |
| | rs1094560, rs6965522, rs2691030, rs12055917, rs12533655, rs12534440, |
| | rs10236884, rs573978635, rs12533548, rs58615003, rs34474025, |
| | rs6971528, rs10952787, rs370285106, rs716817, rs10224999, rs749598. |
| KMT2C | rs2374313, rs11981321, rs11974529, rs10229332, rs4298423, rs2374314, |
| 7:151832010- | rs2374315, rs2374316, rs36113236, rs2888798, rs7804584, rs7804751, |
| 152133090 | rs7805160, rs7805336, rs10259821, rs11769246, rs7794505, rs2374318, |
| 231 SNPs | rs12533555, rs7797169, rs10952336, rs6464189, rs62478447, |
| | rs34012876, rs2178307, rs1969557, rs1534664, rs11978256, rs11974761, |
| | rs13240420, rs10243234, rs4726119, rs4726121, rs6464193, rs10268412, |
| | rs10952338, rs4726122, rs4726123, rs4725439, rs4725441, rs7780792, |
| | rs10464433, rs10464435, rs10464436, rs11763523, rs11763539, |
| | rs11760367, rs58612263, rs60688877, rs10271279, rs9648731, |
| | rs61476266, rs4726126, rs1997337, rs62480038, rs58533459, |
| | rs12532588, rs12670289, rs7792399, rs10952343, rs10952344, |
| | rs4549697, rs13310907, rs11766982, rs12671019, rs12668772, |
| | rs12671038, rs12668794, rs62480046, rs11765654, rs10251856, |
| | rs28588502, rs12703192, rs6959600, rs3800836, rs17173373, |
| | rs34580279, rs201736649, rs113966931, rs112498399, rs10252441, |
| | rs62478359, rs62478861, rs113056872, rs200322838, rs201610768, |
| | rs62495467, rs10228402, rs4726154, rs139032733, rs2360210, |
| | rs201078285, rs77166519, rs75794139, rs73164536, rs28490391, |
| | rs62496960, rs7795612, rs7790805, rs374473557, rs35581269, |
| | rs118100744, rs10257415, rs55641834, rs12671476, rs6956909, |
| | rs6957053, rs28775339, rs28370474, rs11984123, rs11772351, |
| | rs13236976, rs4726160, rs6961745, rs10235893, rs2014611, rs12540082, |
| | rs10952355, rs10240610, rs6464231, rs1107394, rs2098063, rs13242116, |
| | rs10248766, rs11768687, rs11525822, rs13237598, rs6970331, |
| | rs6952647, rs12532839, rs57527051, rs12334083, rs10952359, |
| | rs10952360, rs7805788, rs6464242, rs6945852, rs34845804, rs7384229, |
| | rs7385253, rs7385293, rs10447724, rs10447725, rs10447726, |
| | rs35707820, rs13234619, rs7782901, rs4496878, rs10155833, |
| | rs62492151, rs9640181, rs9640309, rs74527415, rs4726170, rs4725459, |
| | rs4726171, rs13241048, rs4726172, rs10155968, rs10156194, |
| | rs62493976, rs73478743, rs62493977, rs62493978, rs62493979, |
| | rs4726173, rs6464244, rs6947738, rs6965541, rs6947893, rs7802601, |
| | rs11974900, rs10952363, rs35678301, rs3923225, rs3923226, |
| | rs13240889, rs59565877, rs11972599, rs35733735, rs7806336, |
| | rs10248434, rs66588533, rs34837268, rs35148779, rs4725461, |
| | rs35848128, rs4726179, rs4480029, rs35356475, rs6464246, rs10237552, |
| | rs376689295, rs55775284, rs28553547, rs59236586, rs60743890, |
| | rs6464250, rs9770945, rs4425666, rs10259153, rs6978007, rs55888610, |
| | rs6964348, rs1468187, rs6464257, rs17812946, rs11769292, rs7777326, |
| | rs10263704, rs10230824, rs35958991, rs10223998, rs6948801, |
| | rs10228207, rs10228550, rs10257576, rs10257825, rs10228778, |
| | rs6955085, rs6975296, rs6959650, rs6979733, rs6959567, rs6979751, |
| | rs34270800, rs10237225, rs10240358, rs10255877, rs10240532, |
| | rs13236518. |
| NKX3-1 | rs7841609, rs4872122, rs12681931, rs6991750, rs62501410, rs4639518, |
| 8:23536206- | rs56278650, rs28656888, rs6557669, rs4546673, rs67681561, rs3885189, |
| 23540440 | rs10866826, rs4872127, rs12676989, rs7840276, rs7823585, rs4617161, |
| 629 SNPs | rs7840451, rs7828700, rs7017792, rs4872129, rs4540417, rs78226694, |
| | rs28794244, rs59478886, rs6557671, rs4872130, rs4872131, rs17089226, |
| | rs4872133, rs7832251, rs10099856, rs11135736, rs6983320, rs11135737, |
| | rs6557672, rs12549539, rs3817676, rs12678121, rs17089262, |
| | rs17088182, rs11787224, rs2272640, rs3779896, rs12675817, |
| | rs12675370, rs7822769, rs11777391, rs9987295, rs11135738, rs6557673, |
| | rs10088178, rs4872136, rs35719007, rs34309579, rs36098102, |
| | rs35066759, rs11784720, rs4872137, rs13259746, rs7837070, |
| | rs10094683, rs13248759, rs4397412, rs7387553, rs17089283, rs2272687, |
| | rs6557675, rs13267246, rs13259091, rs9314265, rs6990526, rs6989547, |
| | rs732563, rs6992783, rs7015154, rs73226459, rs4282585, rs5002136, |
| | rs4289825, rs4289826, rs4565465, rs1010848, rs4872140, rs7813840, |
| | rs4554474, rs4265197, rs13250356, rs9644076, rs34297248, rs6988938, |
| | rs12548053, rs11135740, rs11775345, rs4260902, rs4871876, rs4871877, |
| | rs7837994, rs2048528, rs7009973, rs11323359, rs7826620, rs7837764, |
| | rs13253974, rs7819197, rs2942211, rs62503281, rs4064993, rs73228205, |
| | rs6557678, rs6557679, rs721183, rs4871879, rs2942208, rs7834536, |
| | rs7834883, rs4593549, rs7842120, rs7842144, rs7819907, rs9314268, |
| | rs10098103, rs10107859, rs10503726, rs4872144, rs10503727, |
| | rs12548753, rs12548201, rs7826247, rs2314682, rs2314683, rs11781298, |
| | rs7814821, rs12549572, rs6987271, rs4872148, rs62503324, rs57933450, |
| | rs28884777, rs28783195, rs28778138, rs28821785, rs11135744, |
| | rs7015469, rs6557686, rs7015818, rs7829094, rs7844887, rs7814501, |
| | rs2314684, rs2314685, rs28366418, rs28692182, rs35691692, |
| | rs61271980, rs60392867, rs7820922, rs2280863, rs2280861, rs2280858, |
| | rs35410017, rs7826544, rs7826545, rs4872152, rs2978486, rs2928673, |
| | rs17089352, rs2928672, rs2928671, rs2978473, rs2928670, rs4872153, |
| | rs2978471, rs2928668, rs2942205, rs2942204, rs2928667, rs4512388, |
| | rs2928666, rs4063941, rs2942203, rs2978470, rs4872154, rs7014619, |
| | rs10098490, rs13254494, rs9644032, rs2978487, rs78046237, rs1567668, |
| | rs2942202, rs2942201, rs2942200, rs7843042, rs7833754, rs7830129, |
| | rs6557687, rs2942199, rs2928665, rs2978475, rs3106374, rs10503728, |
| | rs6986233, rs2942197, rs2928686, rs557098911, rs34138581, rs2978482, |
| | rs12545866, rs2978472, rs2942216, rs7003250, rs2942218, rs1877433, |
| | rs2003976, rs2928681, rs2928680, rs2928679, rs7009914, rs6995705, |
| | rs7009429, rs11135750, rs11996109, rs117779632, rs11135753, |
| | rs13263391, rs2314689, rs4871889, rs4871890, rs4871891, rs4871893, |
| | rs7008096, rs6993660, rs3942243, rs13267262, rs6990373, rs7007950, |
| | rs932044, rs932045, rs4242400, rs4242401, rs1028016, rs13264338, |
| | rs950893, rs10503729, rs73215002, rs10503730, rs73216704, |
| | rs73216705, rs6988205, rs6988412, rs28738943, rs11780097, |
| | rs11776817, rs6994472, rs1877434, rs11781262, rs6992687, rs12545639, |
| | rs12549100, rs59341197, rs13269974, rs6994358, rs28623239, |
| | rs13257043, rs13272418, rs13264439, rs13256009, rs1858401, |
| | rs13266769, rs7841597, rs6993298, rs1512273, rs1849308, rs1849309, |
| | rs9650404, rs11135758, rs11135759, rs11995691, rs13254114, |
| | rs7004793, rs7004327, rs7009775, rs7016047, rs7819881, rs7837631, |
| | rs7837924, rs17089443, rs73216733, rs75541123, rs79366973, |
| | rs2314687, rs11135760, rs4872165, rs4872166, rs4872167, rs907585, |
| | rs907586, rs11135761, rs11986046, rs10780136, rs907587, rs868947, |
| | rs73216741, rs10113131, rs872884, rs13254857, rs7003364, rs6998881, |
| | rs77759117, rs7005012, rs4871896, rs11135762, rs73216756, rs7835802, |
| | rs6557705, rs1949164, rs1949163, rs7829619, rs7830220, rs4872170, |
| | rs1398240, rs73216762, rs1398239, rs13256300, rs13256366, |
| | rs17296662, rs201886514, rs66470425, rs3060153, rs7831442, |
| | rs4242402, rs4242403, rs4872171, rs2315144, rs13262683, rs1512271, |
| | rs1398238, rs7006278, rs13274763, rs56014938, rs1512270, rs7821330, |
| | rs995433, rs11135765, rs995432, rs1606303, rs56193509, rs4872174, |
| | rs11782388, rs13265330, rs1512268, rs13272392, rs1971376, rs1160267, |
| | rs13267884, rs56229168, rs11135766, rs59389126, rs10503733, |
| | rs4872175, rs13253010, rs1866342, rs1866343, rs4871898, rs11786742, |
| | rs4872178, rs1866345, rs2314692, rs10866833, rs2314691, rs4872179, |
| | rs4871899, rs10106897, rs2218060, rs4871900, rs1370852, rs11779951, |
| | rs4872182, rs12550569, rs12543268, rs749146, rs749145, rs7000862, |
| | rs35373423, rs10481332, rs13265089, rs28371464, rs548378981, |
| | rs13259516, rs17782111, rs7834389, rs1438456, rs11989744, rs6557708, |
| | rs7844903, rs7826517, rs7827550, rs7826966, rs6984112, rs7836730, |
| | rs17089546, rs11135768, rs4871902, rs4430099, rs2033788, rs1030913, |
| | rs1030912, rs35693687, rs12675971, rs66935439, rs13269080, |
| | rs57441560, rs12678806, rs6987537, rs7005824, rs6991527, rs6557711, |
| | rs7002330, rs10101061, rs6998928, rs58987911, rs7004766, rs7004910, |
| | rs6981261, rs7001541, rs1438454, rs1438455, rs6993869, rs507678, |
| | rs1436141, rs389212, rs443535, rs407329, rs1658912, rs568890, |
| | rs412403, rs574479, rs690960, rs28595810, rs59088821, rs310289, |
| | rs11985119, rs66700932, rs10106791, rs310287, rs17089602, |
| | rs10109560, rs192842, rs310284, rs310283, rs310282, rs10097518, |
| | rs1212399, rs310280, rs11135772, rs1545087, rs7832345, rs188699, |
| | rs12680083, rs310278, rs419481, rs453974, rs690881, rs691136, |
| | rs750022, rs408929, rs310274, rs310275, rs310276, rs218869, |
| | rs17711076, rs444480, rs450336, rs11990455, rs310272, rs310271, |
| | rs310302, rs310303, rs167456, rs310304, rs310305, rs310306, rs310307, |
| | rs444242, rs310310, rs182563, rs429371, rs218871, rs218872, rs310290, |
| | rs310291, rs310292, rs310293, rs310294, rs310295, rs310296, rs310297, |
| | rs310298, rs310299, rs310300, rs310316, rs310317, rs310323, rs310324, |
| | rs310325, rs310326, rs310327, rs1436146, rs192843, rs692701, |
| | rs167457, rs310318, rs310319, rs182565, rs6982337, rs6999954, |
| | rs490124, rs409783, rs414959, rs569137, rs566429, rs4294189, |
| | rs182564, rs310311, rs1992053, rs7010237, rs61053, rs1347548, |
| | rs423432, rs578094, rs3758088, rs1369836, rs3758087, rs3758086, |
| | rs36071802, rs7839091, rs10112428, rs7007761, rs1436148, rs9314272, |
| | rs7009235, rs7813900, rs11989588, rs9771829, rs28375660, rs6999455, |
| | rs6999484, rs11785713, rs7006967, rs11778129, rs11785244, rs1436149, |
| | rs4872203, rs4871906, rs9314273, rs7812843, rs1397799, rs10110261, |
| | rs819193, rs819194, rs819195, rs819196, rs819197, rs819198, |
| | rs62650414, rs151171324, rs140261699, rs530735693, rs375135943, |
| | rs374305801, rs11135778, rs11135779, rs11135780, rs1963425, |
| | rs34861762, rs7003999, rs10100714, rs708620, rs7010107, rs10109414, |
| | rs1731264, rs819186, rs1632501, rs843904, rs819188, rs819189, |
| | rs847324, rs819190, rs13439227, rs1705689, rs1705690, rs2872991, |
| | rs1731273, rs7834797, rs17089719, rs11777108, rs10103918, rs7816082, |
| | rs13278100, rs1731274, rs1705691, rs1705693, rs1705694, rs1705695, |
| | rs1320880, rs9650405, rs1731267, rs1705696, rs1511387, rs17786744, |
| | rs1705698, rs1705699, rs1705700, rs1705701, rs1320879, rs13267474, |
| | rs4872205, rs1629026, rs1629043, rs1631430, rs1631550, rs4871907, |
| | rs1731265, rs1705702, rs1731263, rs59657583, rs562579190. |
| CLU | rs2241650, rs2241651, rs3824104, rs17376255, rs2322718, rs2115805, |
| 8:27454434- | rs2163174, rs2163175, rs4732722, rs67432684, rs2059968, rs2059969, |
| 27472548 | rs60795589, rs7005183, rs1367088, rs1367089, rs11135991, rs3779633, |
| 250 SNPs | rs725788, rs976175, rs6996432, rs28840432, rs73681119, rs6988123, |
| | rs6993151, rs6994446, rs2322714, rs7828688, rs750536, rs750537, |
| | rs750538, rs750539, rs10087410, rs881188, rs2003246, rs56330356, |
| | rs882397, rs3779634, rs13268201, rs35609762, rs7846568, rs3779635, |
| | rs13254138, rs13257998, rs7827965, rs41276295, rs11988406, |
| | rs2303881, rs2303882, rs34900744, rs12056620, rs1896960, rs2241654, |
| | rs7005954, rs11995441, rs2241655, rs10091396, rs3736527, rs3736526, |
| | rs3736525, rs9657294, rs2241659, rs2241660, rs2241661, rs10113756, |
| | rs1991570, rs9657295, rs4733063, rs11135993, rs10098202, rs11776693, |
| | rs7000364, rs7846626, rs7831523, rs4733065, rs751018, rs10093964, |
| | rs7007145, rs752994, rs752993, rs919494, rs748283, rs2741343, |
| | rs2565048, rs2741341, rs7819756, rs2741338, rs2741336, rs12548501, |
| | rs36081056, rs2640726, rs2640725, rs2741354, rs1873933, rs2741352, |
| | rs2640722, rs2640721, rs2741350, rs2741349, rs2741348, rs17466060, |
| | rs4732724, rs9632863, rs1565736, rs1316801, rs1000108, rs7831810, |
| | rs7814146, rs1532279, rs10780145, rs4352801, rs17057419, rs7828131, |
| | rs4732728, rs6983452, rs7012010, rs7012217, rs17057438, rs7817366, |
| | rs7821500, rs4732729, rs9331908, rs11136000, rs11787077, rs1532276, |
| | rs9331896, rs538181, rs569205, rs484377, rs520186, rs2582367, |
| | rs1073743, rs1073742, rs569214, rs491672, rs492587, rs485902, |
| | rs894019, rs894020, rs545111, rs548561, rs553290, rs575282, rs779959, |
| | rs2582370, rs561690, rs577803, rs2640732, rs560397764, rs2640733, |
| | rs480291, rs519311, rs492786, rs516125, rs542876, rs542794, rs541955, |
| | rs505295, rs75320, rs503508, rs559251, rs512941, rs555300, rs7001584, |
| | rs3103908, rs1961369, rs1826549, rs1961368, rs7464621, rs13272497, |
| | rs542715, rs541661, rs11778402, rs509475, rs514196, rs11779395, |
| | rs693841, rs62497991, rs521426, rs112045356, rs7823813, rs554687, |
| | rs17384485, rs522881, rs553014, rs477060, rs563890, rs12155773, |
| | rs10216990, rs11778467, rs4352802, rs7465373, rs4585703, rs898477, |
| | rs17385236, rs13249159, rs1809285, rs2010937, rs898475, rs2010903, |
| | rs2010763, rs7830136, rs7833799, rs3735751, rs13273652, rs11782418, |
| | rs11784867, rs11136006, rs7825085, rs12681592, rs34283399, |
| | rs12334989, rs7831555, rs10103758, rs7016934, rs2294028, rs7013429, |
| | rs7013435, rs10216777, rs1073235, rs718422, rs725360, rs725361, |
| | rs4146307, rs10866862, rs11782593, rs11782624, rs2280884, rs1832, |
| | rs10101568, rs10091801, rs10101588, rs12545651, rs7831769, |
| | rs4538825, rs7818903, rs12679973, rs3937606, rs9693289, rs35951849, |
| | rs11787128, rs10105720, rs2272730, rs3802154, rs4470991, rs7842005, |
| | rs9773688, rs6558015, rs4732754, rs10086140. |
| NCOA2 | rs7814296, rs10087968, rs59736991, rs192397053, rs55786188, |
| 8:71021997- | rs67493286, rs4738050, rs13259481, rs13260145, rs13262185, |
| 71316040 | rs11986451, rs7013896, rs7843395, rs7827151, rs7843437, rs12679440, |
| 245 SNPs | rs7822729, rs16936566, rs35804085, rs2380608, rs111919154, |
| | rs7835834, rs7836437, rs7836534, rs12679592, rs1466167, rs4607655, |
| | rs7017913, rs28409961, rs28609895, rs1562897, rs6472498, rs12335262, |
| | rs12335268, rs12335270, rs6472499, rs67358028, rs7844954, rs4738052, |
| | rs10957511, rs6997086, rs7015329, rs4316181, rs7814786, rs7836791, |
| | rs6472500, rs6472501, rs10106857, rs6994052, rs4738053, rs6985748, |
| | rs10090560, rs10105638, rs10504462, rs1809104, rs7821231, |
| | rs13258645, rs2060789, rs2044888, rs2580205, rs2599391, rs2253212, |
| | rs7825216, rs2580204, rs10103470, rs2044885, rs7825431, rs4737294, |
| | rs10092351, rs6993479, rs6994514, rs10957514, rs6999884, rs7000079, |
| | rs2380622, rs7017578, rs7009598, rs4738062, rs4738063, rs12678469, |
| | rs12676337, rs10101704, rs4738065, rs7822888, rs7844675, rs1982413, |
| | rs4412398, rs3750226, rs10112802, rs11785095, rs6472507, rs6472508, |
| | rs7010162, rs7829148, rs9969596, rs12335295, rs11986044, rs60917951, |
| | rs12543553, rs28664277, rs4364664, rs10504466, rs7824126, rs9773950, |
| | rs9773974, rs13248740, rs35069822, rs67822483, rs3892266, rs1106334, |
| | rs12674927, rs12674943, rs4546698, rs4571760, rs3763523, rs4545135, |
| | rs13260857, rs2957094, rs2272670, rs2979633, rs877181, rs16936790, |
| | rs4738075, rs9298156, rs7003804, rs4738076, rs7015854, rs10087049, |
| | rs10091910, rs62530460, rs4737301, rs11775170, rs4738078, rs4484728, |
| | rs11785897, rs10086357, rs4236984, rs2977985, rs2977986, rs2926719, |
| | rs2977988, rs2926715, rs2958365, rs2926713, rs2926712, rs2958366, |
| | rs2958367, rs2926706, rs2926703, rs2926701, rs2977980, rs1473409, |
| | rs7008415, rs6472514, rs17677348, rs16936881, rs6472517, rs7015784, |
| | rs6997367, rs7837694, rs7001707, rs7815765, rs7815798, rs34017261, |
| | rs1380896, rs1014160, rs16936926, rs12680132, rs12676045, |
| | rs10504473, rs7841047, rs7812666, rs7818350, rs6472520, rs6472522, |
| | rs6986140, rs12679364, rs13276919, rs6472523, rs6991199, rs7813786, |
| | rs7823221, rs11775808, rs11775822, rs6472527, rs6472528, rs10504477, |
| | rs35014916, rs16937001, rs28689781, rs28715739, rs10089804, |
| | rs10957521, rs2170304, rs28455631, rs72665732, rs10091864, |
| | rs10092365, rs6997427, rs12156267, rs9657093, rs1481045, rs1481046, |
| | rs7816549, rs10096078, rs2380651, rs59211000, rs7460301, rs28507404, |
| | rs7838853, rs10091374, rs10087358, rs12678139, rs6472533, |
| | rs73685630, rs2622646, rs36163735, rs28394070, rs4738094, rs2622647, |
| | rs4077393, rs7830427, rs7830997, rs4351447, rs10481244, rs10106858, |
| | rs10481212, rs12542675, rs4556114, rs6472537, rs3954895, rs12679081, |
| | rs9886439, rs268589, rs268584, rs389105, rs389106, rs268582, |
| | rs268561, rs268562, rs268624, rs268592, rs268593, rs268594, rs268596. |
| MYC | rs12386846, rs4407842, rs149744296, rs7011405, rs7825312, rs7845403, |
| 8:128747680- | rs6470530, rs6470531, rs4284014, rs7011721, rs7843328, rs7829243, |
| 128753674 | rs7813874, rs4301412, rs6470532, rs4285449, rs75741226, rs76780643, |
| 267 SNPs | rs7386544, rs7387259, rs10110900, rs4313119, rs62514015, rs12681454, |
| | rs57683201, rs9642879, rs11786518, rs4733766, rs4733767, rs28427044, |
| | rs7387723, rs6470538, rs13282785, rs7819582, rs6415496, rs6415497, |
| | rs6470540, rs6470541, rs7834068, rs6470543, rs13252781, rs13260610, |
| | rs13253038, rs4129665, rs13259282, rs7831558, rs13253976, |
| | rs34476315, rs4526325, rs4310167, rs7826669, rs6470544, rs4733841, |
| | rs4733597, rs111911808, rs7815587, rs9643228, rs9643229, rs7004068, |
| | rs7004497, rs7004982, rs7004743, rs6986983, rs7005795, rs7010032, |
| | rs6470546, rs6470547, rs6470548, rs7840283, rs7843970, rs12547587, |
| | rs11990665, rs13272676, rs6989071, rs7009079, rs6989214, rs6993044, |
| | rs7013852, rs6470549, rs7824930, rs7825061, rs7843324, rs4551308, |
| | rs4581009, rs4551309, rs6999106, rs6470556, rs4733854, rs7818517, |
| | rs4354269, rs13260699, rs13262702, rs13264787, rs62514053, |
| | rs6985831, rs62514055, rs36061397, rs60990950, rs10110022, |
| | rs4733867, rs13269551, rs4348452, rs56189189, rs10956380, |
| | rs10956381, rs4733878, rs58886745, rs13280784, rs11995971, |
| | rs11995972, rs57054592, rs13278845, rs56397659, rs7817632, |
| | rs10956382, rs4517084, rs7003158, rs7003603, rs6985681, rs4626562, |
| | rs4385433, rs4609164, rs7018023, rs13250163, rs9297765, rs368162161, |
| | rs13248476, rs58335898, rs13268237, rs60367803, rs61693355, |
| | rs35406750, rs34150073, rs16902304, rs56036131, rs4733658, |
| | rs10109982, rs6991559, rs4733664, rs6986806, rs6470559, rs4733671, |
| | rs7845292, rs10091370, rs7831838, rs7820103, rs7820252, rs10109160, |
| | rs4351385, rs4582519, rs4733676, rs6984323, rs4357254, rs10111134, |
| | rs10096900, rs4478537, rs9642880, rs10094872, rs7841229, rs4733548, |
| | rs16902359, rs3891248, rs12680047, rs11782002, rs4733550, |
| | rs62515815, rs6470574, rs6988558, rs13277355, rs10112382, |
| | rs10956389, rs7831499, rs4073927, rs4326353, rs6470575, rs7825248, |
| | rs6470576, rs7007699, rs4733759, rs4524749, rs10956390, rs6990534, |
| | rs7841347, rs6470579, rs10108514, rs7833614, rs7815347, rs12216820, |
| | rs12216822, rs6470581, rs4733778, rs10481168, rs4733779, rs28617188, |
| | rs4733789, rs17188737, rs13272369, rs12543944, rs10089888, |
| | rs4733577, rs4733795, rs4733796, rs4733798, rs55939879, rs7825479, |
| | rs56209901, rs4463382, rs4332094, rs16902434, rs13259137, |
| | rs72609868, rs78212463, rs150443181, rs146186111, rs139562860, |
| | rs13280024, rs6998562, rs9297769, rs890440, rs13261159, rs28459758, |
| | rs4427136, rs2114353, rs2114354, rs10956392, rs6470588, rs6470589, |
| | rs12679245, rs10808560, rs6986878, rs6999585, rs7017386, rs7016286, |
| | rs62512785, rs2555114, rs7387614, rs7387606, rs890442, rs10808561, |
| | rs7834600, rs7835090, rs10956396, rs10956397, rs13254020, |
| | rs10956398, rs3739378, rs3739379, rs7014406, rs7015082, rs10441527, |
| | rs10104535, rs11782062, rs11782761, rs28475595, rs28608914, |
| | rs13269522, rs4733581, rs13277076, rs13278957, rs4733583, rs1365371, |
| | rs2392827, rs6992539, rs11782183, rs13263872, rs890436, rs11990664. |
| CDKN2A | rs3849929, rs7029036, rs10121899, rs1341866, rs7037577, rs9886831, |
| 9:21967751- | rs2152273, rs1414229, rs7466000, rs10965128, rs10811615, rs935053, |
| 21995300 | rs958219, rs7863942, rs1345024, rs1345023, rs1345022, rs1345021, |
| 233 SNPs | rs1345020, rs1542074, rs7049092, rs7018772, rs7038708, rs7022754, |
| | rs7022856, rs10965133, rs10965134, rs10965135, rs10811617, |
| | rs10811618, rs1542075, rs1542076, rs12344842, rs10119568, |
| | rs10119693, rs4364717, rs935055, rs10757257, rs10965141, rs67159519, |
| | rs10965143, rs10965145, rs10811623, rs72691561, rs72691562, |
| | rs72691563, rs72691564, rs58377678, rs7023329, rs7023680, |
| | rs10114559, rs7023474, rs7027989, rs3928894, rs4431674, rs7852450, |
| | rs10121449, rs6475585, rs3922992, rs3927737, rs7868783, rs7046060, |
| | rs10811629, rs4977733, rs11523031, rs4977567, rs4478653, rs7039105, |
| | rs7047648, rs4977737, rs10965171, rs4977738, rs4977739, rs4607709, |
| | rs3928893, rs4607704, rs4593639, rs4977569, rs10118406, rs7044807, |
| | rs7028211, rs12236681, rs10811637, rs4977746, rs7852128, rs7852234, |
| | rs6475597, rs4977747, rs4977748, rs2188125, rs7864029, rs2518714, |
| | rs10965197, rs2106117, rs2106118, rs2382894, rs10757260, rs10757261, |
| | rs12335941, rs2518720, rs3731197, rs2811710, rs3218020, rs3218012, |
| | rs10811640, rs10757263, rs10811641, rs2106120, rs2106119, rs643319, |
| | rs7044859, rs10757264, rs490005, rs568447, rs567453, rs504318, |
| | rs496892, rs10811643, rs10811644, rs1591136, rs7049105, rs4977753, |
| | rs10115049, rs2151280, rs1360590, rs1333036, rs10120806, rs7027950, |
| | rs7028268, rs7028570, rs10757265, rs10757266, rs944799, rs10757267, |
| | rs10965219, rs7027048, rs7874604, rs2383204, rs10120688, rs10811647, |
| | rs1333039, rs10965224, rs10811648, rs10811649, rs10811650, |
| | rs10811651, rs4977756, rs4645630, rs9632885, rs10757270, rs1831733, |
| | rs1333040, rs1537370, rs10738606, rs10738607, rs10757272, |
| | rs10757274, rs4977574, rs2891168, rs7859727, rs1537372, rs10511701, |
| | rs2383206, rs1537375, rs1537376, rs1333045, rs10217586, rs10738610, |
| | rs1333046, rs10757277, rs10811656, rs10757278, rs10757279, |
| | rs1333048, rs1333049, rs1333050, rs1889086, rs10811658, rs2891169, |
| | rs7045889, rs10217762, rs10757282, rs10757283, rs7019778, |
| | rs12555274, rs7018475, rs10757284, rs4977761, rs2065501, rs7026735, |
| | rs2065504, rs6475610, rs7849199, rs7849302, rs2151285, rs2151283, |
| | rs2151282, rs12341394, rs7856219, rs6475614, rs182266453, rs7029976, |
| | rs7046298, rs7030278, rs12343752, rs7028213, rs1333052, rs10122243, |
| | rs7864275, rs10757290, rs7854623, rs7041895, rs12348411, rs17694933, |
| | rs1333054, rs6475617, rs1412837, rs7863846, rs1537377, rs954399, |
| | rs2779747, rs828582, rs10811669, rs9987548, rs1981046, rs9987689, |
| | rs10738612, rs2767408, rs1095899, rs1095901, rs78682034, rs2767409, |
| | rs1095896. |
| FANCG | rs9298960, rs10814177, rs276677, rs276678, rs276680, rs72722903, |
| 9:35073832- | rs12683157, rs6476468, rs2455088, rs7847764, rs7864656, rs57543330, |
| 35080013 | rs112832076, rs201025511, rs10972254, rs2455089, rs628789, |
| 245 SNPs | rs12686575, rs6476469, rs10123308, rs10814178, rs10429551, rs504164, |
| | rs7854429, rs276670, rs593680, rs10124273, rs12005698, rs2134103, |
| | rs643537, rs7022120, rs12005235, rs12001305, rs60706299, rs7043303, |
| | rs11792360, rs611137, rs7849669, rs2782403, rs7047186, rs7021062, |
| | rs11794043, rs2050362, rs10814183, rs7861385, rs10758284, |
| | rs16932081, rs4879836, rs11792836, rs1582067, rs7020615, rs10758285, |
| | rs2173897, rs10120934, rs200174687, rs1827332, rs1580849, rs2173898, |
| | rs10116521, rs10814184, rs10117371, rs58109322, rs1334756, rs905104, |
| | rs10758287, rs4879839, rs10972259, rs10972261, rs4878593, rs4879841, |
| | rs10972262, rs10738935, rs10814187, rs11792835, rs7848532, |
| | rs7864127, rs7848782, rs6476471, rs7851977, rs77499912, rs11536750, |
| | rs62548014, rs11790647, rs7031733, rs7025995, rs7039317, rs7039450, |
| | rs10972273, rs10972274, rs7874491, rs10122262, rs10116097, |
| | rs7048812, rs10123919, rs7047205, rs7045765, rs11789226, rs4879846, |
| | rs2297647, rs4879847, rs17293327, rs4879849, rs10814191, rs10814192, |
| | rs10972278, rs7856118, rs10972281, rs10972282, rs4878595, |
| | rs10972283, rs4879852, rs10972284, rs4879853, rs4878596, rs4878597, |
| | rs7859094, rs12002486, rs4879854, rs10814193, rs12237131, rs581927, |
| | rs1634951, rs2252749, rs679739, rs679787, rs10972297, rs2802384, |
| | rs813422, rs642296, rs506641, rs10814194, rs511228, rs540535, |
| | rs565070, rs595429, rs684562, rs562381, rs2258240, rs607671, |
| | rs514492, rs622945, rs637885, rs739844, rs587118, rs554098, rs504082, |
| | rs584040, rs546982, rs576376, rs2525081, rs527459, rs505297, |
| | rs568300, rs10758291, rs2802385, rs633027, rs527365, rs2905844, |
| | rs35428985, rs525052, rs666549, rs586017, rs630961, rs4127857, |
| | rs10972320, rs7852999, rs10123856, rs10972321, rs10738936, |
| | rs1571954, rs1571953, rs10972324, rs11791629, rs10758292, |
| | rs10758293, rs10758294, rs10814197, rs4878604, rs10116368, |
| | rs4879865, rs10814198, rs10814199, rs4879867, rs10814200, |
| | rs36169391, rs58479853, rs10972334, rs10814202, rs28371950, |
| | rs10972338, rs11789592, rs10814203, rs10972339, rs10814204, |
| | rs7852146, rs4879869, rs4879870, rs10972341, rs7046814, rs61508727, |
| | rs4879873, rs10972342, rs7856842, rs7857051, rs4879874, rs10972345, |
| | rs7862024, rs59405789, rs10972346, rs10972348, rs10972350, |
| | rs7028146, rs4878605, rs10972352, rs10972353, rs13285401, |
| | rs58046590, rs10081672, rs11521201, rs12352790, rs13293564, |
| | rs10465027, rs10814211, rs10121073, rs2381221, rs3904435, rs3904434, |
| | rs7031461, rs10972381, rs7032682, rs7036061, rs12685290, rs10283768, |
| | rs2381301, rs2381302, rs10814215, rs7851161, rs10118130, rs10972406, |
| | rs10814217, rs7025623, rs10758301, rs1930361, rs10121009, |
| | rs10114937. |
| FANCC | rs10993387, rs12347649, rs12338859, rs10821413, rs2183958, |
| 9:97861336- | rs4744403, rs16911824, rs12235316, rs10993397, rs10993398, |
| 98079991 | rs7853195, rs4744411, rs4744412, rs7870160, rs10821415, rs7865239, |
| 236 SNPs | rs2404295, rs10993427, rs10993436, rs7048237, rs10993453, |
| | rs10761364, rs7873479, rs4744422, rs4744423, rs7865144, rs3802456, |
| | rs10761366, rs7851173, rs1113102, rs3802454, rs1564234, rs7022452, |
| | rs1571895, rs1571894, rs7043705, rs4744424, rs962229, rs4744425, |
| | rs7021351, rs10761369, rs6479591, rs6479592, rs2061800, rs7854831, |
| | rs4537362, rs2404305, rs200077633, rs7857679, rs4744426, rs4743979, |
| | rs7862156, rs10761370, rs10761372, rs10993463, rs7871218, rs7873567, |
| | rs7863291, rs10993464, rs10119512, rs7048786, rs7048941, rs4743980, |
| | rs4743981, rs4743982, rs4743984, rs4744428, rs10117370, rs1004373, |
| | rs4744430, rs2404456, rs4744431, rs4744432, rs4744433, rs10512253, |
| | rs10123819, rs10121760, rs4744435, rs4744436, rs7020709, rs7042320, |
| | rs2169819, rs10761374, rs7034586, rs7023722, rs7042293, rs7046682, |
| | rs7033633, rs10821446, rs10491560, rs4743988, rs1011784, rs1045276, |
| | rs6479598, rs4744442, rs10761375, rs7863582, rs9299428, rs10821450, |
| | rs10821451, rs3780561, rs3824486, rs2282216, rs3780562, rs879492, |
| | rs2404457, rs4647534, rs3780564, rs881791, rs667946, rs3780567, |
| | rs3780568, rs3277, rs2166402, rs565293, rs3737142, rs10821453, |
| | rs7850958, rs2297597, rs2297596, rs10821454, rs200056699, rs7034538, |
| | rs7023007, rs4647475, rs4744446, rs963774, rs10739992, rs6479601, |
| | rs4647462, rs4647459, rs34045846, rs10761377, rs1979022, rs7854570, |
| | rs17686583, rs10993490, rs4647443, rs356667, rs34532504, rs7021497, |
| | rs4744451, rs3844341, rs3852402, rs4647376, rs356665, rs356664, |
| | rs7027204, rs2031599, rs554879, rs473825, rs4744454, rs7026047, |
| | rs1027710, rs10821458, rs10821459, rs10821460, rs4744455, rs4743993, |
| | rs2810915, rs10761382, rs6479603, rs113532927, rs35081772, |
| | rs7858493, rs10761387, rs10821461, rs10821462, rs2036516, rs421224, |
| | rs6479605, rs357550, rs357549, rs357548, rs357547, rs173347, |
| | rs35431736, rs405235, rs372958, rs357543, rs357542, rs9695482, |
| | rs357540, rs357539, rs905615, rs357532, rs357533, rs189614, |
| | rs10993529, rs357536, rs10993534, rs12004780, rs413848, rs10821464, |
| | rs913711, rs2404693, rs357528, rs6479607, rs7388704, rs7389278, |
| | rs28471674, rs28778897, rs62558314, rs2036515, rs2036514, |
| | rs28677810, rs357557, rs35041950, rs28459853, rs357552, rs357551, |
| | rs2896944, rs28417125, rs357522, rs357563, rs28620532, rs28701981, |
| | rs2236406, rs2274692, rs2066829, rs2236407, rs2297086, rs2297088, |
| | rs28557305, rs28831479, rs28504650, rs10512248, rs28457763, |
| | rs28629854, rs16909919, rs4448343, rs28620668, rs1355620, rs3758302, |
| | rs28496034, rs28591501. |

^{†}gene-region loci are provided with reference to the Genome Reference Consortium Human Build 37 (GRCh37), which is herein also referred to as “hg19”. The loci are provide in the following form: X:Y-Z, wherein X is the chromosome name, Y is the start position of the gene-region, and Z is the end position of the gene-region.

The gene-regions recited above in Table 1 are gene-regions that contain genes mutated in prostate cancer and/or altered in signalling pathways known or suspected of being druggable pathways for treating a prostate cancer. The gene-regions defined in Table 1 are hereinafter referred as “target gene-regions”.

TABLE 2
Control gene-regions and SNPs

| gene-region | |
|---|---|
| ref/ gene- | |
| region loci^{†}/ | |
| number of | |
| SNPs | SNPs (rsids) |
| PTAFR | rs6683984, rs4908384, rs4243820, rs6699701, rs33981147, rs7529792, |
| 1:28292661- | rs11247731, rs12040932, rs77809679, rs12031542, rs12031544, |
| 28709972 | rs12139137, rs11247734, rs7549174, rs7521893, rs12140070, |
| 199 SNPs | rs12144891, rs72656597, rs2815708, rs7551152, rs11581035, |
| | rs11247739, rs4908389, rs546355, rs525844, rs6687671, rs2476547, |
| | rs490149, rs1769703, rs528866707, rs139162912, rs10902684, |
| | rs11247742, rs6661800, rs11583358, rs521908, rs551355, rs12137852, |
| | rs72658347, rs12034833, rs577830, rs2790717, rs559346, rs560958, |
| | rs9786989, rs11807799, rs12144063, rs12144104, rs11588733, rs477175, |
| | rs2989472, rs55714013, rs11582538, rs61786971, rs6678050, |
| | rs11247752, rs548558, rs2481981, rs536132, rs6598919, rs6598920, |
| | rs11585562, rs540424, rs6689346, rs516140, rs572673, rs495223, |
| | rs506922, rs6689073, rs55763932, rs72658395, rs9701904, rs515649, |
| | rs1769711, rs12039701, rs12039737, rs12040792, rs10902686, |
| | rs35675074, rs6673554, rs10902688, rs11247756, rs10794512, |
| | rs10751727, rs313153, rs313150, rs61786981, rs111715719, rs2481974, |
| | rs2456267, rs61787028, rs905908, rs1049504, rs12095236, rs4387200, |
| | rs61787029, rs510379, rs61787033, rs12096842, rs571131, rs796848, |
| | rs537713, rs567086, rs547100, rs559509, rs482249, rs12129745, |
| | rs545197, rs12408077, rs12406992, rs580800, rs517085, rs508772, |
| | rs503048, rs502273, rs543240, rs487437, rs7532379, rs11247766, |
| | rs7526084, rs6669686, rs12132464, rs61542967, rs12134320, |
| | rs12040403, rs12564741, rs12754828, rs12563430, rs12564304, |
| | rs11247770, rs7523070, rs11247771, rs872641, rs10794514, rs56032811, |
| | rs11247776, rs12144013, rs11247777, rs4908399, rs11247779, |
| | rs11247781, rs12117842, rs111627638, rs67271330, rs72880354, |
| | rs12118825, rs9438602, rs9662425, rs9660304, rs9659340, rs9659586, |
| | rs66603661, rs12062157, rs74664341, rs12076477, rs12062670, |
| | rs11247783, rs11247784, rs11247785, rs12124673, rs11247786, |
| | rs7514960, rs7515051, rs111273182, rs68149322, rs9660549, rs7533130, |
| | rs7533232, rs11247787, rs11247788, rs6682922, rs12143408, |
| | rs12124751, rs4908345, rs4908401, rs4908346, rs4908347, rs112818191, |
| | rs6703793, rs6703997, rs6657867, rs6657880, rs67856516, rs12118457, |
| | rs11247792, rs4908411, rs111662128, rs4908412, rs4908413, |
| | rs12137056, rs4908415, rs12071848, rs12076528, rs10902699, |
| | rs10794517, rs6663700, rs6679034, rs6690026, rs11247795 |
| FCER1A | rs2518565, rs2814760, rs2814759, rs2078723, rs2814757, rs2852723, |
| 1:159070032- | rs2852727, rs1894044, rs1122609, rs2518568, rs2518569, rs2518570, |
| 159467034 | rs2814779, rs1103577, rs863027, rs1737978, rs863030, rs863032, |
| 214 SNPs | rs863035, rs863036, rs863039, rs3026943, rs2213949, rs2814775, |
| | rs3026946, rs2814774, rs965358, rs926634, rs2249581, rs876694, |
| | rs2249866, rs2249872, rs3026968, rs1474747, rs2814773, rs2852714, |
| | rs724363, rs2253114, rs2281299, rs2852715, rs28695249, rs2852716, |
| | rs16841832, rs2281300, rs2253837, rs862988, rs862989, rs862991, |
| | rs862995, rs862998, rs863004, rs863006, rs34127467, rs863007, |
| | rs3027044, rs3027045, rs34023441, rs34982367, rs3027048, rs3027051, |
| | rs3027052, rs12034864, rs35105792, rs10437335, rs3027055, rs3027056, |
| | rs3027061, rs3027063, rs863008, rs35921532, rs12084987, rs11265146, |
| | rs3027074, rs863009, rs863011, rs4083420, rs201577285, rs3845624, |
| | rs2875781, rs2325917, rs2325918, rs7537391, rs7546146, rs2325920, |
| | rs2325921, rs12067482, rs10908702, rs7531546, rs2494256, rs2494257, |
| | rs34587336, rs12121203, rs60069993, rs11265159, rs2325924, |
| | rs199828328, rs200757971, rs61267648, rs12075558, rs2875783, |
| | rs12083313, rs12097396, rs12087219, rs11265163, rs11265164, |
| | rs67443337, rs60861070, rs59012264, rs10908703, rs2494262, |
| | rs2511213, rs2511214, rs2511215, rs2247584, rs112553301, rs12137347, |
| | rs12134063, rs12135788, rs2494264, rs7548864, rs6685532, rs2252226, |
| | rs2494251, rs12061273, rs11265166, rs12724895, rs6678381, rs4282816, |
| | rs4466650, rs140001158, rs12028986, rs11265171, rs7546296, |
| | rs10908708, rs12128960, rs12132451, rs28479088, rs12136402, |
| | rs10218647, rs10908709, rs11265174, rs10908710, rs10908711, |
| | rs4443888, rs9782926, rs4402126, rs11265178, rs12044815, rs4517343, |
| | rs4537549, rs6692378, rs6692609, rs6677678, rs10908713, rs10908714, |
| | rs4400605, rs7548736, rs4130236, rs6702853, rs6687840, rs7542509, |
| | rs6701545, rs4405137, rs12402605, rs11582600, rs4446959, |
| | rs144025833, rs3122631, rs3122630, rs3122629, rs3131547, rs3122628, |
| | rs12741297, rs4625277, rs3122633, rs111405935, rs9427349, rs9427018, |
| | rs4409648, rs12034969, rs11265190, rs56276965, rs4360519, |
| | rs11265191, rs11265194, rs12145079, rs10908720, rs12048482, |
| | rs4656235, rs11589258, rs4261112, rs34388918, rs12023055, |
| | rs11265196, rs6675789, rs12133577, rs11265204, rs12034243, |
| | rs4339862, rs11265205, rs10797035, rs4233351, rs4233352, rs12734524, |
| | rs12127080, rs7521220, rs10797037, rs61821535, rs55960614, |
| | rs6680456, rs6683999, rs4656833, rs4253910, rs6685392 |
| APBB1IP | rs1889690, rs1889689, rs1889688, rs1537744, rs1537743, rs1335542, |
| 10:26667426- | rs2992285, rs3006787, rs1537742, rs1537741, rs2210789, rs6482558, |
| 26894404 | rs10764607, rs3006783, rs2992288, rs3006782, rs2050448, rs11015080, |
| 222 SNPs | rs10828985, rs17528774, rs867354, rs10764608, rs6482559, rs7917375, |
| | rs10764609, rs2992300, rs3006832, rs2992301, rs9418541, rs2992305, |
| | rs945298, rs868093, rs870884, rs3006831, rs2992306, rs3006830, |
| | rs3006829, rs2992312, rs35468784, rs9418544, rs9417470, rs12766023, |
| | rs113156607, rs2992313, rs2992314, rs2992315, rs2992316, rs2992317, |
| | rs2992318, rs3006828, rs7902283, rs7919365, rs7919521, rs7919527, |
| | rs7902565, rs3006827, rs3006826, rs9418548, rs3006825, rs10828989, |
| | rs11015097, rs2992329, rs4641362, rs2992330, rs7901944, rs56075553, |
| | rs57958218, rs61838397, rs4749124, rs4747555, rs11015103, rs7895480, |
| | rs7899165, rs7895970, rs7910335, rs10828990, rs7900319, rs1889694, |
| | rs7096910, rs10828992, rs4747557, rs1537746, rs1932253, rs7919155, |
| | rs4749127, rs7100078, rs7099764, rs7099792, rs7069919, rs10828993, |
| | rs10828994, rs10828995, rs4120547, rs11015110, rs11015111, |
| | rs7900211, rs4749128, rs4749130, rs4749131, rs6482560, rs6482561, |
| | rs4747559, rs4749132, rs4749133, rs4749134, rs10828996, rs10828997, |
| | rs10764611, rs10764612, rs10764613, rs10764614, rs10764615, |
| | rs10828998, rs10764617, rs10828999, rs10764618, rs10764619, |
| | rs10764620, rs10829000, rs61838426, rs4749135, rs11015113, |
| | rs11015114, rs10764621, rs10741109, rs1414285, rs1414286, rs1414287, |
| | rs10829001, rs10829002, rs4601668, rs7903528, rs7907042, rs7908121, |
| | rs4749137, rs10764622, rs4747562, rs11015116, rs11015117, |
| | rs11015118, rs11015119, rs10047347, rs10047348, rs10047274, |
| | rs10047359, rs10047360, rs2184564, rs1360844, rs3920764, rs4237377, |
| | rs4237378, rs4354610, rs4518996, rs4275518, rs7068804, rs4749142, |
| | rs4747563, rs4747564, rs4749143, rs945291, rs10829005, rs4749144, |
| | rs4747565, rs4749146, rs4747566, rs4617486, rs4375347, rs4375348, |
| | rs4237379, rs4749147, rs4749148, rs4747567, rs7076518, rs10829006, |
| | rs2065753, rs2065754, rs10829007, rs1537748, rs1537749, rs10741110, |
| | rs1537751, rs1335541, rs947719, rs947720, rs947721, rs11015122, |
| | rs35950573, rs12243470, rs7902872, rs702996, rs10764627, rs10764628, |
| | rs12783959, rs10829015, rs10764629, rs787069, rs787067, rs787051, |
| | rs787050, rs787049, rs2488350, rs1753347, rs1621334, rs1617047, |
| | rs10764631, rs10829021, rs7902011, rs7917257, rs10741113, |
| | rs10764632, rs10741114, rs787011, rs787006, rs796820, rs1775241, |
| | rs6482566, rs2985464, rs2448102, rs2477267, rs7903286, rs10829032, |
| | rs12241186 |
| CSTF2T | rs12415593, rs10823113, rs10823114, rs113824835, rs4375388, |
| 10:53271368- | rs10762184, rs10997890, rs2026559, rs2026557, rs10823129, rs1475362, |
| 53648567 | rs10997931, rs10998013, rs10998031, rs71508084, rs12263906, |
| 221 SNPs | rs7901431, rs12761104, rs10998080, rs6480332, rs4145285, rs55862651, |
| | rs28821505, rs10740303, rs11592637, rs955719, rs10823243, |
| | rs12262193, rs10823253, rs10823255, rs10762241, rs2339796, |
| | rs1922139, rs10740313, rs10998486, rs10823275, rs4118256, rs6480375, |
| | rs10762252, rs12414562, rs7915186, rs12248374, rs7911842, rs4935276, |
| | rs7911086, rs10823327, rs10762272, rs1922140, rs7922112, rs7895839, |
| | rs1922129, rs7902515, rs10762289, rs11595391, rs4575213, rs10998789, |
| | rs4146929, rs6480412, rs10740321, rs67928122, rs16921608, rs3740228, |
| | rs10508961, rs10762325, rs10762326, rs4454675, rs4400745, |
| | rs10999069, rs10999070, rs10823456, rs10740325, rs10823457, |
| | rs7908240, rs10999072, rs55807700, rs10999073, rs10999075, |
| | rs10999076, rs10762328, rs10762329, rs10762330, rs10762331, |
| | rs10762332, rs10740326, rs10762334, rs10823461, rs10823462, |
| | rs10740327, rs10823465, rs11594325, rs10999094, rs10823469, |
| | rs10823471, rs10823473, rs201483328, rs4369342, rs7914283, |
| | rs10762341, rs7069857, rs7914289, rs4431975, rs4431976, rs7896914, |
| | rs7897574, rs4466778, rs10999234, rs4279960, rs4568954, rs4505007, |
| | rs7909578, rs10999270, rs7921307, rs7921318, rs7918335, rs7921717, |
| | rs7070360, rs55981767, rs12256912, rs12264471, rs12243606, |
| | rs12268492, rs7908711, rs7919612, rs7068214, rs7068671, rs61849971, |
| | rs61849972, rs12258583, rs10999327, rs10999330, rs10999331, |
| | rs10999332, rs10999335, rs34606567, rs1574738, rs1937723, |
| | rs10999342, rs1937648, rs7894026, rs11819300, rs4935020, rs4935281, |
| | rs4644602, rs1937661, rs12266397, rs1937656, rs2105441, rs7898813, |
| | rs7902238, rs11592752, rs10999538, rs1937655, rs12098392, rs4415704, |
| | rs11813335, rs10823626, rs1937652, rs10762409, rs11814234, |
| | rs2154278, rs1937651, rs1937650, rs1937649, rs9787480, rs61850003, |
| | rs1937688, rs1937722, rs1937705, rs2226254, rs1937689, rs1937690, |
| | rs12569525, rs10508958, rs61850005, rs2186148, rs58289711, |
| | rs1937698, rs7477477, rs11592926, rs11599504, rs10823710, rs7100647, |
| | rs1937707, rs1937711, rs7913796, rs10999799, rs7096733, rs10823739, |
| | rs10823742, rs10823750, rs12570531, rs7924294, rs7094997, rs1937726, |
| | rs7921347, rs7921365, rs7921498, rs7069812, rs7069974, rs7092679, |
| | rs10740387, rs6480538, rs7920834, rs7920975, rs7903969, rs7921102, |
| | rs7893289, rs10762464, rs6480539, rs7908692, rs7894564, rs6480540, |
| | rs7909767, rs10762466, rs10823817, rs10999944, rs10823823, |
| | rs6480545, rs4472881, rs7912935, rs7913064 |
| RSF1 | rs7109645, rs73496890, rs12286502, rs3019243, rs3133294, rs3018485, |
| 11:77186242- | rs10899380, rs3133293, rs6592723, rs3019254, rs3019252, rs3019251, |
| 77719550 | rs3133456, rs3019238, rs3133458, rs3018480, rs7115727, rs112693639, |
| 210 SNPs | rs3018477, rs3018475, rs3018474, rs3018473, rs10899384, rs3019244, |
| | rs12278944, rs4074161, rs6592724, rs7102156, rs11237211, rs2511809, |
| | rs10793251, rs2105803, rs1945753, rs4945176, rs1945754, rs1945749, |
| | rs6592725, rs7951578, rs7118876, rs6592728, rs4945178, rs11237216, |
| | rs7943055, rs7114468, rs2156843, rs12290511, rs1945752, rs7943742, |
| | rs28451110, rs4366508, rs12788763, rs1945747, rs10899393, |
| | rs10793257, rs12801662, rs4945185, rs3087668, rs12807030, rs7934977, |
| | rs34924092, rs12418929, rs200761558, rs12225188, rs10899396, |
| | rs12418931, rs10899397, rs11237264, rs7925289, rs4945198, rs7121559, |
| | rs7111625, rs4145700, rs10899400, rs501153, rs10899401, rs567814, |
| | rs7949468, rs12294668, rs665363, rs648842, rs529181, rs681411, |
| | rs12418335, rs2186562, rs650636, rs113680047, rs35711367, rs1783545, |
| | rs7932547, rs7117813, rs585721, rs601551, rs7115444, rs634477, |
| | rs609151, rs694893, rs1783547, rs685474, rs600968, rs675925, |
| | rs599962, rs603462, rs667213, rs7112913, rs591283, rs611671, |
| | rs608539, rs598933, rs636041, rs616354, rs611906, rs608389, rs658398, |
| | rs108843, rs582866, rs679155, rs682324, rs1793335, rs7929313, |
| | rs111378364, rs7936105, rs2276349, rs7483572, rs1790244, rs1793344, |
| | rs1790245, rs1790256, rs2510966, rs2511458, rs10751278, rs10899415, |
| | rs11534589, rs3018158, rs1793331, rs1790238, rs9704476, rs1790258, |
| | rs1793333, rs12419855, rs1624540, rs2511454, rs3018156, rs1790250, |
| | rs2510964, rs949171, rs2510965, rs3018411, rs3018167, rs7939854, |
| | rs584854, rs1668327, rs629035, rs2510963, rs1790249, rs1630344, |
| | rs607876, rs1668326, rs632412, rs669869, rs632040, rs669327, |
| | rs630711, rs3018412, rs672460, rs656927, rs35707433, rs7127510, |
| | rs11559637, rs1823508, rs12796422, rs7929510, rs7950529, rs7115539, |
| | rs6592751, rs6592752, rs6592753, rs12786047, rs11237339, rs2187564, |
| | rs7101610, rs11823006, rs10899419, rs7124835, rs10899420, rs7124008, |
| | rs10793267, rs12801444, rs11237343, rs6592754, rs6592755, |
| | rs11601908, rs7110670, rs7938133, rs4945228, rs3819256, rs2276441, |
| | rs7120314, rs12417494, rs1124589, rs11237348, rs10899421, rs7927063, |
| | rs72941385, rs4945232, rs10899424, rs7946108, rs7949200, rs7949412, |
| | rs7931300, rs7931381 |
| FAM60A | rs71440969, rs66707409, rs75479101, rs7305091, rs2287449, rs4124455, |
| 12:31273488- | rs57068704, rs4931445, rs2639054, rs10843894, rs4427634, rs55758709, |
| 31633615 | rs12309295, rs60397875, rs4031237, rs3853619, rs4082530, rs10743752, |
| 179 SNPs | rs61919972, rs143750689, rs148148617, rs373433227, rs10843898, |
| | rs10771808, rs11051292, rs61920013, rs10843901, rs7313157, |
| | rs28698042, rs7132540, rs2118007, rs10771817, rs6487981, rs4930961, |
| | rs4930962, rs4931457, rs11051338, rs10843914, rs10843915, |
| | rs10843916, rs10843917, rs12231929, rs12231950, rs7304146, |
| | rs4931458, rs4930963, rs4931459, rs4930964, rs4930965, rs4931460, |
| | rs4931461, rs4930966, rs12813759, rs11614650, rs11610624, |
| | rs12813960, rs12814110, rs4931462, rs4931464, rs4931465, rs4931466, |
| | rs6487982, rs7296533, rs67283458, rs12305049, rs12305050, rs7960330, |
| | rs7975465, rs7303845, rs7301941, rs1529911, rs2374041, rs11051353, |
| | rs2164502, rs7315309, rs659020, rs670945, rs617372, rs78649856, |
| | rs76781202, rs618403, rs624871, rs625227, rs639902, rs634331, |
| | rs807403, rs620649, rs676301, rs650001, rs792593, rs792592, |
| | rs374858308, rs4931477, rs11051389, rs793168, rs2139724, rs9971825, |
| | rs10843928, rs11051398, rs10843930, rs2090772, rs2090773, |
| | rs11051412, rs2947173, rs708205, rs793190, rs2568901, rs7978277, |
| | rs7303447, rs66521153, rs11615961, rs2888762, rs2892638, rs7294574, |
| | rs3741877, rs10843937, rs7975761, rs10771826, rs10843938, |
| | rs10771828, rs793162, rs11051418, rs7301326, rs3926419, rs9738849, |
| | rs9737629, rs10843939, rs7303848, rs77269317, rs4931488, rs1560634, |
| | rs2163730, rs1469673, rs7968291, rs7313889, rs7302698, rs6416254, |
| | rs10843942, rs7959877, rs7962896, rs7297997, rs7314404, rs7313689, |
| | rs73080572, rs11051428, rs7300106, rs10843947, rs10771830, |
| | rs10771831, rs7314417, rs10771832, rs4931492, rs34498192, |
| | rs10771833, rs7308202, rs7976644, rs10771834, rs7963161, rs7967832, |
| | rs11051437, rs10843953, rs10843954, rs10771837, rs11051439, |
| | rs10771838, rs7135856, rs3844573, rs7977453, rs4931501, rs7954023, |
| | rs7136559, rs7136943, rs7137406, rs10843955, rs11051441, rs3741878, |
| | rs7133082, rs6487991, rs6487992 |
| KRT85 | rs1024321, rs2857681, rs2857686, rs916162, rs2001038, rs2085503, |
| 12:52654547- | rs2608016, rs2014459, rs715874, rs4761783, rs4761853, rs4761854, |
| 52860679 | rs4761855, rs2125119, rs949390, rs7297756, rs7312403, rs10876260, |
| 232 SNPs | rs10876261, rs10876262, rs10876265, rs10876266, rs10876267, |
| | rs10876268, rs61914253, rs10783507, rs10876270, rs10876271, |
| | rs10876272, rs4237896, rs4761856, rs7300801, rs3187034, rs7300941, |
| | rs7954186, rs12809407, rs949387, rs4325349, rs17655636, rs368759595, |
| | rs12578632, rs12832364, rs3741734, rs11612715, rs6580876, rs4076515, |
| | rs1137013, rs870081, rs58810524, rs1540092, rs949376, rs2009914, |
| | rs949378, rs949379, rs949380, rs4761859, rs4761860, rs12825729, |
| | rs12301094, rs2857672, rs2852469, rs2852468, rs2248473, rs2270266, |
| | rs11611330, rs56118466, rs55722266, rs2270267, rs2257286, rs2857669, |
| | rs2852463, rs2857668, rs2852462, rs2277384, rs2852460, rs2857666, |
| | rs2852459, rs2852458, rs2857664, rs998820, rs998821, rs3741717, |
| | rs2187512, rs3741715, rs2857662, rs10876277, rs4237897, rs4237898, |
| | rs2852467, rs2852466, rs7297675, rs1540095, rs2857676, rs2857675, |
| | rs1540094, rs1540093, rs2857661, rs12318087, rs17656443, rs1945296, |
| | rs9919675, rs7133284, rs1573514, rs11170091, rs10783509, rs10747637, |
| | rs7313346, rs10783510, rs7956725, rs7957344, rs12425225, rs7311277, |
| | rs7311523, rs2226907, rs1945294, rs4381405, rs11170093, rs4547151, |
| | rs10747638, rs1945288, rs4761865, rs2852457, rs978378, rs978379, |
| | rs7303826, rs3901682, rs3858625, rs3858626, rs1894036, rs2857680, |
| | rs55680000, rs1791643, rs1732315, rs1791648, rs2105789, rs1732321, |
| | rs1791622, rs4761867, rs1732323, rs12831423, rs7969516, rs2857678, |
| | rs2658665, rs15667, rs7968466, rs1791627, rs10876280, rs2885136, |
| | rs1791624, rs3782493, rs1791619, rs1732283, rs2857677, rs1791618, |
| | rs1732286, rs1791617, rs1732288, rs1732289, rs1732290, rs1791615, |
| | rs1945297, rs2245217, rs949385, rs1573516, rs11170108, rs2001037, |
| | rs2001036, rs2852473, rs2658658, rs2701136, rs2658659, rs2701135, |
| | rs34479712, rs2857660, rs2857659, rs928993, rs1732272, rs1791633, |
| | rs1732273, rs928995, rs2852451, rs11170116, rs383882, rs395871, |
| | rs7969958, rs1610446, rs1791660, rs3847827, rs10876289, rs664354, |
| | rs401926, rs1614460, rs400719, rs396831, rs1798640, rs732181, |
| | rs298110, rs381518, rs380538, rs371202, rs1798642, rs1798643, |
| | rs409929, rs391323, rs429561, rs375539, rs373608, rs597340, rs597830, |
| | rs621164, rs1798639, rs387717, rs373992, rs388626, rs406857, |
| | rs417466, rs673602, rs365763, rs422012, rs443143, rs369557, rs447003, |
| | rs11832606, rs371335, rs421007, rs438099, rs443058, rs1701773, |
| | rs1701776, rs200538936, rs396003, rs404443 |
| MTIF3 | rs6491198, rs7331765, rs9507866, rs9507867, rs7981639, rs9512595, |
| 13:27840739- | rs2380298, rs9579077, rs9512600, rs7996078, rs7990599, rs4771090, |
| 28193965 | rs9512602, rs9512604, rs9319353, rs9319354, rs9319355, rs2380300, |
| 225 SNPs | rs2380301, rs879566, rs59661486, rs1006089, rs1007944, rs7330689, |
| | rs1831798, rs2380305, rs4771091, rs4771092, rs6491201, rs6491202, |
| | rs6491203, rs7329818, rs7329727, rs4771095, rs4771096, rs4771097, |
| | rs7328442, rs9507873, rs9512615, rs12865865, rs11149471, rs7328605, |
| | rs9553992, rs2839916, rs1556538, rs1537436, rs9553998, rs9553999, |
| | rs9319360, rs9579080, rs34210081, rs17615105, rs1815365, rs9554003, |
| | rs9512637, rs7991733, rs9319361, rs7987436, rs7329655, rs7330421, |
| | rs7997379, rs9512652, rs9319362, rs8000509, rs9319364, rs1759867, |
| | rs1218839, rs1218838, rs1218836, rs2780348, rs7334449, rs2780346, |
| | rs1218790, rs1218786, rs1218809, rs1218807, rs9512670, rs1040988, |
| | rs9512671, rs9512673, rs9652090, rs9507889, rs9512677, rs9554028, |
| | rs56105318, rs35202265, rs1974200, rs9554029, rs1218831, rs9512681, |
| | rs1218826, rs1218798, rs1218796, rs2760901, rs1746070, rs1218793, |
| | rs1010371, rs1746068, rs1746067, rs1237710, rs2760900, rs2760899, |
| | rs2780337, rs1218824, rs1218822, rs9512696, rs7988222, rs17085633, |
| | rs7995741, rs9581861, rs9581862, rs9581863, rs1885991, rs1885990, |
| | rs9579085, rs9507902, rs9581864, rs7317179, rs9507903, rs9512712, |
| | rs9512713, rs1006353, rs9554047, rs9554048, rs9512716, rs4771130, |
| | rs7981333, rs9554051, rs12870718, rs927577, rs9554054, rs7992877, |
| | rs9554056, rs7991497, rs1107726, rs1218901, rs1218898, rs9512729, |
| | rs942348, rs1218897, rs1218892, rs1218891, rs1218890, rs1218889, |
| | rs2386685, rs2892092, rs2892093, rs9554063, rs9512733, rs2181776, |
| | rs1028707, rs2181777, rs2181778, rs2181779, rs1237713, rs4337146, |
| | rs1218887, rs1218886, rs1238897, rs1218885, rs9512736, rs1218883, |
| | rs9579098, rs1218882, rs1218881, rs1234660, rs1218877, rs9579101, |
| | rs9507908, rs2057521, rs2057522, rs2386688, rs7332765, rs4769560, |
| | rs9512748, rs9507909, rs9507911, rs8002697, rs7985768, rs7986978, |
| | rs6491217, rs4771141, rs7330269, rs9512750, rs1323967, rs2181775, |
| | rs7984825, rs7989021, rs9507912, rs8000941, rs8001325, rs9512752, |
| | rs9507913, rs3928110, rs7986743, rs8002709, rs11842827, rs9512753, |
| | rs2386689, rs6491218, rs7989803, rs57104715, rs57100189, rs942346, |
| | rs4769561, rs1122547, rs7996690, rs1886204, rs506717, rs654042, |
| | rs510342, rs833095, rs833094, rs833092, rs833091, rs833090, rs656112, |
| | rs562378, rs534150, rs6491220, rs1218962, rs1218961, rs577566, |
| | rs1218960, rs1218959 |
| ATG14 | rs10140801, rs10140063, rs8011288, rs11547116, rs943590, rs10139337, |
| 14:55730359- | rs10139678, rs10142803, rs10143136, rs7146748, rs8021583, rs7155054, |
| 55985073 | rs11158034, rs60248556, rs11848456, rs11848575, rs8010848, |
| 215 SNPs | rs67386901, rs11625001, rs7140628, rs72715769, rs17740741, |
| | rs68046603, rs7158314, rs12050099, rs61210954, rs66696758, |
| | rs67514154, rs58260073, rs8014216, rs7153645, rs11628437, |
| | rs68138420, rs73263927, rs4580079, rs59629906, rs78205412, |
| | rs7157308, rs10139083, rs10145203, rs7151581, rs7151754, rs7153612, |
| | rs2026633, rs56949365, rs4496026, rs4366639, rs66551709, rs10134317, |
| | rs10140869, rs11625423, rs67795323, rs11625968, rs7150234, |
| | rs17741542, rs2209808, rs9919923, rs9919932, rs11851169, rs28612464, |
| | rs28430823, rs17741831, rs10144418, rs3742569, rs10150760, |
| | rs7144737, rs28493647, rs17674463, rs10147765, rs17674563, |
| | rs8008119, rs1572611, rs3783648, rs11626751, rs10136596, rs10142448, |
| | rs7150763, rs8013713, rs2016931, rs28428911, rs10137307, rs10131562, |
| | rs7145727, rs10139000, rs10147254, rs67412516, rs3783652, rs6573019, |
| | rs17128440, rs6573020, rs7153619, rs946057, rs1538257, rs10138446, |
| | rs11624331, rs1009648, rs946056, rs946055, rs7156360, rs8010013, |
| | rs8014526, rs9635231, rs9635232, rs4901560, rs2341537, rs8019209, |
| | rs72717779, rs2341538, rs7145182, rs7145183, rs11845228, rs12888181, |
| | rs12888361, rs61976568, rs61976569, rs11158039, rs8014621, |
| | rs8020152, rs2152975, rs7142204, rs61976571, rs7149622, rs1572613, |
| | rs4243600, rs4898865, rs12886848, rs12887846, rs12886860, |
| | rs56280202, rs12888234, rs10146713, rs11627405, rs10134905, |
| | rs12893964, rs12893000, rs4901564, rs4901565, rs4901567, rs4901568, |
| | rs4901569, rs12431542, rs6573028, rs6573029, rs6573030, rs6573031, |
| | rs6573032, rs7146170, rs10144034, rs10144130, rs11158040, rs8009706, |
| | rs4901570, rs10150596, rs10150617, rs7143800, rs998871, rs6573033, |
| | rs34652660, rs955256, rs955257, rs10483642, rs1890255, rs12893917, |
| | rs10138608, rs12879987, rs1953738, rs11621728, rs60622880, |
| | rs79060736, rs7144510, rs946064, rs12432729, rs946062, rs4901572, |
| | rs9989245, rs7492661, rs2341900, rs72719413, rs12434870, rs12434901, |
| | rs12587011, rs10873089, rs2341882, rs10139709, rs10139855, |
| | rs56242887, rs10130143, rs12586614, rs12587189, rs56092754, |
| | rs76851055, rs76284892, rs7146055, rs7150431, rs1307315, rs1307316, |
| | rs1307317, rs1188069, rs57052990, rs1188070, rs1188072, rs1188073, |
| | rs1188074, rs1188075, rs1188076, rs1307318, rs56295045, rs1188078, |
| | rs1009714, rs1188079, rs1188080, rs2147117, rs2147118, rs2181740, |
| | rs2341883 |
| MESDC2 | rs4778854, rs941984, rs7176363, rs8041798, rs12372942, rs3848178, |
| 15:81066486- | rs4778855, rs7179631, rs925111, rs7174749, rs28629347, rs28515529, |
| 81462214 | rs17325183, rs11634845, rs16972377, rs56067815, rs4778856, |
| 227 SNPs | rs55977687, rs11072943, rs16972386, rs2903414, rs11072944, |
| | rs1077540, rs750570, rs2627310, rs16972404, rs1385085, rs28468684, |
| | rs8028001, rs2055713, rs8024455, rs28594184, rs4260012, rs1507664, |
| | rs1553650, rs7168261, rs8034963, rs11072948, rs7181254, rs8026100, |
| | rs35519924, rs12438768, rs12913158, rs7173382, rs7173733, rs7173789, |
| | rs75020038, rs76432766, rs28405322, rs11072949, rs11072950, |
| | rs11631317, rs11631360, rs16972445, rs16972446, rs813, rs1104876, |
| | rs2089476, rs906433, rs906435, rs4277309, rs7172022, rs7172456, |
| | rs28685158, rs25431, rs1105039, rs7182793, rs12595345, rs12901524, |
| | rs12913146, rs11852715, rs12906003, rs7172166, rs4353448, |
| | rs12442871, rs2866982, rs4145805, rs11629703, rs11629743, |
| | rs11629889, rs1876435, rs61177170, rs28384338, rs12439898, |
| | rs8025349, rs8025378, rs7164719, rs10152744, rs12915985, rs6495528, |
| | rs55732378, rs6495530, rs2271159, rs8028511, rs11631032, rs12594920, |
| | rs28576300, rs2271164, rs4778868, rs12441164, rs11072963, rs8030638, |
| | rs28588248, rs11072964, rs11630929, rs4778871, rs62035118, |
| | rs12593367, rs4778874, rs4778875, rs1320325, rs11636846, rs11072974, |
| | rs11638221, rs11629917, rs1320321, rs1320323, rs8028732, rs11072975, |
| | rs7162420, rs10468068, rs7174596, rs11635158, rs11630428, rs7171900, |
| | rs12324040, rs7163340, rs1509547, rs1546468, rs1546466, rs7168984, |
| | rs1123029, rs1472954, rs11636914, rs2460855, rs2683263, rs1509558, |
| | rs1509559, rs1509560, rs2663924, rs9744476, rs2663921, rs2683264, |
| | rs2663920, rs2663945, rs2663944, rs2663922, rs2683238, rs1317060, |
| | rs2663937, rs1533055, rs2280440, rs2250029, rs2663939, rs2683239, |
| | rs2663936, rs2663935, rs2663934, rs2663933, rs2663932, rs2048269, |
| | rs2683240, rs2663930, rs2663929, rs2663928, rs2663927, rs2683241, |
| | rs2663926, rs2683242, rs2136455, rs58280869, rs60369951, rs2460851, |
| | rs2460852, rs2460853, rs2457381, rs2663917, rs2663916, rs2683244, |
| | rs2457380, rs2663914, rs2663913, rs2683245, rs2663912, rs2683246, |
| | rs2663911, rs2663910, rs1605865, rs2683248, rs2683249, rs1848706, |
| | rs2663909, rs2683254, rs2683255, rs2048271, rs2683256, rs2663947, |
| | rs2460854, rs11072989, rs9989357, rs7174034, rs4778881, rs6495539, |
| | rs6495540, rs12899903, rs7180904, rs55965536, rs34400233, |
| | rs34165852, rs12594790, rs1876809, rs7183232, rs11638013, |
| | rs12916883, rs7169204, rs7169794, rs7173934, rs11638368, rs8030975, |
| | rs7176843, rs7176848, rs7177879, rs6495543, rs1509550, rs1509549, |
| | rs11072991, rs11072992 |
| AQP8 | rs1806846, rs34844403, rs7197568, rs6497791, rs6497792, rs6497793, |
| 16:25068481- | rs6497794, rs8058650, rs874562, rs8049443, rs8049030, rs12051463, |
| 25404038 | rs12708667, rs9921120, rs7202826, rs7203042, rs17784585, rs9935047, |
| 220 SNPs | rs10852283, rs2078112, rs1972764, rs6497809, rs9930827, rs9933296, |
| | rs11074678, rs277908, rs277911, rs277912, rs277913, rs277915, |
| | rs277917, rs277886, rs12917728, rs4787694, rs277897, rs277900, |
| | rs277902, rs277903, rs277904, rs277891, rs277893, rs277889, |
| | rs2881053, rs12446641, rs62034915, rs11641293, rs12325483, |
| | rs12448929, rs889117, rs962974, rs9888821, rs11649311, rs7499578, |
| | rs2287799, rs2287796, rs7499985, rs4787701, rs2345122, rs7184511, |
| | rs7206019, rs8056232, rs8062383, rs11643089, rs9922806, rs12921284, |
| | rs9923063, rs4787310, rs12927671, rs12929379, rs12928606, |
| | rs10400925, rs61507547, rs12920836, rs8049535, rs6497815, rs7197206, |
| | rs55750994, rs56138888, rs55729542, rs12935685, rs8050682, |
| | rs9934149, rs9936376, rs9646251, rs1559184, rs1559185, rs2112814, |
| | rs918537, rs6497817, rs6497818, rs28478555, rs4787704, rs9935405, |
| | rs9926115, rs9935700, rs7197703, rs918536, rs7205208, rs7204744, |
| | rs2059143, rs12918493, rs12918380, rs9923664, rs9923895, rs9923865, |
| | rs7193649, rs7193533, rs11074681, rs6497820, rs6497821, rs8044198, |
| | rs4787706, rs12931519, rs1833782, rs1833781, rs7198649, rs11074682, |
| | rs7187642, rs7187998, rs7188322, rs4580165, rs7189740, rs4438309, |
| | rs4420531, rs4356483, rs4608341, rs9930838, rs9940437, rs9923168, |
| | rs7201854, rs7203443, rs7201921, rs7185265, rs7206788, rs9926291, |
| | rs1895370, rs1895369, rs1895368, rs7191174, rs7191203, rs7192743, |
| | rs7191545, rs7185506, rs9931742, rs9931676, rs9922352, rs9931845, |
| | rs4787712, rs4787713, rs6497822, rs6497823, rs2345123, rs2345124, |
| | rs2881055, rs2345125, rs6497825, rs8056431, rs13330567, rs13335804, |
| | rs13330703, rs72773203, rs79126035, rs62032301, rs62032302, |
| | rs9925035, rs1624, rs2059142, rs1895366, rs1895365, rs6497826, |
| | rs67248738, rs12930659, rs12930409, rs12931493, rs4461077, |
| | rs2345129, rs2345130, rs7190566, rs7194927, rs2042274, rs2042273, |
| | rs4347633, rs11864284, rs7199409, rs13331720, rs9938605, rs9938400, |
| | rs7196537, rs17625422, rs1974809, rs7185551, rs4036467, rs1118458, |
| | rs1118456, rs6497828, rs8047687, rs7201014, rs6497831, rs11074684, |
| | rs6497833, rs12149259, rs11074685, rs12102722, rs1974486, |
| | rs12446252, rs8182224, rs9930052, rs12446967, rs11647618, rs7201464, |
| | rs35121326, rs1858855, rs17794794, rs9933669, rs8046021, rs9928337, |
| | rs2345543, rs7191489, rs11639561, rs6497848 |
| TK2 | rs233560, rs62037211, rs61489015, rs7193705, rs6499071, rs62037212, |
| 16:66377324- | rs8051828, rs7198579, rs6499072, rs2079284, rs34978821, rs62037215, |
| 66775957 | rs62037244, rs13380587, rs1865975, rs1865976, rs7191443, rs7191465, |
| 235 SNPs | rs61250463, rs12920982, rs1370263, rs8057351, rs7198762, rs1370264, |
| | rs9783760, rs9783759, rs1972836, rs4783575, rs11864740, rs6499077, |
| | rs2344423, rs1110644, rs1110645, rs1110646, rs1110647, rs4625720, |
| | rs1123494, rs1123495, rs59442025, rs55823757, rs56130044, |
| | rs55933663, rs12597663, rs35983101, rs12600111, rs67147325, |
| | rs3743871, rs10852432, rs11646157, rs12446898, rs13330048, |
| | rs2344564, rs7499886, rs62637758, rs111067759, rs62637759, |
| | rs7403990, rs7404333, rs7404334, rs7404339, rs7404401, rs7404372, |
| | rs62037250, rs58044782, rs7500475, rs6499078, rs7189589, rs1130844, |
| | rs13336559, rs2304527, rs3785286, rs11640843, rs11647390, rs1865977, |
| | rs1972838, rs11649312, rs202080108, rs1073584, rs1865978, rs1968502, |
| | rs6499080, rs2289148, rs1049970, rs1972839, rs6499081, rs2880989, |
| | rs6499082, rs113672170, rs1063438, rs7199714, rs11863488, |
| | rs11863521, rs2289149, rs2289150, rs9788905, rs11075630, rs1837022, |
| | rs75066722, rs7405005, rs10852433, rs4247350, rs28397059, rs8050957, |
| | rs9972792, rs11075631, rs11647964, rs8045810, rs8046348, rs11075632, |
| | rs2344997, rs11643524, rs7185033, rs4783586, rs1837024, rs719941, |
| | rs3855650, rs34285678, rs8055250, rs8055422, rs1122595, rs8058656, |
| | rs6499084, rs893198, rs4247349, rs11075633, rs7194892, rs35883744, |
| | rs2344917, rs3891911, rs9673874, rs7191512, rs7198295, rs8063396, |
| | rs7196165, rs7203133, rs9926387, rs6499087, rs6499088, rs6499089, |
| | rs11075634, rs11075635, rs4783599, rs2344998, rs1549610, rs67504868, |
| | rs11075636, rs1346480, rs8049640, rs1428106, rs10492904, rs37176, |
| | rs37177, rs28626568, rs13331952, rs13331838, rs3743716, rs725131, |
| | rs37178, rs6499092, rs8060922, rs7193473, rs11867053, rs2290182, |
| | rs34644564, rs35097605, rs2044791, rs4783695, rs61066285, |
| | rs12448207, rs72790434, rs3743719, rs355951, rs48573, rs8051710, |
| | rs355968, rs356506, rs165202, rs12597676, rs355976, rs4258603, |
| | rs78154682, rs78614449, rs35597021, rs34213783, rs355919, rs355918, |
| | rs355917, rs183063, rs12921077, rs583003, rs57228450, rs8044499, |
| | rs8048779, rs60053363, rs34853493, rs12929840, rs4783729, |
| | rs12920542, rs7199583, rs11075640, rs754217, rs4783730, rs7206715, |
| | rs9932105, rs7196093, rs12600097, rs7201865, rs6499095, rs6499096, |
| | rs2305310, rs2345388, rs2345389, rs7191357, rs6499097, rs6499098, |
| | rs11075642, rs12599882, rs2122492, rs2068942, rs2067040, rs2122491, |
| | rs935760, rs8043912, rs9922030, rs12919973, rs9922207, rs7200231, |
| | rs7201266, rs7204805, rs12932199, rs12933365, rs12918990, rs1877141, |
| | rs9927512, rs9930091 |
| OR3A3 | rs231683, rs804226, rs804227, rs804229, rs2625464, rs2519714, |
| 17:3208278- | rs12947906, rs202133898, rs200466402, rs4790127, rs149890845, |
| 3444814 | rs406408, rs404501, rs405923, rs1090086, rs392510, rs230464, |
| 186 SNPs | rs192042, rs179475, rs151191, rs230469, rs230474, rs230475, |
| | rs12603746, rs162256, rs151133, rs371750846, rs3110078, rs371244284, |
| | rs199555313, rs230401, rs1090068, rs78204474, rs230403, rs230404, |
| | rs230406, rs230413, rs227981, rs227980, rs227979, rs227978, rs227976, |
| | rs227973, rs150976, rs150996, rs150995, rs150978, rs150988, rs226357, |
| | rs226356, rs150987, rs150985, rs150982, rs150981, rs150980, rs150979, |
| | rs4790139, rs151130, rs227796, rs227795, rs28653680, rs28432695, |
| | rs230437, rs227790, rs769432, rs227787, rs7212078, rs7212081, |
| | rs11078449, rs138250742, rs62089861, rs4790140, rs2318033, |
| | rs11657615, rs9303174, rs9675016, rs17222411, rs1586929, rs17822657, |
| | rs1488689, rs1488690, rs8067312, rs1948171, rs4790485, rs4790487, |
| | rs4790488, rs66798047, rs36099874, rs36005564, rs140962445, |
| | rs148928609, rs34287415, rs8070169, rs35771277, rs11870084, |
| | rs60882589, rs2318035, rs2171582, rs8079223, rs35156865, rs7216031, |
| | rs4790142, rs4790494, rs7209613, rs12603990, rs12602217, rs12947767, |
| | rs56128631, rs1971292, rs1493282, rs9899946, rs9901015, rs9916804, |
| | rs9902229, rs9904040, rs9910285, rs9902120, rs9904168, rs2318036, |
| | rs2029352, rs2029351, rs7220559, rs4790496, rs2029350, rs1964623, |
| | rs923375, rs923376, rs6502727, rs6502728, rs8068225, rs2035938, |
| | rs2035939, rs11869485, rs72825816, rs12867, rs12450574, rs2271158, |
| | rs1507614, rs10852860, rs7209047, rs7208811, rs7219780, rs7218756, |
| | rs12451131, rs9897180, rs8081785, rs72634008, rs72634009, |
| | rs12946883, rs9906883, rs12944105, rs9895933, rs9895699, rs77804554, |
| | rs12602192, rs4790143, rs7212403, rs2135867, rs2135868, rs4790145, |
| | rs403064, rs4790510, rs4790512, rs1810045, rs1810046, rs1810048, |
| | rs374416, rs365350, rs448402, rs373255, rs454738, rs395357, rs101476, |
| | rs2102213, rs2654686, rs401643, rs566063439, rs371836642, |
| | rs12948220, rs322943, rs322942, rs2654683, rs167737, rs2640076, |
| | rs322941, rs322940 |
| CACNG5 | rs9897506, rs9896549, rs16959909, rs6504446, rs17688563, rs7208562, |
| 17:64661285- | rs957944, rs957946, rs9910390, rs11079664, rs11867695, rs17688881, |
| 65047764 | rs11079665, rs7222680, rs11651627, rs2052193, rs8070293, rs7224351, |
| 229 SNPs | rs2286958, rs8072920, rs7220480, rs7221981, rs7222101, rs1010544, |
| | rs7211505, rs721429, rs35810462, rs759117, rs7217618, rs7224459, |
| | rs9908758, rs9893009, rs9894916, rs8081512, rs8072842, rs9890708, |
| | rs28733563, rs56410769, rs4567763, rs7226221, rs4630584, rs9910868, |
| | rs8074707, rs7218822, rs35199350, rs7219495, rs7220836, rs3889608, |
| | rs7215972, rs7215747, rs11079667, rs9890581, rs9891104, rs3890137, |
| | rs3889537, rs9303511, rs8074909, rs35740833, rs4257241, rs8067877, |
| | rs6504450, rs6504451, rs6504452, rs6504453, rs3803822, rs3803821, |
| | rs4556825, rs4644888, rs8070561, rs4791042, rs6504456, rs4293414, |
| | rs9893695, rs61687889, rs4261586, rs7223528, rs9914792, rs7210818, |
| | rs8076355, rs4381631, rs61762766, rs17710992, rs55792688, rs7214457, |
| | rs12947799, rs4790904, rs56007627, rs9901261, rs4144640, rs4144639, |
| | rs6504458, rs8073185, rs28537782, rs555198187, rs7342847, |
| | rs62070968, rs12603061, rs4791032, rs12451335, rs11870024, |
| | rs7216222, rs4791029, rs28704757, rs60080729, rs55694847, rs8077519, |
| | rs4514706, rs4791028, rs12603665, rs758659, rs2159456, rs4791024, |
| | rs10221247, rs9912397, rs9889789, rs3848414, rs4791021, rs11079669, |
| | rs3848411, rs4791020, rs56813767, rs2048137, rs11650059, rs3760265, |
| | rs1002327, rs2286676, rs1395823, rs9910608, rs9909222, rs11659136, |
| | rs2286678, rs7215831, rs9901824, rs11079671, rs12948256, rs874368, |
| | rs874369, rs7216057, rs6504469, rs1995695, rs1605443, rs1605444, |
| | rs12453324, rs9896901, rs2041328, rs9916665, rs9893996, rs9894545, |
| | rs7222956, rs4791010, rs722859, rs722860, rs2363181, rs2363182, |
| | rs2363183, rs973075, rs973074, rs2018192, rs1984921, rs9905217, |
| | rs9903838, rs1032970, rs740802, rs740803, rs11651549, rs9906636, |
| | rs7215762, rs1876423, rs34566539, rs9916111, rs576706106, |
| | rs539284663, rs9896647, rs9896074, rs4791008, rs9911994, rs9912950, |
| | rs7211879, rs9900952, rs1005381, rs1008365, rs1395826, rs6416952, |
| | rs6504474, rs4791006, rs4791004, rs6504477, rs2363246, rs1109087, |
| | rs12450010, rs12602403, rs3760260, rs72846227, rs35445085, |
| | rs12451912, rs9916833, rs4791001, rs3785599, rs3826347, rs740559, |
| | rs36104009, rs3785598, rs3785595, rs4790896, rs3826345, rs8065294, |
| | rs3848407, rs8065024, rs4790998, rs713572, rs8070678, rs6504483, |
| | rs1019105, rs3826343, rs4516270, rs12603674, rs35835247, rs2363838, |
| | rs740558, rs35666945, rs67391978, rs2240648, rs7219283, rs4790994, |
| | rs4790992, rs4790894, rs4790991, rs11651301, rs7213999 |
| NAPG | rs206447, rs11665296, rs7232386, rs206441, rs206440, rs4796891, |
| 18:10439904- | rs8090659, rs2847351, rs2847350, rs11665244, rs2847348, rs28379930, |
| 10627613 | rs28484275, rs6505558, rs7234471, rs957772, rs480588, rs199967743, |
| 206 SNPs | rs564991, rs948355, rs477052, rs479771, rs1941162, rs539412, |
| | rs693304, rs515248, rs564394, rs476792, rs646410, rs9952598, |
| | rs9952934, rs666064, rs651346, rs498703, rs522393, rs522593, |
| | rs525355, rs647254, rs1786682, rs503840, rs500368, rs10744998, |
| | rs6505560, rs489837, rs91958, rs91959, rs570738, rs550247, rs622760, |
| | rs580258, rs538795, rs1045584, rs505893, rs593078, rs653575, |
| | rs529749, rs500222, rs638170, rs577418, rs493201, rs545857, rs568934, |
| | rs568812, rs484349, rs509416, rs9303706, rs505665, rs660030, |
| | rs34409745, rs658288, rs581369, rs653239, rs527948, rs9951952, |
| | rs525254, rs522420, rs6505563, rs622870, rs513827, rs628896, |
| | rs8098941, rs642133, rs556347, rs500029, rs6505569, rs8090251, |
| | rs8091430, rs8090935, rs9959985, rs492820, rs671624, rs542735, |
| | rs8096132, rs485686, rs519783, rs607946, rs565913, rs610092, |
| | rs3916028, rs563331, rs62093530, rs645477, rs485375, rs573168, |
| | rs578369, rs550223, rs512710, rs2290279, rs12962763, rs12968077, |
| | rs693679, rs485954, rs533675, rs538188, rs652521, rs641723, rs483995, |
| | rs495484, rs522208, rs550627, rs551586, rs580216, rs491946, rs499169, |
| | rs510110, rs540701, rs611684, rs571229, rs571279, rs523000, rs552180, |
| | rs643757, rs555446, rs616139, rs547010, rs10207, rs665604, rs653624, |
| | rs510558, rs518271, rs516472, rs575325, rs1442689, rs11877825, |
| | rs11877777, rs80194120, rs79653006, rs2099871, rs8088257, rs1545251, |
| | rs8091601, rs8093189, rs28394448, rs8088386, rs4625785, rs4617932, |
| | rs12606867, rs9952259, rs11664432, rs11664499, rs11662036, |
| | rs6505575, rs7227293, rs59197413, rs8088403, rs8089027, rs8088773, |
| | rs8088787, rs78686979, rs527278469, rs2187418, rs8099315, rs8099696, |
| | rs8099699, rs8099323, rs9954579, rs67469490, rs35761048, rs34242238, |
| | rs8086299, rs8086886, rs4797446, rs4797447, rs4797449, rs113999025, |
| | rs8091799, rs8091806, rs8092364, rs7241435, rs6505579, rs6505580, |
| | rs6505581, rs8096782, rs12606062, rs7230096, rs2259817, rs920664, |
| | rs10502407, rs1561872, rs28660002, rs73389496, rs2851223, rs4797455, |
| | rs2276371, rs34866058, rs651050 |
| RNF125 | rs1815822, rs11081718, rs10468902, rs12604640, rs4513172, rs7239098, |
| 18:29416105- | rs3737374, rs12969126, rs10853415, rs7237879, rs12957331, rs6506946, |
| 29818222 | rs62095545, rs7233234, rs7226713, rs7231114, rs7231505, rs7234798, |
| 202 SNPs | rs11081722, rs12606882, rs11662341, rs8088396, rs11877256, |
| | rs9635917, rs7229979, rs7234341, rs35275842, rs67874605, rs28639693, |
| | rs2048244, rs12954934, rs12607039, rs12965971, rs2421178, rs9950155, |
| | rs4799610, rs8097835, rs8098106, rs6506952, rs9949272, rs2162378, |
| | rs67236500, rs61740662, rs9963036, rs6506954, rs974124, rs2009538, |
| | rs972358, rs7504605, rs28562612, rs4395150, rs8095428, rs7227644, |
| | rs12957054, rs4799615, rs34087997, rs56350026, rs55924589, |
| | rs6506955, rs11081735, rs11081736, rs55782858, rs56266871, |
| | rs8085043, rs17718913, rs12606297, rs11662928, rs12454708, |
| | rs9958121, rs9949944, rs9947471, rs7236547, rs11875344, rs4799623, |
| | rs66467030, rs12458564, rs12455890, rs4799624, rs12458003, |
| | rs4799626, rs7226543, rs8089092, rs2433377, rs168058, rs336276, |
| | rs336275, rs2006768, rs28621808, rs2911636, rs2433379, rs2433380, |
| | rs606590, rs686271, rs11661763, rs13381311, rs373667, rs454768, |
| | rs641777, rs62093943, rs12455805, rs12455807, rs336273, rs189209, |
| | rs170861, rs2432699, rs336277, rs336279, rs336280, rs336281, |
| | rs8086733, rs661717, rs889888, rs627346, rs9962565, rs12185373, |
| | rs9955978, rs11081741, rs4799325, rs4799326, rs4799628, rs8095867, |
| | rs7238559, rs9967524, rs4799631, rs9953463, rs4799632, rs8085407, |
| | rs10775450, rs12605137, rs11663563, rs2048245, rs10853418, |
| | rs10853419, rs10048285, rs10048287, rs1054667, rs2018380, rs8092272, |
| | rs4799633, rs11081747, rs8082996, rs1364468, rs1816141, rs9949868, |
| | rs67929870, rs9952622, rs9964960, rs7240197, rs9944759, rs11081750, |
| | rs62095576, rs62095578, rs62095579, rs72639475, rs12457134, |
| | rs12457138, rs9959235, rs7232533, rs62095587, rs68120106, |
| | rs72942705, rs9947918, rs17804730, rs10502582, rs17735506, |
| | rs11081751, rs2000671, rs2000670, rs4799636, rs4799637, rs12959368, |
| | rs11661056, rs7234231, rs12962060, rs9947993, rs34313491, rs9960741, |
| | rs7233927, rs16962815, rs16962818, rs1561816, rs1561815, rs2161824, |
| | rs9953120, rs4799330, rs335519, rs335518, rs335516, rs335515, |
| | rs173032, rs200650335, rs202046902, rs616114, rs680321, rs673408, |
| | rs620982, rs641476, rs147164267, rs200222486, rs638936, rs625188, |
| | rs677887 |
| NOTCH3 | rs11085933, rs11085934, rs12609249, rs12973019, rs12462456, |
| 19:15123619- | rs11085935, rs11085936, rs11085937, rs11085938, rs56957978, |
| 15447984 | rs12977108, rs12976225, rs3887111, rs73019024, rs725461, rs68003033, |
| 209 SNPs | rs2097835, rs11085941, rs11085942, rs10854138, rs58957649, |
| | rs12979044, rs11881113, rs4405675, rs10408703, rs757464, rs10424547, |
| | rs8112667, rs10404014, rs7508186, rs6512003, rs111771862, rs2079252, |
| | rs12978589, rs8107136, rs8110862, rs8111608, rs10423378, rs11668717, |
| | rs4808862, rs4808865, rs12982319, rs10411400, rs10417620, rs7251606, |
| | rs74539850, rs4808877, rs4808880, rs12608863, rs714919, rs714920, |
| | rs7507459, rs4581830, rs33933508, rs56084418, rs12984290, |
| | rs12983360, rs12984433, rs12977509, rs12976496, rs12978660, |
| | rs59482560, rs4808898, rs10407982, rs10402505, rs10402567, |
| | rs4808901, rs1034751, rs10402971, rs10402409, rs10426994, |
| | rs10401353, rs10403720, rs10403122, rs10404122, rs4808175, |
| | rs12710294, rs12974625, rs12981586, rs12974134, rs3181304, |
| | rs35785415, rs7256205, rs10406273, rs2366726, rs2158468, rs3181305, |
| | rs3181307, rs2366725, rs3181161, rs3181162, rs717309, rs12973502, |
| | rs73020344, rs11883196, rs4808189, rs35788356, rs35821586, |
| | rs34964429, rs12981565, rs12974713, rs12975987, rs10406683, |
| | rs12985457, rs12985453, rs6512013, rs34795188, rs12610130, |
| | rs8103280, rs4808195, rs7251658, rs7256401, rs6512014, rs10419983, |
| | rs67711400, rs7245995, rs12608898, rs34857561, rs8105737, |
| | rs59400600, rs7255422, rs10221538, rs28608656, rs7250048, |
| | rs10408949, rs7246549, rs12609513, rs12611103, rs12980862, |
| | rs2040858, rs2240298, rs8109071, rs8109089, rs12359, rs13301, |
| | rs2074261, rs2074262, rs2074263, rs875803, rs12972958, rs1468198, |
| | rs2074264, rs9749309, rs8100069, rs2074265, rs2074266, rs718100, |
| | rs4141356, rs2074267, rs59955878, rs1476527, rs1476530, rs72992037, |
| | rs67072532, rs4340433, rs4578789, rs8107358, rs8104289, rs61070853, |
| | rs61302414, rs76313578, rs12974057, rs10411446, rs4809009, |
| | rs4809012, rs62116498, rs2188960, rs59262739, rs59953204, |
| | rs11879608, rs61745957, rs61746401, rs10403029, rs10427025, |
| | rs10403708, rs10401373, rs1114312, rs11666626, rs55719765, |
| | rs12463011, rs11667033, rs11670085, rs768168, rs7254899, rs1044123, |
| | rs1044116, rs1044055, rs1044009, rs757473, rs2886698, rs56061231, |
| | rs2074621, rs11669950, rs11669982, rs1043997, rs4809043, rs1043996, |
| | rs2238643, rs11665884, rs34547756, rs4809067, rs12609457, rs4808272, |
| | rs56825447, rs10426034, rs10426083, rs11673318, rs12610461, |
| | rs10405035 |
| EPN1 | rs10417266, rs531794, rs645973, rs59914207, rs1712775, rs7247510, |
| 19:56037363- | rs55974933, rs310454, rs57198421, rs8106498, rs694398, rs2459752, |
| 56370829 | rs673519, rs656097, rs7247650, rs10411415, rs10414140, rs2431796, |
| 192 SNPs | rs2459744, rs7250117, rs34077313, rs12327839, rs4801402, rs4801403, |
| | rs10421563, rs2012778, rs10402124, rs4801413, rs556639704, |
| | rs73068914, rs11668147, rs12185511, rs2014329, rs6509945, |
| | rs12462275, rs10421911, rs58320161, rs59409081, rs4801428, |
| | rs28379489, rs12972695, rs2292812, rs7257888, rs12974235, |
| | rs11084403, rs11084404, rs588115, rs10451452, rs693289, rs377722803, |
| | rs1077806, rs484519, rs564622, rs2431798, rs57314044, rs8110652, |
| | rs34907477, rs685225, rs579496, rs596075, rs643368, rs485152, |
| | rs1808393, rs2864434, rs889092, rs310475, rs11233, rs79432053, |
| | rs504850, rs3786648, rs540607, rs74330152, rs664684, rs8791, |
| | rs7249837, rs4801264, rs4801265, rs28498398, rs6509955, rs11673193, |
| | rs58619412, rs17886456, rs8105023, rs12973879, rs10408969, |
| | rs1020947, rs1020948, rs1019488, rs10418120, rs10419893, |
| | rs202099356, rs8106988, rs7256523, rs35349001, rs755804, rs10417878, |
| | rs7259060, rs7255520, rs8100685, rs6509957, rs73053986, rs2868062, |
| | rs12973665, rs8106001, rs8106255, rs8106481, rs2547255, rs73933330, |
| | rs8109382, rs12151283, rs180966869, rs2019946, rs140087406, |
| | rs147984855, rs149792632, rs79703173, rs2547256, rs28695271, |
| | rs12972819, rs7246344, rs1820105, rs2161477, rs2067104, rs299183, |
| | rs7250432, rs299184, rs4801628, rs6509959, rs8108895, rs2547294, |
| | rs299185, rs2616937, rs2616938, rs10424927, rs299187, rs299188, |
| | rs8106437, rs6509960, rs299189, rs299190, rs2616940, rs10422937, |
| | rs11665811, rs57293981, rs2547273, rs299175, rs299173, rs299171, |
| | rs299167, rs299166, rs299164, rs12610400, rs35064274, rs8101591, |
| | rs4801631, rs4801281, rs2086096, rs2616947, rs2576448, rs2616946, |
| | rs2616945, rs7253098, rs4516329, rs4264497, rs4619507, rs4548988, |
| | rs151183015, rs7248235, rs1423068, rs2616943, rs2547285, rs302476, |
| | rs302473, rs302472, rs10403638, rs302470, rs177352, rs113393290, |
| | rs62127059, rs567292924, rs35969546, rs62128164, rs644733, |
| | rs12977263, rs381809, rs382657, rs11673189, rs435148, rs441827, |
| | rs395503, rs421810, rs9284431 |
| SPDYA | rs11894900, rs2272387, rs57817040, rs3088281, rs2272386, rs6738334, |
| 2:28865438- | rs6741437, rs2049690, rs899156, rs55655550, rs17007147, rs2126890, |
| 29210522 | rs17007156, rs12477114, rs6753015, rs56328298, rs60857672, |
| 226 SNPs | rs13010554, rs11694841, rs6547865, rs35161467, rs6724999, |
| | rs11127186, rs55819302, rs6709295, rs11690065, rs62129808, |
| | rs55772556, rs35244334, rs57183123, rs55902327, rs56089836, |
| | rs56390569, rs58717377, rs10192375, rs11127187, rs6726829, |
| | rs6727225, rs4666113, rs4666114, rs11682523, rs6547867, rs899153, |
| | rs899154, rs4666115, rs11674854, rs2049691, rs7565187, rs7594379, |
| | rs7586405, rs374164723, rs7594363, rs12614723, rs55836936, |
| | rs11683296, rs745645, rs17007191, rs6547870, rs4665431, rs55721532, |
| | rs9309664, rs4408686, rs12714240, rs7562170, rs55835850, rs11690423, |
| | rs4666118, rs4665432, rs11688129, rs72782294, rs13025377, |
| | rs13394863, rs2045884, rs2169751, rs7589860, rs10198789, rs10174533, |
| | rs12714241, rs7607844, rs4549034, rs11127188, rs6707130, rs4372836, |
| | rs966250, rs7598876, rs4666119, rs4665434, rs4233727, rs4233728, |
| | rs6547872, rs6547873, rs6547874, rs7579321, rs10865507, rs4558548, |
| | rs6707848, rs6709674, rs6710080, rs6710082, rs12472549, rs11127189, |
| | rs11686412, rs13007183, rs10180266, rs7579277, rs7580078, rs6547875, |
| | rs10178852, rs10204329, rs3768665, rs6547877, rs6742291, rs6747852, |
| | rs6547878, rs6419696, rs6547880, rs3190, rs12475612, rs4132617, |
| | rs7601934, rs7567861, rs6547882, rs11127190, rs61407258, rs6718662, |
| | rs10865508, rs11685682, rs67410623, rs61226201, rs4611608, |
| | rs60790187, rs66904522, rs72784066, rs55785599, rs11684695, |
| | rs4665436, rs4233729, rs4666122, rs4563180, rs4638745, rs56178008, |
| | rs4358081, rs4289133, rs6744612, rs66518107, rs12465314, rs12613887, |
| | rs4533438, rs10201633, rs4666126, rs7569020, rs11695230, rs7593028, |
| | rs7584370, rs9677133, rs9677140, rs9678533, rs13426929, rs6729601, |
| | rs6730523, rs4666130, rs6547892, rs12623400, rs6727420, rs6742478, |
| | rs4456657, rs10175980, rs7419369, rs4464230, rs4497831, rs6710697, |
| | rs6757913, rs6547895, rs6547896, rs13008915, rs9653591, rs10164596, |
| | rs4666140, rs6755185, rs1140697, rs7573904, rs62132028, rs12477630, |
| | rs4398230, rs6753809, rs6757653, rs4666143, rs6707606, rs35041836, |
| | rs35481047, rs56237105, rs55717610, rs36076377, rs6757842, |
| | rs12464147, rs13008304, rs13013647, rs10177429, rs1128641, |
| | rs6738504, rs13020792, rs34019733, rs58402186, rs7577169, rs7591793, |
| | rs11127195, rs7595944, rs3936096, rs80335385, rs6744539, rs11674944, |
| | rs13388668, rs55762746, rs55650865, rs4586599, rs6742711, rs4361080, |
| | rs56063472, rs4075266, rs12467459, rs11682639, rs6728661, |
| | rs13406322, rs7569861, rs7609305, rs7570068 |
| IL1RL2 | rs76160345, rs12712126, rs10181425, rs4850997, rs13382466, |
| 2:102696388- | rs6745511, rs11123908, rs11123909, rs11123910, rs2041749, |
| 102966138 | rs17818291, rs12712128, rs1861285, rs1861282, rs874954, rs1558643, |
| 233 SNPs | rs1030022, rs6704593, rs1967313, rs1004548, rs4603782, rs6736803, |
| | rs12712130, rs6713468, rs60084385, rs4851541, rs2310185, rs7588201, |
| | rs4851544, rs4851546, rs6706048, rs956730, rs2234650, rs12613301, |
| | rs7580384, rs11883987, rs4851547, rs4851000, rs3755295, rs1978330, |
| | rs3917225, rs2287049, rs2287047, rs3917243, rs3917265, rs3917267, |
| | rs3917273, rs3917279, rs2160227, rs13020778, rs951192, rs3917304, |
| | rs3917318, rs7582198, rs1030021, rs10181869, rs10184597, rs3771199, |
| | rs3771197, rs3821207, rs3771195, rs3771194, rs3771193, rs3771192, |
| | rs56117350, rs12474258, rs11678651, rs1922302, rs72820147, |
| | rs7574448, rs7583664, rs7558268, rs74597567, rs72820152, rs1960510, |
| | rs79901634, rs55935523, rs1922299, rs1922298, rs1922297, rs1922296, |
| | rs4851557, rs4851558, rs56112655, rs2141865, rs2141864, rs2178675, |
| | rs2310236, rs2310237, rs2310238, rs2871457, rs4471910, rs4471912, |
| | rs11677606, rs6543110, rs1465326, rs2871458, rs7569116, rs7566395, |
| | rs7605715, rs11123913, rs34074031, rs2080310, rs1922289, rs1922290, |
| | rs1922291, rs1922292, rs6743219, rs35917382, rs4851001, rs4851560, |
| | rs4851002, rs3815517, rs1922295, rs11696062, rs917994, rs2041753, |
| | rs2041752, rs2160226, rs11687459, rs72820175, rs6715919, rs995514, |
| | rs3729564, rs3771187, rs3771185, rs12472872, rs12473710, rs12472862, |
| | rs12476585, rs34778121, rs35139442, rs17637748, rs2310241, |
| | rs2302621, rs3821205, rs10167431, rs11692230, rs12468355, rs1558626, |
| | rs1558624, rs1035131, rs17689452, rs12712133, rs10186746, |
| | rs12464251, rs56389410, rs12470560, rs12474199, rs1420093, |
| | rs10200410, rs56144230, rs1115282, rs11683213, rs7600901, rs1420091, |
| | rs4399750, rs2110660, rs1420090, rs7565653, rs7568913, rs4090473, |
| | rs12476925, rs12476968, rs6721346, rs10178436, rs11685424, |
| | rs11685480, rs6733174, rs6543118, rs1558622, rs1558621, rs10189202, |
| | rs10191914, rs10189711, rs12712135, rs1558620, rs1558619, |
| | rs12996505, rs13020793, rs10183388, rs953934, rs950880, rs1968171, |
| | rs1968170, rs11123918, rs10182639, rs11690443, rs12712136, rs974389, |
| | rs4142132, rs971764, rs1420088, rs66780767, rs11123919, rs11123920, |
| | rs6706844, rs11674832, rs11675988, rs11679900, rs11676075, |
| | rs11676124, rs11123921, rs12992762, rs12998412, rs11123922, |
| | rs12053422, rs183138805, rs4490207, rs4455171, rs13007344, |
| | rs76520363, rs76278109, rs76886731, rs150341880, rs138087973, |
| | rs76498201, rs12996772, rs1420102, rs12466380, rs12712140, |
| | rs1997467, rs1997466, rs1362350, rs1362349, rs12712141, rs59247511, |
| | rs1420101, rs13001714, rs12712142, rs6543119, rs13017455, rs1921622 |
| CST7 | rs2424673, rs2424675, rs2424677, rs6076273, rs61190607, rs910242, |
| 20:24740871- | rs1535065, rs1535066, rs2424680, rs6083620, rs6132765, rs11698988, |
| 25126325 | rs226664, rs226667, rs226668, rs226669, rs226670, rs226671, rs226672, |
| 213 SNPs | rs226673, rs226674, rs226675, rs226676, rs226677, rs226678, rs226679, |
| | rs226680, rs2424688, rs2424689, rs226681, rs226687, rs85804, |
| | rs226688, rs226697, rs914305, rs6114877, rs6036912, rs35326290, |
| | rs6138396, rs62215832, rs6050070, rs6083656, rs1331290, rs6083658, |
| | rs6050074, rs6036951, rs1331285, rs1590974, rs1576648, rs6036955, |
| | rs1113872, rs1113870, rs6083660, rs6083662, rs6036958, rs7266572, |
| | rs7266683, rs6050086, rs6036959, rs6050087, rs6083664, rs1572066, |
| | rs6083669, rs6083670, rs7272869, rs7273098, rs7273006, rs6050092, |
| | rs6036962, rs6114904, rs6050093, rs6050095, rs6106960, rs6050096, |
| | rs6106961, rs6106962, rs6036963, rs6036965, rs6050097, rs6050100, |
| | rs6050101, rs6050102, rs6050105, rs6050106, rs6114908, rs1831483, |
| | rs1411894, rs1411895, rs11905160, rs7269563, rs7269564, rs12151907, |
| | rs6114915, rs6036967, rs6036968, rs6036972, rs6114921, rs6050114, |
| | rs6050115, rs1029031, rs6106969, rs4502500, rs6050127, rs885402, |
| | rs4462847, rs6138406, rs11087486, rs4594518, rs2387366, rs6083684, |
| | rs6083685, rs6083686, rs6083688, rs6106975, rs6138417, rs4813541, |
| | rs4815337, rs4815339, rs6050177, rs2179729, rs2179730, rs6050179, |
| | rs6050181, rs6132773, rs6132774, rs926661, rs926662, rs4815340, |
| | rs4815341, rs6138423, rs6138431, rs735886, rs6050198, rs34419834, |
| | rs6050201, rs13041643, rs12625319, rs4815351, rs6138437, rs6083699, |
| | rs4815355, rs2387572, rs8183891, rs2008275, rs4815357, rs6138453, |
| | rs6083709, rs6138461, rs6114992, rs1007881, rs6138478, rs910527, |
| | rs6132792, rs6106989, rs6115015, rs4815364, rs3646, rs3746331, |
| | rs6050290, rs1005409, rs11696120, rs13036777, rs2387577, rs4815366, |
| | rs743017, rs743018, rs6050299, rs4813546, rs4813547, rs2207632, |
| | rs6037014, rs6050300, rs4813548, rs6083734, rs6083737, rs2207633, |
| | rs56157240, rs12480307, rs4815368, rs6050304, rs58747756, |
| | rs61304082, rs6050306, rs6083739, rs6050308, rs6115024, rs56369722, |
| | rs55651034, rs62215277, rs6115026, rs2207635, rs2892313, rs6083745, |
| | rs6115037, rs6083749, rs6138488, rs4311121, rs2144372, rs6050337, |
| | rs6037024, rs6050342, rs8115505, rs8122274, rs4815374, rs35910458, |
| | rs2057042, rs6050348, rs6050353, rs6037032, rs6037033, rs6037034, |
| | rs910102, rs4482560 |
| L3MBTL1 | rs6030803, rs2206465, rs2143253, rs12480018, rs4812700, rs6065604, |
| 20:41986436- | rs4812701, rs4812702, rs6103254, rs6103256, rs6103257, rs4812703, |
| 42291628 | rs6073070, rs6093817, rs6030806, rs6030807, rs141889737, rs6124552, |
| 211 SNPs | rs4812704, rs4812705, rs4812706, rs6030811, rs6017021, rs6017022, |
| | rs6017023, rs6065606, rs1983595, rs6017024, rs4812708, rs1883544, |
| | rs6103265, rs6124554, rs6103266, rs6103267, rs6093822, rs6124555, |
| | rs2206464, rs4812709, rs369316432, rs57127408, rs16985285, |
| | rs62225381, rs6103270, rs6130362, rs11697029, rs4142387, rs1569623, |
| | rs9989924, rs6103272, rs6103273, rs2903631, rs2867661, rs2903632, |
| | rs2867662, rs2867663, rs6103274, rs6130364, rs6103277, rs6103278, |
| | rs6103279, rs6103280, rs6103281, rs926495, rs2179470, rs1810480, |
| | rs2007331, rs2007333, rs909889, rs1569624, rs7345270, rs4991542, |
| | rs742447, rs6130366, rs6103285, rs6103289, rs6103291, rs6103292, |
| | rs6103293, rs6103294, rs6017043, rs6030843, rs6103299, rs1997749, |
| | rs6030846, rs6130373, rs6093830, rs1120059, rs1120058, rs6017052, |
| | rs6017053, rs6030851, rs6030853, rs35090386, rs6030855, rs6124559, |
| | rs8117350, rs6130378, rs1014732, rs6017060, rs6124562, rs6017064, |
| | rs6017066, rs6017067, rs13041897, rs34234940, rs6030864, rs6093835, |
| | rs56405994, rs6093836, rs941441, rs4271820, rs12481245, rs6103325, |
| | rs4810385, rs13038619, rs4476404, rs6017074, rs11086876, rs7270264, |
| | rs12479575, rs12481043, rs761040, rs761038, rs11697316, rs11086877, |
| | rs6103329, rs2064279, rs6073098, rs6030889, rs2064280, rs6030899, |
| | rs4812712, rs6017090, rs6065612, rs6017092, rs6017093, rs6073109, |
| | rs6073111, rs6103348, rs2867664, rs1124805, rs1569622, rs765147, |
| | rs765148, rs11697203, rs765149, rs4812714, rs2867665, rs35177566, |
| | rs909893, rs6030926, rs4812716, rs6065615, rs6124569, rs6030931, |
| | rs6103362, rs57208610, rs6017098, rs6030935, rs4494826, rs79911777, |
| | rs4812717, rs6030941, rs2071972, rs2071970, rs2269625, rs3746526, |
| | rs8116134, rs11086881, rs12479637, rs2269623, rs2269622, rs6030952, |
| | rs7508895, rs2105941, rs1076943, rs1077832, rs79748838, rs62224798, |
| | rs6017117, rs3135415, rs3135416, rs4812721, rs2067061, rs6017119, |
| | rs6093855, rs916410, rs763227, rs1894667, rs6073133, rs6065629, |
| | rs3752562, rs4810386, rs6073142, rs6073143, rs146734714, rs6103393, |
| | rs6103395, rs6130427, rs4810390, rs2093139, rs6130428, rs6030998, |
| | rs6073162, rs6031007, rs439115, rs6103413, rs397553, rs6017135, |
| | rs396373, rs6065643 |
| MIS18A | rs7281152, rs2833647, rs62214195, rs62214196, rs11088204, rs2833649, |
| 21:33453534- | rs2409435, rs762168, rs1004033, rs2012957, rs3890244, rs2000452, |
| 33840197 | rs4816419, rs2833655, rs7275371, rs734988, rs6517078, rs2833658, |
| 231 SNPs | rs915560, rs876382, rs2186267, rs9979997, rs2833659, rs2833660, |
| | rs4817463, rs35219708, rs4817464, rs2032085, rs8130712, rs2833662, |
| | rs2833663, rs9977871, rs2833664, rs7278865, rs2833665, rs2833666, |
| | rs13049602, rs11700886, rs7283540, rs1041744, rs2409440, rs4816422, |
| | rs28428726, rs4817468, rs1475905, rs1475906, rs1892575, rs1892574, |
| | rs2154424, rs9983812, rs17560508, rs4816424, rs4816426, rs12482791, |
| | rs7278211, rs12151995, rs2898186, rs2409445, rs6517079, rs11700761, |
| | rs2051186, rs2051187, rs10854355, rs1892651, rs1892652, rs12483332, |
| | rs12483200, rs2409446, rs8134885, rs2409447, rs7364070, rs4817471, |
| | rs2186313, rs9978000, rs8127542, rs6517082, rs8127706, rs2211677, |
| | rs2226292, rs2226291, rs4817472, rs4817473, rs4817474, rs4817475, |
| | rs2833690, rs2833691, rs4143359, rs9653707, rs12627314, rs11088205, |
| | rs11088206, rs2833692, rs2833693, rs2211676, rs2211675, rs2226290, |
| | rs2409448, rs2833695, rs4817476, rs8132567, rs8131399, rs2833697, |
| | rs2833699, rs2833700, rs8130652, rs2833711, rs2833715, rs2833717, |
| | rs7282049, rs7283591, rs79250184, rs9980501, rs9982765, rs2833727, |
| | rs7283647, rs2833736, rs1015040, rs762223, rs762224, rs2017856, |
| | rs762171, rs2017901, rs8130297, rs11088212, rs8131253, rs8131400, |
| | rs8126620, rs8126627, rs8134946, rs8127678, rs2833737, rs4817477, |
| | rs2154422, rs4816428, rs11088213, rs2040106, rs2833746, rs79971561, |
| | rs2833747, rs2070374, rs2070375, rs2070376, rs3818, rs7281536, |
| | rs6517089, rs8129993, rs7283034, rs9977661, rs730997, rs28495532, |
| | rs11088215, rs4816430, rs12482323, rs8127916, rs4817480, rs8132833, |
| | rs8128242, rs10427546, rs2833756, rs8130762, rs7279998, rs8133919, |
| | rs8134971, rs2833773, rs2833776, rs743306, rs2833786, rs743307, |
| | rs2096490, rs2833793, rs2833796, rs2833798, rs7279677, rs9305503, |
| | rs2833799, rs12627711, rs1974380, rs2211788, rs8131515, rs2833800, |
| | rs2226338, rs2211789, rs8134043, rs2226289, rs7278946, rs2255822, |
| | rs2833802, rs2833803, rs2833804, rs2833806, rs2833808, rs2833809, |
| | rs2833812, rs2833813, rs2833818, rs6517096, rs9977024, rs2833819, |
| | rs1888448, rs2833825, rs77135980, rs201604762, rs7276272, rs2833827, |
| | rs2833828, rs2833834, rs9982003, rs2833839, rs2010550, rs2010585, |
| | rs2010586, rs8134814, rs9978264, rs9980314, rs8127011, rs8128908, |
| | rs7276260, rs7276253, rs7276257, rs2833840, rs1041824, rs2833841, |
| | rs762173, rs762174, rs2833842, rs113304276, rs11911900, rs11909401, |
| | rs2833844, rs4816434, rs4816435 |
| TOP3B | rs2266967, rs5749986, rs5749998, rs9607287, rs111736196, rs8141020, |
| 22:22156707- | rs8141851, rs4516540, rs28566555, rs28697123, rs4821339, rs3788332, |
| 22496962 | rs2266969, rs55891647, rs2283793, rs9607298, rs6518986, rs1892846, |
| 208 SNPs | rs1892848, rs9610374, rs9610375, rs5999749, rs56398890, rs9610400, |
| | rs5755694, rs8136867, rs9610458, rs11913721, rs5999823, rs2283794, |
| | rs4821402, rs5750113, rs9610500, rs9610505, rs9607340, rs55762115, |
| | rs750942, rs171277, rs240621, rs240049, rs240051, rs240053, rs240054, |
| | rs6000067, rs6000070, rs240058, rs56094396, rs738858, rs240064, |
| | rs2329884, rs894095, rs2083565, rs2027790, rs94194, rs5995268, |
| | rs5995269, rs240068, rs240067, rs1005579, rs381523, rs381849, |
| | rs239918, rs239917, rs5756306, rs5756323, rs238783, rs238775, |
| | rs366116, rs4821629, rs2213141, rs986643, rs986645, rs4145410, |
| | rs5750444, rs5756706, rs762470, rs762471, rs5756721, rs714973, |
| | rs5750456, rs6000820, rs5756751, rs1894252, rs1894253, rs6000857, |
| | rs5756762, rs5750469, rs5750471, rs4821687, rs4821692, rs4992280, |
| | rs4435546, rs5750483, rs5750484, rs5756797, rs2876994, rs4239888, |
| | rs743530, rs5750487, rs2329928, rs1894248, rs1894249, rs5756806, |
| | rs2329929, rs713638, rs713636, rs1894250, rs5750496, rs4821718, |
| | rs5756833, rs4516538, rs6000949, rs6000952, rs5756871, rs5756876, |
| | rs12172019, rs5756881, rs3939015, rs9619771, rs4820311, rs9622862, |
| | rs7293130, rs2282670, rs5756928, rs6001024, rs5756937, rs5756938, |
| | rs5756939, rs5756941, rs5756942, rs5750553, rs5756944, rs762482, |
| | rs5756949, rs5756960, rs11704547, rs11704841, rs11704787, |
| | rs13057383, rs13056557, rs13053134, rs13057970, rs13057981, |
| | rs6001052, rs5995548, rs5995549, rs61663167, rs5756978, rs34375722, |
| | rs34963809, rs35626600, rs34228957, rs13054756, rs4821768, |
| | rs4821769, rs4821770, rs4821771, rs4821772, rs4821773, rs4821774, |
| | rs5756988, rs5756989, rs5756990, rs5756991, rs5756992, rs9622919, |
| | rs9619793, rs9619794, rs9622920, rs6001072, rs6001073, rs6001075, |
| | rs6001076, rs5995560, rs79274271, rs5757003, rs6001078, rs5750574, |
| | rs5757004, rs5757005, rs5757006, rs5757007, rs5757011, rs5757013, |
| | rs5757014, rs5757017, rs5757018, rs59356111, rs5757019, rs5750579, |
| | rs11089925, rs5757024, rs5757026, rs5757027, rs5750582, rs5757028, |
| | rs2213156, rs2213157, rs2213158, rs2213159, rs9622955, rs4821781, |
| | rs5750588, rs5750589, rs5757038, rs5750594, rs5757051, rs5757056 |
| ATXN7 | rs6784922, rs6798144, rs2201582, rs1390442, rs6808984, rs6764278, |
| 3:63660184- | rs4688422, rs1390443, rs35558339, rs4688423, rs7629447, rs7651717, |
| 64179000 | rs35583240, rs6785678, rs35914199, rs6785901, rs6789160, rs6414560, |
| 222 SNPs | rs57160532, rs6777280, rs1495908, rs9809255, rs1491582, rs4688177, |
| | rs141652375, rs7635168, rs971576, rs971575, rs9859021, rs1472338, |
| | rs1472337, rs35966189, rs1491581, rs67608305, rs1873565, rs1565549, |
| | rs1565550, rs6804524, rs73121252, rs17700493, rs17654983, |
| | rs11919532, rs11927676, rs113386200, rs832187, rs116300182, |
| | rs534372659, rs6779372, rs3733120, rs854622, rs832195, rs704371, |
| | rs704368, rs704367, rs704364, rs832197, rs55917937, rs7627690, |
| | rs6798742, rs59971314, rs3774713, rs7615475, rs3774720, rs1005811, |
| | rs3774723, rs2241823, rs7624403, rs3774726, rs13272, rs11922435, |
| | rs9865811, rs2037119, rs56030924, rs9875198, rs3816156, rs9879045, |
| | rs831692, rs831691, rs40610, rs17344417, rs35837, rs35838, |
| | rs12497119, rs35840, rs35841, rs6785145, rs6764606, rs166229, |
| | rs35370588, rs56369425, rs6445389, rs55969752, rs42456, rs704375, |
| | rs254858, rs66629090, rs1450985, rs27386, rs35835, rs40517, rs26935, |
| | rs26936, rs704376, rs254857, rs7636957, rs254856, rs254855, |
| | rs1007234, rs254854, rs831566, rs831565, rs34693199, rs153734, |
| | rs153730, rs26939, rs26937, rs161654, rs161656, rs36237, rs40158, |
| | rs36239, rs153712, rs6765188, rs6789721, rs152244, rs153716, |
| | rs704377, rs830356, rs1096264, rs1096262, rs1096261, rs1104152, |
| | rs1069985, rs1069984, rs1069983, rs1069982, rs1069981, rs1096171, |
| | rs1069980, rs1096170, rs1096169, rs1096168, rs1096167, rs704378, |
| | rs704379, rs697264, rs830011, rs704381, rs704386, rs704388, rs830010, |
| | rs985280, rs865511, rs1069978, rs1069976, rs830007, rs830005, |
| | rs1024895, rs696009, rs696010, rs696223, rs152294, rs161657, |
| | rs161658, rs161659, rs152296, rs161660, rs161661, rs9873216, |
| | rs9878228, rs1835898, rs1432586, rs1432587, rs696011, rs696012, |
| | rs697266, rs704391, rs695935, rs696013, rs697267, rs697269, |
| | rs149913940, rs6445392, rs1078398, rs1078397, rs888406, rs753562, |
| | rs13094124, rs13072811, rs2111684, rs13090987, rs62249933, |
| | rs11720610, rs2306380, rs17664202, rs11707013, rs17664266, |
| | rs17664296, rs13088453, rs184452, rs1642561, rs62249937, rs13080962, |
| | rs167787, rs7637364, rs149105, rs153737, rs13068250, rs697276, |
| | rs704394, rs153717, rs153718, rs153719, rs153721, rs153723, rs153724, |
| | rs11914401, rs161666, rs2160827, rs185295, rs697279, rs697280 |
| NIT2 | rs528787640, rs9849632, rs9289981, rs9865713, rs2130369, |
| 3:99866030- | rs370340178, rs720688, rs9814549, rs6798114, rs7619080, rs2316263, |
| 100264961 | rs12629505, rs1017967, rs6776885, rs6776886, rs6810262, rs1872609, |
| 212 SNPs | rs7623587, rs9290003, rs35170554, rs9847283, rs7631174, rs9868170, |
| | rs9850382, rs9860514, rs9860561, rs6414385, rs11707768, rs6780609, |
| | rs7625101, rs13078920, rs7611566, rs12633821, rs4928163, rs59233020, |
| | rs11915985, rs931332, rs6802765, rs9810039, rs4928115, rs1979540, |
| | rs6764957, rs6765158, rs7615300, rs11709563, rs6772774, rs34384409, |
| | rs6774538, rs10936147, rs1609481, rs7638503, rs13074365, rs3806616, |
| | rs3772701, rs6791887, rs4928119, rs13075241, rs13079881, rs16842060, |
| | rs4928121, rs35141973, rs11710065, rs11709240, rs61095099, |
| | rs16847139, rs4928122, rs12633745, rs2028494, rs67403774, |
| | rs68094410, rs79519930, rs967608, rs4552390, rs4263320, rs28577907, |
| | rs6787301, rs3772698, rs3772696, rs1036770, rs13072367, rs6764125, |
| | rs12629226, rs4928168, rs13098188, rs3772694, rs6762794, rs4928169, |
| | rs34700788, rs78896720, rs6787142, rs34314126, rs10511178, rs277657, |
| | rs17279967, rs10511179, rs1045206, rs1214375, rs9873033, rs277647, |
| | rs1214377, rs2316701, rs1661287, rs369943296, rs2289500, rs2316264, |
| | rs6797836, rs7434167, rs11712077, rs4928071, rs6797397, rs7434244, |
| | rs9840370, rs1132022, rs4928050, rs56253850, rs7616574, rs4928073, |
| | rs11922527, rs2068018, rs57018910, rs59299896, rs7429072, rs7429604, |
| | rs61636409, rs7640470, rs12631075, rs7616839, rs12634371, |
| | rs78669257, rs62276520, rs7630198, rs62276521, rs62276522, |
| | rs4928097, rs4928098, rs67774605, rs6789867, rs60071944, rs58059387, |
| | rs12636805, rs9851429, rs73152126, rs11923942, rs11920006, |
| | rs11927516, rs59771628, rs6763079, rs6763178, rs11926280, rs4132537, |
| | rs7430698, rs7430715, rs62276526, rs9813069, rs12639465, rs6441300, |
| | rs7650523, rs7653196, rs9810780, rs6762170, rs6767301, rs6764699, |
| | rs60066171, rs12107958, rs10936218, rs28853091, rs59596155, |
| | rs371514790, rs62276556, rs67600559, rs60272820, rs56779007, |
| | rs56402168, rs7429392, rs4075020, rs4073343, rs10936221, rs6441308, |
| | rs74420888, rs7429277, rs7426815, rs6780654, rs6792014, rs6777810, |
| | rs60511852, rs58689996, rs55789829, rs6773445, rs6786070, rs4928110, |
| | rs6441316, rs6441317, rs6441318, rs6799879, rs6799882, rs6799992, |
| | rs6802787, rs62278361, rs7433589, rs11719592, rs55764247, rs7653126, |
| | rs6441321, rs66828943, rs56736439, rs67348505, rs66747627, |
| | rs6808426, rs4928112, rs9855549, rs9819246, rs9814643 |
| NFXL1 | rs3792711, rs2351784, rs5026911, rs10029980, rs35736575, rs12643755, |
| 4:47664665- | rs17601341, rs2271036, rs2271037, rs10517195, rs13111339, |
| 48106708 | rs13131207, rs28667091, rs17463016, rs13135995, rs13118104, |
| 224 SNPs | rs13137804, rs7675211, rs3792710, rs3792709, rs13135597, rs7665841, |
| | rs7661414, rs4441728, rs4558839, rs4271983, rs4271984, rs4273435, |
| | rs7681242, rs6447580, rs2001935, rs13125059, rs4473628, rs6835814, |
| | rs4591570, rs565912251, rs10049713, rs4695278, rs4695280, rs4518214, |
| | rs16860663, rs1440227, rs6823698, rs1866689, rs7663935, rs6823184, |
| | rs2289433, rs6447586, rs6447587, rs1062858, rs56001837, rs1371731, |
| | rs6820636, rs1440221, rs17463666, rs9291317, rs200424428, rs6822668, |
| | rs12504018, rs10025104, rs10030927, rs4695290, rs2033898, rs4529019, |
| | rs28573115, rs4565052, rs7687363, rs12641871, rs12186152, rs6414687, |
| | rs6447589, rs2033893, rs4695292, rs978094, rs28412313, rs2289435, |
| | rs9997555, rs4694872, rs12506632, rs12507439, rs10938503, rs7690562, |
| | rs2053404, rs28583602, rs10938504, rs6844286, rs6827508, rs1440228, |
| | rs6811177, rs8180146, rs10938505, rs10938506, rs4695303, rs6447592, |
| | rs2352145, rs6447593, rs12651301, rs9994561, rs10028155, rs1822030, |
| | rs1371730, rs12503789, rs4695306, rs60515154, rs28636596, |
| | rs28460483, rs6858440, rs201258817, rs10008818, rs4435717, |
| | rs9995122, rs1465885, rs4695317, rs1371729, rs1012844, rs17573709, |
| | rs4405986, rs2352470, rs1984713, rs10805160, rs1440223, rs12510380, |
| | rs7687722, rs10938510, rs12507658, rs12499841, rs12512437, |
| | rs2352471, rs2352472, rs12640473, rs10938511, rs10938512, rs7668132, |
| | rs6814656, rs1371732, rs7659539, rs11722611, rs10938514, rs7689884, |
| | rs7693648, rs7677851, rs10461076, rs55896433, rs7694104, rs11936897, |
| | rs3857072, rs59252780, rs10517202, rs11939754, rs6447603, rs1507922, |
| | rs2581490, rs321620, rs11734881, rs2033896, rs1848341, rs1848342, |
| | rs2661541, rs2581494, rs3098642, rs62300274, rs6816850, rs1961011, |
| | rs59363169, rs3113878, rs12505197, rs321636, rs321645, rs2661513, |
| | rs6836669, rs7667425, rs321628, rs321630, rs321631, rs2048157, |
| | rs321622, rs13140968, rs13142905, rs13125949, rs62300320, |
| | rs11733284, rs6832503, rs2352413, rs2352414, rs7690405, rs13146880, |
| | rs4694879, rs11722463, rs11736427, rs4695332, rs13107767, rs4340770, |
| | rs906487, rs113802042, rs11930716, rs13135988, rs199501877, |
| | rs140084414, rs9685570, rs1507925, rs28667740, rs28650378, |
| | rs6447612, rs6447613, rs6839501, rs33964061, rs12511511, rs6829490, |
| | rs6447614, rs2230594, rs9996535, rs875179, rs6447615, rs3805187, |
| | rs6850108, rs6854382, rs6833403, rs10007288, rs13118073, rs3749504, |
| | rs1532967, rs6822209, rs1472865, rs71611579 |
| UGT2B17 | rs6552179, rs57105149, rs11726194, rs11727878, rs7666993, |
| 4:69204672- | rs13124352, rs28656921, rs4860942, rs4860943, rs4860296, rs4860297, |
| 69579402 | rs11722236, rs11726027, rs11726831, rs564259802, rs11735363, |
| 210 SNPs | rs11735400, rs149380078, rs144708168, rs148531523, rs113476533, |
| | rs28820893, rs28794577, rs28864819, rs35283635, rs35744988, |
| | rs35057908, rs34595700, rs2708661, rs2045097, rs62317937, |
| | rs62317938, rs146077283, rs2603149, rs62317939, rs370878539, |
| | rs3109121, rs9884394, rs9884395, rs9884396, rs367549135, rs62317941, |
| | rs62317942, rs7681047, rs7663653, rs7663807, rs7668901, rs62317943, |
| | rs4860953, rs200441348, rs200442134, rs62317957, rs62317959, |
| | rs13119626, rs13147617, rs13121164, rs13119652, rs13121169, |
| | rs7691211, rs4860955, rs4860956, rs10016138, rs10213381, rs10212906, |
| | rs10212950, rs10212951, rs10213260, rs10213263, rs12507990, |
| | rs12503212, rs12511559, rs10029605, rs10213031, rs4401527, |
| | rs4260611, rs4262052, rs62316965, rs28882999, rs4860959, rs4860960, |
| | rs4860961, rs4860962, rs13104824, rs13105976, rs10028934, |
| | rs12498676, rs12506348, rs12501703, rs4860298, rs4860299, rs4860963, |
| | rs10026143, rs10004435, rs34138969, rs10026428, rs12511328, |
| | rs10471127, rs71615059, rs4388157, rs10471128, rs10471154, |
| | rs12509927, rs13143417, rs62316968, rs62316969, rs13117021, |
| | rs34572119, rs62316970, rs10023390, rs28792733, rs4576088, |
| | rs4591686, rs11726244, rs11726303, rs7697138, rs7671342, rs56194147, |
| | rs61232256, rs71615060, rs7439528, rs28510329, rs10025771, |
| | rs10005524, rs10028532, rs7440951, rs7438491, rs11723145, |
| | rs11731661, rs28853931, rs62316984, rs12502808, rs62316987, |
| | rs7435827, rs7436962, rs9996186, rs62316988, rs9999599, rs7434408, |
| | rs7438492, rs62316989, rs28828487, rs10015614, rs11731760, |
| | rs11732705, rs11732803, rs12505007, rs7442348, rs10025125, |
| | rs62317000, rs59678213, rs571314519, rs62317004, rs6817882, |
| | rs62317007, rs62317008, rs10029892, rs10049812, rs55786146, |
| | rs28660525, rs6829482, rs6817347, rs12503703, rs13144491, |
| | rs13111422, rs13111428, rs13101504, rs13101719, rs7435918, |
| | rs13139305, rs13116034, rs7441376, rs7441381, rs7442458, rs12501434, |
| | rs62317020, rs6816553, rs4860967, rs4860968, rs4860969, rs4860970, |
| | rs4860971, rs12504563, rs71615069, rs6811340, rs10049758, rs6838228, |
| | rs7441310, rs12509800, rs7440742, rs6552188, rs7660551, rs28481629, |
| | rs6825318, rs6812826, rs28462160, rs7437092, rs9994240, rs10025294, |
| | rs9997405, rs10028930, rs9992446, rs28820590, rs28843700, rs7670012, |
| | rs28769424, rs12500806, rs10049957, rs28562740, rs6825560, |
| | rs1840285 |
| ENPEP | rs7682430, rs404620, rs244046, rs1487261, rs9990948, rs244041, |
| 4:111219276- | rs244040, rs244038, rs244035, rs244034, rs244033, rs244032, |
| 111556487 | rs6851645, rs6828529, rs244030, rs183616, rs62336636, rs406653, |
| 218 SNPs | rs6855198, rs6829462, rs244055, rs244054, rs453198, rs12108663, |
| | rs6837599, rs13134685, rs13135761, rs13113943, rs244027, |
| | rs111303744, rs143272709, rs13150369, rs7684414, rs7662662, |
| | rs7684937, rs13111771, rs13139237, rs112834969, rs112623444, |
| | rs13120157, rs28472706, rs2190572, rs21806, rs7689037, rs7689043, |
| | rs244021, rs244019, rs2029226, rs244014, rs244001, rs243997, |
| | rs173403, rs56912199, rs243995, rs243992, rs243989, rs243988, |
| | rs243985, rs400993, rs410208, rs400966, rs243984, rs243981, rs46716, |
| | rs243979, rs243978, rs243977, rs243976, rs243975, rs243973, rs243972, |
| | rs243970, rs243969, rs243965, rs243964, rs12508135, rs243961, |
| | rs171502, rs243960, rs487979, rs7692954, rs243957, rs243956, |
| | rs243952, rs243950, rs243949, rs243946, rs243945, rs243944, rs243942, |
| | rs243939, rs243938, rs243937, rs243935, rs243934, rs243923, |
| | rs12498628, rs34206896, rs1487256, rs2220328, rs2348205, rs28680830, |
| | rs12502066, rs1487263, rs9993965, rs4834674, rs13110467, rs34817391, |
| | rs10017211, rs1386477, rs1386476, rs1906228, rs7680879, rs2348213, |
| | rs2348211, rs2348210, rs2129568, rs2129567, rs71603076, rs13123641, |
| | rs11729635, rs11734086, rs7682795, rs12506286, rs6829637, rs2348209, |
| | rs199860222, rs2087160, rs1879056, rs1879057, rs6533515, rs9994289, |
| | rs9997893, rs6818198, rs13136326, rs4834497, rs10021430, rs10021437, |
| | rs10021439, rs62336816, rs10434005, rs4834529, rs10001506, |
| | rs10033071, rs10000348, rs10004154, rs6825911, rs6848906, rs6823889, |
| | rs6831174, rs7685862, rs6533521, rs4833586, rs4833587, rs1879053, |
| | rs6839421, rs6813719, rs13118687, rs6533524, rs6812955, rs4277845, |
| | rs2001255, rs1126483, rs12503640, rs2348427, rs17041826, rs2881913, |
| | rs12505584, rs35269579, rs2348428, rs2348429, rs17551888, |
| | rs16997154, rs6842486, rs2348430, rs10015807, rs12506732, rs2348431, |
| | rs13134008, rs6813802, rs3796889, rs3796888, rs3733596, rs2348433, |
| | rs1996604, rs6811162, rs1448808, rs17138, rs639194, rs522426, |
| | rs1375298, rs35400392, rs670014, rs599866, rs478454, rs482254, |
| | rs604612, rs3796881, rs2029073, rs2290105, rs1375300, rs11935471, |
| | rs10028326, rs11944376, rs12641993, rs1448813, rs1448812, |
| | rs10019582, rs10021237, rs376981618, rs976568, rs994978, rs2723329, |
| | rs2595105, rs2595104, rs2739206, rs2723321, rs2595100 |
| PTGER4 | rs7736920, rs28548545, rs1876144, rs1876142, rs1876141, rs1505994, |
| 5:40520133- | rs6873494, rs954687, rs954688, rs4512170, rs10043301, rs6896271, |
| 40848867 | rs4957319, rs6451515, rs6451516, rs7732405, rs1587960, rs7720834, |
| 219 SNPs | rs7706240, rs2371721, rs2888140, rs6451519, rs6885108, rs6451521, |
| | rs6451522, rs12656271, rs13160426, rs4957322, rs6451523, rs6451525, |
| | rs1995601, rs7735058, rs12697413, rs13167534, rs11750156, rs4016332, |
| | rs7737253, rs2135331, rs10055860, rs11742349, rs10075020, rs1948901, |
| | rs1948902, rs9292781, rs1122433, rs7708395, rs7713102, rs2036597, |
| | rs9292782, rs13179528, rs6897467, rs906062, rs6878874, rs6879632, |
| | rs10473203, rs13163402, rs10941518, rs1979937, rs10214209, |
| | rs4957138, rs4957326, rs1354673, rs7729332, rs11739531, rs7726047, |
| | rs6892418, rs10064394, rs7714305, rs7719046, rs6879329, rs7716982, |
| | rs4957333, rs6451533, rs6882769, rs6893430, rs62358056, rs10071870, |
| | rs115118919, rs10057182, rs10038251, rs10035697, rs10043891, |
| | rs10038367, rs10044538, rs28435248, rs72747928, rs10941522, |
| | rs10078260, rs4957334, rs4957335, rs4957336, rs4957337, rs10065482, |
| | rs11744569, rs12513529, rs7730020, rs2888173, rs62358800, rs1395084, |
| | rs10473215, rs4314453, rs4607392, rs4412150, rs10074117, rs4434423, |
| | rs45437592, rs4133101, rs10039983, rs45613037, rs4546432, |
| | rs11957406, rs13354346, rs10036575, rs28540420, rs10055925, |
| | rs7726237, rs4957342, rs7730368, rs10064175, rs10056345, |
| | rs528625539, rs6867618, rs63470160, rs7708680, rs61576290, |
| | rs62356468, rs57806386, rs59069340, rs4509070, rs59920139, |
| | rs4285273, rs4315965, rs6896993, rs4957344, rs6872282, rs4573034, |
| | rs60770691, rs7716285, rs1345778, rs7726270, rs6873054, rs10038769, |
| | rs1692252, rs369940, rs447810, rs249414, rs7705504, rs10071679, |
| | rs249423, rs59585832, rs837106, rs3805497, rs432071, rs2329353, |
| | rs77439436, rs6868517, rs249425, rs29744, rs3805495, rs7702883, |
| | rs4957350, rs12517210, rs249428, rs1002424, rs1002423, rs9800106, |
| | rs193864, rs257009, rs154275, rs58692207, rs59338019, rs28544962, |
| | rs154281, rs10053664, rs112287054, rs56046506, rs171606, rs4957351, |
| | rs152372, rs4957352, rs72747984, rs154284, rs10074991, rs58751240, |
| | rs13361707, rs12514352, rs555245038, rs28515155, rs152375, rs154268, |
| | rs3805487, rs466108, rs7711806, rs59133000, rs7722448, rs62357564, |
| | rs2008763, rs166073, rs1122655, rs6884484, rs1001684, rs11956019, |
| | rs11956047, rs6897470, rs193866, rs10043245, rs73084490, rs7725810, |
| | rs6876367, rs7717357, rs10054398, rs3816959, rs10065570, rs3763074, |
| | rs6899032, rs173969, rs837388, rs323565, rs323563 |
| DRD1 | rs11960551, rs35507411, rs11960265, rs2471016, rs2471015, rs2247169, |
| 5:174748969- | rs7445817, rs2445809, rs2445808, rs1870062, rs2001793, rs897748, |
| 174987537 | rs2471014, rs2445805, rs7449230, rs4424010, rs2471012, rs2471011, |
| 226 SNPs | rs4242185, rs4242186, rs4868511, rs4868512, rs2471010, rs2471009, |
| | rs4333332, rs1563556, rs1563557, rs4349731, rs7378688, rs6861238, |
| | rs2445812, rs10041114, rs2445811, rs251927, rs10078829, rs4868517, |
| | rs265942, rs703746, rs2249205, rs265943, rs265944, rs2607179, |
| | rs2544726, rs265945, rs265948, rs265950, rs265951, rs265952, |
| | rs265953, rs1809809, rs265954, rs265955, rs2264262, rs140212424, |
| | rs115416478, rs147721734, rs265957, rs265959, rs2544727, rs924092, |
| | rs924093, rs1457086, rs265960, rs265962, rs2431135, rs265963, |
| | rs265964, rs265965, rs1585154, rs1585155, rs2431136, rs251949, |
| | rs251948, rs251947, rs168772, rs251945, rs251944, rs251943, rs185193, |
| | rs251941, rs251940, rs116353285, rs265982, rs265983, rs265984, |
| | rs265985, rs265987, rs2544738, rs1457087, rs11954565, rs2607170, |
| | rs1582421, rs13167257, rs266004, rs11739696, rs265967, rs265968, |
| | rs2453737, rs265972, rs265973, rs265974, rs265975, rs863126, |
| | rs835541, rs265978, rs11749676, rs251937, rs4867798, rs686, rs267412, |
| | rs267413, rs267415, rs267416, rs267417, rs267418, rs267421, rs267405, |
| | rs267406, rs267407, rs2457089, rs267409, rs1355077, rs1508761, |
| | rs1496133, rs1508763, rs1508764, rs1508765, rs931939, rs931940, |
| | rs2662179, rs2662180, rs2644645, rs1022039, rs1022044, rs1463781, |
| | rs2662181, rs2644647, rs2644648, rs2662182, rs2662183, rs2453742, |
| | rs2960272, rs2960271, rs10476158, rs10476159, rs174820, rs169135, |
| | rs267357, rs267356, rs2662184, rs2662185, rs267355, rs267354, |
| | rs267352, rs267350, rs267349, rs267348, rs267347, rs267346, |
| | rs2940478, rs267367, rs267368, rs267370, rs267371, rs2644658, |
| | rs267387, rs267388, rs267389, rs267390, rs267391, rs836047, rs267373, |
| | rs267375, rs267376, rs267377, rs267378, rs267379, rs267380, rs267381, |
| | rs267382, rs267383, rs6874723, rs7718943, rs6556213, rs7724819, |
| | rs66797059, rs7447236, rs35499915, rs1496134, rs201325847, |
| | rs2652194, rs28549349, rs11134927, rs2662159, rs884457, rs1109984, |
| | rs1109983, rs2644677, rs2662168, rs2644671, rs28480555, rs157472, |
| | rs157473, rs157475, rs157476, rs156374, rs156373, rs6897275, |
| | rs273078, rs156372, rs156371, rs156370, rs267399, rs267398, rs198037, |
| | rs267397, rs156378, rs157479, rs2453746, rs13353968, rs2644662, |
| | rs267393, rs12109728, rs157481, rs10036863, rs2940480 |
| ZBTB9 | rs213201, rs213200, rs213199, rs213203, rs213204, rs213205, rs213206, |
| 6:33232441- | rs213207, rs120626, rs213215, rs469064, rs455567, rs446735, rs462093, |
| 33614813 | rs463260, rs464865, rs456261, rs3130018, rs2073524, rs2073525, |
| 181 SNPs | rs461964, rs461887, rs420714, rs467065, rs376006, rs62407903, |
| | rs459927, rs437874, rs442745, rs411136, rs2480487, rs2772387, |
| | rs9767795, rs2772384, rs34550603, rs210192, rs210177, rs210179, |
| | rs210180, rs210182, rs210185, rs1794683, rs210186, rs210188, |
| | rs190003, rs1794685, rs449242, rs1794687, rs372469, rs372846, |
| | rs447418, rs371155, rs9469481, rs380163, rs367408, rs368716, |
| | rs396516, rs367897, rs404778, rs386879, rs405126, rs3916269, |
| | rs9461874, rs73404347, rs62405927, rs9469483, rs210197, rs9357160, |
| | rs210201, rs210202, rs210203, rs210204, rs210205, rs210206, rs169740, |
| | rs9469485, rs210207, rs526728, rs526726, rs210208, rs210209, |
| | rs210210, rs210150, rs191901, rs10947409, rs210156, rs210160, |
| | rs9366824, rs210170, rs9380369, rs210130, rs6910233, rs6914422, |
| | rs210132, rs210133, rs9380371, rs210134, rs511515, rs513349, |
| | rs9296095, rs210138, rs210139, rs5745587, rs5745582, rs210141, |
| | rs210144, rs210145, rs210146, rs530878, rs6900408, rs549888, |
| | rs563751, rs394199, rs395671, rs407710, rs420361, rs420628, rs430655, |
| | rs375555, rs422907, rs386906, rs10947410, rs6929774, rs6930081, |
| | rs6929819, rs547391, rs57831685, rs59600351, rs493871, rs576166, |
| | rs529661, rs543555, rs12213869, rs1980772, rs12202102, rs565654, |
| | rs495659, rs508298, rs4713637, rs510153, rs511007, rs501084, |
| | rs525819, rs487358, rs574186, rs493063, rs210212, rs210214, rs210215, |
| | rs374216, rs2988057, rs2988058, rs184113, rs10947413, rs210120, |
| | rs210121, rs210122, rs3916279, rs444697, rs407415, rs9348921, |
| | rs9461893, rs9469527, rs9368768, rs6903502, rs9348922, rs6926568, |
| | rs7768202, rs4713644, rs9469532, rs9461894, rs12662525, rs4713646, |
| | rs55792355, rs55642707, rs9469537, rs4713648, rs10947420, rs9394158, |
| | rs6935686, rs7753658 |
| VNN3 | rs9389009, rs4273706, rs4305747, rs4305748, rs7765050, rs4320395, |
| 6:132915856- | rs4543387, rs4467795, rs9402430, rs72987618, rs143834899, rs9402431, |
| 133185375 | rs9402432, rs7772235, rs12195718, rs9493395, rs12200430, rs9399036, |
| 230 SNPs | rs3813352, rs3813351, rs9389012, rs9389013, rs9385617, rs79531069, |
| | rs9375901, rs9493403, rs9389016, rs9385619, rs12213507, rs4285349, |
| | rs11961682, rs11966589, rs10872394, rs7739700, rs11962787, |
| | rs4418213, rs4256456, rs62427292, rs17061564, rs7754356, rs7765058, |
| | rs7747513, rs4897596, rs2745419, rs2745420, rs2840808, rs2840809, |
| | rs2745421, rs2745422, rs2840811, rs2745424, rs2745426, rs1044593, |
| | rs2840813, rs2745427, rs2251455, rs2251449, rs2745429, rs2745430, |
| | rs742516, rs909975, rs909977, rs2840815, rs2745435, rs2840816, |
| | rs2840817, rs2840819, rs2745436, rs2745437, rs2262437, rs2840820, |
| | rs2262438, rs2840821, rs2840822, rs2840823, rs2745439, rs2745440, |
| | rs4897601, rs4897604, rs9689939, rs2247285, rs2247284, rs2247269, |
| | rs2223613, rs2840824, rs2745441, rs2745442, rs2745443, rs2745444, |
| | rs2840825, rs2745445, rs2840826, rs990499, rs990500, rs9399038, |
| | rs6936833, rs2327275, rs4897605, rs6938196, rs6938355, rs9399039, |
| | rs1073954, rs2300077, rs4897608, rs4897609, rs7742200, rs3823026, |
| | rs3798792, rs2294757, rs3756975, rs12211414, rs6569826, rs6569827, |
| | rs4897614, rs7752016, rs12526754, rs6569843, rs2267952, rs6925157, |
| | rs6937989, rs6938188, rs1883617, rs9493423, rs9493425, rs2300079, |
| | rs56739181, rs1883613, rs11758216, rs11154695, rs7751879, rs6917833, |
| | rs761125, rs6920271, rs2327278, rs4275081, rs2209154, rs10457594, |
| | rs3756969, rs3756968, rs9493450, rs12202302, rs9493451, rs9389034, |
| | rs2236382, rs9399043, rs9321374, rs9399044, rs2327279, rs4599669, |
| | rs9402457, rs9375921, rs6569853, rs6569854, rs755895, rs1544060, |
| | rs6921262, rs755894, rs62425134, rs62425136, rs6907421, rs6927761, |
| | rs6569855, rs7764152, rs7768634, rs12192765, rs12206120, rs7774585, |
| | rs9321377, rs9493458, rs9493459, rs12195295, rs12215143, rs12215255, |
| | rs111884616, rs9493460, rs7761504, rs9373030, rs9321379, rs9321380, |
| | rs7766944, rs7768053, rs7768470, rs12200907, rs12193918, rs12207260, |
| | rs6569856, rs1359142, rs7759900, rs6569857, rs6569858, rs6569859, |
| | rs1965895, rs1001018, rs7751333, rs7751359, rs7755422, rs7756588, |
| | rs9402460, rs9402461, rs1124450, rs6925225, rs1408062, rs1408063, |
| | rs9375924, rs9375925, rs9402462, rs7743635, rs7744235, rs7764252, |
| | rs7744426, rs9493464, rs7764726, rs9493465, rs9321382, rs9321383, |
| | rs10872399, rs7749419, rs7750176, rs4897616, rs4897617, rs4897618, |
| | rs9389037, rs9389038, rs1009687, rs9385623, rs1555621, rs12200483, |
| | rs12200485, rs9493468, rs9493470 |
| FAM183B | rs6951641, rs3823593, rs3807415, rs10277748, rs3807412, rs4720287, |
| 7:38535089- | rs4720288, rs9639796, rs10233502, rs2043787, rs2043788, rs4723762, |
| 38914878 | rs4723763, rs3807410, rs3807406, rs11316401, rs7783972, rs7788738, |
| 235 SNPs | rs2028209, rs62443506, rs10951560, rs62443507, rs12536311, |
| | rs12536339, rs6957726, rs6462848, rs7812192, rs7812098, rs10236438, |
| | rs10262613, rs10247916, rs3778875, rs3807399, rs3807397, rs3800797, |
| | rs3800796, rs3800795, rs3807394, rs3807393, rs2392580, rs2392581, |
| | rs4720289, rs10237576, rs41478945, rs7778286, rs76190784, rs1561308, |
| | rs2392582, rs10228705, rs6976444, rs35099182, rs4385366, rs893413, |
| | rs920268, rs2118460, rs2164838, rs2118462, rs6942910, rs1540480, |
| | rs1540481, rs7785499, rs6462849, rs6974116, rs4427070, rs4427071, |
| | rs10264751, rs13245847, rs7776809, rs1987195, rs58580109, |
| | rs10269961, rs6965722, rs6948732, rs7811852, rs10247532, rs6962805, |
| | rs6945114, rs10263645, rs7811508, rs12701640, rs10271913, rs9987017, |
| | rs6973823, rs7807867, rs2043260, rs9918553, rs11763211, rs7781415, |
| | rs12701647, rs2392589, rs2893564, rs6951440, rs6944287, rs12701649, |
| | rs10244270, rs2392593, rs34159696, rs2392594, rs6462857, rs10951569, |
| | rs11984273, rs11981077, rs6978220, rs6977810, rs6978226, rs6977813, |
| | rs6977993, rs6978759, rs6978500, rs6945492, rs6945629, rs2116666, |
| | rs11980087, rs12701651, rs12701652, rs1864886, rs7810938, rs6964269, |
| | rs6968795, rs12701654, rs10227687, rs10951571, rs7794371, rs7811599, |
| | rs7778570, rs7779329, rs1978172, rs1978171, rs17700072, rs7794030, |
| | rs35791079, rs1859494, rs7783546, rs1978170, rs7797871, rs35253728, |
| | rs66796288, rs17767770, rs17700172, rs3735347, rs1468348, rs2189855, |
| | rs6462862, rs4723783, rs62444078, rs17700262, rs733150, rs886646, |
| | rs4720298, rs10247503, rs4720299, rs2107743, rs2286106, rs2286105, |
| | rs2286104, rs2286102, rs10486677, rs17700392, rs10486678, |
| | rs11971020, rs10254567, rs2240355, rs2240354, rs2240353, rs2240351, |
| | rs2286100, rs2286099, rs2286098, rs2286097, rs10271176, rs12672885, |
| | rs2286095, rs2286092, rs2286091, rs2286090, rs3801146, rs10232480, |
| | rs3801145, rs7795874, rs2074655, rs12533557, rs28548028, rs17768153, |
| | rs3801144, rs3779126, rs2392598, rs3801143, rs3801142, rs3801141, |
| | rs17768212, rs2240350, rs7786389, rs7785911, rs62444107, rs4723785, |
| | rs2286087, rs2286086, rs2286085, rs2286084, rs2286083, rs2286082, |
| | rs3801140, rs3801139, rs1017028, rs2240352, rs6961672, rs2074657, |
| | rs2286081, rs17700752, rs2074656, rs6462864, rs6957565, rs4723793, |
| | rs2392601, rs4720306, rs3801135, rs56002981, rs3801132, rs7788664, |
| | rs7798033, rs1473137, rs1859937, rs3801129, rs73357792, rs10259755, |
| | rs6956146, rs12674115, rs34724372, rs17768711, rs2893567, rs7803397, |
| | rs17768753, rs35995292, rs4720308, rs10266891 |
| LRRC17 | rs149123527, rs9769052, rs28822408, rs28792100, rs28837792, |
| 7:102371901- | rs11983917, rs11980235, rs185514513, rs36188109, rs10225238, |
| 102774146 | rs10238871, rs10228568, rs28878294, rs28857600, rs10225809, |
| 198 SNPs | rs10226171, rs11975013, rs2159839, rs2109996, rs2109995, rs2109994, |
| | rs3987952, rs3987955, rs2411045, rs10254446, rs10262368, rs10233095, |
| | rs62484863, rs6943840, rs10250314, rs10227992, rs10273564, |
| | rs10258071, rs2539366, rs28478484, rs2539353, rs6945867, rs12386747, |
| | rs55796765, rs2539313, rs2539312, rs3987948, rs2261257, rs77490360, |
| | rs2411046, rs6979964, rs6980434, rs2430070, rs10464611, rs4489255, |
| | rs149805825, rs12672488, rs9691949, rs199970642, rs9718444, |
| | rs56356294, rs7778418, rs7778423, rs7778426, rs7800387, rs4727556, |
| | rs74708264, rs2539217, rs3972456, rs7778760, rs7778519, rs142264290, |
| | rs7782908, rs3933460, rs10228495, rs6975388, rs80313245, rs75387186, |
| | rs6969054, rs10257317, rs57253646, rs7780981, rs6946773, rs10279449, |
| | rs11981980, rs75197737, rs1541519, rs17136090, rs12668434, |
| | rs59544295, rs10271184, rs76165167, rs7800548, rs59709436, |
| | rs7779145, rs7804322, rs56269620, rs6967389, rs12666764, rs2411056, |
| | rs60524601, rs56894117, rs6945369, rs140064303, rs143740918, |
| | rs181753416, rs57350619, rs58680618, rs57776797, rs9690210, |
| | rs60599650, rs3779029, rs2215040, rs6976005, rs12667218, rs6465876, |
| | rs11983456, rs11983521, rs11973870, rs11973896, rs11980643, |
| | rs73406284, rs10267275, rs11982616, rs11972644, rs59278165, |
| | rs58191643, rs57545514, rs56921864, rs12540384, rs11970869, |
| | rs80263722, rs12530741, rs11978670, rs11972019, rs11978768, |
| | rs11978799, rs6953980, rs6465877, rs6465878, rs6465879, rs6957434, |
| | rs7794668, rs6947387, rs2159022, rs10435335, rs10241865, rs10271157, |
| | rs10242028, rs10257543, rs10273725, rs10250490, rs12537977, |
| | rs1100044, rs1100046, rs847647, rs847648, rs847649, rs847652, |
| | rs847656, rs847658, rs10499952, rs12056296, rs6465882, rs73408250, |
| | rs6947403, rs6465885, rs6968713, rs12532495, rs13242493, rs35479298, |
| | rs6960428, rs57616389, rs13233521, rs4620204, rs9718453, rs13242403, |
| | rs199543678, rs11514917, rs12671803, rs2228686, rs1608651, |
| | rs7789252, rs4729860, rs869332, rs11972261, rs11976237, rs6963104, |
| | rs7785272, rs10808120, rs2222539, rs4729866, rs1829957, rs13222980, |
| | rs12666317, rs2298854, rs4729873, rs6978630, rs1861729, rs13232194, |
| | rs10953380, rs11770319, rs10953383 |
| ADAM2 | rs1584683, rs11785708, rs7460073, rs11785546, rs1376720, rs7464400, |
| 8:39483598- | rs3824144, rs12708194, rs13278646, rs3852338, rs3852339, rs3864654, |
| 39813985 | rs11998440, rs4297011, rs7815141, rs7820447, rs7842919, rs2060802, |
| 222 SNPs | rs6474161, rs6474162, rs10958560, rs11985161, rs35893648, rs2100310, |
| | rs10106405, rs34113924, rs6995642, rs7835711, rs12548363, rs4733923, |
| | rs7820085, rs9657181, rs9657175, rs7838350, rs2198204, rs7461047, |
| | rs6474169, rs12114080, rs201962, rs6474170, rs7832208, rs10958562, |
| | rs12548069, rs201996, rs4440676, rs10958563, rs12542470, rs12547283, |
| | rs201991, rs4634692, rs12682366, rs12674707, rs12676113, rs4733921, |
| | rs9643843, rs12548872, rs10958564, rs12548963, rs12549508, |
| | rs2122987, rs12550182, rs79471707, rs7011836, rs9643871, rs2122988, |
| | rs1026234, rs201977, rs201976, rs201975, rs201972, rs201971, |
| | rs1160383, rs201970, rs201968, rs661832, rs673382, rs674299, |
| | rs28374483, rs423931, rs12541801, rs10091293, rs10105827, |
| | rs12548821, rs7017903, rs10097616, rs10087294, rs1972168, |
| | rs10808923, rs10105703, rs13272881, rs13280279, rs10808924, |
| | rs3853758, rs10096756, rs2122992, rs202007, rs10091156, rs2290301, |
| | rs12681344, rs13253873, rs1947319, rs10100159, rs13265483, |
| | rs3779718, rs7822312, rs5891067, rs2100309, rs1451745, rs1451741, |
| | rs1901386, rs10106311, rs13274165, rs7388612, rs10108837, rs7463448, |
| | rs34190387, rs28569468, rs7005636, rs13253377, rs6984521, rs6993167, |
| | rs10105530, rs28477916, rs7836577, rs6474179, rs10112921, |
| | rs12708198, rs10093420, rs7825536, rs7012629, rs28445172, rs6998587, |
| | rs6998739, rs2032722, rs2032721, rs6981259, rs6999730, rs6981266, |
| | rs2032720, rs7002831, rs6474180, rs6474181, rs6474182, rs35350994, |
| | rs6989075, rs7004950, rs10113041, rs7006647, rs59547557, rs9773640, |
| | rs36007304, rs11781681, rs10283355, rs13278716, rs28797203, |
| | rs34208092, rs7825999, rs7839240, rs7831046, rs12155609, rs5019014, |
| | rs6474189, rs10958571, rs10086641, rs2879895, rs4472503, rs7816397, |
| | rs7838952, rs7838957, rs28375138, rs888448, rs6980572, rs7017446, |
| | rs6474192, rs9657182, rs2111742, rs3808606, rs3824259, rs7010461, |
| | rs3739319, rs11779313, rs7000868, rs62512638, rs10504013, |
| | rs11992749, rs11994259, rs11781217, rs11787449, rs11773909, |
| | rs11773975, rs11777082, rs11779697, rs11786337, rs11779700, |
| | rs10958573, rs10958574, rs10755980, rs59445010, rs7824841, |
| | rs7832080, rs7845003, rs7828760, rs7845418, rs55818481, rs7003411, |
| | rs6474194, rs6474195, rs6474196, rs6474197, rs6983463, rs6984887, |
| | rs6983940, rs56838465, rs28841363, rs3927941, rs10113563, |
| | rs10085935, rs12677574, rs10958576, rs7013824, rs2729484, |
| | rs12542808 |
| PXDNL | rs28411163, rs4500070, rs11776636, rs1370445, rs6473596, rs1437593, |
| 8:52232145- | rs1219679, rs3097707, rs7460590, rs994127, rs62506862, rs2977016, |
| 52722043 | rs2977018, rs2116349, rs17264278, rs34452992, rs2392898, rs10109706, |
| 212 SNPs | rs10103767, rs2392899, rs2392900, rs1017350, rs4404903, rs10112486, |
| | rs28829107, rs13254540, rs1583195, rs11780995, rs1431618, rs1431617, |
| | rs7004521, rs994588, rs12545563, rs1431636, rs11985980, rs1367819, |
| | rs13263053, rs1814121, rs1594498, rs12675815, rs13254318, |
| | rs12675403, rs1431632, rs13271514, rs13277264, rs11784863, |
| | rs4873195, rs11785786, rs4873555, rs1821115, rs11998376, rs7465120, |
| | rs201466262, rs10958272, rs376341233, rs527720702, rs552682907, |
| | rs7832472, rs755311, rs4873556, rs4873196, rs10755971, rs12541541, |
| | rs12544806, rs10090481, rs2915499, rs4299996, rs2915500, rs2979122, |
| | rs3097698, rs4873557, rs10097136, rs9792278, rs3097708, rs2915453, |
| | rs2979119, rs35889669, rs35377291, rs55677072, rs10958273, |
| | rs5891420, rs7820615, rs6995112, rs10106944, rs4873560, rs4595114, |
| | rs4407870, rs10089190, rs4431586, rs7816253, rs10105910, rs4449780, |
| | rs4320537, rs4295653, rs10105959, rs4873562, rs4873563, rs4333587, |
| | rs4336603, rs4463410, rs6473624, rs34125996, rs6982309, rs4501568, |
| | rs6998491, rs7838984, rs13271101, rs6473629, rs10096851, rs12114812, |
| | rs6997338, rs6984427, rs6998159, rs6994137, rs10109681, rs16916598, |
| | rs16916610, rs12677741, rs35039242, rs34430723, rs62505329, |
| | rs10958283, rs4873566, rs6998137, rs10097683, rs36070314, |
| | rs10106672, rs10958285, rs10958286, rs34228216, rs13264256, |
| | rs12543754, rs78097453, rs146236450, rs71513540, rs11782339, |
| | rs111571782, rs4873568, rs10958293, rs12548130, rs6981117, |
| | rs7000785, rs6981313, rs4469447, rs12677123, rs34906892, rs7836565, |
| | rs7823666, rs5000162, rs34276635, rs16916673, rs4873199, rs9643785, |
| | rs4291264, rs11781376, rs13265125, rs7007297, rs13251959, |
| | rs28549751, rs10109862, rs9650303, rs4873572, rs4873573, rs35153909, |
| | rs6981260, rs10958295, rs6999693, rs6986571, rs6986605, rs10958296, |
| | rs6991118, rs11775179, rs4400372, rs7007671, rs7007804, rs16916741, |
| | rs13277004, rs13249404, rs13274101, rs36075262, rs10111115, |
| | rs11783684, rs76538418, rs199547991, rs16916756, rs2976209, |
| | rs4873579, rs4275221, rs13268711, rs13268396, rs4326377, rs1317774, |
| | rs12548147, rs16916763, rs7005788, rs4242472, rs6982363, rs4573263, |
| | rs12680147, rs13270008, rs13249855, rs4988584, rs7009748, rs7013360, |
| | rs718251, rs722495, rs6990379, rs4242475, rs4242476, rs13279123, |
| | rs13268494, rs13252785 |
| GRHPR | rs566936, rs623871, rs12554976, rs563132, rs664587, rs7869485, |
| 9:37239674- | rs7044356, rs10973268, rs4593644, rs687135, rs13296908, rs4629963, |
| 37566534 | rs10973277, rs555230, rs595800, rs308496, rs308495, rs13289199, |
| 215 SNPs | rs1977756, rs12380446, rs2790063, rs308492, rs10973292, rs3780334, |
| | rs6476622, rs12555207, rs10814519, rs7041291, rs834356, rs308523, |
| | rs7846944, rs495863, rs308499, rs506981, rs13301916, rs997318, |
| | rs10814520, rs6476623, rs526325, rs520762, rs506775, rs503897, |
| | rs56190611, rs34951930, rs501096, rs570874, rs482317, rs476898, |
| | rs483236, rs645928, rs512880, rs502152, rs475331, rs7048697, |
| | rs7048347, rs2009954, rs973671, rs7022351, rs7025491, rs7025809, |
| | rs13288832, rs7042128, rs10814523, rs13300875, rs116894424, |
| | rs117241552, rs10814524, rs10814525, rs10814526, rs10814527, |
| | rs7046378, rs10814529, rs7032564, rs7046921, rs7046910, rs765567, |
| | rs1989660, rs2004605, rs901984, rs7864443, rs744985, rs172622, |
| | rs11506619, rs10814530, rs10814531, rs12552764, rs687400, |
| | rs72739615, rs66867891, rs4878681, rs4878682, rs4878683, rs4878684, |
| | rs4878685, rs35419631, rs525462, rs471579, rs16934059, rs12553453, |
| | rs12553471, rs10046799, rs560903, rs535263, rs13292185, rs7859027, |
| | rs7873048, rs7873051, rs7873057, rs7873219, rs7873371, rs7859637, |
| | rs1927151, rs1927152, rs483327, rs2768653, rs513616, rs2768652, |
| | rs34726045, rs308510, rs182543, rs308506, rs12236363, rs10814535, |
| | rs10814538, rs10973326, rs12235347, rs59967810, rs60485412, |
| | rs66689976, rs7868587, rs7853540, rs7856656, rs10973327, rs10973328, |
| | rs10973329, rs111987946, rs6476631, rs7872991, rs4472607, rs2768659, |
| | rs309457, rs10814542, rs2148870, rs2148871, rs28575219, rs913279, |
| | rs10973358, rs17553670, rs57583560, rs1571234, rs10973361, |
| | rs1887455, rs10814545, rs3758226, rs7860865, rs7860878, rs10814549, |
| | rs7045332, rs10733487, rs754951, rs10814567, rs10814568, rs2274479, |
| | rs2274481, rs7863996, rs7867542, rs4878694, rs112248189, rs7045170, |
| | rs6476635, rs7865901, rs10814569, rs10814570, rs7848968, rs2277161, |
| | rs2296775, rs2296778, rs753968, rs1806857, rs10973387, rs12237351, |
| | rs10814574, rs4457420, rs4878157, rs10758437, rs10973394, rs1475414, |
| | rs10973395, rs12001352, rs7044153, rs7048273, rs10814575, rs4878697, |
| | rs7023107, rs7031794, rs10973409, rs10122666, rs10117312, rs7039920, |
| | rs6476640, rs10118277, rs10758439, rs7044233, rs7850177, rs10814577, |
| | rs10973414, rs10121333, rs10118103, rs1854160, rs7866748, |
| | rs12347889, rs12338759, rs7018980, rs10973436, rs2381714 |
| C9orf47 | rs2799692, rs2818250, rs2653809, rs2653808, rs2174057, rs1502715, |
| 9:91457423- | rs2799699, rs2653812, rs2653811, rs1150388, rs10780207, rs2316807, |
| 91716607 | rs2316806, rs4401949, rs1393568, rs11137433, rs10867126, rs6559299, |
| 223 SNPs | rs6559300, rs1393570, rs10867129, rs200691731, rs7862973, rs4877024, |
| | rs2316799, rs7867270, rs4877025, rs4877026, rs4877028, rs10746567, |
| | rs10746568, rs10780214, rs9410244, rs9410398, rs7022234, rs7029849, |
| | rs7038679, rs10119244, rs4294260, rs4294261, rs7030635, rs4375080, |
| | rs4468011, rs4602976, rs9410405, rs9410246, rs9410247, rs10119025, |
| | rs2316800, rs10117386, rs4877029, rs10118302, rs11137447, |
| | rs11137448, rs3897826, rs4061192, rs4128575, rs4876965, rs4876966, |
| | rs7863020, rs7863057, rs1910266, rs6559311, rs1157007, rs1157008, |
| | rs9314663, rs7036867, rs7026993, rs1910268, rs1910269, rs12682875, |
| | rs1502694, rs1502695, rs4877031, rs7862978, rs7036480, rs7036574, |
| | rs7046367, rs1875620, rs7045912, rs6559313, rs10780217, rs2174055, |
| | rs7035231, rs1502696, rs1502697, rs9410258, rs7854945, rs7021651, |
| | rs9410260, rs9410414, rs9410261, rs2316804, rs2316803, rs4598330, |
| | rs9410415, rs7021587, rs7038956, rs7039077, rs9410263, rs9410264, |
| | rs7028197, rs1818143, rs9410271, rs11137463, rs9410273, rs7047296, |
| | rs7041435, rs7029413, rs1910271, rs1910272, rs28852385, rs9410277, |
| | rs66551258, rs67308794, rs9410278, rs9410423, rs28667788, |
| | rs77690878, rs28622390, rs28425709, rs1502699, rs1502700, rs9410424, |
| | rs9410280, rs11525966, rs11525967, rs9410425, rs9410426, rs9410427, |
| | rs9410428, rs59019708, rs56261853, rs57961801, rs59420663, |
| | rs11790735, rs11792354, rs11792366, rs11790801, rs9410430, |
| | rs9410431, rs9410432, rs7032526, rs7019938, rs7032662, rs10780218, |
| | rs7044951, rs7020528, rs9410283, rs6559317, rs7024847, rs7037603, |
| | rs9410435, rs9410436, rs9410285, rs6559318, rs7041991, rs6559319, |
| | rs7042353, rs78122438, rs75088308, rs3934638, rs4242598, rs6559320, |
| | rs12338537, rs10867146, rs12552799, rs7854677, rs6559324, rs7027496, |
| | rs7031378, rs7031515, rs10780219, rs7031490, rs7025787, rs4373596, |
| | rs4275288, rs4275289, rs4292775, rs7048253, rs34869600, rs7029679, |
| | rs4282643, rs6559326, rs6559327, rs6559328, rs7033637, rs4297102, |
| | rs4344158, rs4297103, rs7469103, rs6559330, rs11137469, rs7047562, |
| | rs7467476, rs4877036, rs4877037, rs7857344, rs7869712, rs10867148, |
| | rs11137473, rs4489384, rs11137477, rs4075163, rs11137478, |
| | rs11793996, rs28733578, rs11791652, rs7043095, rs7022797, |
| | rs11137480, rs4877039, rs7863572, rs6559331, rs10867149, rs3934594, |
| | rs10122011, rs10780221, rs2316318, rs9410299, rs562738231, |
| | rs4877048, rs2873165 |

^{†}gene-region loci are provided with reference to the Genome Reference Consortium Human Build 37 (GRCh37), which is herein also referred to as “hg19”. The loci are provide in the following form: X:Y-Z, wherein X is the chromosome name, Y is the start position of the gene-region, and Z is the end position of the gene-region.

The gene-regions recited above in Table 2 are gene-regions that contain genes that are known to not be commonly mutated in a cancer. Table 2 also includes gene-regions that contain the genes, UGT2B17 and ZBTB9. UGT2B17 and ZBTB9 are known to exhibit high frequency germline copy number losses. Gene-regions containing UGT2B17 and ZBTB9 are included as positive controls for the method of the invention because they are known to commonly contain asCNAs. If a user of the present method determined that the gene-regions containing UGT2B17 and ZBTB9 did not contain any asCNAs, it would suggest that the results for that biological sample are inconclusive and/or unreliable. The gene-regions defined in Table 2 are hereinafter referred to as the “control gene-regions”.

Tables 1 and 2 recite the SNPs within each gene-region defined in Tables 1 and 2. The present inventors selected the SNPs recited in Tables 1 and 2 after discovering that selecting high MAF SNPs in the bespoke gene-regions of the selected genes lead to accurate and sensitive results for establishing allelic imbalance and copy number loss in samples from subjects having cancer. The inventors determined that the SNPs of Tables 1 and 2 have a high MAF, and a high MAF across subjects of different ethnicities, using the 1000 Genomes Project Genotype Data (release 20130502; PMID: 26432245). The SNPs recited in Tables 1 and 2 are located within the exonic regions, intronic regions or intergenic flanking regions of the gene-regions defined in Table 1 or 2.

In certain embodiments, step ii) of the present method comprises detecting at least one (for example 1, 2, 3, 4 or 5; typically at least 2, at least 3 or at least 4) target gene-region selected from the group consisting of FOXA1, FOXP1, HSD3B1, NCOA2 and ZBTB16 as defined in Table 1. Genetic aberrations in such gene-regions are known or suspected of being associated with the function in the androgen (receptor) signalling pathway.

In certain embodiments, step ii) of the present method comprises detecting at least one (for example 1, 2, 3, 4, 5, 6, 7, 8, 9, 10, 12, 15 or 17; typically at least 2, at least 3 or at least 4) target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 as defined in Table 1. Genetic aberrations in such gene-regions are known or suspected of being associated with cell cycle dysfunction.

In certain embodiments, step ii) of the present method comprises detecting at least one (for example 1, 2, 3, 4, 5, 6, 7 or 8; typically at least 2, at least 3 or at least 4) target gene-region selected from the group consisting of AKT1, AKT2, AKT3, MET, PIK3C, PIK3CB, PIK3R1 and PTEN as defined in Table 1. Genetic aberrations in such gene-regions are known or suspected of being associated with the phosphoinositide 3-kinase (PI3K) signalling pathway.

In certain embodiments, step ii) of the present method comprises detecting at least one (for example 1, 2, 3, 4, 5, 6, 7 or 8; typically at least 2, at least 3 or at least 4) target gene-region selected from the group consisting of ARID1A, CHD1, KDM6A, MED12, SMARCA1, KMT2C, KMT2D and RYBP as defined in Table 1. Genetic aberrations in such gene-regions are known or suspected of being associated with chromatic remodeling dysfunction.

In certain embodiments, step ii) of the present method comprises detecting at least one (for example 1, 2, 3, 4, 5, 6, 8, 10, 12, 15, 20 or 22; typically at least 2, at least 3 or at least 4) target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1. Genetic aberrations in such gene-regions are known or suspected of being associated with DNA repair dysfunction.

In certain embodiments, step ii) of the present method comprises detecting at least one (for example 1, 2, 3, 4, 5, 6, 8, 10, or 12; typically at least 2, at least 3 or at least 4) target gene-region selected from the group consisting of ASXL1, CLU, CYLD, ERG_TMPRSS2, GNAS, IDH1, IDH2, NFE2L2, NKX3-1, RUNX1, SPOP and ZFHX3 as defined in Table 1. Genetic aberrations in such gene-regions are known to be associated with prostate cancer, although their precise role in the development and/or progression of prostate cancer of a subject are currently unknown.

In certain embodiments, step ii) of the present method comprises detecting at least one (for example 1, 2, or 3; typically at least 2) target gene-region selected from the group consisting of APC, CTNNB1 and RNF43 as defined in Table 1. Genetic aberrations in such gene-regions are known or suspected to dysregulate the Wnt signalling pathway.

In certain preferred embodiments, step ii) of the present method comprises detecting at least one (for example 1, 2, 3, 4, 5, or 6; typically at least 2, at least 3, at least 4, at least 5 or 6) target gene-region selected from the group consisting of BRCA2, ATM, RB1, NKX3-1, TP53, and PTEN as defined in Table 1. In certain embodiments, preferably step ii) of comprises detecting at least 3 of the target gene-regions selected from the group consisting of BRCA2, ATM, RB1, NKX3-1, TP53, and PTEN as defined in Table 1. More preferably, step ii) of the present method comprises detecting the gene-regions of at least BRCA2, ATM, RB1, NKX3-1, TP53, and PTEN as defined in Table 1.

In certain embodiments, step ii) of the present method comprises detecting at least one target gene-region selected from the group consisting of FOXA1, FOXP1, HSD3B1, NCOA2 and ZBTB16; and/or at least one target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53; and/or at least one target gene-region selected from the group consisting of ARID1A, CHD1, KMT2C, KMT2D and RYB; and/or at least one target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C; and/or at least one target gene-region selected from the group consisting of ASXL1, CLU, CYLD, ERG_TMPRSS2, GNAS, IDH2, NFE2L2, NKX3-1, RUNX1, SPOP and ZFHX3; and/or at least one target gene-region selected from the group consisting of AKT1, AKT2, AKT3, MET, PIK3C, PIK3CB, PIK3R1 and PTEN; and/or at least one target gene-region selected from the group consisting of APC, CTNNB1 and RNF43.

In certain embodiments, step ii) of the present method comprises detecting at least two, at least three or at least four target gene-region selected from the group consisting of FOXA1, FOXP1, HSD3B1, NCOA2 and ZBTB16; and/or at least two, at least three or at least four target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53; and/or at least two, at least three or at least four target gene-region selected from the group consisting of ARID1A, CHD1, KMT2C, KMT2D and RYB; and/or at least two, at least three or at least four target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C; and/or at least two, at least three or at least four target gene-region selected from the group consisting of ASXL1, CLU, CYLD, ERG_TMPRSS2, GNAS, IDH2, NFE2L2, NKX3-1, RUNX1, SPOP and ZFHX3; and/or at least two, at least three or at least four target gene-region selected from the group consisting of AKT1, AKT2, AKT3, MET, PIK3C, PIK3CB, PIK3R1 and PTEN; and/or at least one, at least two or each target gene-region selected from the group consisting of APC, CTNNB1 and RNF43.

In certain embodiments, step ii) comprises detecting the presence of SNPs in the non-tumor DNA present in a biological sample at:
at least 10%, at least 15%, at least 20%, at least 25%, at least 30%, at least 40%, at least 50%, at least 60%, at least 70%, at least 80%, at least 90% or all of the SNP loci defined in Table 1 for each of at least 5 target gene-regions defined in Table 1;
and optionally at least 10%, at least 15%, at least 20%, at least 25%, at least 30%, at least 40%, at least 50%, at least 60%, at least 70%, at least 80%, at least 90% or all of the SNP loci defined in Table 2 for each of at least 5 control gene-regions defined in Table 2.

In certain embodiments, step ii) comprises detecting the presence of SNPs in the non-tumor DNA present in a biological sample at:
at least 10%, at least 15%, at least 20%, at least 25%, at least 30%, at least 40%, at least 50%, at least 60%, at least 70%, at least 80%, at least 90% or all of the SNP loci defined in Table 1 for each of at least 5 target gene-regions defined in Table 1;
and at least 10%, at least 15%, at least 20%, at least 25%, at least 30%, at least 40%, at least 50%, at least 60%, at least 70%, at least 80%, at least 90% or all of the SNP loci defined in Table 2 for each of at least 5 control gene-regions defined in Table 2.

In one preferred embodiments, step ii) comprises detecting the presence of SNPs in the non-tumor DNA present in a biological sample at:
at least 20%, at least 25%, at least 30%, at least 40%, at least 50%, at least 60%, at least 70%, at least 80%, at least 90% or all of the SNP loci defined in Table 1 for each of at least 5 target gene-regions defined in Table 1;
and optionally at least 20%, at least 25%, at least 30%, at least 40%, at least 50%, at least 60%, at least 70%, at least 80%, at least 90% or all of the SNP loci defined in Table 2 for each of at least 5 control gene-regions defined in Table 2.

In another preferred embodiments, step ii) comprises detecting the presence of SNPs in the non-tumor DNA present in a biological sample at:
at least 20%, at least 25%, at least 30%, at least 40%, at least 50%, at least 60%, at least 70%, at least 80%, at least 90% or all of the SNP loci defined in Table 1 for each of at least 5 target gene-regions defined in Table 1;
and at least 20%, at least 25%, at least 30%, at least 40%, at least 50%, at least 60%, at least 70%, at least 80%, at least 90% or all of the SNP loci defined in Table 2 for each of at least 5 control gene-regions defined in Table 2.

In certain preferred embodiments, step ii) comprises detecting the presence of SNPs in the non-tumor DNA at;
and at least 5% I of the SNP loci defined in Table 1 for each of at least 10, at least 15, at least 20, at least 25, at least 30, at least 40, at least 50, at least 60, at least 70 or all of the target gene-regions defined in Table 1;
and optionally at least 5% of the SNP loci defined in Table 2 for each of at least 10, at least 15, at least 20, at least 25, at least 30, at least 35, or all of the control gene-regions defined in Table 2.

In certain preferred embodiments, step ii) comprises detecting the presence of SNPs in the non-tumor DNA at:
at least 10%, at least 15%, at least 20%, at least 25%, at least 30%, at least 40%, at least 50%, at least 60%, at least 70%, at least 80%, at least 90% or all of the SNP loci defined in Table 1 for each of at least 10, at least 15, at least 20, at least 25, at least 30, at least 40, at least 50, at least 60, at least 70 or all of the target gene-regions defined in Table 1;
and optionally at least 10%, at least 15%, at least 20%, at least 25%, at least 30%, at least 40%, at least 50%, at least 60%, at least 70%, at least 80%, at least 90% or all of the SNP loci defined in Table 2 for each of at least 10, at least 15, at least 20, at least 25, at least 30, at least 35, or all of the control gene-regions defined in Table 2.

In certain preferred embodiments, step ii) comprises detecting the presence of SNPs in the non-tumor DNA at:
at least 10%, at least 15%, at least 20%, at least 25%, at least 30%, at least 40%, at least 50%, at least 60%, at least 70%, at least 80%, at least 90% or all of the SNP loci defined in Table 1 for each of at least 10, at least 15, at least 20, at least 25, at least 30, at least 40, at least 50, at least 60, at least 70 or all of the target gene-regions defined in Table 1;
and at least 10%, at least 15%, at least 20%, at least 25%, at least 30%, at least 40%, at least 50%, at least 60%, at least 70%, at least 80%, at least 90% or all of the SNP loci defined in Table 2 for each of at least 10, at least 15, at least 20, at least 25, at least 30, at least 35, or all of the control gene-regions defined in Table 2.

In certain preferred embodiments, step ii) comprises detecting the presence of SNPs in the non-tumor DNA at:
at least 20%, at least 25%, at least 30%, at least 40%, at least 50%, at least 60%, at least 70%, at least 80%, at least 90% or all of the SNP loci defined in Table 1 for each of at least 10, at least 15, at least 20, at least 25, at least 30, at least 40, at least 50, at least 60, at least 70 or all of the target gene-regions defined in Table 1;
and optionally at least 20%, at least 25%, at least 30%, at least 40%, at least 50%, at least 60%, at least 70%, at least 80%, at least 90% or all of the SNP loci defined in Table 2 for each of at least 10, at least 15, at least 20, at least 25, at least 30, at least 35, or all of the control gene-regions defined in Table 2.

In one preferred embodiment, step ii) comprises detecting the presence of SNPs in the non-tumor DNA at:
at least 20%, at least 25%, at least 30%, at least 40%, at least 50%, at least 60%, at least 70%, at least 80%, at least 90% or all of the SNP loci defined in Table 1 for each of at least 10, at least 15, at least 20, at least 25, at least 30, at least 40, at least 50, at least 60, at least 70 or all of the target gene-regions defined in Table 1;
and at least 20%, at least 25%, at least 30%, at least 40%, at least 50%, at least 60%, at least 70%, at least 80%, at least 90% or all of the SNP loci defined in Table 2 for each of at least 10, at least 15, at least 20, at least 25, at least 30, at least 35, or all of the control gene-regions defined in Table 2.

Typically, step ii) comprises detecting the presence of SNPs in the non-tumor DNA at about 10 to all of the SNP loci defined in Table 1 a gene-region. In one embodiment, step ii) comprises detecting the presence of SNPs in the non-tumor DNA at least 30 of the SNP loci defined in Table 1 a gene-region. In one preferred embodiment, step ii) comprises detecting the presence of SNPs in the non-tumor DNA at least 60 of the SNP loci defined in Table 1 a gene-region. In one preferred embodiment, step ii) comprises detecting the presence of SNPs in the non-tumor DNA at least 90 of the SNP loci defined in Table 1 a gene-region.

In one embodiment, step ii) comprises detecting the presence of SNPs in the non-tumor DNA at:
at least 30, at least 60, or at least 90 of the SNP loci defined in Table 1 for each of at least 5 of the target gene-regions defined in Table 1;
and optionally at least 30, at least 60, or at least 90 of the SNP loci defined in Table 2 for each of at least 5, control gene-regions defined in Table 2.

Typically, step ii) comprises detecting the presence of SNPs in the non-tumor DNA at about 10 to all of the SNP loci defined in Table 1 for each of at least 10, at least 25, at least 50, or all of the target gene-regions defined in Table 1. For example, depending on the number of SNPs in a gene region, from 10 up to 500, 10 up to 400, 30 up to 400, 30 up to 300, 30 up to 200, or 30 up to 100 of the SNP loci for each of at least 30, at least 60, at least 90, or all of the target gene-regions defined in Table 1 may be detected in the non-tumor DNA. The present inventors have found that the method of the present invention is especially effective and informative about the asCNAs present in the tumor DNA of a sample when at least 30, at least 60, at least 90 or at least 100 of the SNP loci defined in Table 1 are detected for each of at least 10, at least 25, at least 50, or all of the target gene-regions defined in Table 1 (and in particular when at least 90 or at least 100 of the SNP loci defined in Table 1 are detected for each of at least 10, at least 25, at least 30, or all of the target gene-regions defined in Table 1).

In one embodiment, step ii) comprises detecting the presence of SNPs in the non-tumor DNA at:
at least 30, at least 60, at least 90, or at least 100 of the SNP loci defined in Table 1 for each of at least 5, at least 10, at least 15, at least 20, at least 25, at least 30, at least 40, at least 50, at least 60, at least 70 or all of the target gene-regions defined in Table 1.

In certain preferred embodiments, step ii) comprises detecting the presence of SNPs in the non-tumor DNA at target gene-regions and at control gene-regions. For example, step ii) may further comprise detecting the presence of SNPs in the genomic non-tumor DNA at:
at least 10%, at least 20%, at least 30%, at least 40%, at least 60%, or at least 80% of the SNP loci defined in Table 2 for each of at least 10, at least 20, at least 30, or all of the control gene-regions defined in Table 2.

In embodiments wherein step ii) comprises detecting the presence of SNPs in the non-tumor DNA at control gene-regions, typically the step also comprises detecting the presence of SNPs in the non-tumor DNA at about 10 to all of the SNP loci defined in Table 2 for each of at least 10, at least 25, at least 50, or all of the target gene-regions defined in Table 2. For example, depending on the number of SNPs in a gene region, from 10 up to 500, 10 up to 400, 30 up to 400, 30 up to 300, 30 up to 200, or 30 up to 100 of the SNP loci for each of at least 10, at least 25, at least 30, or all of the control gene-regions defined in Table 2 may be detected in the non-tumor DNA. The present inventors have found that the method of the present invention is especially effective and informative about the asCNAs present in the tumor DNA of a sample when at least 30, at least 60, at least 90 or at least 100 of the SNP loci defined in Table 2 are detected for each of at least 10, at least 25, at least 30, or all of the target gene-regions defined in Table 2 (and in particular when at least 90 or at least 100 of the SNP loci defined in Table 1 are detected for each of at least 10, at least 25, at least 30, or all of the target gene-regions defined in Table 2).

In one embodiment, step ii) comprises detecting the presence of SNPs in the non-tumor DNA at:
at least 30, at least 60, at least 90, or at least 100 of the SNP loci defined in Table 1 for each of at least 5, at least 10, at least 15, at least 20, at least 25, at least 30, at least 40, at least 50, at least 60, at least 70 or all of the target gene-regions defined in Table 1;
and at least 30, at least 60, at least 90, or at least 100 of the SNP loci defined in Table 2 for each of at least 5, at least 10, at least 15, at least 20, at least 25, at least 30, at least 35, or all of the control gene-regions defined in Table 2.

In certain embodiments, step ii) comprises a step of providing a set of probes, wherein said set of probes is capable of specifically hybridizing to:
at least 5% of the SNP loci defined in Table 1 for each of at least 5 target gene-regions defined in Table 1,
and optionally at least 5% of the SNP loci defined in Table 2 for each of at least 3 control gene-regions defined in Table 2;

Preferably, each probe in the set of probes is an oligonucleotide probe that is complementary to, and capable of hybridizing to, one or more SNP loci defined in Table 1 or 2. More preferably, each probe is complementary to only one SNP loci defined in Table 1 or 2.

In certain embodiments, step ii) comprises a step of providing a set of probes, wherein said set of probes is capable of specifically hybridizing to any embodiment or combination of embodiments described above, i.e. capable of specifically hybridizing to
a % or number of SNP loci defined in Table 1 for target gene-regions recited above and/or a number of target gene-regions defined in Table 1 recited above; and/or
a % or number of SNP loci defined in Table 2 for control gene-regions recited above and/or a number of control gene-regions defined in Table 2 recited above.

For example, step ii) comprises a step of providing a set of probes, wherein said set of probes is capable of specifically hybridizing to
at least 10%, at least 15%, at least 20%, at least 25%, at least 30%, at least 40%, at least 50%, at least 60%, at least 70%, at least 80%, at least 90% or all of the SNP loci defined in Table 1 for each of at least 5, at least 10, at least 15, at least 20, at least 25, at least 30, at least 40, at least 50, at least 60, at least 70 or all of the target gene-regions defined in Table 1;
and at least 10%, at least 15%, at least 20%, at least 25%, at least 30%, at least 40%, at least 50%, at least 60%, at least 70%, at least 80%, at least 90% or all of the SNP loci defined in Table 2 for each of at least 5, at least 10, at least 15, at least 20, at least 25, at least 30, at least 35, or all of the control gene-regions defined in Table 2.

In certain preferred embodiments, step ii) comprises detecting the presence of at least one SNP in the intronic region of a gene-region and/or at least one SNP in the flanking region of a gene-region.

In certain preferred embodiments, step ii) comprises detecting the presence of at least one SNP in the intronic region and at least one SNP in the flanking regions of a gene-region.

For example, step ii) comprises detecting the presence of single nucleotide polymorphisms (SNPs) in the non-tumor DNA at:
at least 5%, at least 10%, at least 15%, at least 20%, at least 25%, at least 30%, at least 40%, at least 50%, at least 60%, at least 70%, at least 80%, at least 90% or all of the SNP loci defined in Table 1 for each of at least 5, at least 10, at least 15, at least 20, at least 25, at least 30, at least 40, at least 50, at least 60, at least 70 or all of the target gene-regions defined in Table 1, wherein at least one SNP locus for each target gene region is in the intronic region of a gene-region, and/or at least one SNP locus for each target gene-region is in the flanking regions of a gene-region;
and optionally at least 5%, at least 10%, at least 15%, at least 20%, at least 25%, at least 30%, at least 40%, at least 50%, at least 60%, at least 70%, at least 80%, at least 90% or all of the SNP loci defined in Table 2 for each of at least 5, at least 10, at least 15, at least 20, at least 25, at least 30, at least 35, or all of the control gene-regions defined in Table 2, wherein at least one SNP locus for each control gene region is in the intronic region of a gene-region, and/or at least one SNP locus for each control gene-region is in the flanking regions of a gene-region.

In certain preferred embodiments, step ii) comprises detecting the presence of at least 10%, at least 20%, at least 30%, at least 50%, at least 75% or at least 90% of SNP in the intronic region of a gene-region and/or at least 10%, at least 20%, at least 30%, at least 50%, at least 75% or at least 90% of SNP in the flanking region of a gene-region.

In certain preferred embodiments, step ii) comprises detecting the presence of at least 10%, at least 20%, at least 30%, at least 50%, at least 75% or at least 90% of SNP in the intronic region of a gene-region and at least 10%, at least 20%, at least 30%, at least 50%, at least 75% or at least 90% of SNP in the flanking region of a gene-region.

For example, step ii) comprises detecting the presence of single nucleotide polymorphisms (SNPs) in the non-tumor DNA at:
at least 5%, at least 10%, at least 15%, at least 20%, at least 25%, at least 30%, at least 40%, at least 50%, at least 60%, at least 70%, at least 80%, at least 90% or all of the SNP loci defined in Table 1 for each of at least 5, at least 10, at least 15, at least 20, at least 25, at least 30, at least 40, at least 50, at least 60, at least 70 or all of the target gene-regions defined in Table 1, wherein at least 10%, at least 20%, at least 30%, or at least 50% of SNP loci for each target gene region are in the intronic region of a gene-region, and/or at least 10%, at least 20%, at least 30%, or at least 50% of SNP loci for each target gene region is in the flanking regions of a gene-region;
and optionally at least 5%, at least 10%, at least 15%, at least 20%, at least 25%, at least 30%, at least 40%, at least 50%, at least 60%, at least 70%, at least 80%, at least 90% or all of the SNP loci defined in Table 2 for each of at least 5, at least 10, at least 15, at least 20, at least 25, at least 30, at least 35, or all of the control gene-regions defined in Table 2, wherein at least 10%, at least 20%, at least 30%, or at least 50% of SNP loci for each control gene region is in the intronic region of a gene-region, and/or at least 10%, at least 20%, at least 30%, or at least 50% of SNP loci for each control gene region is in the flanking regions of a gene-region.

In one embodiment, the subject is female and step ii) comprises,
ii) detecting the presence of single nucleotide polymorphisms (SNPs) in the non-tumor DNA at:
   at least 5% of the SNP loci defined in Table 1 for each of at least 5 target gene-regions defined in Table 1,
   at least 5% of the SNP loci in the target gene-region of at least one of AR, MED12, SMARCA1, and KDM6A, wherein for AR, MED12, SMARCA1, and KDM6A the target gene-region is the exonic and intronic region of the gene;
   and optionally at least 5% of the SNP loci defined in Table 2 for each of at least 3 control gene-regions defined in Table 2,
   and optionally at least 5% of the SNP loci in the control gene-region of at least one of TEX11 and HDAC8, wherein for TEX11 and HDAC8 the control gene-region is the exonin and intronic region of the gene.

In such embodiments, the presence and/or alteration of one or more asCNA in a target gene-region selected from the group consisting of MED12, SMARCA1, and KDM6A as defined above indicates that the subject would benefit from treatment with one or more of a DNA methyltransferase 1 (DNMT1) inhibitor (for example 5-azacitidine) and the histone deacetylase (HDAC) inhibitor (for example vorinostat and romidepsin).

In such embodiments, the presence and/or alteration of one or more asCNA in the target gene-region of AR as defined above, indicates that the subject would benefit from ceasing or altering treatment with an hormonal agent, such as a LHRH agonist (for example leuprolide, goserelin, triptorelin, or histrelin), LHRH antagonist (for example degarelix), androgen blockers (for example abiraterone or ketoconazole), anti-androgen (for example flutamide, bicalutamide, nilutamide, enzalutamide, apalutamide or darolutamide), androgen synthesis inhibitor (for example abiraterone), estrogen or steroid (for example prednisone or dexamethasone). It also indicates that a subject would benefit from ceasing or altering treatment with one or more class of drug that is known or suspected of targeting AR function from treatment with one or more alternative cancer treatment, for example a chemotherapy such as a taxane (for example docetaxel and cabazitaxel) or a platinum-based antineoplastic drugs (for example carboplatin).

Step iii):

The method of the present invention comprises a step of identifying which of the SNPs present in the non-tumor DNA are informative SNPs (iSNPs) for the subject, wherein an iSNP is a SNP that is heterozygous in the non-tumor DNA for the subject. For the avoidance of doubt, when used herein, the term iSNP refers to a SNP that is heterozygous in the genomic non-tumor DNA or tumor DNA for the subject, and in particular has an allelic fraction (AF) of between about 0.05 to about 0.95. In one preferred embodiment, the term iSNP refers to a SNP that is heterozygous in the non-tumor DNA or tumor DNA for the subject and has an allelic fraction (AF) of between about 0.2 to about 0.8. The present inventors have found that by selecting iSNP in step iii) of the present method, it is possible to more accurately determine the type of asCNA present in the tumor DNA of the subject.

In certain embodiments, step iii) comprises the following steps:
contacting the biological sample comprising non-tumor and/or tumor DNA with the set of probes under conditions suitable for one or more of the probes to specifically hybridize to a SNP locus;
capturing the non-tumor and/or tumor DNA molecules in the biological sample that have hybridized to one or more of the probes;

Step iii) may further comprise a step of determining the nucleotide sequences of the captured DNA molecules and analysing the nucleotide sequences to identify which of the SNPs present in the non-tumor DNA are informative SNPs (iSNPs) for the subject.

### Step iv:

The method of the present invention comprises a step of determining the allelic imbalance for each target gene-region, and optionally for each control gene-region, of the tumor DNA by reference to the iSNPs for the subject in each gene-region (i.e. step iv-a).

The method of the present invention also comprises a step of determining the copy number for each target gene-region, and optionally for each control gene-region, in the tumor DNA (i.e. step iv-b).

In certain embodiments, the step iv-b) of the method of the invention further comprises a step of estimating the tumor content (TC) of the sample comprising tumor DNA and/or estimating the ploidy of the sample comprising tumor DNA.

In certain embodiments, the method further comprises a step of detecting in the tumor DNA the presence of somatic and/or germline mutations in the exonic region of one or more target gene-region defined in Table 1. In certain embodiments, the method further comprises a step of detecting in the tumor DNA the presence of somatic and/or germline mutations, for example insertions, deletions and single nucleotide variant mutations, in the exonic region of one or more target gene-region defined in Table 1. Typically, the method further comprises a step of detecting in the tumor DNA the presence of somatic and/or germline single nucleotide variant mutations in the exonic region of one or more target gene-region defined in Table 1

The method may further comprise optionally detecting in the tumor DNA the presence of somatic and/or germline mutations in the exonic region of one or more of gene selected from the group consisting of AR, MED12, SMARCA1, IDH1 and KDM6A. For example, the method may further comprise optionally detecting in the tumor DNA the presence of somatic and/or germline gains, losses and/or single nucleotide variant mutations in the exonic region of one or more of gene selected from the group consisting of AR, MED12, SMARCA1, IDH1 and KDM6A. In particular, the method may further comprise optionally detecting in the tumor DNA the presence of somatic and/or germline gains, losses and/or single nucleotide variant mutations in the exonic region of one or more of gene selected from the group consisting of AR, MED12, SMARCA1, and KDM6A; and/or optionally detecting in the tumor DNA the presence of somatic and/or germline single nucleotide variant mutations in the exonic region of/DH1. In one embodiment, the method may further comprise optionally detecting in the tumor DNA the presence of somatic and/or germline gains, losses and/or single nucleotide variant mutations in the exonic region of one or more of gene selected from the group consisting of MED12, SMARCA1, and KDM6A; and/or optionally detecting in the tumor DNA the presence of somatic and/or germline gains and/or single nucleotide variant mutations in the exonic region of AR; and/or optionally detecting in the tumor DNA the presence of somatic and/or germline single nucleotide variant mutations in the exonic region of/DH1.

In embodiments wherein the method comprises a step of detecting in the tumor DNA the presence of somatic and/or germline mutations in the exonic region of one or more target gene-region, the presence of one or more somatic and/or germline mutations indicates that the subject would benefit from treatment with one or more cancer treatments, has benefited, or is benefiting, from one or more cancer treatments and/or would benefit from ceasing or altering one or more cancer treatments.

### Step v):

The method of the present invention comprises a step of analyzing the allelic imbalance and copy number for each target gene-region, and optionally for each control gene-region, to determine the presence, absence, and/or alteration of one or more allele-specific copy number aberration (asCNA) at each target gene-region in the tumor DNA.

The presence, absence, and/or alteration of one or more asCNA in the tumor DNA indicates that the subject would benefit from treatment with one or more cancer treatments, has benefited, or is benefiting, from one or more cancer treatments and/or would benefit from ceasing or altering one or more cancer treatments.

### Optional Method Steps:

In certain embodiments, the method of the invention further comprises step vi):
determining one or more cancer treatment the subject would benefit from.

In certain embodiments, the method of the invention further comprises step vii):
treating the subject with one or more cancer treatment determined in step vi), and thereby treating the subject.

In certain embodiments, the method of the invention further comprises the following steps:
I) providing a further biological sample obtained from the subject during or after the subject has undergone a treatment for cancer, wherein said sample comprises tumor DNA;
II) performing steps iii) to v) of the method of the invention using the further biological sample provided in step I); and
   wherein the presence, absence, and/or alteration of one or more asCNA in the further biological sample comprising tumor DNA compared to the biological sample comprising tumor DNA indicates that the subject would benefit from treatment with one or more cancer treatments, has benefited, or is benefiting, from one or more cancer treatments and/or would benefit from ceasing or altering one or more cancer treatments.

In such embodiments, preferably the further biological sample obtained from the subject during or after the subject has undergone a treatment for cancer, wherein said sample comprises tumor DNA, is a sample of the same type as the biological sample comprising tumor DNA provided in step i). For example, if the biological sample comprising tumor DNA provided in step i) is a blood sample, preferably the further biological sample comprising tumor DNA is a blood sample; or if the biological sample comprising tumor DNA provided in step i) is a plasma sample, preferably the further biological sample comprising tumor DNA is a plasma sample.

In embodiments where the method of the invention further comprises:
I) providing a further biological sample obtained from the subject during or after the subject has undergone a treatment for cancer, wherein said sample comprises tumor DNA;
   preferably the further biological sample of step I) is obtained at a later time point to the biological sample comprising tumor DNA of step i). For example, the further biological sample of step I) may be obtained around 1 week, around 2 weeks, around 3, weeks, around 4, weeks, around 1 month, around 6 weeks, around 2 months, around 3 months, around 4 months, around 6 months, around 9 months, around 1 year, or more than 1 year after the biological sample comprising tumor DNA of step i) was obtained.

In embodiments where the method of the invention further comprises steps I) and II), steps I) and II) may be repeated on one or more additional further biological samples (for example 1, 2, 3, 4, 5, 10, 15 or more additional further biological samples). For example, the steps may be repeated on one or more additional further biological samples each obtained at different time points during or after the subject has undergone a treatment for cancer compared to the time point the further biological sample was obtained. For example, an additional further biological may be obtained around 1 week, around 2 weeks, around 3, weeks, around 4, weeks, around 1 month, around 6 weeks, around 2 months, around 3 months, around 4 months, around 6 months, around 9 months, around 1 year, or more than 1 year after the further biological sample comprising tumor DNA of step i) was obtained.

In certain embodiments, the method of the invention further comprises a step of treating the subject for a cancer using a therapeutic agent for the treatment of cancer; or ceasing or altering treatment with a therapeutic agent for the treatment of a cancer; or initiating a non-therapeutic agent treatment for cancer (for example initiation of treatment by surgery or radiation).

### Detecting SNPs:

Typically, steps ii) and iii) of the method of the present invention are performed by analysing the nucleotide sequence of the DNA in the biological sample.

A variety of procedures suitable for determining the nucleotide sequence of a DNA molecule are known in the art and may be used to practice the methods disclosed herein. Sequencing methods suitable for use in the present invention include, for example, Sanger sequencing, Polony sequencing, 454 pyrosequencing, Combinatorial probe anchor synthesis, SOLiD sequencing, Ion Torrent semiconductor sequencing, DNA nanoball sequencing, Heliscope single molecule sequencing, Single molecule real time (SMRT) sequencing, Nanopore DNA sequencing, Microfluidic Sanger sequencing and Illumina dye sequencing.

The method of the present invention may further comprise aligning the nucleotide sequences with a reference genome for the subject, for example by aligning the nucleotide sequences with hg38, hg19, hg18, hg17 or hg16. The alignment can, for example, be carried out using a variety of techniques known in the art. For example, a DNA sequence alignment tool, (e.g., BWA-MEM (for example version BWA-MEM 0.7.17-r1188) (Li, H. and Durbin, R. (2009) Bioinformatics, 25, 1754-1760); BBMap (Bushnell, B. (2014). BBMap: A Fast, Accurate, Splice-Aware Aligner. Lawrence Berkeley National Laboratory); HISAT (PMID: 258751142); Bowtie 2 (PMID: 22388286); and FSVA (PMID: 28155631)) can be used to align the reads to the reference genome (for example hg38, hg19, hg18, hg17 or hg16). In exemplary embodiments, the method described by Li, H. and Durbin, R., 2009 (Bioinformatics, 25, 1754-1760) is used (i.e. by using BWA-MEM 0.7.17-r1188).

The genomic location assigned to each nucleotide sequence in the alignment is based on the reference genome adopted. The genomic locations listed in Tables 1 and 2 disclosed herein correspond to reference genome hg19. The corresponding locations in a different reference genome can be found using public available tools known in the art. An example of such a tool is LiftOver (http://genome.ucsc.edu/).

In certain embodiments, the method comprises removing duplications of reads of the same DNA molecule (e.g. duplications of reads of the same cfDNA molecule). Sequence reads having exactly the same sequence and start and end base pairs (i.e. the same unclipped alignment start and unclipped alignment end of the sequence) are typically removed, as they are likely to be duplicate sequence reads of the same sequence (i.e. duplicate of reads of the same cfDNA molecule). For example, PCR duplications can be removed as part of the aligning step, such as using Picard tools v2.1.0 (http://broadinstitute.github.io/picard).

In certain embodiments, each SNP loci detected in step ii) is covered by at least 2 sequence reads in step iii), for example at least 2, 3, 4, 5, 6, 7, 8, 9, 10, 12, 15, 20, 25, 50, 100, 200, 300, 400, 500, 1000, or 10,000 sequence reads in step iii). Preferably, each SNP loci detected in step ii) is covered by at least 5 sequence reads in step iii), for example at least 6, 7, 8, 9, 10, 12, 15, 20, 25, 50, 100, 200, 300, 400, 500, 1000, or 10,000 sequence reads. More preferably, each SNP loci detected in step ii) is covered by at least 10 sequence reads in step iii), for example at least 12, 15, 20, 25, 50, 100, 200, 300, 400, 500, 1000, or 10,000 sequence reads. Estimation of the read-depth of each gene-region, and therefore the SNPs within each region, can be performed using methods known in the art, for example by using the method described in Carreira et al. (2014) (Science Translational Medicine, 6, 254ra125), which is incorporated herein by reference.

Determining the Presence, Absence, and/or Alteration Allele-Specific Copy Number Aberrations (asCNAs):

To determine the presence, absence, and/or alteration allele-specific copy number aberrations (asCNAs), it is first necessary to determine the allelic imbalance for the target gene-regions, and optionally control gene-regions, in the tumor DNA, and to determine the copy number for each gene-region in the tumor DNA.

Typically, to determine allelic imbalance, it is necessary to first determine a reference model using a set of control samples. Control samples may comprise genomic DNA from healthy subjects and/or healthy cells, for example white blood cells. Further examples of suitable control samples include:
a cfDNA sample from a healthy subject, for example a healthy age and/or gender matched subject;
a tissue sample from a healthy subject, for example a prostate tissue sample from a healthy subject;
a characterized genome sequence of a white blood cell;
a characterized genome sequence of a non-cancerous cell, such as a non-cancerous prostate cell.

An exemplary method for generating a reference model using a control sample is described in the Examples section.

Allelic imbalance may be determined for each gene-region in the tumor DNA and non-tumor DNA for a subject. For example, this may be achieved by reference to a reference model. Using a reference model can further improve the sensitivity of the methods of the invention.

A reference model may be a collection of statistics obtained from a two or more control samples (for example 2 or more, 3 or more, 4 or more, 5 or more, 8 or more, 10 or more, 15 or more, 20 or more, 30 or more, 40 or more, or more than 50 samples), in particular two or more (for example 2 or more, 3 or more, 4 or more, 5 or more, 8 or more, 10 or more, 15 or more, 20 or more, 30 or more, 40 or more, or more than 50 samples) control samples that do not comprise tumor DNA. For example, to build a reference model two or more control samples comprising non-tumor DNA and not comprising tumor DNA may be used. Biological samples comprising non-tumor DNA provided for step i) of the method of the invention for different subjects may be used as a sample for the reference model if each biological sample does not comprise tumor DNA (for example if each biological sample comprising non-tumor DNA is a WBC sample). Additionally, or alternatively, one or more control sample as described above may be used to build the reference model. The control samples are sequenced. Preferably, control samples for use in building a reference model are sequenced at the same institution and/or sequenced using the same sequencer. The sequences of the samples are analysed to provide a collection of statistics including local (per each single SNP) and global (aggregating SNPs by similar local coverages observed in control samples used for model building) metrics of SNPs. For each control sample that is used to build the model, only SNPs that are informative (i.e. heterozygous SNPs that are present in a gene-region in the genome of the subject that provided the control sample) are used to compute the statistics. When testing for the presence of imbalance in a biological sample comprising tumor DNA from a subject, the statistics for each SNP that is informative for the subject are extracted and used to generate allelic fraction (AF) distributions mimicking distributions that could be extracted from a pure non-tumor DNA sample and from a mix of non-tumor and tumor DNA at different proportions (for example 0% and 100%, 1% and 99%, 2% and 98%, 3% and 97%, N % and (100-N) % (wherein N is each integer between 3 and 97), 97% and 3%, 98% and 2%, 99% and 1% and 100 and 0%, of non-tumor and tumor DNA respectively). When comparing the observed iSNPs AF distribution from a biological sample comprising tumor DNA against reference model derived distributions, differences between the AF distribution in the biological sample and the model derived distributions indicate the presence of allele imbalance tumor and, if allele imbalance is present, the quantity of the allele imbalance.

An exemplary method for determining allelic imbalance in the tumor DNA with reference to a reference model is described in the Examples section. An exemplary method for determining allelic imbalance in the tumor DNA without reference to a reference model is also described in the Examples section.

The copy number of a gene-region in the tumor DNA or non-tumor DNA may be determined, for example, by integrating the read-depth estimations and allelic imbalance calls. Methods for determining the focal copy number of parts of a gene-region are described herein. Methods for determining the focal copy number of parts of a gene-region are also known in the art, for example DNAcopy (10.18129/B9.bioc.DNAcopy) and the methods reported in Zare, F., et al. BMC Bioinformatics 18, 286 (2017).

Allele-specific copy number aberrations (asCNAs) are alterations in the total copy number of a gene-region, or part thereof, and the specific number of copies of each chromosome. Types of asCNA include: copy number gain (for example balanced or unbalanced copy number gain), copy number loss (for example homodeletions and hemideletions), and loss of heterozygosity (LOH) events (for example copy number-neutral loss of heterozygosity). The asCNA status for each gene-region may be determined by integrating read-depth estimation for each gene-region and allelic imbalance status for each gene-region. The asCNA status may then be corrected for the ploidy and purity of the tumor DNA in the biological sample. Method for correcting the asCNA for ploidy and purity are known in the art. For example, CLONETv2 (Prandi et al., 2019, Curr Protoc Bioinformatics. September; 67(1):e81)), FACETS (PMID: 27270079), ASCAT (PMID: 20837533), Sequenza (PMID: 25319062), or CNVkit (PMID: 27100738). In exemplary embodiments, the method described by Prandi et al., 2019 (Curr Protoc Bioinformatics. 2019 September; 67(1):e81) is used (i.e. by using the CLONETv2 algorithm).

As described in the Examples section, the presence, absence, and/or alteration of one or more asCNA in the tumor DNA indicates that the subject would benefit from treatment with one or more cancer treatments, has benefited, or is benefiting, from one or more cancer treatments and/or would benefit from ceasing or altering one or more cancer treatments.

### In Vitro Assay:

In certain embodiments, the method of the present invention are performed using an in vitro assay of the present invention. The in vitro assay of the present invention comprises the method steps of:
a) providing a biological sample obtained from the subject, wherein said sample comprises tumor DNA, and a biological sample obtained from the subject, wherein said sample comprises genomic non-tumor DNA;
b) providing a set of probes, wherein said set of probes are capable of specifically hybridizing to:
   at least 5% of the SNP loci defined in Table 1 for each of at least 5 target gene-regions defined in Table 1,
   and optionally at least 5% of the SNP loci defined in Table 2 for each of at least 3 control gene-regions defined in Table 2;
c) contacting the biological sample comprising non-tumor and/or tumor DNA with the set of probes under conditions suitable for one or more of the probes to specifically hybridize to a SNP locus;
d) capturing the non-tumor DNA and/or tumor DNA molecules in the biological sample that have hybridized to one or more of the probes, and determining the nucleotide sequence of the captured DNA molecules;
e) analysing the nucleotide sequences to identify which of the SNPs present in the non-tumor DNA are informative SNPs (iSNPs) for the subject, wherein an iSNP is a SNP that is heterozygous in the non-tumor DNA for the subject;
f) determining the allelic imbalance for each target gene-region, and optionally for each control gene-region, of the tumor DNA by reference to the iSNPs for the subject in each gene-region; and determining the copy number for each target gene-region, and optionally for each control gene-region, in the tumor DNA; and
g) analysing the allelic imbalance and copy number for each target gene-region, and optionally for each control gene-region, to determine the presence, absence, and/or alteration of one or more allele-specific copy number aberration (asCNA) at each target gene-region in the tumor DNA;
   wherein the presence, absence, and/or alteration of one or more asCNA in the tumor DNA indicates that the subject would benefit from treatment with one or more cancer treatments, has benefited, or is benefiting, from one or more cancer treatments and/or would benefit from ceasing or altering one or more cancer treatments.

Steps e) and f) of the in vitro assay are performed using the same processes as described herein for steps iv) and v) of the in vitro method of the present invention.

### Probes:

The present invention also provides a set of probes suitable for use in the method of the present invention, wherein the set of probes are capable of hybridizing to:
at least 5% (for example, at least 10%, at least 20%, at least 30%, at least 40%, at least 60%, or at least 80%) of the SNP loci defined in Table 1 for each of at least 5, at least 10, at least 25, at least 50, or all of the target gene-regions defined in Table 1,
and optionally at least 5% (for example, at least 10%, at least 20%, at least 30%, at least 40%, at least 60%, or at least 80%) of the SNP loci defined in Table 2 for each of at least 5, at least 10, at least 25, at least 50, or all of the target gene-regions defined in Table 2.

In certain embodiments, a set of probes suitable for use in the method of the present invention are capable of hybridizing to at least one (for example 1, 2, 3, 4 or 5; typically at least 2, at least 3 or at least 4) target gene-region selected from the group consisting of FOXA1, FOXP1, HSD3B1, NCOA2 and ZBTB16 as defined in Table 1. Genetic aberrations in such gene-regions are known or suspected of being associated with the function in the androgen (receptor) signalling pathway.

In certain embodiments, a set of probes suitable for use in the method of the present invention are capable of hybridizing to at least one (for example 1, 2, 3, 4, 5, 6, 7, 8, 9, 10, 11, 12, 13, 14, 15, 16 or 17; typically at least 2, at least 3 or at least 4) target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 as defined in Table 1. Genetic aberrations in such gene-regions are known or suspected of being associated with cell cycle dysfunction.

In certain embodiments, a set of probes suitable for use in the method of the present invention are capable of hybridizing to at least one (for example 1, 2, 3, 4, 5, 6, 7 or 8; typically at least 2, at least 3 or at least 4) target gene-region selected from the group consisting of AKT1, AKT2, AKT3, MET, PIK3C, PIK3CB, PIK3R1 and PTEN as defined in Table 1. Genetic aberrations in such gene-regions are known or suspected of being associated with with the phosphoinositide 3-kinase (PI3K) signalling pathway.

In certain embodiments, a set of probes suitable for use in the method of the present invention are capable of hybridizing to at least one (for example 1, 2, 3, 4, 5, 6, 7 or 8; typically at least 2, at least 3 or at least 4) target gene-region selected from the group consisting of ARID1A, CHD1, KDM6A, MED12, SMARCA1, KMT2C, KMT2D and RYBP as defined in Table 1. Genetic aberrations in such gene-regions are known or suspected of being associated with chromatic remodeling dysfunction.

In certain embodiments, a set of probes suitable for use in the method of the present invention are capable of hybridizing to at least one (for example 1, 2, 3, 4, 5, 6, 8, 10, 12, 15, 20 or 22; typically at least 2, at least 3 or at least 4) target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1. Genetic aberrations in such gene-regions are known or suspected of being associated with DNA repair dysfunction.

In certain embodiments, a set of probes suitable for use in the method of the present invention are capable of hybridizing to at least one (for example 1, 2, 3, 4, 5, 6, 8, 10, or 12; typically at least 2, at least 3 or at least 4) target gene-region selected from the group consisting of ASXL1, CLU, CYLD, ERG_TMPRSS2, GNAS, IDH1, IDH2, NFE2L2, NKX3-1, RUNX1, SPOP and ZFHX3 as defined in Table 1. Genetic aberrations in such gene-regions are known to be associated with prostate cancer, although their precise role in the development and/or progression of prostate cancer of a subject are currently unknown.

In certain embodiments, a set of probes suitable for use in the method of the present invention are capable of hybridizing to at least one (for example 1, 2, or 3; typically at least 2) target gene-region selected from the group consisting of APC, CTNNB1 and RNF43 as defined in Table 1. Genetic aberrations in such gene-regions are known or suspected to dysregulate the Wnt signalling pathway.

In certain preferred embodiments, a set of probes suitable for use in the method of the present invention are capable of hybridizing to at least one (for example 1, 2, 3, 4, 5, or 6; typically at least 2, at least 3, at least 4, at least 5 or 6) target gene-region selected from the group consisting of BRCA2, ATM, RB1, NKX3-1, TP53, and PTEN as defined in Table 1. In certain embodiments, a set of probes suitable for use in the method of the present invention are capable of hybridizing to at least 3 of the target gene-regions selected from the group consisting of BRCA2, ATM, RB1, NKX3-1, TP53, and PTEN as defined in Table 1. More preferably, a set of probes suitable for use in the method of the present invention are capable of hybridizing to the gene-regions of at least BRCA2, ATM, RB1, NKX3-1, TP53, and PTEN as defined in Table 1.

In certain embodiments, a set of probes suitable for use in the method of the present invention are capable of hybridizing to at least one target gene-region selected from the group consisting of FOXA1, FOXP1, HSD3B1, NCOA2 and ZBTB16; and/or at least one target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53; and/or at least one target gene-region selected from the group consisting of ARID1A, CHD1, KMT2C, KMT2D and RYB; and/or at least one target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C; and/or at least one target gene-region selected from the group consisting of ASXL1, CLU, CYLD, ERG_TMPRSS2, GNAS, IDH2, NFE2L2, NKX3-1, RUNX1, SPOP and ZFHX3; and/or at least one target gene-region selected from the group consisting of AKT1, AKT2, AKT3, MET, PIK3C, PIK3CB, PIK3R1 and PTEN; and/or at least one target gene-region selected from the group consisting of APC, CTNNB1 and RNF43.

In certain embodiments, a set of probes suitable for use in the method of the present invention are capable of hybridizing to at least two, at least three or at least four target gene-region selected from the group consisting of FOXA1, FOXP1, HSD3B1, NCOA2 and ZBTB16; and/or at least two, at least three or at least four target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53; and/or at least two, at least three or at least four target gene-region selected from the group consisting of ARID1A, CHD1, KMT2C, KMT2D and RYB; and/or at least two, at least three or at least four target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C; and/or at least two, at least three or at least four target gene-region selected from the group consisting of ASXL1, CLU, CYLD, ERG_TMPRSS2, GNAS, IDH2, NFE2L2, NKX3-1, RUNX1, SPOP and ZFHX3; and/or at least two, at least three or at least four target gene-region selected from the group consisting of AKT1, AKT2, AKT3, MET, PIK3C, PIK3CB, PIK3R1 and PTEN; and/or at least one, at least two or each target gene-region selected from the group consisting of APC, CTNNB1 and RNF43.

Preferably, said set of probes is capable of specifically hybridizing to any embodiment or combination of embodiments described herein for step ii) of the in vitro method of the present invention, i.e. capable of specifically hybridizing to
a % or number of SNP loci defined in Table 1 for target gene-regions recited herein and/or a number of target gene-regions defined in Table 1 recited herein for step ii) of the in vitro method of the present invention; and/or
a % or number of SNP loci defined in Table 2 for control gene-regions recited herein and/or a number of control gene-regions defined in Table 2 recited herein for step ii) of the in vitro method of the present invention.

For example, said set of probes are capable of hybridizing to:
at least 10%, at least 15%, at least 20%, at least 25%, at least 30%, at least 40%, at least 50%, at least 60%, at least 70%, at least 80%, at least 90% or all of the SNP loci defined in Table 1 for each of at least 5, at least 10, at least 15, at least 20, at least 25, at least 30, at least 40, at least 50, at least 60, at least 70 or all of the target gene-regions defined in Table 1;
and optionally at least 10%, at least 15%, at least 20%, at least 25%, at least 30%, at least 40%, at least 50%, at least 60%, at least 70%, at least 80%, at least 90% or all of the SNP loci defined in Table 2 for each of at least 5, at least 10, at least 15, at least 20, at least 25, at least 30, at least 35, or all of the control gene-regions defined in Table 2.

Or, said set of probes are capable of hybridizing to:
at least 20%, at least 25%, at least 30%, at least 40%, at least 50%, at least 60%, at least 70%, at least 80%, at least 90% or all of the SNP loci defined in Table 1 for each of at least 5, at least 10, at least 15, at least 20, at least 25, at least 30, at least 40, at least 50, at least 60, at least 70 or all of the target gene-regions defined in Table 1;
and optionally at least 20%, at least 25%, at least 30%, at least 40%, at least 50%, at least 60%, at least 70%, at least 80%, at least 90% or all of the SNP loci defined in Table 2 for each of at least 5, at least 10, at least 15, at least 20, at least 25, at least 30, at least 35, or all of the control gene-regions defined in Table 2.

Or, said set of probes are capable of hybridizing to:
at least 30, at least 60, at least 90, or at least 100 of the SNP loci defined in Table 1 for each of at least 5, at least 10, at least 15, at least 20, at least 25, at least 30, at least 40, at least 50, at least 60, at least 70 or all of the target gene-regions defined in Table 1;
and optionally at least 30, at least 60, at least 90, or at least 100 of the SNP loci defined in Table 2 for each of at least 5, at least 10, at least 15, at least 20, at least 25, at least 30, at least 35, or all of the control gene-regions defined in Table 2.

In one embodiment, the set of probes comprises at least 10, at least 20, or at least 30 (for example, at least 100, at least 300, at least 400, at least 500) probes that are capable of specifically hybridizing to at least 10, at least 20, or at least 30 (for example, at least 100, at least 300, at least 400, at least 500) of the SNP loci defined in Tables 1 for each of at least 5 target gene-regions in Table 1. In certain embodiments, the set of probes further comprises at least 10, at least 20, or at least 30 (for example, at least 100, at least 300, at least 400, at least 500) probes that are capable of specifically hybridizing to at least 10, at least 20, or at least 30 (for example, at least 100, at least 300, at least 400, at least 500) of the SNP loci defined in Tables 2 for each of at least 5 control gene-regions in Table 2. Preferably, each probe in the set of probes provided in step ii) is capable of hybridizing to only one of the SNP loci defined in Table 1 or 2. Typically, the total number of different probes in the set is at least 50, at least 500, at least 1000, at least 3000, at least 5000, at least 10,000, at least 15,000, or at least 17,500.

In certain preferred embodiments, the set of probes is capable of hybridizing to:
at least 80% of the SNP loci defined in Table 1 for each of at least 5, at least 10, at least 25, at least 50, or all of the target gene-regions defined in Table 1.
and optionally at least 80% of the SNP loci defined in Table 2 for each of at least 5, at least 10, at least 25, at least 50, or all of the target gene-regions defined in Table 2.

In certain preferred embodiments, the set of probes is capable of hybridizing to:
at least 80% of the SNP loci defined in Table 1 for each of at least 5, at least 10, at least 25, at least 50, or all of the target gene-regions defined in Table 1.
and at least 80% of the SNP loci defined in Table 2 for each of at least 5, at least 10, at least 25, at least 50, or all of the target gene-regions defined in Table 2.

In another embodiment, the set of probes is capable of hybridizing to:
at least 90%, at least 95%, or at least 98% of the SNP loci defined in Table 1 for each of at least 5, at least 10, at least 25, at least 50, at least 70, or all of the target gene-regions defined in Table 1.
and optionally at least 90%, at least 95%, or at least 98% of the SNP loci defined in Table 2 for each of at least 5, at least 10, at least 25, at least 30, or all of the target gene-regions defined in Table 2.

Probes suitable for use in the in vitro method of the present invention include, but are not limited to, small molecules, peptides (including cyclic peptides), proteins, nucleic acids (e.g. DNA and RNA nucleotides including, but not limited to, antisense nucleotide sequences, triple helices, siRNA or miRNA, and nucleotide sequences encoding biologically active proteins, polypeptides or peptides), synthetic or natural inorganic molecules and synthetic or natural organic molecules that specifically bind to one or more SNP loci defined in Table 1 or 2.

In preferred embodiments, the set of probes provided by the present invention is a set of oligonucleotide probes. In such embodiments, the set of oligonucleotide probes is complementary to, and capable of hybridizing to, one or more SNP loci defined in Table 1 or 2, and preferably, each probe is complementary to only one SNP loci defined in Table 1 or 2.

Probes suitable for use in the present invention may comprise a “label” which is suitable for capturing DNA comprising one or more of the SNPs defined in Table 1 or 2.

Suitable labels for capturing DNA molecules in a sample that comprise one or more of the SNPs include, for example, biotin. The type of label chosen will depend on the desired capture method used. For convenience, the probe may be immobilised on a solid phase support including resins (such as polyacrylamides), carbohydrates (such as sepharose), plastics (such as polycarbonate), and latex beads.

The probes may be bound to a solid matrix as discussed above or packaged with reagents for binding them to the matrix. The solid matrix or substrate may be in the form of beads, plates, tubes, dip sticks, strips or biochips. Biochips or plates with addressable locating and discreet microtitre plates are particularly useful.

### Kits:

The term “kit” refers to any item of manufacture (e.g. a package or container) comprising at least one reagent, e.g. a probe or small molecule, for specifically detecting one or more SNPs of Table 1 and/or Table 2. The kit may be promoted, distributed, or sold as a unit for performing the methods of the present invention.

The kit of the invention comprises means for probing the biological sample to determine the presence of SNPs in a sample obtained from the subject. Preferably, the kit of the invention comprises a set of probes of the present invention.

The kit may also include additional components to facilitate the particular application for which the kit is designed. For example, the kit may additionally contain means of detecting a label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents of the necessary grade for use in a method of the disclosed invention in a health care setting. Non-limiting examples include agents to reduce non-specific binding, such as a carrier protein or a detergent.

In certain embodiments the kit comprises one or more containers and may also include sampling equipment, for example, bottles, bags (such as intravenous fluid bags), vials, syringes, and test tubes. Other components may include needles, diluents, wash reagents and buffers. Usefully, the kit may include at least one container comprising a pharmaceutically-acceptable buffer, such as phosphate-buffered saline, Ringer's solution and dextrose solution.

In one preferred embodiment, the kit comprises instructions for use. In certain embodiments, the kit comprises instructions for staging, classification, screening, monitoring, stratification, selecting treatment for, ascertaining whether treatment is working in, and/or prognostication of cancer in a subject using the kit. For example, the kit comprises instructions for use which define how to determine the present of SNPs and/or identify the presence of iSNPs in the non-tumor DNA in a sample, for example by following a method of the invention defined herein.

In one preferred embodiment, the kit comprises a computer product or a computer-executable software for staging, classification, screening, monitoring, stratification, selecting treatment for, ascertaining whether treatment is working in, and/or prognostication of cancer in a subject using the kit. In certain embodiments, the computer product comprises a non-transitory computer readable medium storing a plurality of instructions that when executed control a computer system to perform a method of the invention. In certain embodiments, the computer-executable software comprises software for performing a method of the invention.

### Cancers:

The method of the present invention is for detecting, screening, monitoring, staging, classification, selecting treatment for, ascertaining whether treatment is working in, and/or prognostication of a cancer. The present invention also provides methods for treating cancer. The cancer may be selected from the group consisting of prostate cancer, breast cancer, ovarian cancer, pancreatic cancer, bladder cancer, and metastatic cancer.

In certain embodiments, the cancer is prostate cancer. The prostate cancer may be any type of prostate cancer. Typically, it may be acinar adenocarcinoma prostate cancer, ductal adenocarcinoma prostate cancer, transitional cell cancer of the prostate, squamous cell cancer of the prostate or small cell prostate cancer. For example, it may be acinar adenocarcinoma prostate cancer or ductal adenocarcinoma prostate cancer. Alternatively, or additionally, the prostate cancer may be hormone sensitive prostate cancer (HSPC) or castration resistant prostate cancer (CRPC). Alternatively, or additionally, the prostate cancer may be metastatic prostate cancer, or it may be non-metastatic prostate cancer. In certain embodiments, it may be metastatic prostate cancer. In certain embodiments, the prostate cancer may be metastatic castration resistant prostate cancer or non-metastatic castration resistant prostate cancer. In certain embodiments, the prostate cancer may be metastatic hormone sensitive prostate cancer (mHSPC) or non-metastatic hormone sensitive prostate cancer.

In certain embodiments, the cancer is metastatic cancer (i.e. a cancer that has metastasised). Metastatic cancer is cancer that has spread from the primary site of origin into one or more different areas of the body. For example the cancer may be metastatic prostate cancer (i.e. cancer which has spread from the primary prostate site of origin into one or more different areas of the body), or another form of metastatic cancer, such as metastatic breast cancer, metastatic ovarian cancer, metastatic pancreatic cancer, or metastatic bladder cancer.

The method is especially suitable for the detecting, screening, monitoring, staging, classification, selecting treatment for, ascertaining whether treatment is working in, and/or prognostication of prostate cancer, and more especially metastatic prostate cancer.

Methods of treatment of the present invention are especially suitable for the treating prostate cancer, and more especially metastatic prostate cancer.

### Methods of Treatment:

The methods of the invention can be used for determining a suitable treatment regimen (i.e. one or more drug for the treatment of cancer) for a subject in need of cancer treatment.

The screening of subjects using the methods, kits and assays of the present invention, allow the full potential benefits of cancer treatments to be obtained by selecting one or more treatment that have a high likelihood of benefitting the patient in view of asCNA harboured in the subject's tumor. Alternatively, or additionally, screening of subjects using the methods, kits and assays of the present invention allow the full potential benefits of cancer treatments to be obtained by ceasing or altering one or more cancer treatments, thus minimizing side effects and exposure of a subject to unnecessary and/or potentially harmful treatments.

The methods of the invention can be used for determining that a subject would benefit from treatment with one or more cancer treatments selected from the group consisting of a ATR inhibitor, CDK inhibitor, chemotherapy (such as taxanes, platinum-based antineoplastic drugs), WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, WEE1 inhibitor, platinum-based antineoplastic drug, taxane, c-Met inhibitor, radionuclide and radiation therapy, DNMT1 inhibitor, HDAC inhibitor, BET inhibitor, PI3K inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid. The methods of the invention can be used for determining that a subject would benefit from treatment with one or more cancer treatments selected from the group consisting of a chemotherapy (such as a taxane (in particular docetaxel and cabazitaxel) and platinum-based antineoplastic drug (in particular carboplatin)), PARP inhibitor, or hormonal agent, such as a LHRH agonist, LHRH antagonist, anti-androgen, androgen synthesis inhibitor, estrogen or steroid.

Alternatively, or additionally, methods of the invention can be used for determining that a subject has benefited, or is benefiting, from one or more cancer treatments selected from the group consisting of ATR inhibitor, CDK inhibitor, Chemotherapy (such as taxanes and platinum-based antineoplastic drugs), WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, WEE1 inhibitor, platinum-based antineoplastic drug, taxane, c-Met inhibitor, radionuclide and radiation therapy, DNMT1 inhibitor, HDAC inhibitor, BET inhibitor, PI3K inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid. Alternatively, or additionally, methods of the invention can be used for determining that a subject has benefited, or is benefiting, from one or more cancer treatments selected from the group consisting of a chemotherapy (such as a taxane (in particular docetaxel and cabazitaxel) and platinum-based antineoplastic drug (in particular carboplatin)), PARP inhibitor, or hormonal agent, such as a LHRH agonist, LHRH antagonist, anti-androgen, androgen synthesis inhibitor, estrogen or steroid.

Alternatively, or additionally methods of the invention can be used for determining that a subject would benefit from ceasing or altering one or more cancer treatments selected from the group consisting of a ATR inhibitor, CDK inhibitor, Chemotherapy (such as taxanes, platinum-based antineoplastic drugs and c-Met inhibitors), WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, WEE1 inhibitor, platinum-based antineoplastic drug, taxane, c-Met inhibitor, radionuclide and radiation therapy, DNMT1 inhibitor, HDAC inhibitor, BET inhibitor, PI3K inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid. Alternatively, or additionally methods of the invention can be used for determining that a subject would benefit from ceasing or altering one or more cancer treatments selected from the group consisting of a chemotherapy (such as a taxane (in particular docetaxel and cabazitaxel) and platinum-based antineoplastic drug (in particular carboplatin)), PARP inhibitor, or hormonal agent, such as a LHRH agonist, LHRH antagonist, anti-androgen, androgen synthesis inhibitor, estrogen or steroid.

Examples of PARP inhibitors include olaparib, rucaparib, niraparib or talazoparib, Veliparib, Pamiparib, Rucaparib, and Veliparib; and in particular example olaparib, rucaparib, niraparib or talazoparib. Examples of ATR inhibitors include Berzosertib, ceralasertib, M4344, and BAY1895344. Examples of CDK inhibitors include Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, and Pyrimidines; and in particular Flavopiridol, Palbociclib, ribociclib and abemaciclib. Examples of DNA-PK inhibitors include AZD7648, M3814, CC-122 and CC-115. Examples of immune checkpoint therapies include PD-1 inhibitors such as pembrolizumab, nivolumab, cemiplimab, and spartalizumab; PD-L1 inhibitors such asatezolizumab, avelumab and durvalumab; and CTLA-4 inhibitors such as ipilimumab. Examples of CHK1 inhibitors include V158411, PF-477736 and AZD7762.

Examples of CHK2 inhibitors include CCT241533 and Aminopyridine 7. Examples of WEE1 inhibitors include adavosertib. Examples of platinum-based antineoplastic drugs include cisplatin, carboplatin, oxaliplatin, nedaplatin, triplatin tetranitrate, picoplatin, satraplatin and phenanthriplatin, and in particular cisplatin, carboplatin, oxaliplatin, nedaplatin. Examples of taxanes include docetaxel, cabazitaxel, and paclitaxel. Examples of radionuclide or radiation therapies include radium-223, 225Ac-Labeled PSMA-617 and 177Lu-Labeled PSMA-617. Examples of CDK inhibitors include Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, and Pyrimidines; and in particular Flavopiridol, Palbociclib, ribociclib and abemaciclib. Examples of chemotherapies include taxanes (for example docetaxel and cabazitaxel), platinum-based antineoplastic drugs (such as cisplatin and carboplatin) and c-Met inhibitors (for example cabozantinib). Examples of Aurora kinase inhibitors include Alisertib, ZM447439, hesperidin, and VX-680. Examples of PI3K inhibitors includeidelalisib, copanlisib, duvelisib, alpelisib, umbralisib, dactolisib, voxtalisib, Taselisib, Idelalisib, Buparlisib, Duvelisib, and Copanlisib and in particular idelalisib, copanlisib, duvelisib, alpelisib, and umbralisib. Examples of mTOR inhibitors include rapamycin, deforolimus, dactolisib, voxtalisib, temsirolimus, everolimus, sapanisertib, AZD8055, and AZD2014. Examples of PORCN inhibitors include WNT974, ETC-1922159 and CGX1321. Examples of FZD antagonists/monoclonal antibodies include Vantictumab, Ipafricept and OTSA101-DTPA-90Y. Examples of inhibitors of Wnt target genes include SM08502. Examples of LHRH agonists include leuprolide, goserelin, triptorelin, and histrelin. Examples of LHRH antagonists include degarelix. Examples of anti-androgens include flutamide, bicalutamide, nilutamide, enzalutamide, apalutamide and darolutamide. Examples of steroids include prednisone and dexamethasone. Example of DNMT1 inhibitors include 5-azacitidine. Examples of HDAC inhibitors include vorinostat and romidepsin. Examples of BET inhibitors include I-BET 151, I-BET 762, OTX-015, TEN-010, CPI-203, CPI-0610, olinone, RVX-208, ABBV-744, AZD5153, MT-1, and MS645. Examples of alkylating agents include nitrogen mustards (such as cyclophosphamide, chlormethine, uramustine, melphalan, chlorambucil, ifosfamide, and bendamustine), nitrosoureas (such as carmustine, lomustine, and streptozocin) and alkyl sulfonates (such as busulfan)).

In embodiments of the invention wherein at least one target gene-region is selected from the group consisting of AR, FOXA1, FOXP1, HSD3B1, NCOA2 and ZBTB16 (in particular FOXA1, FOXP1, HSD3B1, NCOA2 and ZBTB16) as defined in Table 1, the presence and/or alteration of one or more asCNA (and/or the presence of one or more somatic and/or germline mutations) in a target gene-region selected from the group consisting of AR, FOXA1, FOXP1, HSD3B1, NCOA2 and ZBTB16 as defined in Table 1, indicates that the subject would benefit from ceasing or altering treatment with one or more class of drug that is known or suspected of targeting AR function. For example ceasing or altering treatment with a hormonal agent, such as LHRH agonists, LHRH antagonists, androgen blockers, anti-androgens, androgen synthesis inhibitors, estrogens and steroids (in particular androgen blockers, anti-androgens and androgen synthesis inhibitors); and in particular ceasing or altering treatment with one or more of the following cancer treatments: leuprolide, goserelin, triptorelin, histrelin, degarelix, abiraterone, ketoconazole, flutamide, bicalutamide, nilutamide, enzalutamide, apalutamide or darolutamide, estrogens prednisone or dexamethasone (in particular abiraterone, ketoconazole, flutamide, bicalutamide, nilutamide, enzalutamide, apalutamide or darolutamide).

In embodiments of the invention wherein at least one target gene-region is selected from the group consisting of AR, FOXA1, FOXP1, HSD3B1, NCOA2 and ZBTB16 (in particular FOXA1, FOXP1, HSD3B1, NCOA2 and ZBTB16) as defined in Table 1, the presence and/or alteration of one or more asCNA (and/or the presence of one or more somatic and/or germline mutations) in a target gene-region selected from the group consisting of AR, FOXA1, FOXP1, HSD3B1, NCOA2 and ZBTB16 as defined in Table 1, indicates that the subject would benefit from treatment with a chemotherapeutic agent (for example a taxane (such as docetaxel or cabazitaxel) or a platinum-based antineoplastic drug (such as carboplatin)).

In embodiments of the invention wherein at least one target gene-region is selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 as defined in Table 1, the presence and/or alteration of one or more asCNA (and/or the presence of one or more somatic and/or germline mutations) in a target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 as defined in Table 1, indicates that the subject would benefit from treatment with one or more class of drug that is known or suspected of targeting cell cycle perturbations. For example, one or more of an ATR inhibitor, CDK inhibitor, chemotherapy, WEE1 inhibitors, Aurora kinase inhibitors or alkylating agent; in particular one or more of Berzosertib, Berzosertib, ceralasertib, M4344, BAY1895344, Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, Pyrimidines, taxanes (for example docetaxel and cabazitaxel), c-Met inhibitors (for example cabozantinib), adavosertib Alisertib, ZM447439, hesperidin, VX-680, nitrogen mustards (for example cyclophosphamide, chlormethine, uramustine, melphalan, chlorambucil, ifosfamide, and bendamustine), nitrosoureas (for example carmustine, lomustine, and streptozocin) and alkyl sulfonates (for example busulfan).

In embodiments of the invention wherein at least one target gene-region is selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 (and in particular TP53) as defined in Table 1, the presence and/or alteration of one or more asCNA (and/or the presence of one or more somatic and/or germline mutations) in a target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 (and in particular TP53) as defined in Table 1, indicates that the subject would benefit from ceasing or altering treatment with one or more hormonal agent, such as LHRH agonists, LHRH antagonists, anti-androgens, androgen synthesis inhibitors, estrogens and steroids; and in particular ceasing or altering treatment with one or more of the following cancer treatments: leuprolide, goserelin, triptorelin, histrelin, degarelix, abiraterone, ketoconazole, flutamide, bicalutamide, nilutamide, enzalutamide, apalutamide or darolutamide, estrogens prednisone or dexamethasone.

In embodiments of the invention wherein at least one target gene-region is selected from the group consisting of ARID1A, CHD1, KDM6A, MED12, SMARCA1, KMT2C, KMT2D and RYB as defined in Table 1, and wherein the presence and/or alteration of one or more asCNA (and/or the presence of one or more somatic and/or germline mutations) in a target gene-region selected from the group consisting of ARID1A, CHD1, KDM6A, MED12, SMARCA1, KMT2C, KMT2D and RYBP as defined in Table 1, indicates that the subject would benefit from treatment with one or more class of drug that is known or suspected to target chromatin remodelling functions. For example one or more DNMT1 inhibitors, HDAC inhibitors and BET inhibitors; in particular 5-azacitidine, vorinostat, romidepsin, I-BET 151, I-BET 762, OTX-015, TEN-010, CPI-203, CPI-0610, olinone, RVX-208, ABBV-744, AZD5153, MT-1, and MS645.

In embodiments of the invention wherein at least one target gene-region is selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, the presence or alteration of one or more asCNA (and/or the presence of one or more somatic and/or germline mutations) in a target gene-region selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, indicates that the subject would benefit from treatment with one or more class of drug that is known or suspected to target a DNA repair function. For example a PARP inhibitor, ATR inhibitor, DNA-PK inhibitor, immune checkpoint therapy (for example a PD-1 inhibitor, PD-L1 inhibitors, or a CTLA-4 inhibitor), CHK1 inhibitor, WEE1 inhibitor, platinum-based antineoplastic drug, or radionuclide or radiation therapy; in particular olaparib, rucaparib, niraparib or talazoparib, Veliparib, Pamiparib, Rucaparib, Veliparib, Berzosertib, ceralasertib, M4344, and BAY1895344 Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, Pyrimidines, AZD7648, M3814, CC-122, CC-115, pembrolizumab, nivolumab, cemiplimab, spartalizumab, asatezolizumab, avelumab, durvalumab, ipilimumab, V158411, PF-477736, AZD7762, CCT241533, Aminopyridine 7, adavosertib, cisplatin, carboplatin, oxaliplatin, nedaplatin, triplatin tetranitrate, picoplatin, satraplatin phenanthriplatin, radium-223, 225Ac-Labeled PSMA-617 and 177Lu-Labeled PSMA-617.

In one embodiment of the invention wherein at least one target gene-region is selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, the presence or alteration of one or more asCNA (and/or the presence of one or more somatic and/or germline mutations) in a target gene-region selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, indicates that the subject would benefit from treatment with a PARP inhibitor, for example olaparib, rucaparib, niraparib or talazoparib, Veliparib, Pamiparib, Rucaparib, and Veliparib; and in particular olaparib, rucaparib, niraparib or talazoparib

In embodiments of the invention wherein at wherein at least one target gene-region is selected from the group consisting of ASXL1, CLU, CYLD, ERG_TMPRSS2, GNAS, IDH1, IDH2, NFE2L2, NKX3-1, RUNX1, SPOP and ZFHX3 as defined in Table 1, the presence or alteration of one or more asCNA (and/or the presence of one or more somatic and/or germline mutations) in a target gene-region selected from the group consisting of ASXL1, CLU, CYLD, ERG_TMPRSS2, GNAS, IDH1, IDH2, NFE2L2, NKX3-1, RUNX1, SPOP and ZFHX3 as defined in Table 1, indicates that the subject would benefit from treatment with one or more class of drug as follows: ATR inhibitor, CDK inhibitor, Chemotherapy (such as taxanes and platinum-based antineoplastic drugs), WEE1 inhibitor, Aurora kinase inhibitor, PARP inhibitor, DNA-PK inhibitor, immune checkpoint therapies (for example a PD-1 inhibitor, PD-L1 inhibitors, or a CTLA-4 inhibitor), CHK2 inhibitor, WEE1 inhibitors, platinum-based antineoplastic drug, taxane, radionuclide and radiation therapy, PI3K inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid.

In embodiments of the invention wherein at least one target gene-region is selected from the group consisting of AKT1, AKT2, AKT3, MET, PIK3C, PIK3CB, PIK3R1 and PTEN as defined in Table 1, the presence or alteration of one or more asCNA (and/or the presence of one or more somatic and/or germline mutations) in a target gene-region selected from the group consisting of AKT1, AKT2, AKT3, MET, PIK3C, PIK3CB, PIK3R1 and PTEN as defined in Table 1, indicates that the subject would benefit from treatment with one or more class of drug that is known or suspected to target the PI3K signalling pathway. For example a PI3K inhibitor or mTOR inhibitor; in particular idelalisib, copanlisib, duvelisib, alpelisib, umbralisib, dactolisib, voxtalisib, Taselisib, Idelalisib, Buparlisib, Duvelisib, Copanlisib, rapamycin, deforolimus, dactolisib, voxtalisib, temsirolimus, everolimus, sapanisertib, AZD8055, and AZD2014.

In embodiments of the invention wherein at least one target gene-region is selected from the group consisting of AKT1, AKT2, AKT3, MET, PIK3C, PIK3CB, PIK3R1 and PTEN (and in particular PTEN) as defined in Table 1, the presence or alteration of one or more asCNA (and/or the presence of one or more somatic and/or germline mutations) in a target gene-region selected from the group consisting of AKT1, AKT2, AKT3, MET, PIK3C, PIK3CB, PIK3R1 and PTEN (and in particular PTEN) as defined in Table 1, indicates that the subject would benefit from ceasing or altering treatment with one or more hormonal agent, such as LHRH agonists, LHRH antagonists, anti-androgens, androgen synthesis inhibitors, estrogens and steroids; and in particular ceasing or altering treatment with one or more of the following cancer treatments: leuprolide, goserelin, triptorelin, histrelin, degarelix, abiraterone, ketoconazole, flutamide, bicalutamide, nilutamide, enzalutamide, apalutamide or darolutamide, estrogens prednisone or dexamethasone.

In embodiments of the invention wherein at least one target gene-region is selected from the group consisting of APC, CTNNB1 and RNF43 as defined in Table 1, the presence or alteration of one or more asCNA (and/or the presence of one or more somatic and/or germline mutations) in a target gene-region selected from the group consisting of APC, CTNNB1 and RNF43 as defined in Table 1, indicates that the subject would benefit from treatment with one or more class of drug that is known or suspected to target the Wnt signalling pathway. For example a PORCN inhibitor, FZD antagonists/monoclonal antibody, or inhibitor of Wnt target genes; in particular WNT974, ETC-1922159, CGX1321, Vantictumab, Ipafricept, or OTSA101-DTPA-90Y.

The cancer treatments mentioned above, when used or administered in a method of the invention, may be used, for example, in those amounts indicated in the Physicians' Desk Reference (PDR) or as otherwise determined by one of ordinary skill in the art. The amount of the active ingredient of the cancer treatment which is required to achieve a therapeutic effect will, of course, vary with the particular compound, the route of administration, the subject under treatment, including the type, species, age, weight, sex, and medical condition of the subject and the renal and hepatic function of the subject, and the particular cancer being treated, as well as its severity. An ordinarily skilled physician, veterinarian or clinician can readily determine and prescribe the effective amount of the drug required to benefit the subject.

The present invention also provides a method for treating cancer in a subject comprising performing a method of the invention or performing an assay of the invention, and further comprising administering to the subject a therapeutically effective dose of a cancer treatment. A method of treatment of the present invention may be performed before and/or after a method of the invention defined herein has been performed, or an assay has been performed.

In certain embodiments a method for treating cancer of the present invention comprises administering to the subject a therapeutically effective dose of a cancer treatment after a method of the invention has been performed, or after an assay of the invention has been performed, for example after the subject has been determined to benefit from treatment with one or more cancer treatments, or has benefited, or is benefiting, from one or more cancer treatments and/or benefit from ceasing or altering one or more cancer treatments.

In one embodiment, a method for treating cancer of the present invention comprises administering a therapeutically effective dose of a cancer treatment to the subject for at least 1 week, 2 weeks, 3 weeks, 4 weeks, 1 month, 2 months, 3 months, 6 months, 9 months, 12 months, 24 months or 36 months. A therapeutic agent for the treatment of prostate cancer may be administered, for example, daily, every second day, twice per week, weekly or monthly.

A therapeutically effective dose of a cancer treatment may be administered in amounts and at frequencies indicated in the Physicians' Desk Reference (PDR) or as otherwise determined by one of ordinary skill in the art.

The present invention also provides a method for treating a subject having a cancer (for example prostate cancer or metastatic cancer) with cancer therapy, the method comprising:
performing a method for detecting, screening, monitoring, staging, classification, stratification, selecting treatment for, ascertaining whether treatment is working in, and/or prognostication of prostate cancer of the present invention, or
performing an in vitro assay for staging, classification, screening, monitoring, stratification, selecting treatment for, ascertaining whether treatment is working in, and/or prognostication of a cancer in a subject of the present invention,
   administering to the subject determined to benefit from treatment with one or more cancer treatments, or has benefited, or is benefiting, from one or more cancer treatments, a therapeutically effective dose of a cancer treatment, thereby treating the subject.

A cancer treatment may be selected from the group consisting of a ATR inhibitor, CDK inhibitor, Chemotherapy (such as taxanes and platinum-based antineoplastic drugs), WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, WEE1 inhibitor, platinum-based antineoplastic drug, taxane, c-Met inhibitor, radionuclide and radiation therapy, DNMT1 inhibitor, HDAC inhibitor, BET inhibitor, PI3K inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid. In certain embodiments a cancer treatment may be selected from the group consisting of a chemotherapy (such as a taxane (in particular docetaxel and cabazitaxel) and platinum-based antineoplastic drug (in particular carboplatin)), PARP inhibitor, or hormonal agent, such as a LHRH agonist, LHRH antagonist, anti-androgen, androgen synthesis inhibitor, estrogen or steroid.

Alternatively, or additionally, the present invention also provides a method for treating a subject having a cancer (for example prostate cancer or metastatic cancer) with cancer therapy, the method comprising:
performing a method for detecting, screening, monitoring, staging, classification, stratification, selecting treatment for, ascertaining whether treatment is working in, and/or prognostication of prostate cancer of the present invention, or
performing an in vitro assay for staging, classification, screening, monitoring, stratification, selecting treatment for, ascertaining whether treatment is working in, and/or prognostication of a cancer in a subject of the present invention,
   administering to the subject determined to benefit from ceasing or altering (e.g. changing the dose or frequency of the dosing) a therapeutically effective dose of an alternative cancer treatment and/or an adjusted dose of a cancer treatment, thereby treating the subject.

An alternative cancer treatment may be selected from the group consisting of a ATR inhibitor, CDK inhibitor, Chemotherapy (such as taxanes and platinum-based antineoplastic drugs), WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, WEE1 inhibitor, platinum-based antineoplastic drug, taxane, radionuclide and radiation therapy, DNMT1 inhibitor, HDAC inhibitor, BET inhibitor, PI3K inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid. In certain embodiments an alternative cancer treatment may be selected from the group consisting of a chemotherapy (such as a taxane (in particular docetaxel and cabazitaxel) and platinum-based antineoplastic drug (in particular carboplatin)), PARP inhibitor, or hormonal agent, such as a LHRH agonist, LHRH antagonist, anti-androgen, androgen synthesis inhibitor, estrogen or steroid.

The present invention also provides a method of treating a subject in need of treatment with a PARP inhibitor (for example a subject having cancer, prostate cancer or metastatic cancer), comprising
performing a method for detecting, screening, monitoring, staging, classification, stratification, selecting treatment for, ascertaining whether treatment is working in, and/or prognostication of prostate cancer of the present invention, or
performing an in vitro assay for staging, classification, screening, monitoring, stratification, selecting treatment for, ascertaining whether treatment is working in, and/or prognostication of a cancer in a subject of the present invention,
   administering to a subject determined to benefit from treatment with a PARP inhibitor, has benefited, or is benefiting, from treatment with a PARP inhibitor, a therapeutically effective dose of a treatment with a PARP inhibitor, and thereby treating the subject. A PARP inhibitor may be selected form the group consisting of olaparib, rucaparib, niraparib or talazoparib, Veliparib, Pamiparib, Rucaparib, and Veliparib; and in particular olaparib, rucaparib, niraparib or talazoparib.

In such embodiments, preferably at least one, at least 2, at least 3, at least 5, at least 6, at least 10, at least 15 or at least 20 target gene-regions is/are selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C. In such embodiments, the presence or alteration of one or more asCNA (and/or the presence of one or more somatic and/or germline mutations) in a target gene-region selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, indicates that the subject would benefit from treatment with a PARP inhibitor, for example olaparib, rucaparib, niraparib or talazoparib, Veliparib, Pamiparib, Rucaparib, and Veliparib; and in particular olaparib, rucaparib, niraparib or talazoparib.

The present invention also provides a method of treating a subject in need of treatment with an ATR inhibitor (for example a subject having cancer, prostate cancer or metastatic cancer), comprising
performing a method for detecting, screening, monitoring, staging, classification, stratification, selecting treatment for, ascertaining whether treatment is working in, and/or prognostication of prostate cancer of the present invention, or
performing an in vitro assay for staging, classification, screening, monitoring, stratification, selecting treatment for, ascertaining whether treatment is working in, and/or prognostication of a cancer in a subject of the present invention,
   administering to a subject determined to benefit from treatment with an ATR inhibitor, has benefited, or is benefiting, from treatment with an ATR inhibitor, a therapeutically effective dose of a treatment with an ATR inhibitor, and thereby treating the subject. An ATR inhibitor may be Berzosertib, ceralasertib, M4344, and BAY1895344, and in particular Berzosertib and ceralasertib.

In such embodiments, preferably at least one, at least 2, at least 3, at least 5, at least 6, at least 10, at least 15, at least 20, at least 30 or at least 35 of target gene-regions is/are selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B, RAD51C, ASXL1, CLU, CYLD, ERG_TMPRSS2, GNAS, IDH1, IDH2, NFE2L2, NKX3-1, RUNX1, SPOP and ZFHX3. In such embodiments, the presence or alteration of one or more asCNA (and/or the presence of one or more somatic and/or germline mutations) in a target gene-region selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B, RAD51C, ASXL1, CLU, CYLD, ERG_TMPRSS2, GNAS, IDH1, IDH2, NFE2L2, NKX3-1, RUNX1, SPOP and ZFHX3 as defined in Table 1, indicates that the subject would benefit from treatment with a ATR inhibitor, for example Berzosertib, ceralasertib, M4344, and BAY1895344, and in particular Berzosertib and ceralasertib

The present invention also provides a method of treating a subject in need of treatment with an immune checkpoint therapy (for example a subject having cancer, prostate cancer or metastatic cancer), comprising
performing a method for detecting, screening, monitoring, staging, classification, stratification, selecting treatment for, ascertaining whether treatment is working in, and/or prognostication of prostate cancer of the present invention, or
performing an in vitro assay for staging, classification, screening, monitoring, stratification, selecting treatment for, ascertaining whether treatment is working in, and/or prognostication of a cancer in a subject of the present invention,
   administering to a subject determined to benefit from treatment with an immune checkpoint therapy, has benefited, or is benefiting, from treatment with an immune checkpoint therapy, a therapeutically effective dose of a treatment with an immune checkpoint therapy, and thereby treating the subject. An immune checkpoint therapy may be a PD-1 inhibitor such as pembrolizumab, nivolumab, cemiplimab, and spartalizumab; a PD-L1 inhibitor such asatezolizumab, avelumab and durvalumab; or CTLA-4 inhibitor such as ipilimumab.

In such embodiments, preferably at least one, at least 2, at least 3, at least 5, at least 6, at least 10, at least 15, at least 20, at least 30 or at least 35 of target gene-regions is/are selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B, RAD51C, ASXL1, CLU, CYLD, ERG_TMPRSS2, GNAS, IDH1, IDH2, NFE2L2, NKX3-1, RUNX1, SPOP and ZFHX3. In such embodiments, the presence or alteration of one or more asCNA (and/or the presence of one or more somatic and/or germline mutations) in a target gene-region selected from the group consisting of ATM, ATR, BRCA1, BRCA2, ERCC1, ERCC2, ERCC3, ERCC4, ERCC5, FANCA, FANCC, FANCD2, FANCE, FANCF, FANCG, MLH1, MSH2, MSH6, PALB2, RAD51B, RAD51C, ASXL1, CLU, CYLD, ERG_TMPRSS2, GNAS, IDH1, IDH2, NFE2L2, NKX3-1, RUNX1, SPOP and ZFHX3 as defined in Table 1, indicates that the subject would benefit from treatment with an immune checkpoint therapy, for example a PD-1 inhibitor such as pembrolizumab, nivolumab, cemiplimab, and spartalizumab; a PD-L1 inhibitor such asatezolizumab, avelumab and durvalumab; or CTLA-4 inhibitors such as ipilimumab.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. The applicant reserves the right physically to incorporate into this application any and all materials and information from any such articles, patents, patent applications, or other physical and electronic documents.

The following Examples illustrate the invention.

### EXAMPLES

### Example 1: SNP Panel and Methods

### Materials and Methods

### Example 1a

### Selection of Target Genes for Inclusion in the SNP Panel

Target genes were selected for inclusion in the Example 1 SNP panel by the inventors based on at least one of the following criteria: i) recurrent copy number changes or point mutation in localized and/or advanced prostate cancer (PCa) based on N=278 tumors (1-3), ii) potential therapeutic relevance. This lead to the selection of 70 autosomal genes. Further, the genomic region on 21q between TMPRSS2 and ERG that undergoes interstitial deletion as mechanism of gene rearrangement was also selected. The genomic region on 21q between TMPRSS2 and ERG may be referred to as a target “gene” of the Example 1 SNP panel. These target genes are listed in column 1 of Table 1.

### Selection of Control Genes for Inclusion in the Example 1 SNP Panel

To optimize data quality, processing and downstream analysis, the inventors further selected 37 control genes across all chromosomes and having minimal aberration frequency across the 278 PCa reference dataset. Specifically, for each of the 39 chromosomal arms with available data, cytobands adjacent to telomeres or centromeres were first excluded, the remaining genes were then ranked for minimal aberration frequency (abs(log 2(T/N))<0.5), the top three were then considered, and then of those three genes for each arm, one gene was selected based on maximization of high MAF SNPs availability (see below). These genes are listed in column 1 of Table 2.

In addition, UGT2B17 and ZBTB9 genes, located in complex genomic regions and encompassed by high frequency germline copy number losses were also included as control genes (4). These genes are also listed in column 1 of Table 2.

It is noted that in the subsequent parts of this example, “gene-regions” are referred to rather than “genes”, due to the inclusion of flanking regions on a gene basis for enrichment of SNPs, as described in detail below.

### Selection of SNPs Per Gene-Region

To allow for tumor purity and ploidy estimations and to improve somatic copy number computation (5-7), high MAF SNPs for each target and control gene-region were included in the Example 1 SNP panel. Intronic and intergenic SNPs from dbSNP v144 with single reference/alternative bases and MAF>=20% were considered. For each gene an iterative selection strategy was implemented. Specifically, starting from gene coordinates, the number of high MAF SNPs in the selected genomic area was counted and, if lower than a threshold N, the genomic area was iteratively extended of 10 Kbp at both ends to either converge to N SNPs inclusion or to a maximum extension of 200 Kbp per side (the combination of the gene and flanking regions is referred to as the “gene-region” of the gene of interest). The value of N was set to 1,000 for a subset of target genes of special interest: BRCA2, ATM, RB1, NKX3-1, TP53, and PTEN; to 1,000 for the 21q area; and to 400 for the 64 other target genes; and to 300 for control genes. The gene-region of each target gene and control gene are reported in column 1 of Table 1 and Table 2, respectively. To optimize the selection of SNPs presenting high MAF across different ethnic groups, genotype data of ^{˜}2,000 samples from the 1,000 Genome Project data (Genotype Data release 20130502; PMID: 26432245) were considered. For each SNP, the fraction of individuals with heterozygous genotype status (HetFrac) in all 1,000 Genome project dataset and across four different major ethnic groups (Africans, Europeans, South Asian and East Asian) was computed. For each gene, a subset of M<N SNPs was selected; K<M SNPs were selected among the ones with the highest HetFrac across the different ethnic groups, while M-K SNPs were selected among the ones with the highest HetFrac in the overall dataset. M and K were respectively equal to 500 and 200 for BRCA2, ATM, RB1, NKX3-1, TP53 and PTEN and for the 21q area; 200 and 100 for 64 other target genes; and to 150 and 100 for control genes. The numbers of SNPs included by design per gene-region are reported in column 1 of Table 1 for the target genes; and column 1 of Table 2 for the control genes. Each SNP selected for each target gene-region and control gene-region are also listed.

As can be seen from column 1 of Tables 1 and 2, a total of 18,723 SNPs for target genes, of 8,392 SNPs for control genes. The number of SNPs included in the Example 1 SNP panel also allows for ethnicity inference and annotation (8) and for sample identity check (9). The final Example 1 SNP panel covers a total of 109 gene-regions. All coordinates in Tables 1 and 2 refer to the reference genome hg19.

### Other Genes Included in Example 1 SNP Panel

Additionally, exonic (i.e. coding) regions of the following additional target genes on the X-chromosome were included in the panel: AR, MED12, SMARCA1, and KDM6A; exonic (i.e. coding) regions of the following additional target gene was included in the panel: IDH1; and exonic (i.e. coding) regions of the following additional control genes on the X-chromosome were included in the panel: TEX11 and HDAC8.

### Selection of Exonic Regions

Based on ENSEMBL annotations (reference genome hg19), exonic regions for 116 genes were retrieved (70 target genes+21q area+39 control genes+5 additional genes on the X-chromosome and the gene IDH1) and a BED file was compiled collecting exonic regions coordinates and corresponding gene symbols. The total genomic size of all selected exonic regions is 824,144 bp with 645,667 bp for target genes and 178,477 bp for control genes.

### Clinical Cohorts

To test the performance and utility of the Example 1 SNP panel of target and control gene-regions and selected SNPs, the panel was applied to samples from subjects having prostate cancer and samples from healthy volunteers.

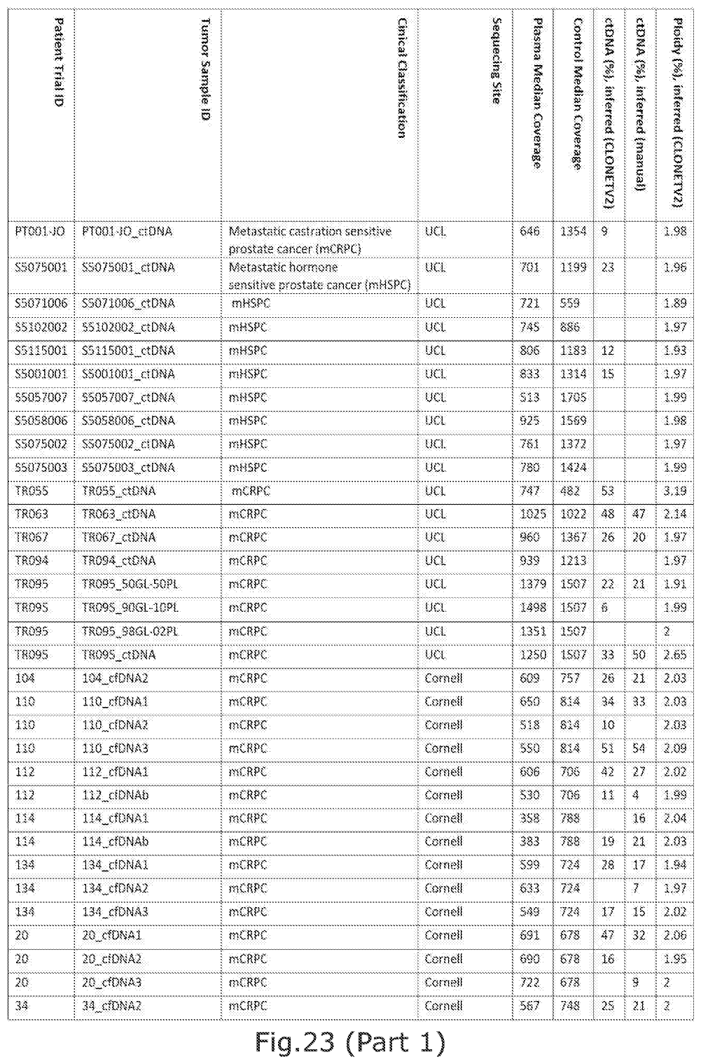

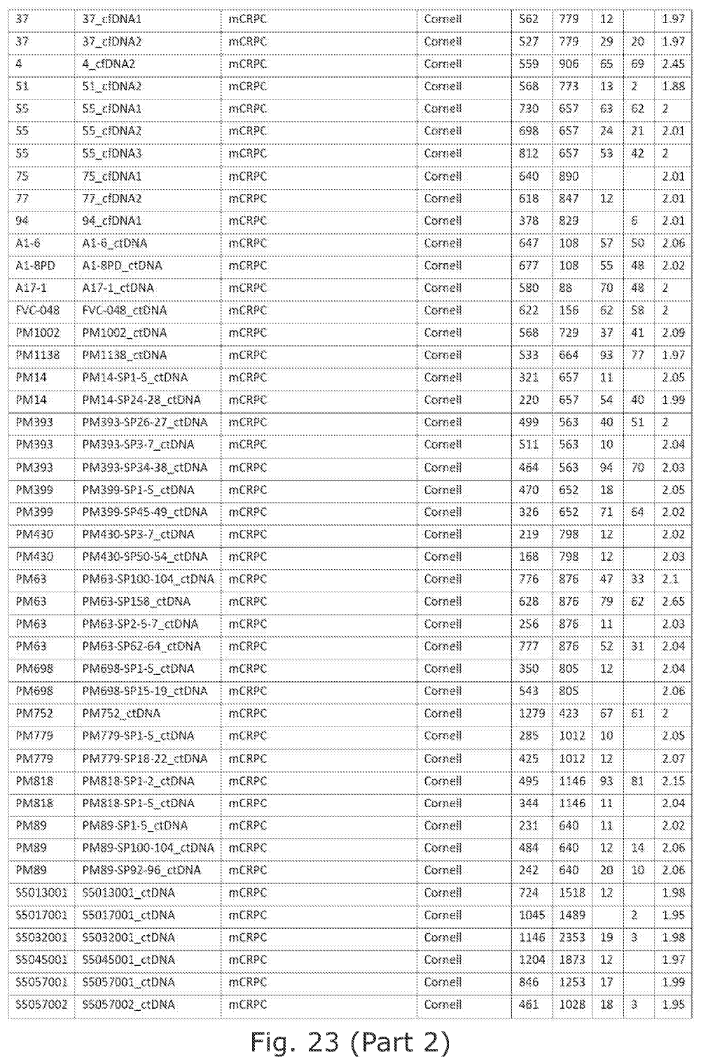

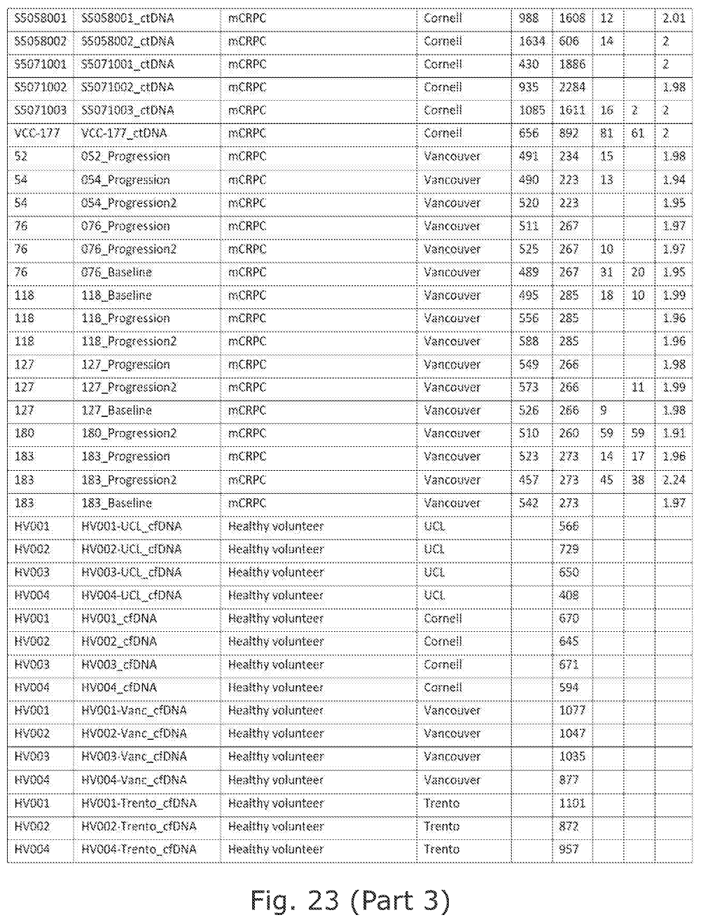

Patients study were enrolled at 3 institutions on Institutional Review Board (IRB)-approved protocols with written informed consent. All subjects signed IRB informed consent protocols at the respective institution. Patients were eligible for this study if they were diagnosed with metastatic prostate cancer. Overall, the study included: 66 cfDNA samples from 44 patients at WCM, 16 cfDNA samples from 7 patients at Vancouver, 18 cfDNA samples from 15 patients at UCL, 26 cfDNA and 6 tissue samples from 3 patients collected under the UCL Biobank protocol Biobank Ethical Review Committee (B-ERC) project reference number and title: NC24.20 Treatment resistance aberrations in prostate cancer. 4 Healthy donor plasmas were purchased from Cambridge Bioscence. Information on the patients in the cohort is provided in FIG. 23. Study samples from patients at UCL were sequenced using a Novaseq 6000 (Illumina) sequencing platform. Study samples from patients at all other institutions were sequenced using a HiSeq (Illumina) sequencing platform.

### Blood Collection, Plasma Separation and Whole Blood Cells Isolation

Blood collection was performed with ethylenediaminetetraacetic acid (EDTA) tubes, kept at 4° C. and processed within 2 hours from collection. Plasma separation was performed with a double spin protocol. First, the whole blood was centrifuged at 1600 rcf for 15 minutes at 4° C., then the separated plasma was collected and centrifuged again at 3000 rcf for 10 minutes at 4° C. The plasma was then divided into 1.8 mL aliquots and stored at −80° C. The buffy coat was collected after the first centrifuge, divided into 250 μL aliquots and stored at −80° C.

cfDNA and gDNA Extraction

Cell free DNA (cfDNA) is extracted starting from 1.8 mL plasma with QIAGEN QIAamp Circulating Nucleic Acid Kit according to the manufacturer's protocol and eluted in 30 μL Tris HCl 10 mM pH 8. The obtained cfDNA is then quantified using Qubit dsDNA High Sensitivity Assay and the quality is assessed with Agilent Bioanalyzer High Sensitivity DNA Kit.

Non-tumor genomic DNA (gDNA) is obtained from white blood cells, and is used as a matched control. The gDNA is extracted from 200 μL buffy coat with QIAGEN QIAamp DNA Mini Blood Kit and eluted in 200 μL Tris HCl 10 mM pH 8. The extracted gDNA is quantified using NanoDrop.

### Libraries Preparation

gDNA for library preparation was fragmented with Covaris M220. Libraries for target sequencing were prepared starting from 25 and 100 ng cfDNA and gDNA respectively with KAPA HyperPrep Kit (Roche) following the SeqCap EZ HyperCap v2.3 protocol with the following modifications. For hybridization of the probes, up to 8 cfDNA or gDNA samples were pooled together to obtain a combined mass of 2 μg and incubated for capture at 47° C. for 72 hours. The captured DNA was then amplified for 13 cycles. Pre- and post-captured libraries were quantified using Qubit dsDNA High Sensitivity Assay and the quality was assessed with Agilent Bioanalyzer High Sensitivity DNA Kit.

### Data Generation and Pre-Processing

Sequencing of study samples (i.e. all samples collecting including patient samples and healthy volunteer samples) was performed at institution facilities at Weill Cornell Medicine (WCM), Vancouver, University College of London (UCL), or University of Trento with the following platforms: Novaseq 6000 (Illumina) for UCL, and HiSeq (Illumina) for all other institutions. Detailed information and sequencing statistics are reported in FIG. 23. Paired-end reads were trimmed to remove adapters using trimmomatic (10) (version 0.32). Alignment to the humanG1Kv37 reference genome was performed using BWA-MEM (11) (version 0.7.17-r1188). Duplicate reads were marked and removed with Picard MarkDuplicates (12) (version 1.92). Realignment and recalibration were performed using GATK(12) (version 3.8.0). MD tags were calculated using samtools calmd (13) (version 1.10) and overlapping read pairs clipped using bamUtil (version 1.0.14). PaCBAM (version 1.5.0) was used to generate pileup files (14).

### Read-Depth Estimation for Wide and Focal Copy Number Aberrations

### Gene-Region Based Read-Depth Estimation for the Detection of Copy Number Aberrations (CNA)

Gene-region based read-depth estimation was performed similarly to as previously described (5). Briefly, mean amplicon depths of coverage are normalized for both GC content and sample mean coverage. Then, let \vec{cov^{T}} and \vec{cov^{C}} the vectors of normalized amplicon depth of coverage spanning a gene-region respectively in the tumor and matched control sample, the CN state (in log 2 scale) of the gene-region was computed using the following formula:

$${Log}_{2} R = {Log}_{2} ( median ( \frac{\vec{{cov}^{T}}}{\vec{{cov}^{C}}} ) )$$

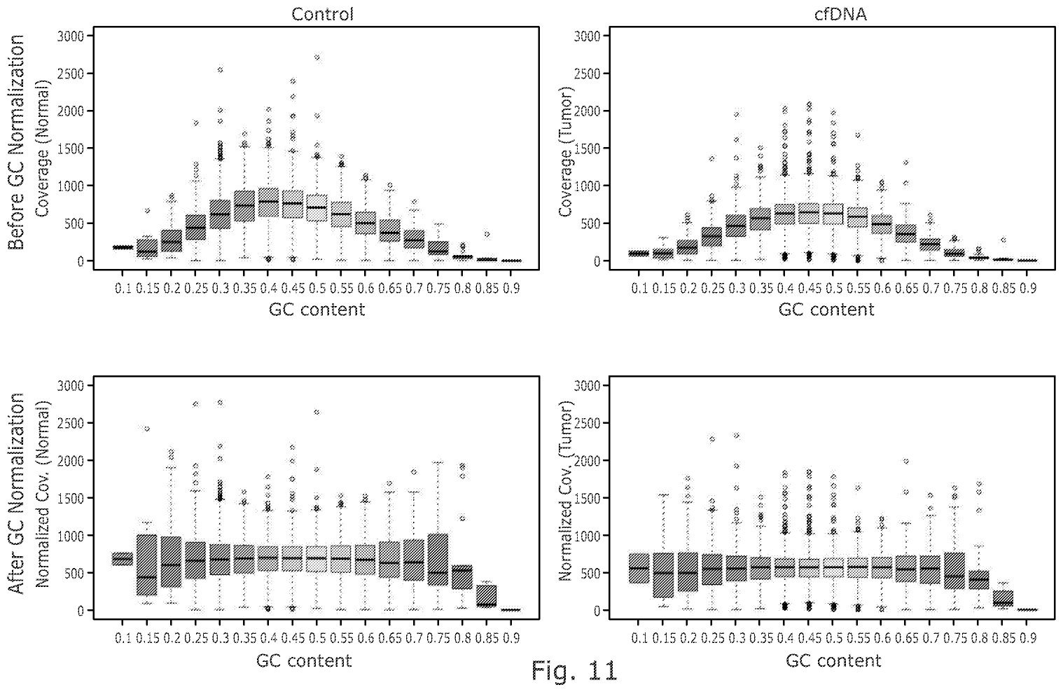

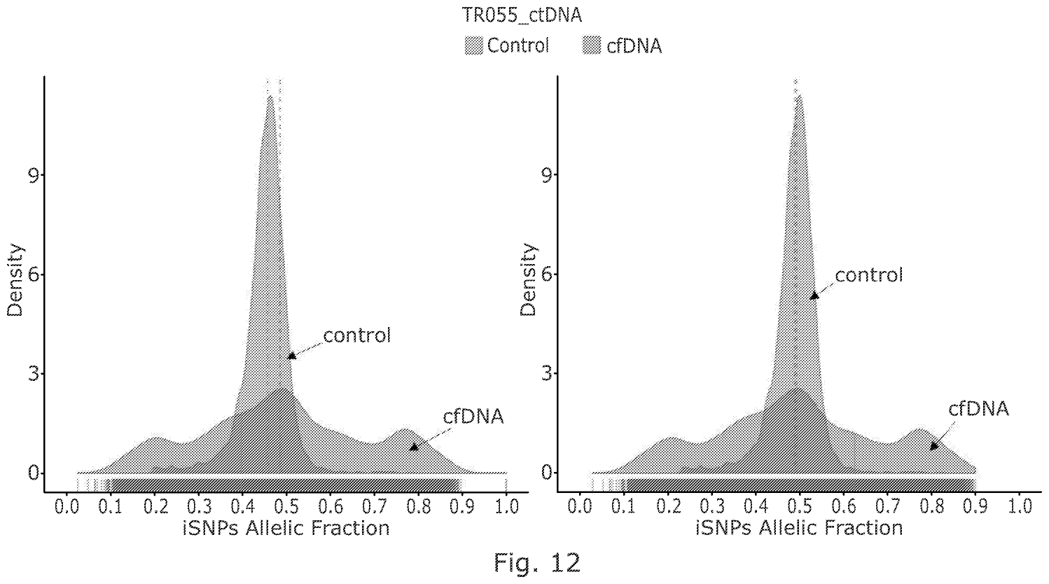

To improve confidence in the assessment of copy-number (CN) states, we adapted a previously developed procedure used to assess AR CN state (PMID: 26537258). That procedure measures the probability that an observed CN is compatible with the presence of aberrations accounting for stochastic noise in CN estimations. By computing control vs. control segmentation on Cornell cohort, the inventors observed that gene-region were associated with specific noise (see FIG. 12). In view of this, the inventors exploited the procedure to define gene-region specific cutoffs (thr_{Log}_{₂}) by integrating such information. For each gene-region, the inventors defined thr_{Log}_{₂} as the value such that: P(cn is gain)≥1−α or P(cn is loss)≥1−α, with α=0.005 (by default)

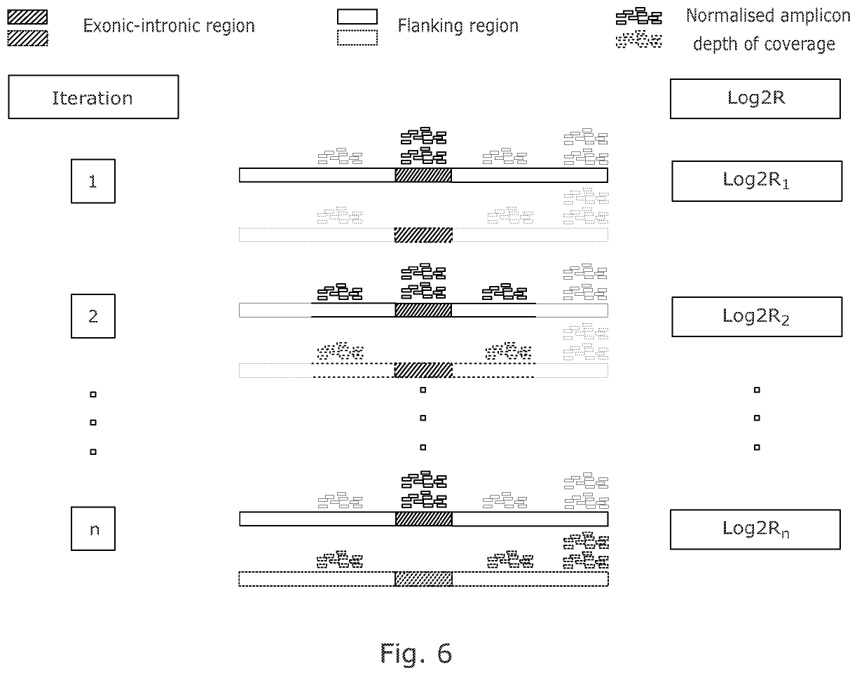

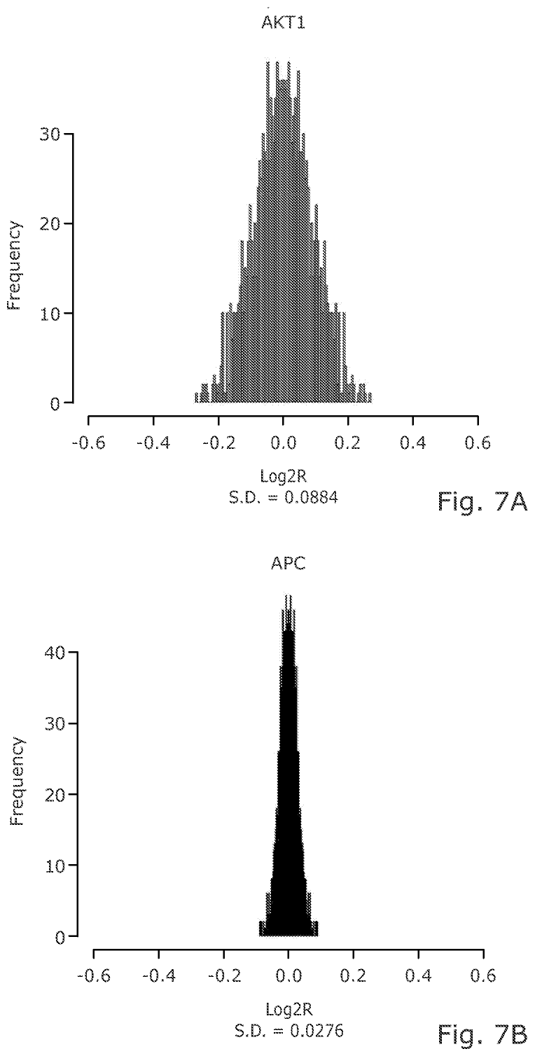

Detection of focal CNA. In order to detect CNAs that span areas smaller than entire gene-regions, a simple iterative process was implemented. Given the set of gene-regions G within the Example 1 SNP panel (including control and target gene-regions, G_{control} and G_{target} respectively), the set of amplicons within each gene-region (amp_{g}) was subdivided into those spanning exonic/intronic regions (amp_{g}_{_{coding}}) and into those within the flanking regions (amp_{g}_{_{flanking}}). For each gene-region g in G, an iterative procedure was applied to compute values of Log 2Rᵢ (see FIG. 6). First, Log 2R₁ was computed calculating the median of all amp_{g}_{_{coding}}; next, for each iteration i (for i=1:(n−1)), the Log 2R₁₊ᵢ was computed by including right and left side windows of size wen (10 Kbp, by default). The last iteration (n) corresponds to the inclusion of the entire gene-region.

All control gene-regions (excluding gene-region containing UGT2B17 or ZBTB9) in G_{control} were then used to compute per-gene-regions differences between the Log 2Rₐ and the respective Log 2R₁ (gene only). The parameters of the reference normal distribution ref.distr with mean equal to mean_{control} and standard deviation (s.d.) equal to sd_{control} were defined by taking the mean and the s.d. of the Log 2R differences, respectively. Last, for each target gene-region in G_{target}, the diff_{target}=|Log 2R₁−Log 2Rₙ| was compared k times (by default 10,000) against simulated distributions (sim.distrᵢ, with i∈[1, k]), each built by sampling 10,000 times from ref.distr. The probability of focal lesion for each target gene was computed as:

$$P ( focal ) = \frac{{\sum }_{i = 1}^{k} {success}_{i}}{k}$$

With successᵢ=1 if diff_{target}>max(sim.distrᵢ) or diff_{target}<min(sim.distrᵢ), 0 otherwise. To minimize noise-induced false positives, a lesion was only considered to be focal if P(focal)=1.

For each detected focal lesion, in order to define the exact boundaries of the lesion, the inventors then proceed as follows: i) identify the lowest (for losses) and highest (for gains) Log 2Rᵢ within the corresponding gene-region and expand boundaries if two adjacent amplicons present monotonic values on both sides.

### Gene-Region Allelic Imbalance Detection

### Reference Model Generation for Allelic Imbalance

The following is a standard procedure to build a reference model using a set of N control samples. For a set of N control samples (genomic DNA from healthy cells, for example white blood cells), SNPs that are informative (a heterozygous call cut off of 0.2<AF_{SNP}<0.8 was applied) in at least two of the N samples are selected. Summary statistics for each informative SNP across control samples are computed. Namely, for the AF distribution, mean, coefficient of variation, and proportion of samples out of N harboring the heterozygous genotype (A̅F̅_{SNP}, AF.cv_{SNP} and AF.freq_{SNP}, respectively); for the local coverage distribution, mean (C̅O̅V̅_{SNP}). Further, AF standard deviations stratified by local coverage quantiles Q (min 0%, max 100%, step 10%, quantile interval q) are also computed (σ_{AF}^{q}). To exclude noisy/low quality SNPs, the following filters were applied:
1. 0.35<A̅F̅_{SNP}<0.65;
2. AF.cv_{SNP}≤0.1;
3. AF.freq_{SNP}<0.8.

The collection of the summary statistics for each SNP was referred to as reference model (Refₓ; where x is one of the statistics). Of note, the reference model needs only to be computed once and can be applied cross-platform (i.e. can be used with different combinations of sequencing machines, reagents and sites where the samples have been processed). The use of control samples with intended sequencing coverage compared to that of plasma samples is suggested. For example, control samples can be obtained from white blood cells of one or more patients having cancer (e.g. matched control samples as described elsewhere) and/or samples from healthy volunteers. The requirement of the control samples in this step is the absence of tumor material in the sample.

### Computation of Allelic Imbalance Per Gene-Region

Allelic imbalance was computed independently for each gene-region and cfDNA sample. First, the set of informative SNPs spanning a gene-region (SNP^{GR}) is defined retaining only SNP positions in the Example 1 SNP panel with heterozygosity of 0.2<AF<0.8 in the matched control (i.e. the genomic DNA from the same patient as the cfDNA sample) and present in the reference model. For each SNP i∈SNP^{GR}, observed local coverages, corresponding local coverage quantile and mirrored allele fractions in cfDNA sample were defined as COVᵢ, qᵢ and AFᵢ, respectively.

The evidence of allelic imbalance for the gene-region was computed as:

$${E ( AI )}_{T} = \frac{{\sum }_{1}^{K} wilcox ( {d}_{T} {D}_{β} )}{K} , with β = 1 and K = 100 ( by default )$$

where β is the proportion of neutral reads(16), d_{T} is the observed mirrored AF distribution in cfDNA sample, D_{β} is a simulated AF distribution generated sampling one time for each i∈SNP^{GR} from a Normal distribution with mean Ref_{A̅F̅}_{ᵢ} and standard deviation

$${E ( AI )}_{T} = \frac{{\sum }_{1}^{K} wilcox ( {d}_{T} , {D}_{β} )}{K} , with β = 1 and K = 100 ( by default )$$

and wilcox is a function returning 1 if the difference between d_{T} and D_{β} applying a Wilcoxon signed-rank test with significance cutoff of 1% is statistically significant, 0 otherwise.

Finally, the beta estimate for the gene-region in cfDNA sample (β_{T}) was computed by comparing d_{T} with simulated distributions mimicking levels of local admixture searching for the most similar one. Formally:

$${β}_{T} = min \{ β \vert W ( {d}_{T} > {D}_{β} ) \} - ( min \{ β \vert W ( {d}_{T} > {D}_{β} ) \} - max \{ β \vert W ( {d}_{T} < {D}_{β} ) \} ) * P$$

$$with$$

$$P = \frac{median ( {d}_{T} ) - min ( {d}_{T} )}{max ( {d}_{T} ) - min ( {d}_{T} )}$$

$$and$$

$$β ∈ [ 0 , 1 ]$$

and where W(d_{T}>D_{β}) is the Wilcoxon signed-rank statistic (significance cutoff of 1%) comparing d_{T} and D_{β}.

Similarly, evidence of allelic imbalance E(AI)_{G} and beta (β_{G}) for each gene-region in the matched control sample are computed by substituting d_{T} with d_{G} in the equations above with d_{G} defined as the observed mirrored AF distribution in matched control sample.

### Reference Mapping Bias Correction

To correct for the Reference Mapping Bias (15) (RMB) and to improve the quality of downstream analysis of allelic fraction (AF) data of informative SNPs (iSNPs), a peak correction was applied separately to matched control and cfDNA samples. Specifically, a Kernel Density Estimation (KDE, performed on R using the function “density” from “stats” package with bw=“Si” (RDocumentation version 3.6.2 https://www.rdocumentation.org/packages/stats/versions/3.6.2)) was applied on the iSNPs AF distribution and peaks extracted by computing the local maxima of the smoothed distribution; the closest peak to RMB (by default 0.47) was extracted and data centered to the 0.5 theoretical value. RMB correction was applied both for the generation of reference model and the computation of allelic imbalance. Distribution of iSNPs allelic fractions in control (green) and cfDNA (red) samples before and after RMB correction is shown in FIG. 12 (left and right panels, respectively). Dashed lines show median allelic fraction of iSNPs. This correction ensures a proper comparison of AF distributions from independent samples.

### Tumor Content and Ploidy Estimation

Tumor content (TC) and ploidy estimations for each sample are performed integrating the outputs of the ad-hoc procedures presented above within the CLONETv2 framework(16). In case of missing estimation by CLONETv2, for example at very low tumor content, the following procedure is applied for tumor content:
1. Log 2R of each target gene-region is corrected for ploidy;
2. Target gene-regions with E(AI)_{T}≠0 and ploidy-corrected Log 2R<−0.05 are retained (i.e. target genes harboring putative hemizygous losses);
3. Tumor content is computed as:

$$T C = 1 - \frac{{β}_{T}^{mean}}{2 - {β}_{T}^{mean}} ,$$

with β_{T}^{mean} equal to the mean beta value of the selected target gene-regions.

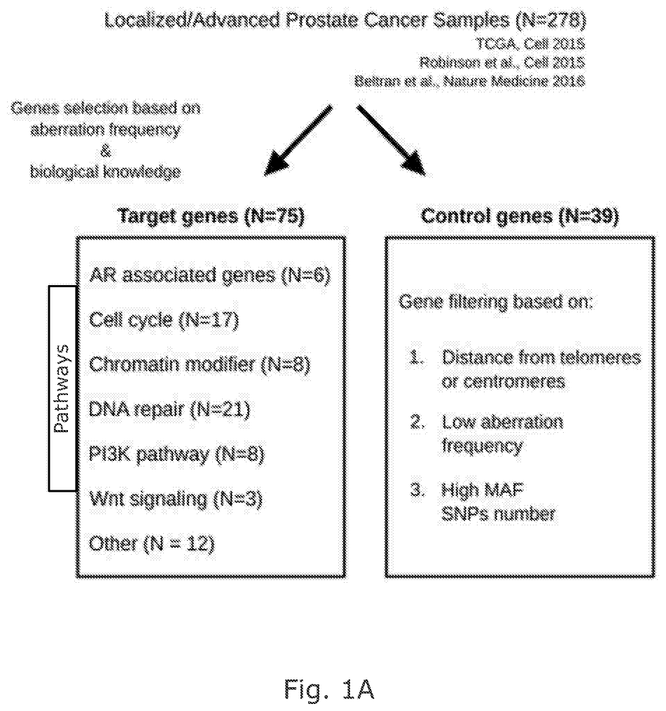

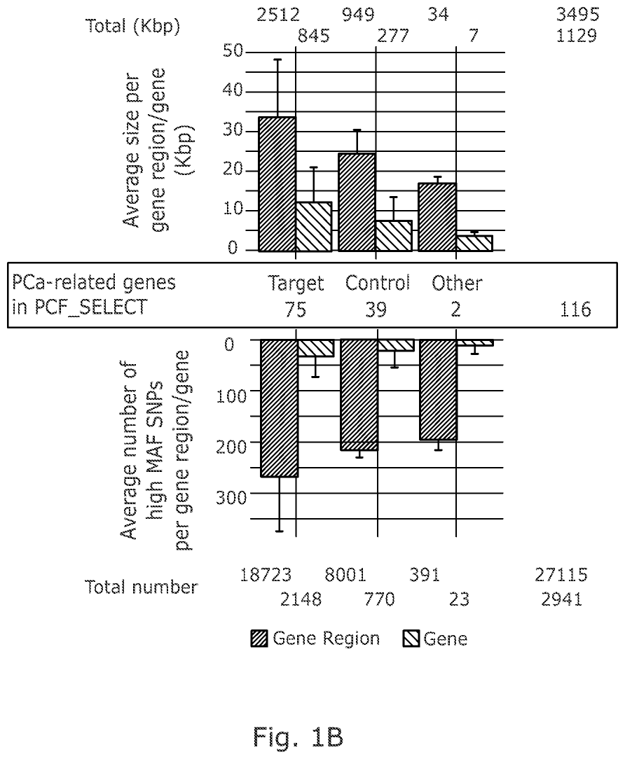

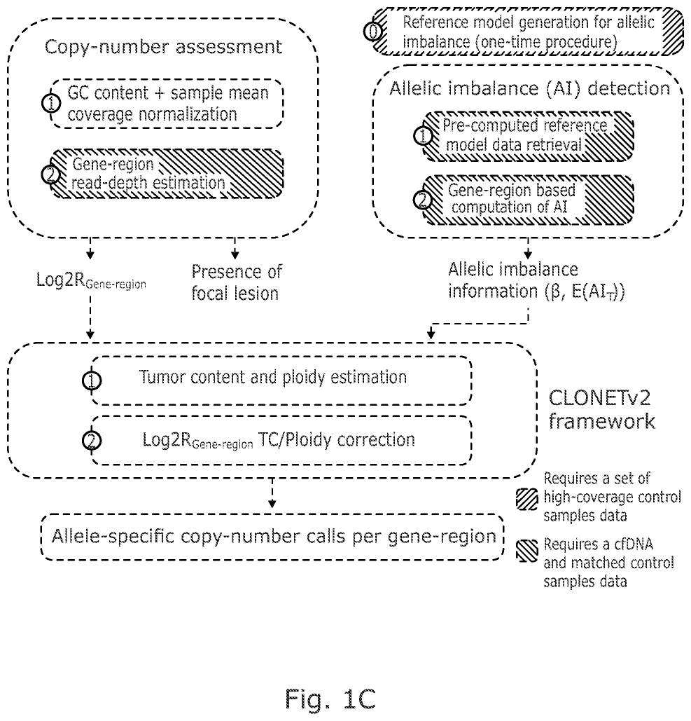

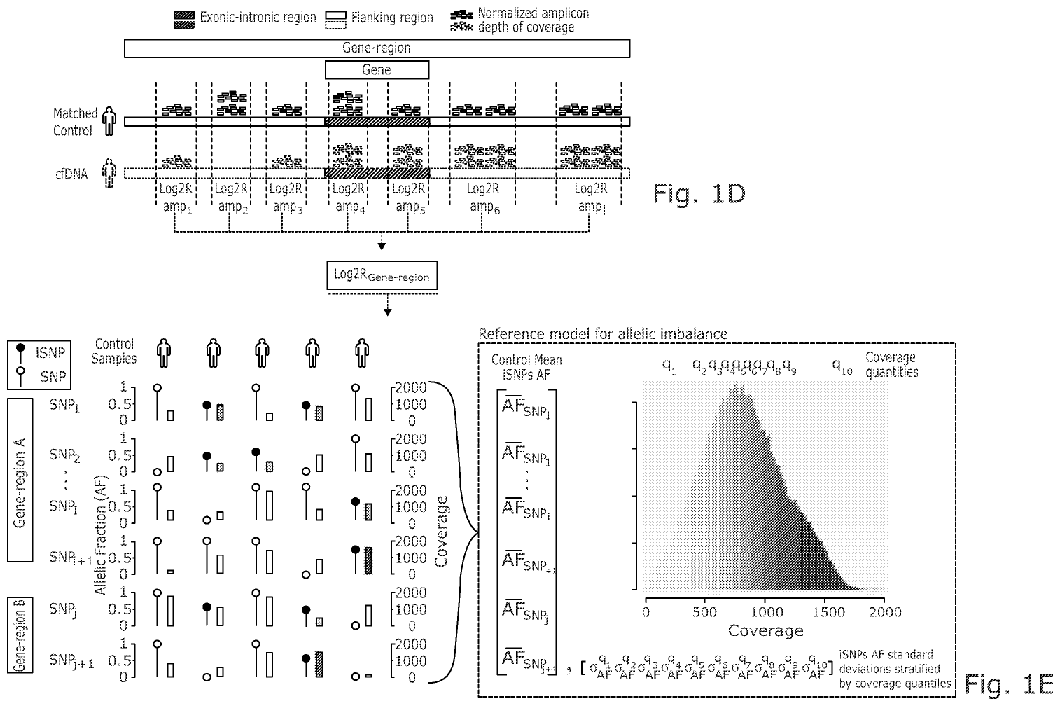

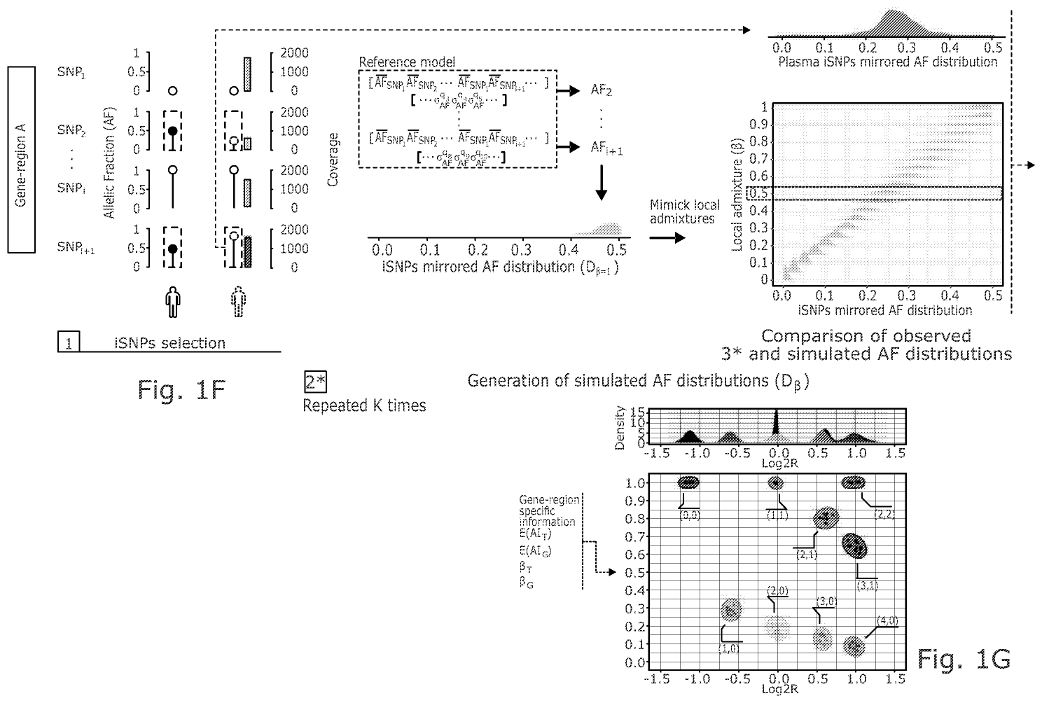

All CLONETv2 ploidy estimates are verified through visual inspection using the Log 2R-beta space (see FIG. 1G).

### Definition of Gene-Regions Allele-Specific Copy Numbers

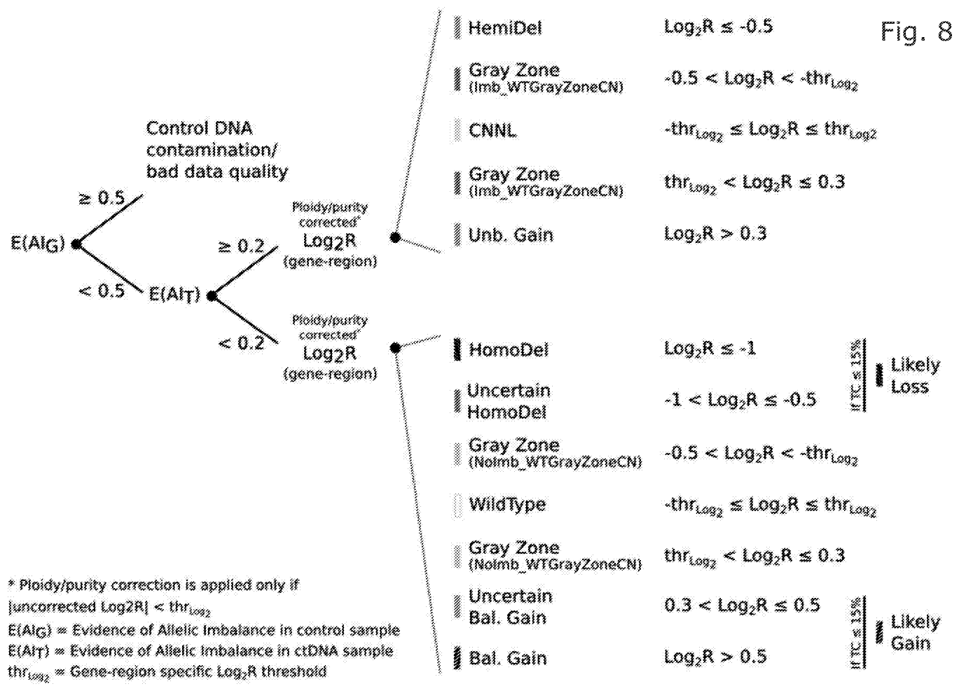

In order to define the allele-specific CN status of each gene-region, the decision tree depicted in FIG. 8 was applied. Briefly, allele-specific CN was defined by integrating read-depth estimations and allelic imbalance calls. First, a check for the quality of the control samples was performed. Then, the presence of allelic imbalance was assessed and Log 2R corrected for ploidy and purity (i.e. ctDNA level/TC) of the sample(16). Note that ploidy/purity correction is only applied if uncorrected signal supports the presence of aberration (i.e. uncorrected Log₂R≥thr_{Log}_{₂}). Moreover, to be conservative if estimated TC≤15% and E(AI)_{T}<0.2 (i.e. no allelic imbalance detected), the method reports the likely presence of aberration in a gene-region not at an allele-specific level. If estimated TC≤15% and allelic imbalance is identified in the gene-region, the method reports the allele-specific copy number.

To obtain the copy number values of the two alleles, cnA and cnB (by design cnA>=cnB) for each gene-region, the following original equations are applied(16):

$$cnA = \frac{( 2 - {β}_{T} ) ( {β}_{T} \times {2}^{Log 2 Rp} - G ) + 2 G ( 1 - {β}_{T} )}{( 1 - G ) {β}_{T}}$$

$$cnB = \frac{{β}_{T} \times {2}^{Log 2 Rp} - G}{1 - G}$$

where Log 2Rp is the ploidy-corrected Log 2R of the gene-region and G is the admixture of the sample (i.e. 1−TC).
Computation of Allele-Specific Ploidy (asP)

Since CLONETv2 ploidy estimate does not recapitulate the actual amount of DNA per cell and in in low TC samples actual polyploid samples may be classified as diploid, the inventors adapted an allele-specific informed ploidy (asP) measure based on the allele-specific CN profile of each sample (Ciani, Y. et al., Allele-Specific Genomics is an Orthogonal Feature in the Landscape of Primary Tumors Phenotypes 4 Feb. 2021 SSRN: https://ssrn.com/abstract=3779554 or http://dx.doi.org/10.2139/ssrn.3779554), computed as the weighted mean of the allele-specific CN of each gene-region gr∈GR in the Example 1 SNP panel, that is:

$$a s P = \frac{{\sum }_{g r ∈ G R} c n A ( g r ) + c n B ( g r ) ) \times w {s}_{g r}}{{\sum }_{g r ∈ G R} w {s}_{g r}}$$

where GR is the set of gene-regions covered by the Example 1 SNP panel and ws is the genomic size of the gene-region.

### Detection of Somatic and Germline Mutations

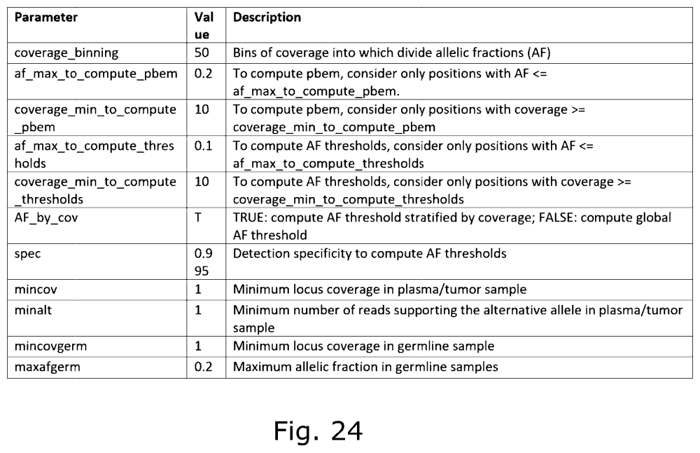

To detect somatic single nucleotide variants (SNVs) we applied ABEMUS(17)), a recently developed method specifically designed for SNVs detection in plasma samples. We ran ABEMUS with parameters reported in FIG. 24. To decrease the impact of false positives, we also applied the following filters:
a. Local coverage in cfDNA sample>50
b. AF in control sample 0.01
c. Exclude positions annotated as SNPs and with MAF>0.01 in dbSNP v144

SNVs were further annotated with Oncotator(18) (version 1.9.6.1) and only non-synonymous SNVs were retained. Germline variants were identified in control samples by looking for positions with AF≥0.15. Only positions annotated as “pathogenic” in ClinVar were retained (19).

Allele-Specific Informed Copy Number Calls without Matched Control Sample

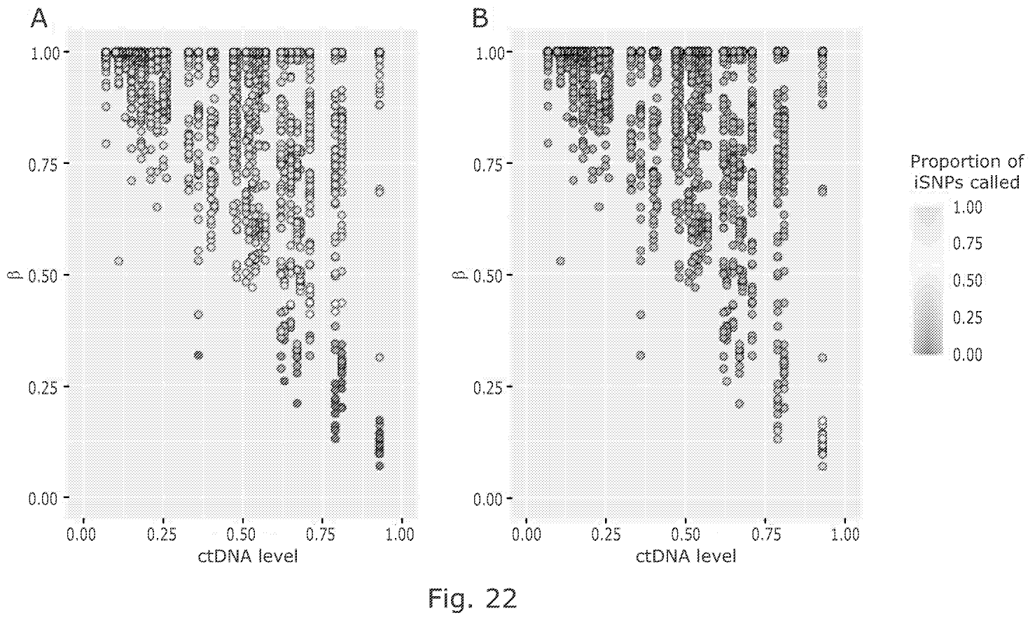

The Example 1 SNP panel may also be used to detect allele-specific copy number also when a matched control sample for the patient is not available. In order to determine allele-specific copy number alterations in absence of a matched control sample the following are needed: a panel of normal (PON) to be used as control in read-depth estimation, a pre-computed reference model for allelic imbalance computation and a procedure for inference of informative SNPs directly from cfDNA sample. The PON can be routinely computed pooling together a set of non-tumor samples with comparable coverages and by computing the mean coverage of each amplicon across the samples selected. iSNPs can be inferred directly from the cfDNA sample, for example by applying thresholds on SNP AFs (e.g. 0.05<AF<0.95). Given the expected low ctDNA level of cfDNA samples, these thresholds guarantee that a discrete proportion of real iSNPs for the patient are recovered (see FIG. 22).

### Results

### A Custom High Density Polymorphisms Panel to Detect Prostate Cancer Aberrations

To enhance the quantification of tumor signal and to enable accurate estimation of copy number changes of prostate cancer relevant genes, the inventors designed a custom targeted sequencing panel (the Example 1 SNP panel) that leverages individuals' genetics across gene-regions of interest for allelic imbalance estimations. The panel design couples information from large scale prostate cancer genomic studies (PMID: 26544944, PMID: 26000489, PMID: 26855148) and human genome polymorphisms features from the 1,000 Genome Project. Specifically, the inventors first identified a set of target genes comprising recurrently aberrant genes in localized and advanced prostate cancer studies and/or involved in frequently altered or targetable pathways, and a set of control genes known to be minimally aberrant in prostate cancer that would provide the backbone structure (e.g. wild-type status) for data analysis, including tumor ploidy and purity estimations (PMID: 31524989) (see FIG. 1A, see Methods).

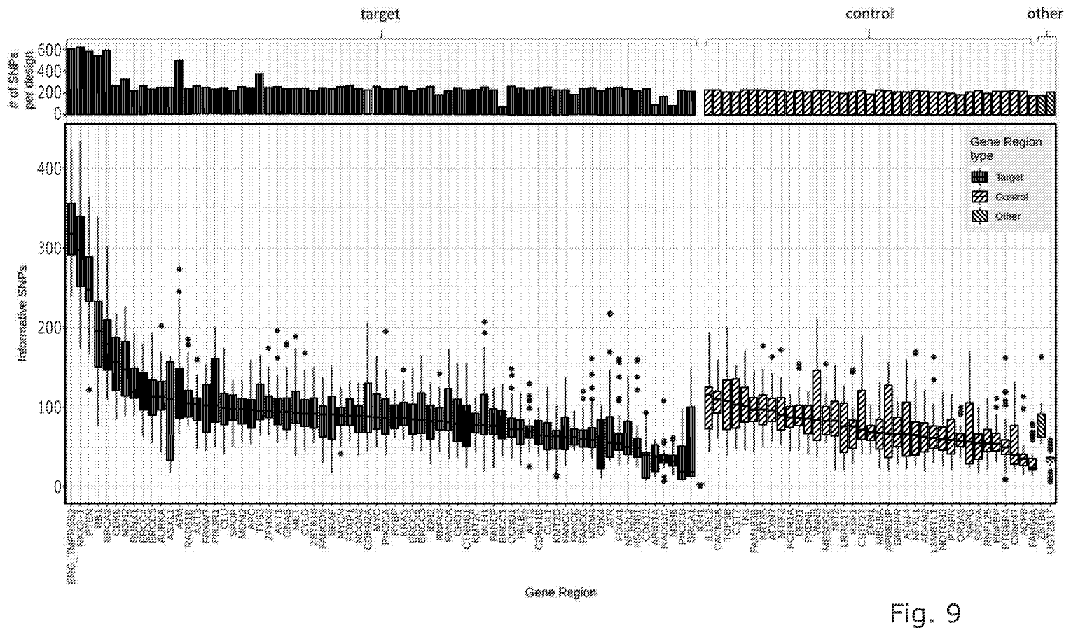

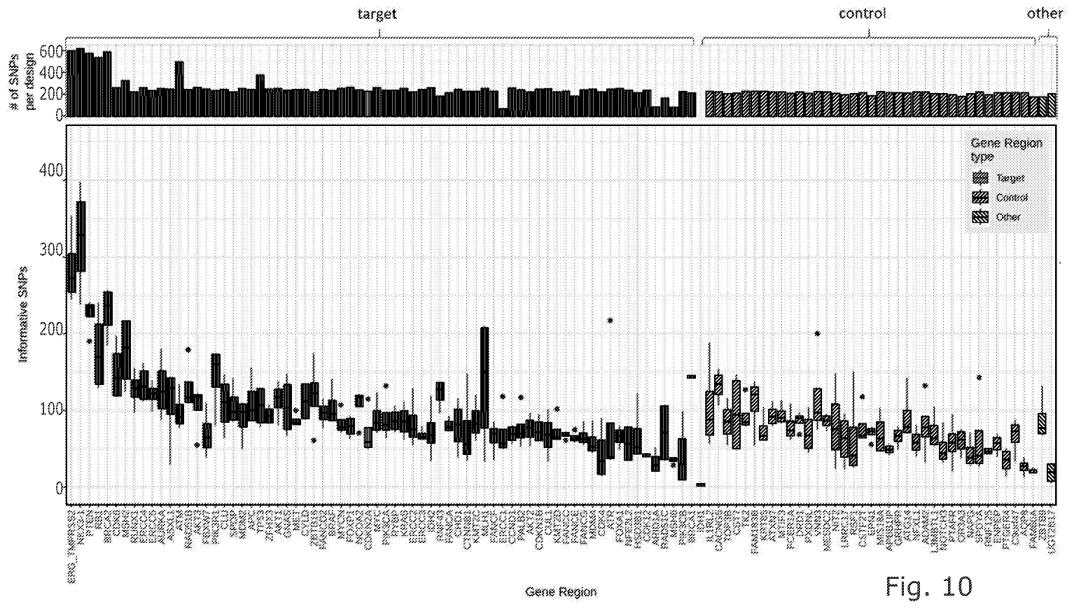

Next, as allelic imbalance can only be measured through heterozygous loci (here the SNPs in the Example 1 SNP panel where a subject has SNP heterozygosity in their genomic non-tumor DNA are referred to as informative SNPs (iSNPs); PMID: 27270079, PMID: 31524989, PMID: 20837533), the inventors designed an optimised panel that is enriched for high minor allele frequency (MAF) SNPs to maximize the detection sensitivity. To cover a high number of potential iSNPs per each targeted genomic region and for each patient, the Example 1 SNP panel was designed to include both exonic and intronic areas of the selected target and control genes and upstream and downstream regions were iteratively added to increase the number of SNPs covered for genes (see Method). The combination of exonic/intronic and flanking regions are referred to as the gene-region of the gene of interest. Overall, the Example 1 SNP panel includes a total of 109 gene-regions and covering 27,115 high MAF SNPs (see FIG. 1B), providing a range of iSNPs per each gene-region and per individual making the panel highly versatile and generalizable (see FIGS. 9 and 10).

Finally, to improve accuracy for assessing copy number state of targeted genes and to assist in increasing the sensitivity in detecting imbalances also in low and moderate ctDNA level samples (<15%), the inventors tailored an ad hoc method for asCNA assessment taking full advantage of the Example 1 SNP panel design. The method integrates i) a read-depth estimation approach modeling gene-specific sequencing coverage noise (see FIG. 1D and FIG. 11) and ii) a gene-region based allelic imbalance detection method leveraging iSNPs upon stringent quality filters (see FIG. 8), followed by their integration and data correction for tumor ploidy and tumor content (i.e. ctDNA level) (see FIG. 1C). Briefly, the allelic imbalance detection step embeds the use of a pre-computed reference model that provides local (mean AF per iSNPs) and global (variation on iSNPs AF by coverage quantiles) statistics of iSNPs and is generated as a one-time procedure using a set of pooled high coverage control samples (see FIG. 1E, see Methods); for each patient and per each gene-region, the observed iSNPs AF distribution is compared against simulated AF distributions to quantify the allelic imbalance (see FIG. 1F). Last, read-depth and allelic imbalance results are fed to the CLONETv2 framework (PMID: 31524989) for ploidy and purity estimation and adjusted values are used to estimate asCNAs (see FIG. 1G, see Method).

### Example 1 SNP Panel Performance

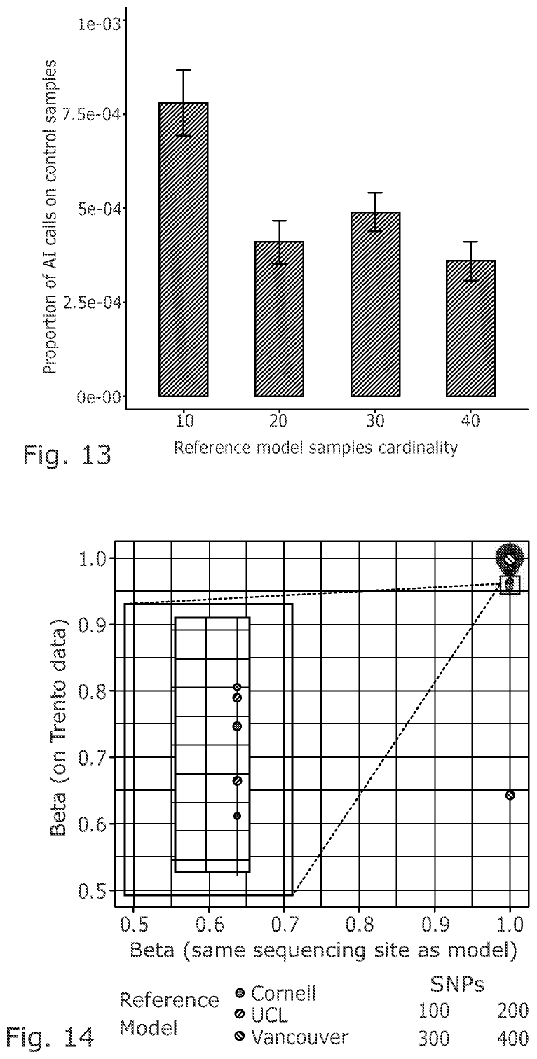

False positive rate (FPR). To assess the performance of the Example 1 SNP panel, serial plasma samples (N=66) and white blood cells (WBCs) were sequenced from 44 individuals with mCRPC (“Cornell dataset”) and plasma samples from 3 healthy volunteers were sequenced independently at four institutions. First the false positive rate (FPR) of the gene-region based method for allelic imbalance detection was evaluated by defining false positives allelic imbalance calls in control samples (for lack of a gold standard). The Example 1 SNP panel FPR in the set of 44 individuals was 0.12%, significantly lower than the rate obtained through a coverage-based only approach (8.5%). Next the impact of the reference model cardinality (i.e. number of control samples used for reference model generation) on the FPR was investigated by iteratively building a total of 80 reference models by randomly selecting 10, 20, 30, and 40 control samples from the Cornell dataset at each iteration (20 reference models per each cardinality). An overall low number of false positives (FPR<0.3%) independent of the reference model cardinality was observed, supporting the benefit of the gene-region based approach (see FIG. 13) and better performances were observed at increasing cardinality with no substantial changes when comparing models built using 20, 30 or 40 control samples (see FIG. 13). Last, healthy volunteers data were used to assess the feasibility of an across-platform reference model (i.e. combination of sequencing platform and sequencing site). Reference models built with control samples sequenced at three institutions (“Cornell”, “UCL”, and “Vancouver”) were applied on 3 healthy volunteer samples sequenced independently at 4 institutions and consistency in gene-region specific information was checked. Inconsistencies were observed only for gene-regions with a low (x-y) number of iSNPs available for the individual (see FIG. 14).

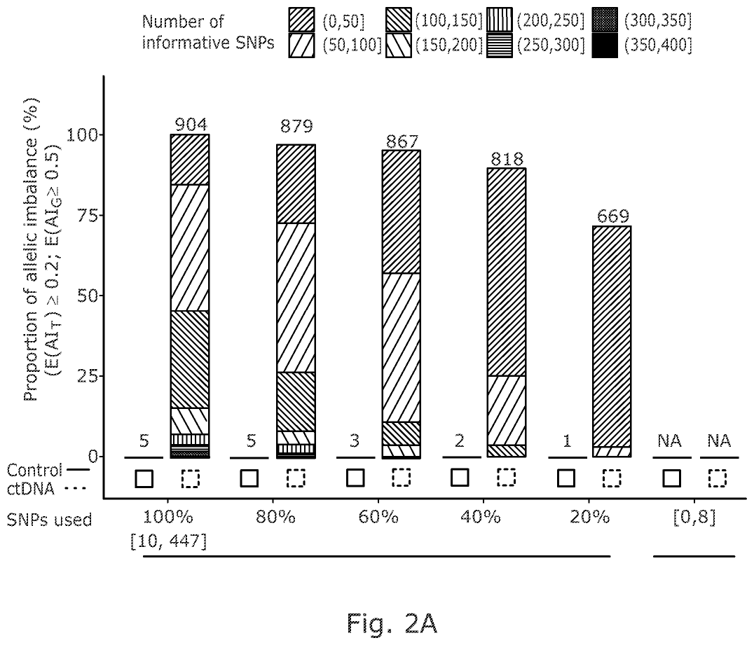

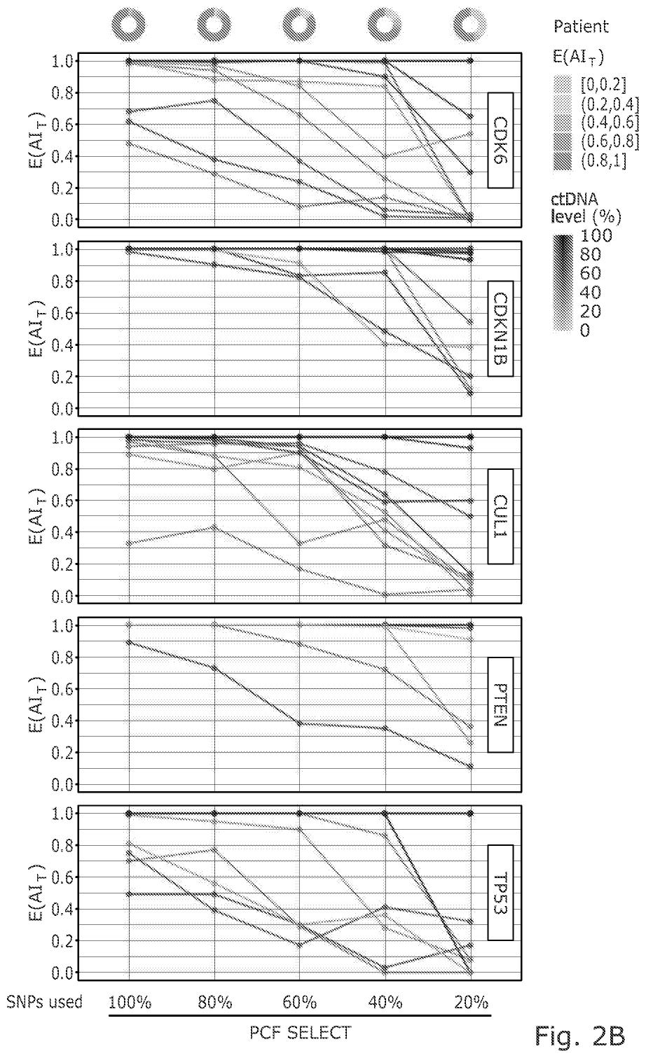

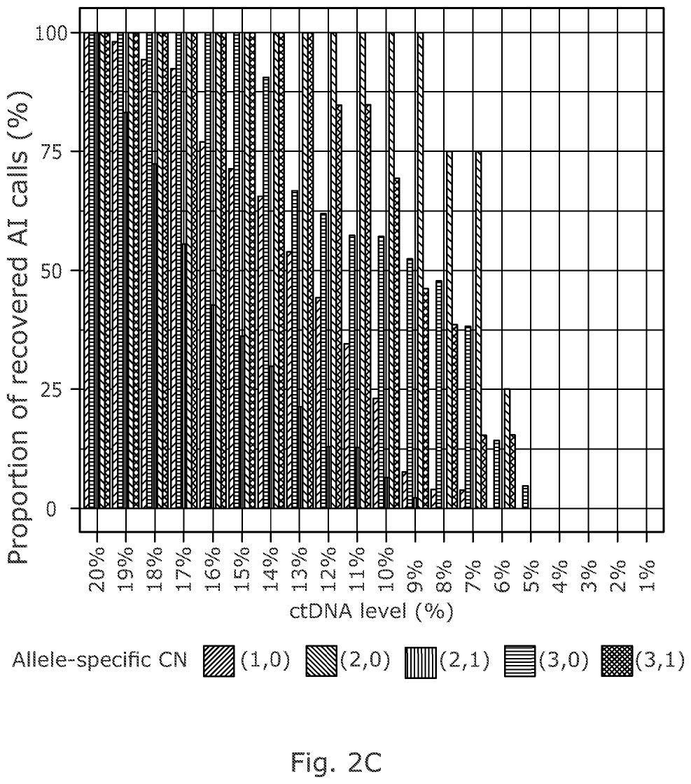

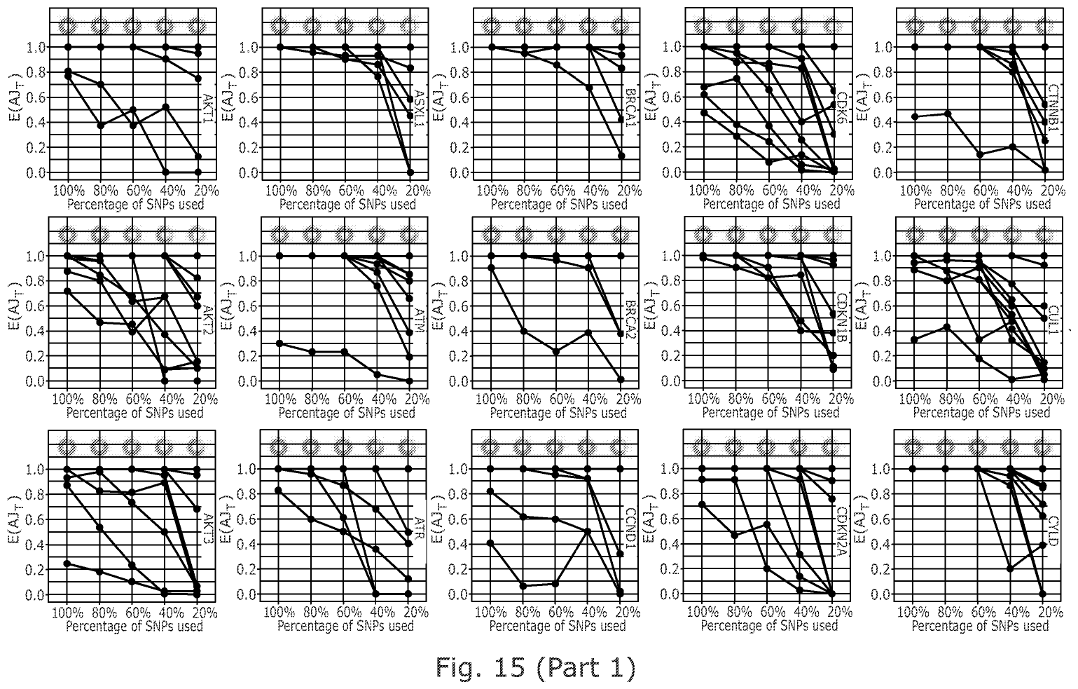

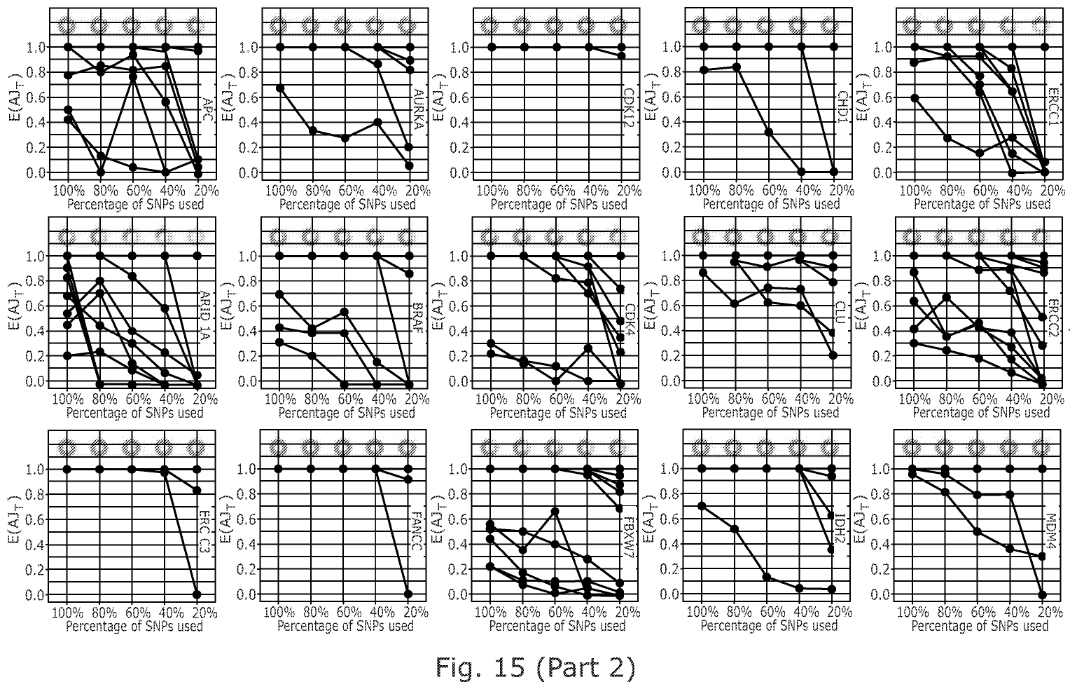

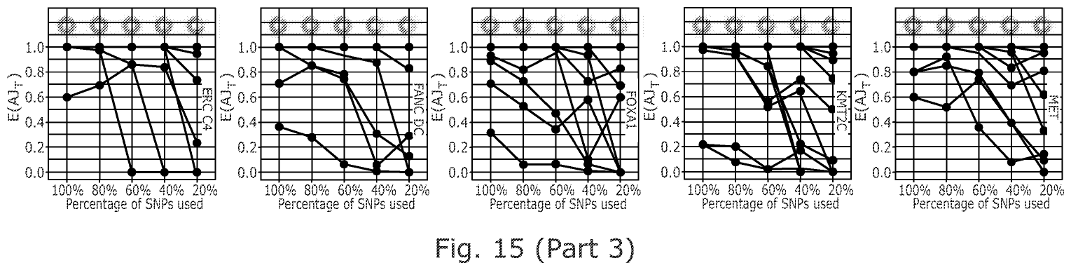

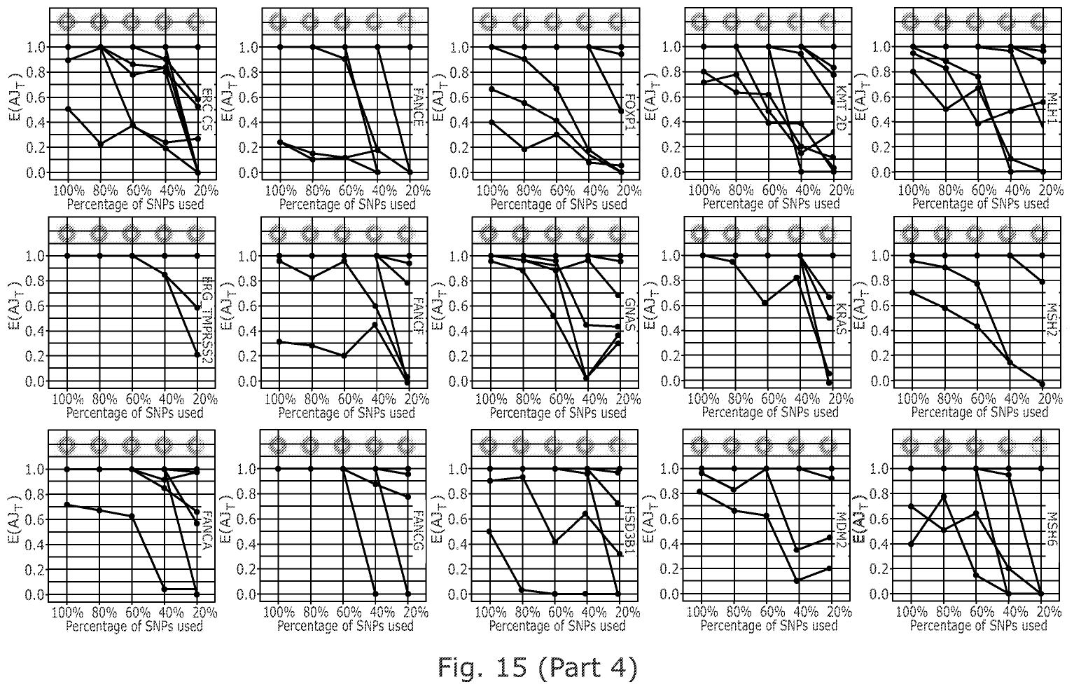

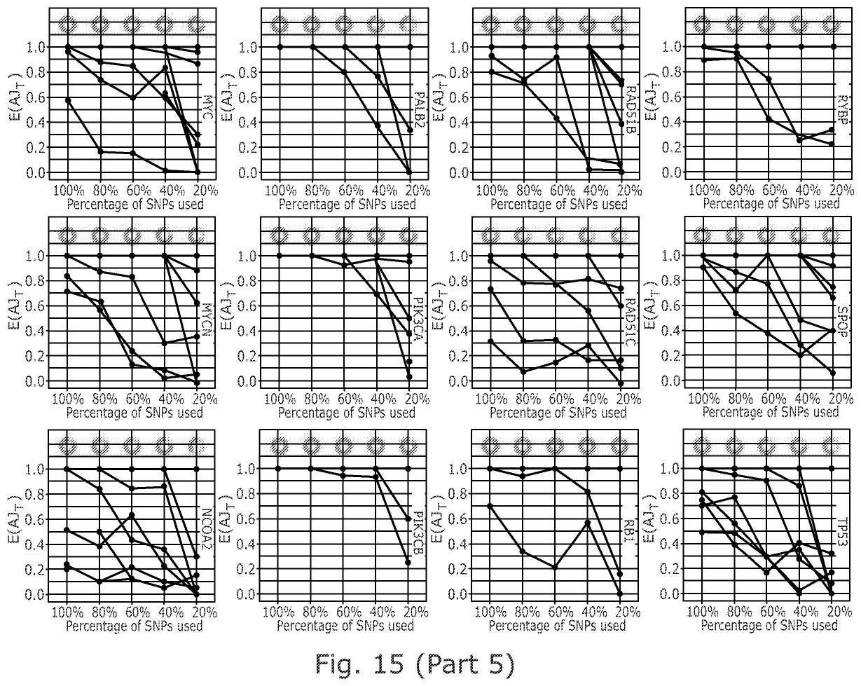

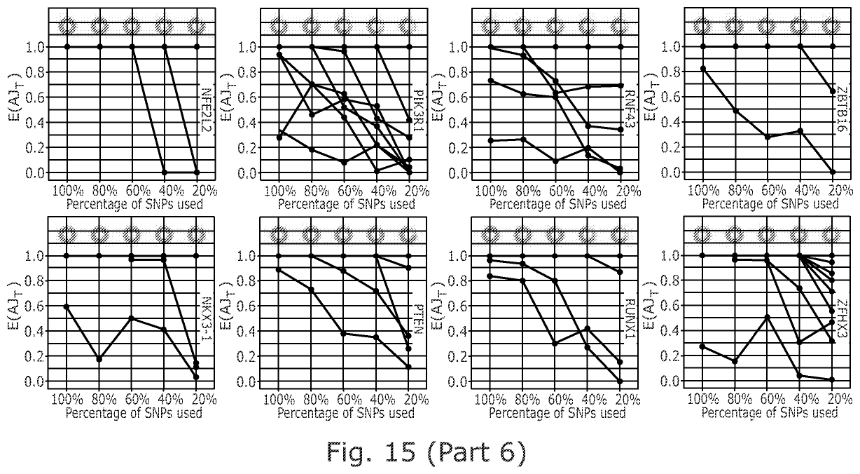

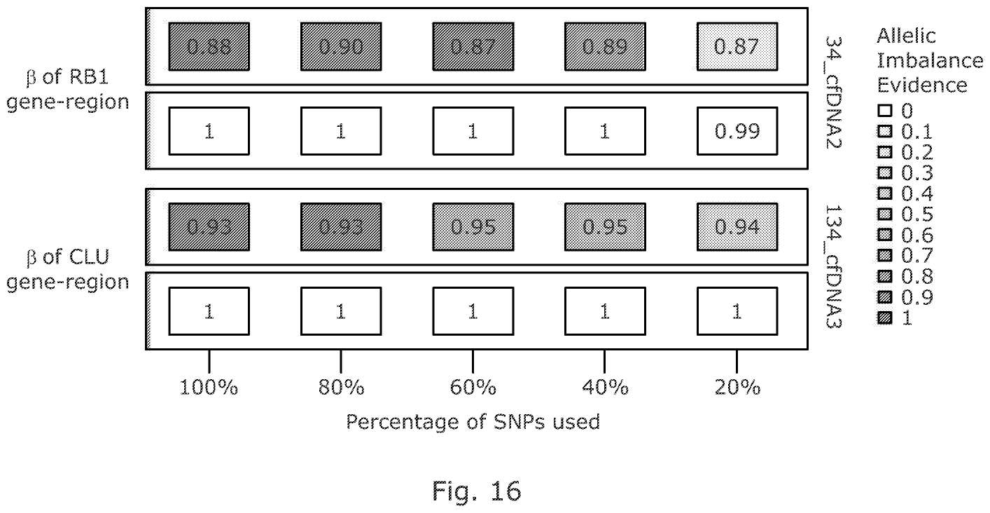

Impact of informative SNPs (iSNP) numerosity on imbalance detection. To measure the impact of the high MAF SNPs-enriched design of the Example 1 SNP panel on allelic imbalance detection, the inventors applied the panel on the Cornell dataset by using for each target gene-region all the available iSNPs per individual (i.e. all heterozygous SNPs that an individual has in the Example 1 SNP panel) and by considering randomly selected subsets (i.e. 20, 40, 60 and 80% of all the available iSNPs per individual). Results confirmed that a higher number of iSNPs is associated with enhanced detection of imbalance events in tumor samples (see FIG. 2A). FIG. 2A shows the proportion of AI calls on the Cornell dataset by varying the percentage of iSNPs compared with whole exome sequencing (WES) data (ranges of SNPs used were reported). Values are shown for both matched control and cfDNA samples (light and dark shaded boxes at the bottom of each bar, respectively). The proportions of AI reported are relative to the number of AI calls obtained using all the informative SNPs available for each gene in the Example 1 SNP panel (i.e. 904). The number of allelic imbalance calls is shown on top of each bar. AI calls were stratified for the number of informative SNPs used (bar shading). Of note, when the Example 1 SNP Panel was applied only on SNPs spanning exonic regions only few positions (min. 0, max. 8) met the selection criteria and in all cases tested there were not sufficient iSNPs for sensitive detection of allelic imbalance. This suggests a bespoke gene-region custom approach is required for sensitive detection of allelic imbalance and the unfeasibility of implementing the approach in a whole-exome sequencing scenario (see FIG. 2A). When we focused on a set of representative gene-regions, including both gene-regions of special interest such as PTEN and TP53, and other genes (e.g. CDK6, CDKN1B, and CUL1), this analysis corroborated the previous results highlighting the impact of the number of iSNPs on allelic imbalance detection. In particular, detection performances decreased significantly by lowering the number of iSNPs (average 6% and 30% reductions with 60% and 20% of available iSNPs, see FIG. 2B and FIG. 15). Moreover, independently of the estimated proportion of tumor reads, a higher number of iSNPs led to higher confidence in allelic imbalance calls (see FIG. 16).

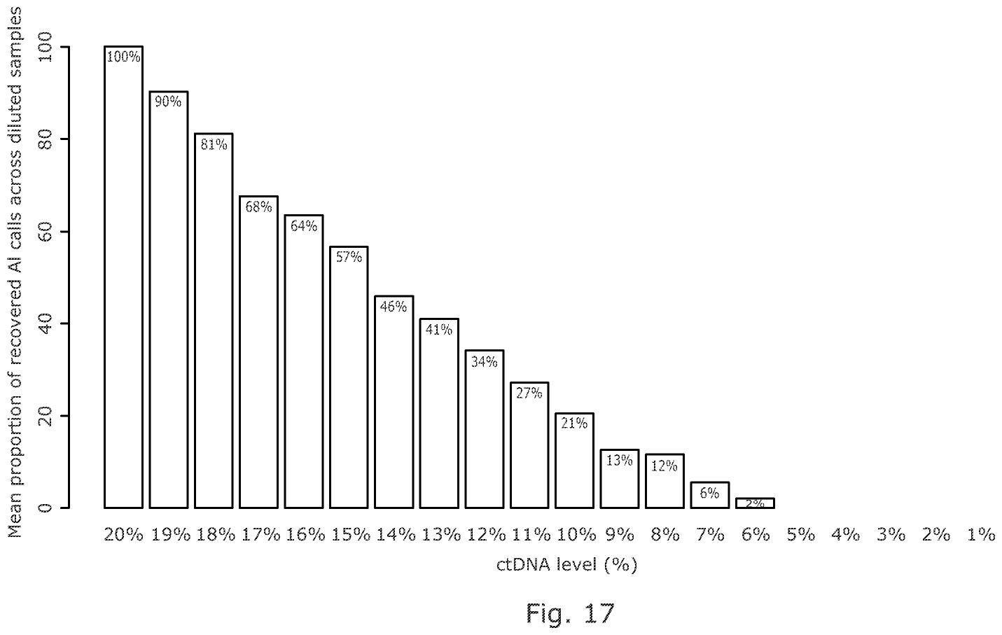

Allelic imbalance detection as function of tumor content via synthetic dilutions. To assess the performances of the method for allelic imbalance detection in the context of varying ctDNA fraction, tumor reads from cfDNA samples from 5 patients from the Cornell cohort were synthetically admixed with reads from each patients' matched control sample. A total of 100 synthetically diluted samples spanning ctDNA levels from 20% to 1% were generated. The ability of the method to consistently detect allelic imbalance at diminishing the ctDNA level was monitored. Of note, the method was able to detect signal of imbalance down to 5% ctDNA level with more than 50% of imbalance calls recovered at 15% ctDNA level (see FIG. 17). Of note, all the imbalance calls were observed in gene-regions with imbalance detected also in the undiluted sample. This result was highly consistent with the low FPR observed in previous analyses and reconfirmed the high specificity of the method in detecting allelic imbalances.

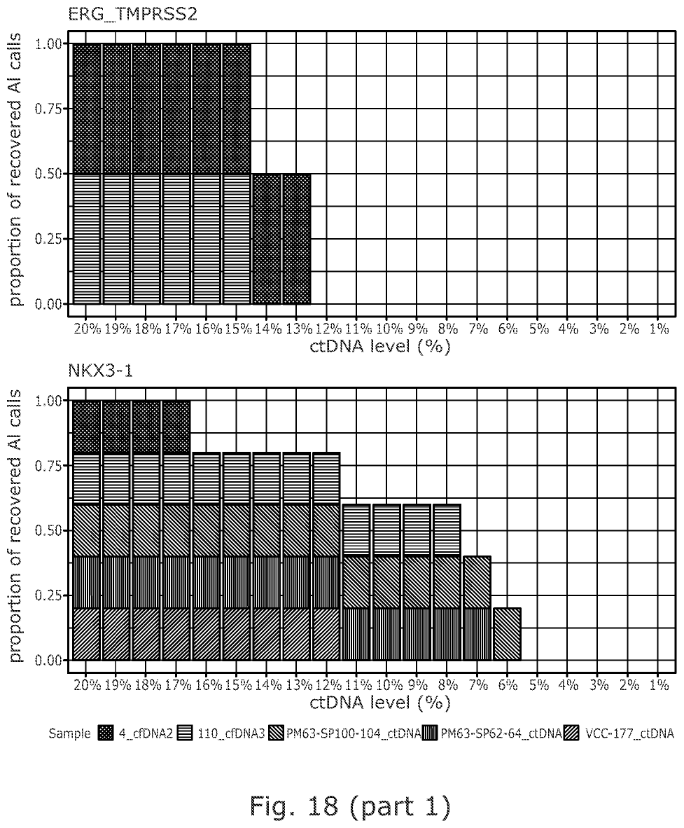

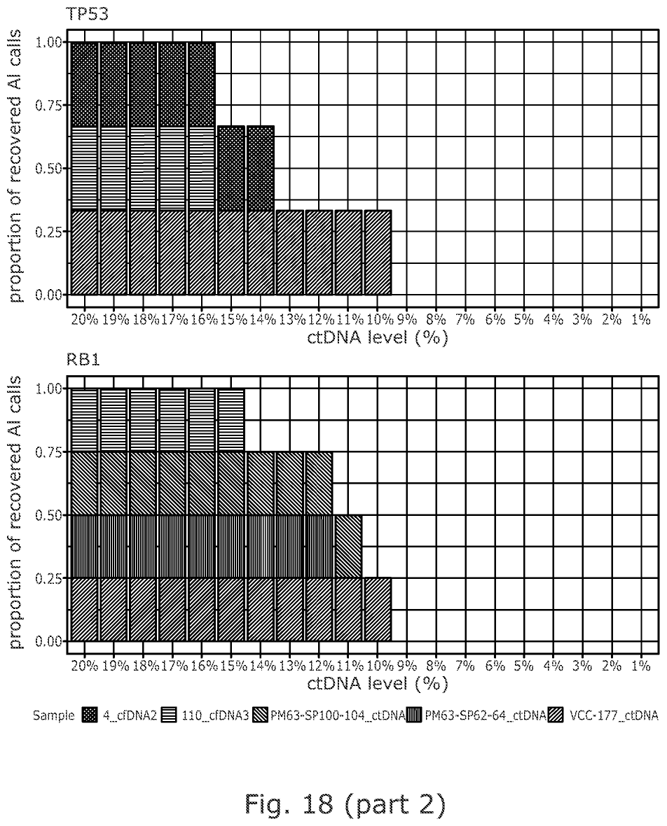

The ability of the method to detect imbalance was further characterized by focusing on 4 gene-regions of the Example 1 SNP Panel (i.e. NKX3-1, TP53, RB1 and ERG-TMPRSS2 regions) and a diverse ability to detect imbalance both across patients and across gene-regions was observed (see FIG. 18). This detection variability was also observed when allelic imbalance calls were stratified by allele-specific CN states of the gene-regions. For instance, we obtained a higher sensitivity on detection of copy neutral loss of heterozygosity (CNNL, i.e. copies of allele A and B equal to 2 and 0, respectively; down to 5% ctDNA level) when compared with hemizygous deletions (i.e. copies of allele A and B equal to 1 and 0, respectively; down to 7% ctDNA level) (see FIG. 2C). Together these results support the presence of a gene- and patient-specific signal affecting the ability of the method to detect imbalance, whereby multiple factors come to play, including the number of available iSNPs in an individual, the lesion clonality (e.g. intra-patient heterogeneity) and the sequencing coverage depths.

Comparison of Example 1 SNP Panel with an Independent Assay

In order to investigate the ability of the Example 1 SNP panel to monitor patients over time, serial samples (N=9) from 3 mCRPC patients originally reported in Annala et al. (PMID: 29367197) were studied and copy number calls reported were compared. Overall, the inventors observed concordant results between the Example 1 SNP panel and the assay described in Annala et al., with a remarkable ability of the Example 1 SNP panel to accurately recapitulate patients' CN aberrations as reported by Annala et al. (105/109 copy number aberrations, 96.33%), as well as to detect complex copy number aberrations and detect aberrations at low ctDNA levels.

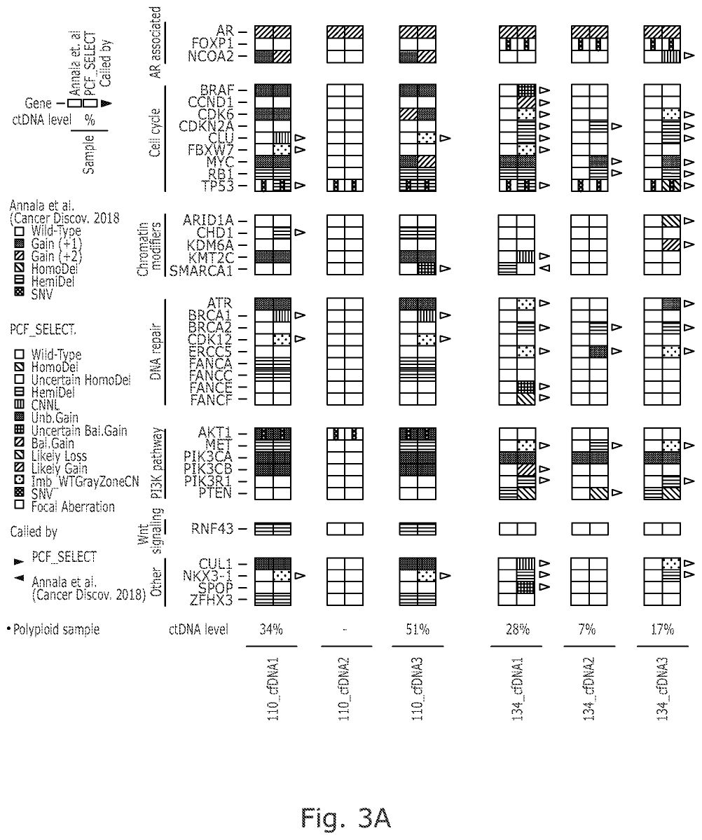

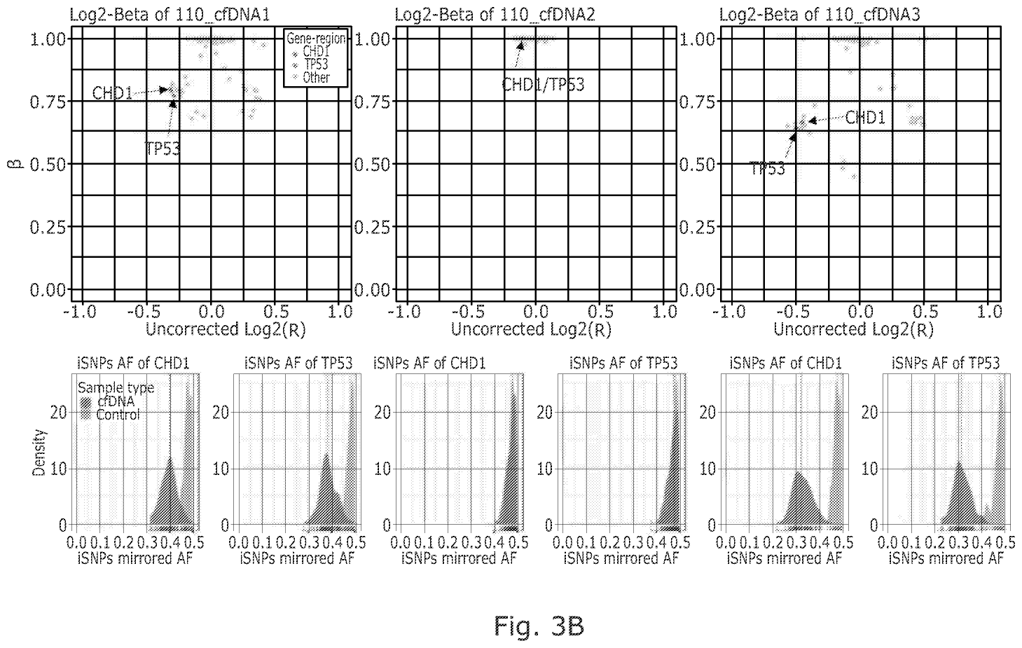

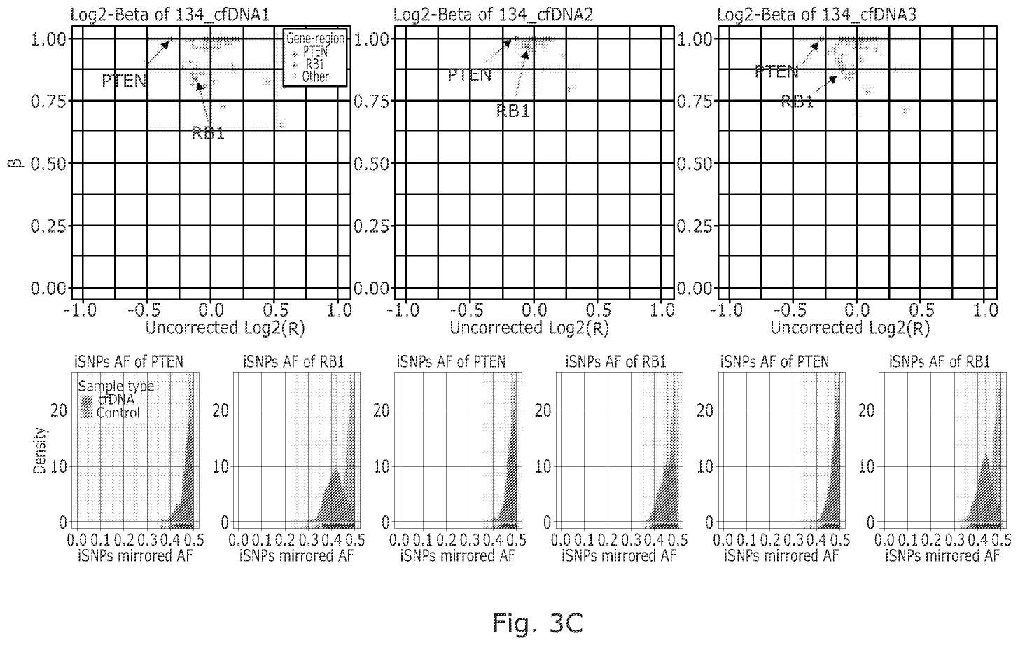

In patient #110, the Example 1 SNP panel was able to detect at the earliest time point (TP-1) the hemizygous deletions of TP53 and CHD1, confirmed at the third time point (TP-3) at higher estimated ctDNA level (51% vs. 34%, for TP-3 and TP-1, respectively) by both the Example 1 SNP panel and the Annala et al. assay (see FIGS. 3A and 3B). The Annala assay did not detect those hemizygous deletions at the earliest time point. Similarly, at the second time point (TP-2) of patient #134, the Example 1 SNP panel was able to confirm copy number changes in 3 genes (i.e. MYC, RB1, and PTEN) concordantly identified by both the Example 1 SNP panel and the Annala et al. assay at TP-1, but missed in the Annala et al assay at TP-2. Moreover, only the Example 1 SNP panel consistently detected the presence of lesions in MET, ERCC5, CDKN2A, and BRCA2 at all time points for patients #134 (see FIG. 3A), whereas no abnormalities were detected using the Annala et al assay. Of note, TP-2 of patient #134 presented a 7% ctDNA level, emphasizing the enhanced sensitivity of the Example 1 SNP panel in detecting lesions in challenging plasma samples. Notably, for patient #134 discordance was observed in reported calls for PTEN where the Example 1 SNP panel consistently detected a homozygous deletion at all time points, whereas Annala et al. reported hemizygous deletions at 2/3 time points; close inspection of the Log 2R-beta space and of the distribution of iSNPs allelic fractions supports the deletion of both alleles, since no significant shift was observed in the distribution across the 3 TPs when comparing cfDNA samples against the matched control samples (see FIG. 3C). This result indicates the Example 1 SNP Panel can detect complex copy number aberration, and do so at low tumor fraction (as noted above at TP-2 the tumor fraction was 7%).

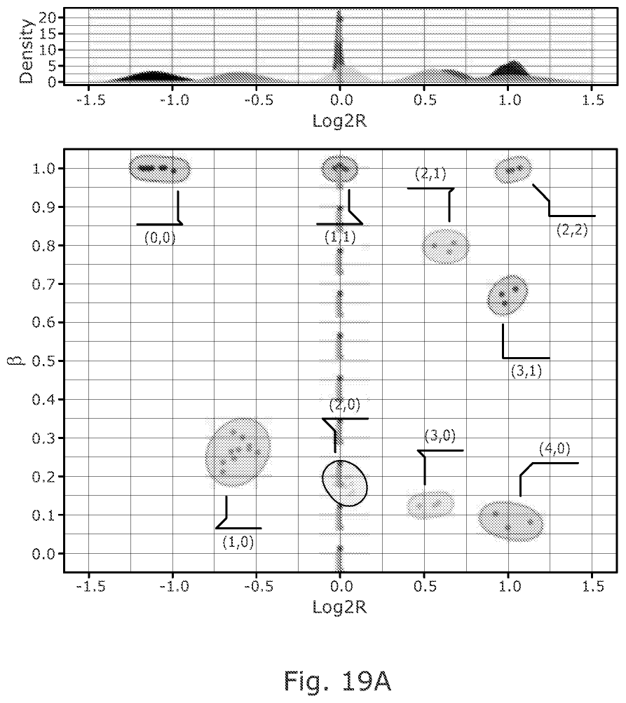

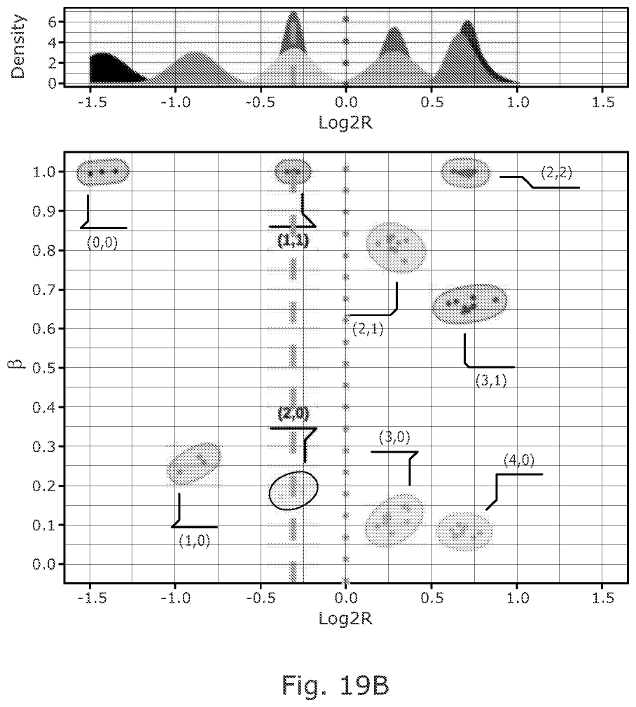

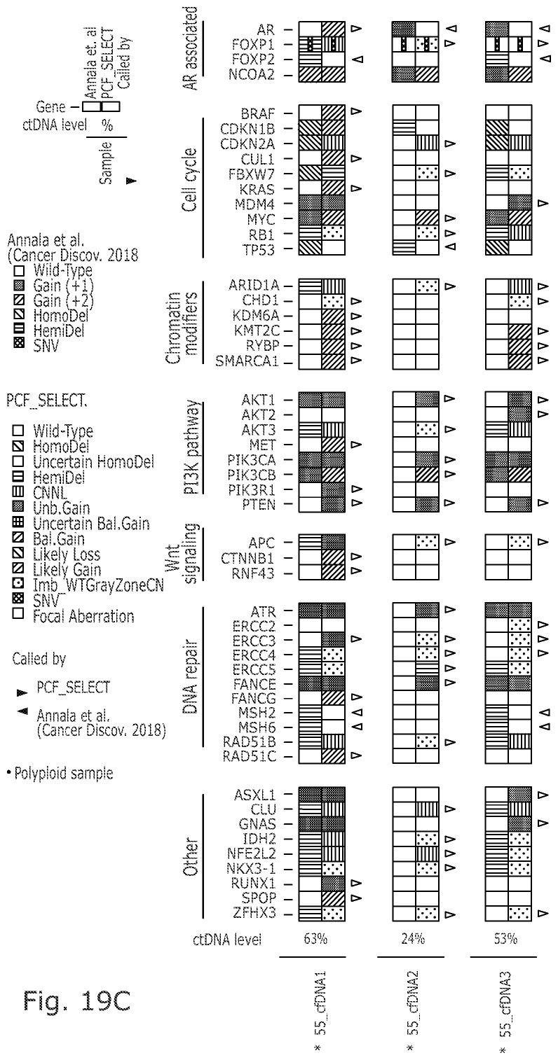

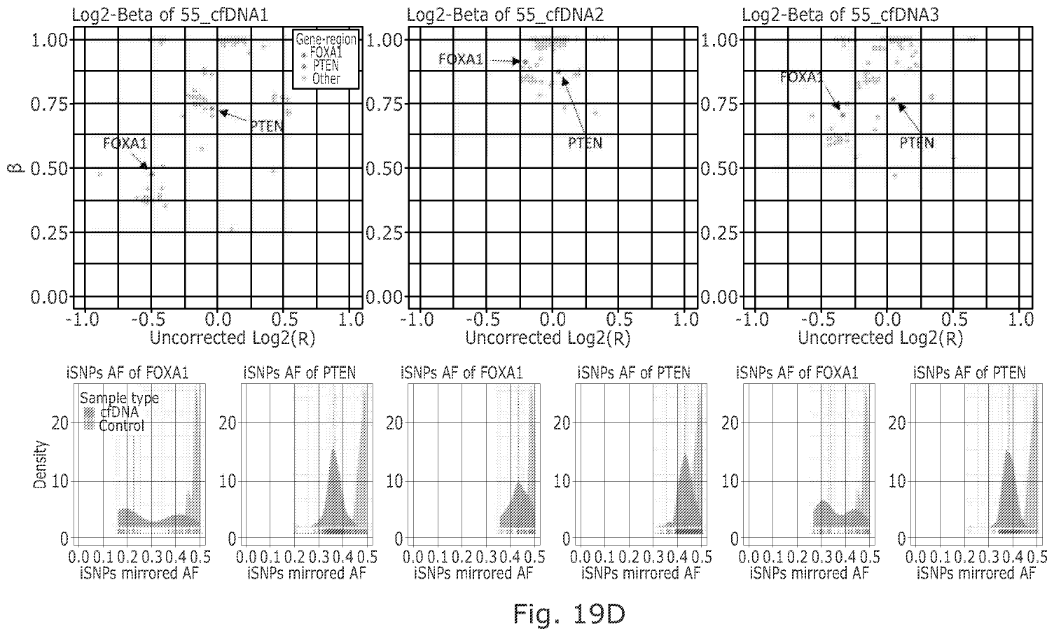

When the Example 1 SNP panel was applied on samples from patient #55, polyploidy signal across all time points was consistently detected (ploidy of 2.78, 2.19, and 2.51 for TP-1, -2, and -3, respectively) (see FIGS. 19A and 19B). This resulted in discordant copy number calls when compared to Annala et al (see FIG. 19C). For instance, the original study reported hemizygous deletions in FOXA1 that was instead classified as CNNL by the Example 1 SNP panel (see FIGS. 19C and 19D), and wild-type PTEN that was instead consistently classified as unbalanced gain as supported by the shift in iSNPs AF distribution (see FIGS. 19C and 19D). The consistency in calls at independent time point, and the shift in the AF, support that the calls for the Example 1 SNP Panel are correct.

The Genomics of Patients Treated with PARP-Inhibitors

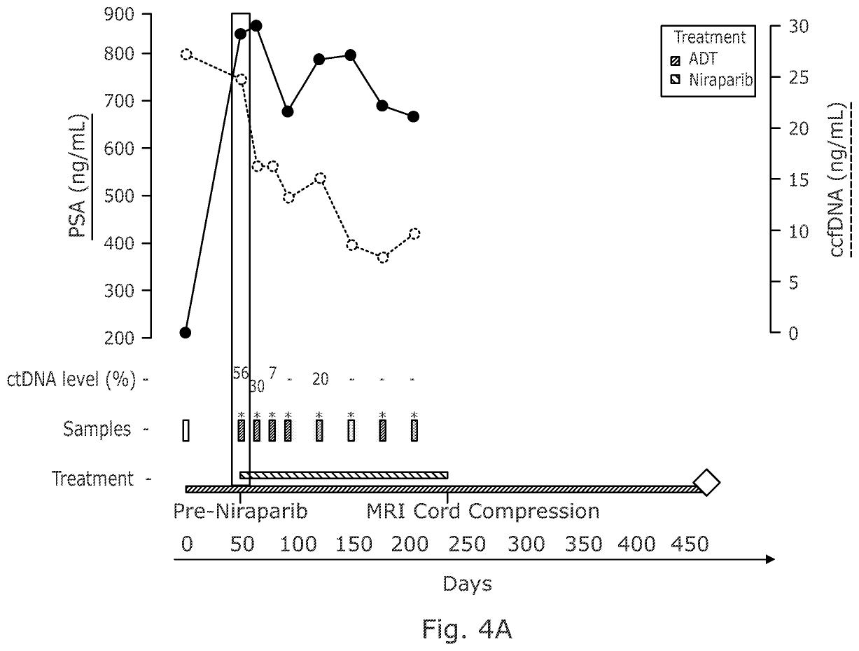

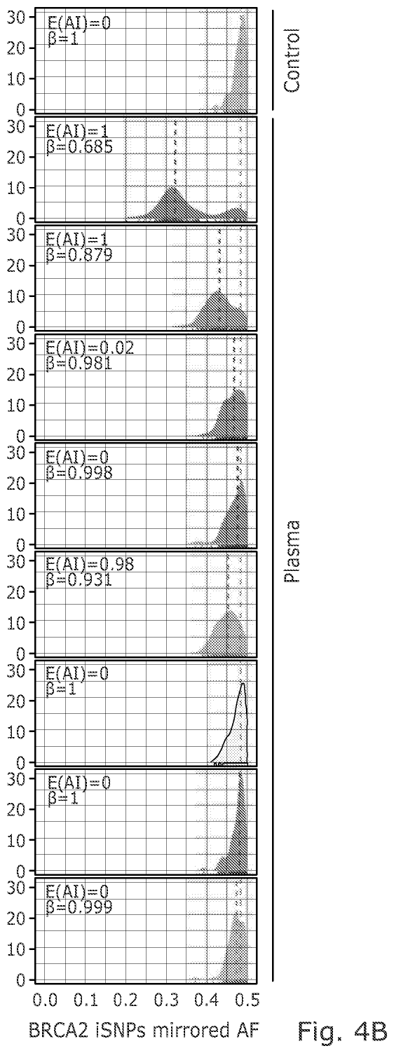

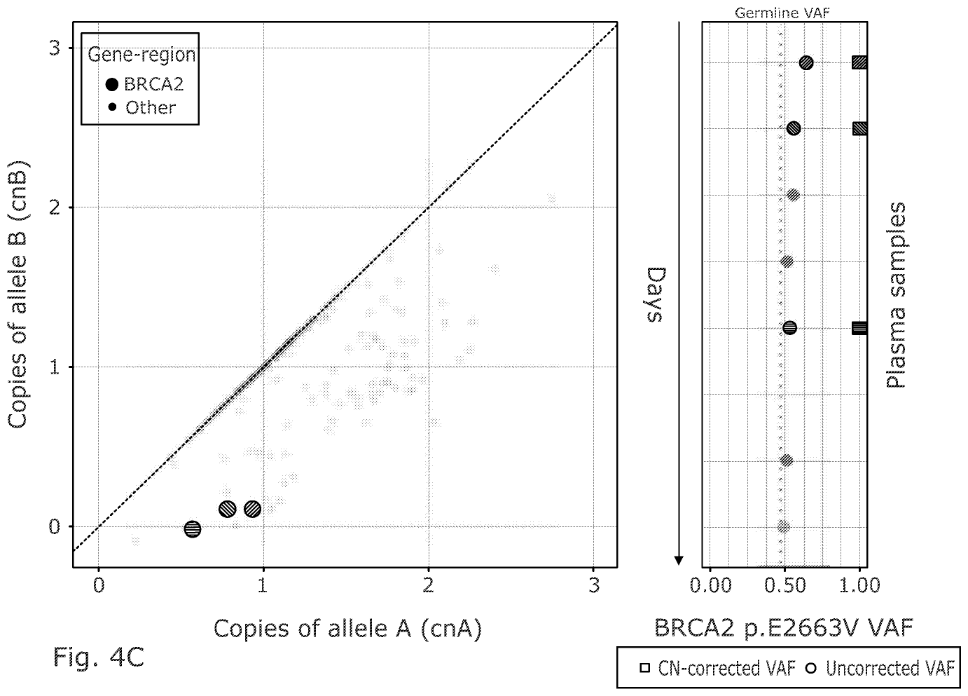

The Example 1 SNP panel was applied on serial samples from 3 patients (1 BRCA mutant+2 ATM mutant patients) treated with a PARP inhibitor. In the BRCA2 mutant patient, the Example 1 SNP panel was applied on 8 of the 9 samples collected (see FIG. 4A). In 4 sequenced samples (50%) with assessable ctDNA level (min. 7%, max. 56%), the Example 1 SNP panel identified the presence of allelic imbalance on the BRCA2 gene-region (see FIG. 4B). In 3/4 samples suitable for allele-specific analysis (i.e. ctDNA level≥15%), the Example 1 SNP panel consistently identified a hemizygous deletion (cnA=1, cnB=0) of BRCA2 gene-region. The deletion was accompanied by a pathogenic missense germline mutation (p.E2663V, VAF=0.47 in control sample; see FIG. 4C). Through CN-based correction of VAF, the inventors determined deletion of the BRCA2 non-mutated allele in the tumor (CN-corrected VAF=1; see FIG. 4C) with predicted loss of functional gene protein. The patient had a PSA decline by 20%, no further cancer growth on CT and bone scans, and a decrease in circulating tumor DNA, in keeping with tumor response to treatment that lasted approximately 9 months. As part of the experimental design, the confidence of the Example 1 SNP Panel to detect the deletion of the non-mutated BRCA2 allele in this patient at decreasing tumor fraction of treatment initiation (FIG. 4C) was tested. The inventors were able to show that the Example 1 SNP Panel allowed high confidence detection of mono-allelic deletions at ctDNA fractions ^{˜}15%. This extends the utility of plasma testing to screen patients for treatment to include subjects presenting with lower plasma tumor fractions. This will, for example, allow for successful screening using plasma for the majority of men with metastatic prostate cancer who present with low levels of plasma tumor fractions.

The results also show that the Example 1 SNP panel can be used track treatment effectiveness both through the lack of allelic imbalance signal for BRCA2 with treatment, indicating depletion of the clone harbouring the BRCA2 loss, and/or the reduction of ctDNA fraction. By using a gene-region design the panel is also able to increase the chance of picking up resistant clones emerging on treatment.

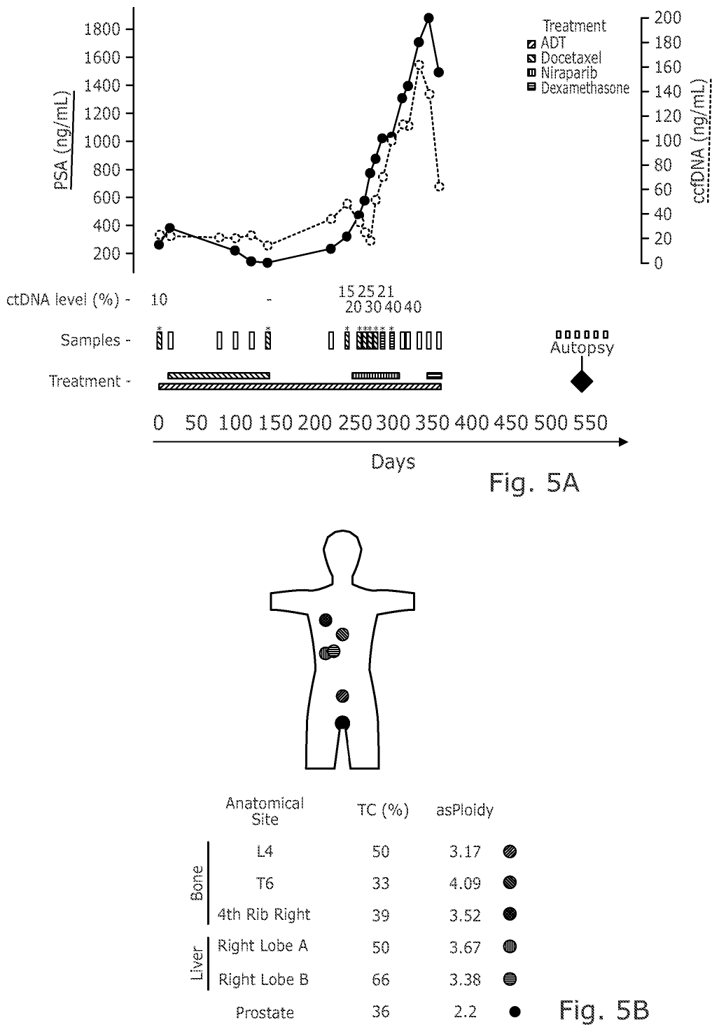

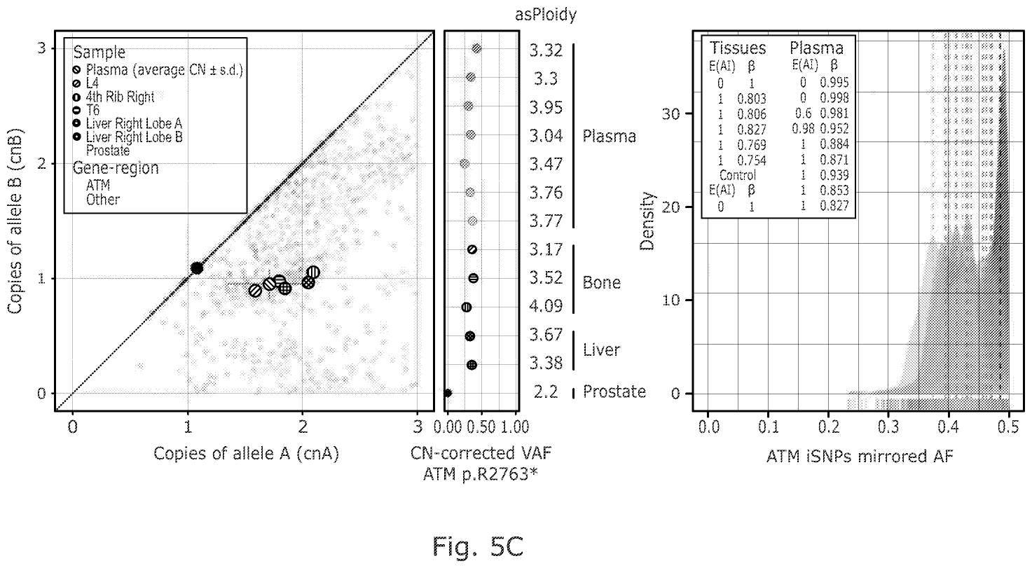

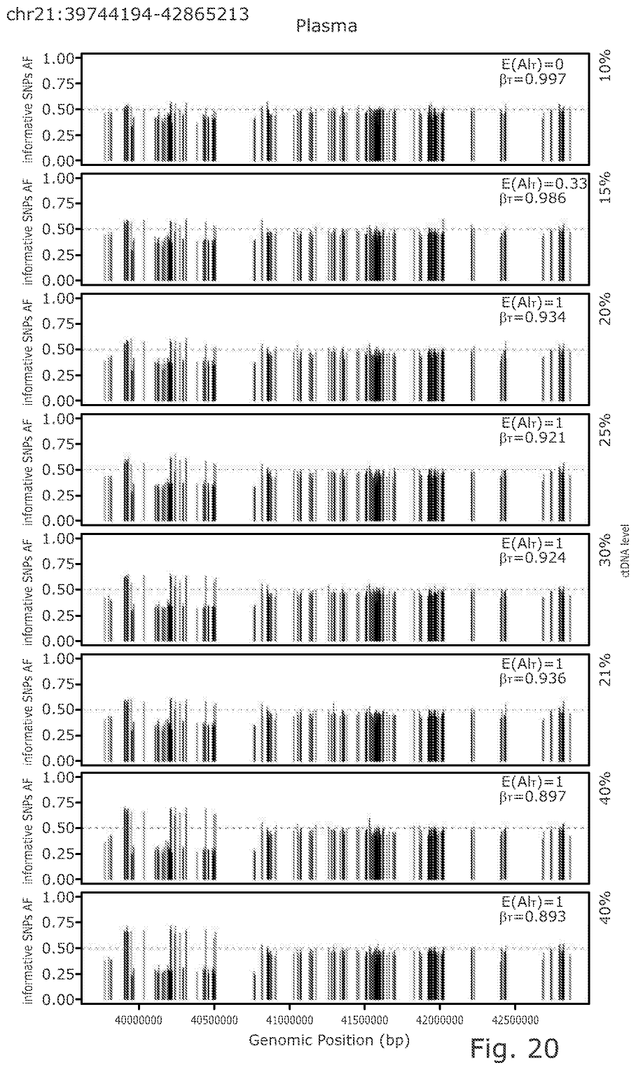

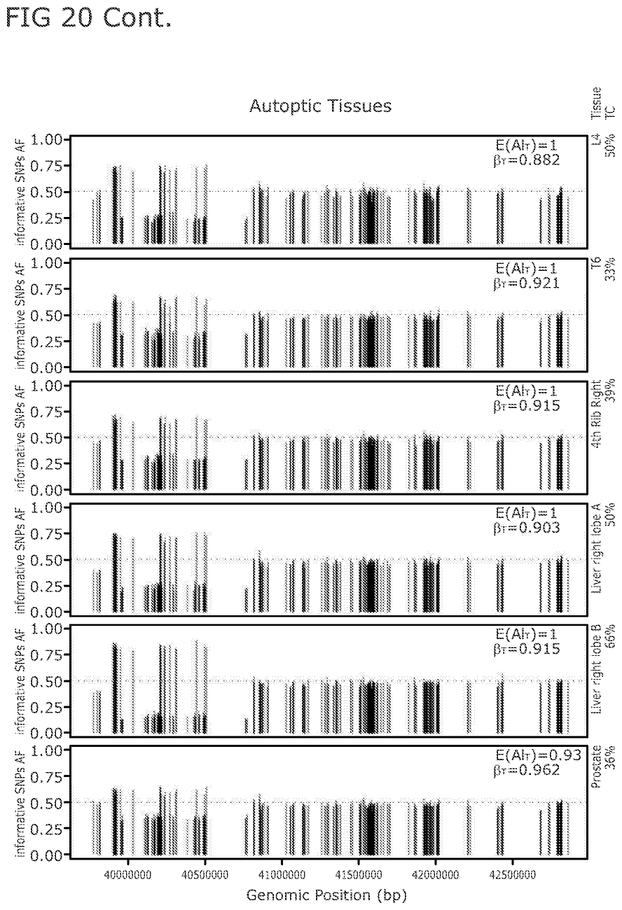

The Example 1 SNP panel was applied on 9/19 plasma samples collected from the first ATM mutant patient. At death, tissue samples from prostate and metastases were collected. A total of 6 tissue samples were sequenced (5 samples collected from metastases and one from the prostate; see FIGS. 5A and B). Allele-specific analysis showed a complex ATM copy number status (2 copies of one allele and 1 of the other) within aneuploid genome (min. asPloidy=3.17, max. asPloidy=4.09) accompanied by a non-sense mutation harboured on the non-gained allele with a CN-corrected VAF=33% (see FIG. 5C). These results were consistently confirmed across all assessable plasma samples (ctDNA level≥15%) and metastatic tissue samples. Of note, the complex ATM CN status was not confirmed in the prostate sample, that showed a wild-type ATM without allelic imbalance and diploid genome (asP=2.2, FIG. 5C). This shows the ability of the approach to detect heterogeneous genomic changes at a gene level. Nonetheless, we observed the same ERG-TMPRSS2 gene fusion across all the sequenced samples known to be an early event in prostate cancer pathogenesis (PMID: 17527075, PMID: 17965219) and supporting the prostatic origin of the metastases (see FIG. 20).

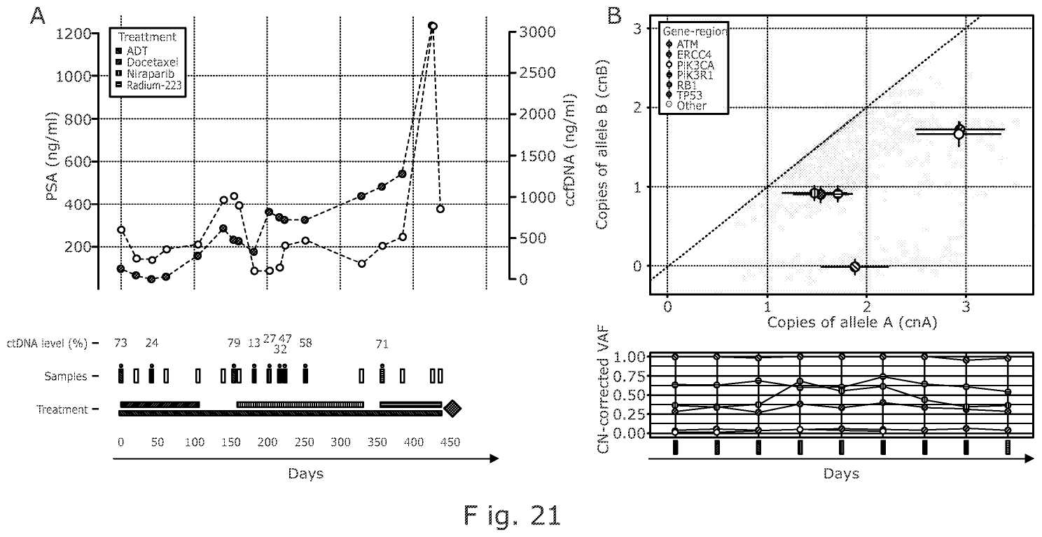

The Example 1 SNP panel was applied on 9 samples collected from the second ATM mutant patient during diverse treatments (see FIG. 21A). Allele-specific analysis showed a complex ATM copy number status (2 copies of one allele and 1 of the other) within aneuploid genome (min. asPloidy=2.51, max. asPloidy=3.80) accompanied by a non-sense mutation harboured on the non-gained allele with a CN-corrected VAF=33% as noted for the previous patient harboring ATM mutations (see FIG. 21B). In addition, recurrent non-synonymous SNVs in 5 genes were identified (p.P114R in ERCC4, p.R175G in TP53, p.R878C in RB1, p.K379E in PIK3R1, p.R93Q in PIK3CA) in complex copy number changes (see FIG. 21B). Of note, we consistently identified a copy-number neutral LOH (CNNL, 2 copies of one allele and 0 of the other) on TP53 accompanied by the missense mutation on both alleles (CN-corrected VAF=100%), suggesting full-impairment of TP53 (see FIG. 21B).

Altogether the findings on both tested ATM mutant patient support a complex ATM copy number state within an aneuploid genome likely incongruent with loss of ATM protein, possibly explaining the resistance to treatment. In this context, the Example 1 SNP Panel could be used to generate a genomic predictor of whether a patient will respond to treatment prior to treatment initiation, thus minimizing side effects and exposure to unnecessary treatment.

### REFERENCES

1. Beltran, H., et al. (2016) Divergent clonal evolution of castration-resistant neuroendocrine prostate cancer. Nat Med, 22, 298-305.
2. Cancer Genome Atlas Research, N. (2015) The Molecular Taxonomy of Primary Prostate Cancer. Cell, 163, 1011-1025.
3. Robinson, D., et al. (2015) Integrative clinical genomics of advanced prostate cancer. Cell, 161, 1215-1228.
4. Setlur, S. R., et al. (2010) Genetic variation of genes involved in dihydrotestosterone metabolism and the risk of prostate cancer. Cancer epidemiology, biomarkers & prevention: a publication of the American Association for Cancer Research, cosponsored by the American Society of Preventive Oncology, 19, 229-239.
5. Carreira, S., et al. (2014) Tumor clone dynamics in lethal prostate cancer. Science translational medicine, 6, 254ra125.
6. Prandi, D., et al. (2014) Unraveling the clonal hierarchy of somatic genomic aberrations.

Genome biology, 15, 439.
7. Romanel, A., et al. (2015) Plasma AR and abiraterone-resistant prostate cancer. Science translational medicine, 7, 312re310.
8. Romanel, A., et al. (2008) SNP panel identification assay (SPIA): a genetic-based assay for the identification of cell lines. Nucleic acids research, 36, 2446-2456.
10. Bolger, A. M., Lohse, M. and Usadel, B. (2014) Trimmomatic: a flexible trimmer for Illumina sequence data. Bioinformatics, 30, 2114-2120.
11. Li, H. and Durbin, R. (2009) Fast and accurate short read alignment with Burrows-Wheeler transform. Bioinformatics, 25, 1754-1760.
12. McKenna, A., et al. (2010) The Genome Analysis Toolkit: a MapReduce framework for analyzing next-generation DNA sequencing data. Genome research, 20, 1297-1303.
13. Li, H., et al. (2009) The Sequence Alignment/Map format and SAMtools. Bioinformatics, 25, 2078-2079.
14. Valentini, S., et al. (2019) PaCBAM: fast and scalable processing of whole exome and targeted sequencing data. BMC Genomics, 20, 1018.
15. Degner, J. F., et al. (2009) Effect of read-mapping biases on detecting allele-specific expression from RNA-sequencing data. Bioinformatics, 25, 3207-3212.
16. Prandi, D. and Demichelis, F. (2019) Ploidy- and Purity-Adjusted Allele-Specific DNA Analysis Using CLONETv2. Curr Protoc Bioinformatics, 67, e81.
17. Casiraghi, N., et al. (2020) ABEMUS: platform-specific and data-informed detection of somatic SNVs in cfDNA. Bioinformatics, 36, 2665-2674.
18. Ramos, A. H., et al. (2015) Oncotator: cancer variant annotation tool. Hum Mutat, 36, E2423-2429.
19. Landrum, M. J., et al. (2014) ClinVar: public archive of relationships among sequence variation and human phenotype. Nucleic acids research, 42, D980-985.

1. An in vitro method for staging, classification, screening, monitoring, stratification, selecting treatment for, ascertaining whether treatment is working in, and/or prognostication of cancer in a subject, said method comprising the steps of:
i) providing a biological sample obtained from the subject, wherein said sample comprises tumor DNA, and a biological sample obtained from the subject, wherein said sample comprises non-tumor DNA;
ii) detecting the presence of single nucleotide polymorphisms (SNPs) in the non-tumor DNA at:
at least 5% of the SNP loci defined in Table 1 for each of at least 5 target gene-regions defined in Table 1,
and optionally at least 5% of the SNP loci defined in Table 2 for each of at least 3 control gene-regions defined in Table 2;
iii) identifying which of the SNPs present are informative SNPs (iSNPs) for the subject, wherein an iSNP is a SNP that is heterozygous in the non-tumor DNA for the subject;
iv-a) determining the allelic imbalance for each target gene-region, and optionally for each control gene-region, of the tumor DNA by reference to the iSNPs for the subject in each gene-region;
iv-b) determining the copy number for each target gene-region, and optionally for each control gene-region, in the tumor DNA; and
v) analysing the allelic imbalance and copy number for each target gene-region, and optionally for each control gene-region, to determine the presence, absence, and/or alteration of one or more allele-specific copy number aberration (asCNA) at each target gene-region in the tumor DNA;
wherein the presence, absence, and/or alteration of one or more asCNA in the tumor DNA indicates that the subject would benefit from treatment with one or more cancer treatments, has benefited, or is benefiting, from one or more cancer treatments and/or would benefit from ceasing or altering one or more cancer treatments.

2. The method of claim 1, wherein the biological sample comprising tumor DNA is a blood sample, urine sample, tissue sample or cerebral spinal fluid sample obtained from the subject; and/or wherein the biological sample comprising non-tumor DNA is a blood sample, urine sample, saliva sample, tissue sample, or cerebral spinal fluid sample obtained from the subject.

3. The method of claim 1 or 2, wherein the biological sample comprising tumor DNA comprises circulating tumor DNA (ctDNA); and/or wherein the biological sample comprising non-tumor DNA comprises non-tumor cell free DNA (cfDNA).

4. The method of any preceding claim, wherein the biological sample comprising tumor DNA and the biological sample comprising non-tumor DNA are the same sample, for example, a blood sample comprising both tumor DNA and non-tumor DNA, for example, a plasma sample comprising ctDNA and non-tumor cfDNA.

5. The method of any preceding claim, wherein the biological sample is a plasma sample comprising cfDNA, for example a plasma sample comprising ctDNA.

6. The method of any preceding claim, wherein step iv-b) further comprises a step of estimating the tumor content (TC) of the sample comprising tumor DNA and/or estimating the ploidy of the sample comprising tumor DNA.

7. The method of any preceding claim, wherein an iSNP is a SNP that is heterozygous in the non-tumor DNA for the subject and has an allelic fraction (AF) of between about 0.05 to about 0.95, for example an AF of between about 0.2 to about 0.8.

8. The method of any preceding claim, wherein the subject is known or suspected of suffering from one or more of the cancers selected from the group consisting of prostate cancer (for example, castration-resistant prostate cancer), breast cancer, ovarian cancer, pancreatic cancer, bladder cancer, and metastatic cancer.

9. The method of any preceding claim, wherein the biological sample comprises cfDNA, and at least about 2%, at least about 3%, at least about 4%, or at least about 5% of the total number of cfDNA molecules in the sample are derived from tumor DNA.

10. The method of any preceding claim, wherein step ii) comprises detecting the presence of SNPs in the non-tumor DNA at:
at least 10%, at least 20%, at least 30%, at least 40%, at least 60%, or at least 80% of the SNP loci defined in Table 1 for each of at least 5 target gene-regions defined in Table 1;
and optionally at least 10%, at least 20%, at least 30%, at least 40%, at least 60%, or at least 80% of the SNP loci defined in Table 2 for each of at least 5 control gene-regions defined in Table 2.

11. The method of any preceding claim, wherein step ii) comprises detecting the presence of SNPs in the non-tumor DNA at:
at least 10%, at least 20%, at least 30%, at least 40%, at least 60%, or at least 80% of the SNP loci defined in Table 1 for each of at least 10, at least 25, at least 50, or all of the target gene-regions defined in Table 1; and/or
at least 10%, at least 20%, at least 30%, at least 40%, at least 60%, or at least 80% of the SNP loci defined in Table 1 for each of at least 10, at least 20, at least 30, or all of the control gene-regions defined in Table 2.

12. The method of any preceding claim, wherein step ii) comprises detecting the presence of SNPs in the genomic non-tumor DNA at target gene-regions and at control gene-regions.

13. The method of any preceding claim, wherein the method further comprises the step of:
vi) determining one or more cancer treatment the subject would benefit from;
and optionally wherein the method further comprises the step of:
vii) administering one or more cancer treatment determined in step vi) to the subject, and thereby treating the subject.

14. The method of any preceding claim, wherein
at least one target gene-region is selected from the group consisting of FOXA1, FOXP1, HSD3B1, NCOA2 and ZBTB16 as defined in Table 1; and/or
at least one target gene-region is selected from the group consisting of as AURKA, BRAF, CCND1, CDK12, CDK4, CDK6, CDKN1B, CDKN2A, CUL1, FBXW7, KRAS, MDM2, MDM4, MYC, MYCN, RB1 and TP53 defined in Table 1; and/or
at least one target gene-region is selected from the group consisting of ARID1A, CHD1, KMT2C, KMT2D and RYBP as defined in Table 1; and/or
at least one target gene-region is selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1; and/or
at least one target gene-region is selected from the group consisting of AKT1, AKT2, AKT3, MET, PIK3C, PIK3CB, PIK3R1 and PTEN as defined in Table 1; and/or
at least one target gene-region is selected from the group consisting of ASXL1, CLU, CYLD, ERG_TMPRSS2, GNAS, IDH2, NFE2L2, NKX3-1, RUNX1, SPOP and ZFHX3 as defined in Table 1; and/or
at least one target gene-region is selected from the group consisting of APC, CTNNB1 and RNF43 as defined in Table 1.

15. The method of any preceding claim, wherein
at least one target gene-region is selected from the group consisting of BRCA2, ATM, RB1, NKX3-1, TP53, and PTEN as defined in Table 1; or
wherein at least 3 of the target gene-regions are selected from the group consisting of BRCA2, ATM, RB1, NKX3-1, TP53, and PTEN as defined in Table 1; or
wherein 6 of the target gene-regions are BRCA2, ATM, RB1, NKX3-1, TP53, and PTEN as defined in Table 1.

16. The method of any preceding claim, further comprising
iv-c) detecting in the tumor DNA the presence of somatic and/or germline mutations in the exonic region of one or more target gene-region defined in Table 1; and optionally detecting in the tumor DNA the presence of somatic and/or germline mutations in the exonic region of one or more of gene selected from the group consisting of AR, MED12, SMARCA1, IDH1 and KDM6A.
wherein the presence of one or more somatic and/or germline mutations in the tumor DNA indicates that the subject would benefit from treatment with one or more cancer treatments, has benefited, or is benefiting, from one or more cancer treatments and/or would benefit from ceasing or altering one or more cancer treatments.

17. The method of any preceding claim, wherein the presence, absence, and/or alteration of one or more asCNA in the tumor DNA indicates that the subject would benefit from treatment with one or more cancer treatments selected from the group consisting of, ATR inhibitor, CDK inhibitor, Chemotherapy, WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, platinum-based antineoplastic drug, taxane, radionuclide and radiation therapy, PI3K inhibitor, mTOR inhibitor, PORCN inhibitor, DNMT1 inhibitor, HDAC inhibitor, BET inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid (for example one or more cancer treatments selected from the group consisting of a chemotherapy (such as taxanes (in particular docetaxel and cabazitaxel) and platinum-based antineoplastic drugs (in particular carboplatin)), PARP inhibitors, or hormonal agent, such as a LHRH agonist, LHRH antagonist, anti-androgen, androgen synthesis inhibitor, estrogen or steroid),
has benefited, or is benefiting, from one or more cancer treatments selected from the group consisting of ATR inhibitor, CDK inhibitor, Chemotherapy, WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, platinum-based antineoplastic drug, taxane, radionuclide and radiation therapy, PI3K inhibitor, mTOR inhibitor, DNMT1 inhibitor, HDAC inhibitor, BET inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid (for example one or more cancer treatments selected from the group consisting of a chemotherapy (such as taxanes (in particular docetaxel and cabazitaxel) and platinum-based antineoplastic drugs (in particular carboplatin)), PARP inhibitors, or hormonal agent, such as a LHRH agonist, LHRH antagonist, anti-androgen, androgen synthesis inhibitor, estrogen or steroid),
and/or would benefit from ceasing or altering one or more cancer treatments selected from the group consisting of ATR inhibitor, CDK inhibitor, Chemotherapy, WEE1 inhibitor, Aurora kinase inhibitor, alkylating agent, PARP inhibitor, DNA-PK inhibitor, immune checkpoint therapies (for example a PD-1 inhibitor, PD-L1 inhibitor, or a CTLA-4 inhibitor), CHK2 inhibitor, platinum-based antineoplastic drug, taxane, radionuclide and radiation therapy, PI3K inhibitor, mTOR inhibitor, DNMT1 inhibitor, HDAC inhibitor, BET inhibitor, PORCN inhibitor, FZD antagonists/monoclonal antibody, inhibitor of Wnt target genes, or hormonal agent, such as a LHRH agonist, LHRH antagonist, anti-androgen, androgen synthesis inhibitor, estrogen or steroid (for example one or more cancer treatments selected from the group consisting of a chemotherapy (such as taxanes (in particular docetaxel and cabazitaxel) and platinum-based antineoplastic drugs (in particular carboplatin)), PARP inhibitors, or hormonal agent, such as a LHRH agonist, LHRH antagonist, anti-androgen, androgen synthesis inhibitor, estrogen or steroid).

18. The method of any preceding claim wherein at least one target gene-region is selected from the group consisting of FOXA1, FOXP1, HSD3B1, NCOA2 and ZBTB16 as defined in Table 1, and wherein the presence and/or alteration of one or more asCNA, and/or the presence of one or more somatic and/or germline mutations, in a target gene-region selected from the group consisting of FOXA1, FOXP1, HSD3B1, NCOA2 and ZBTB16 as defined in Table 1, indicates that the subject would benefit from ceasing or altering treatment with an hormonal agent, such as a LHRH agonist (for example leuprolide, goserelin, triptorelin, or histrelin), LHRH antagonist (for example degarelix), androgen blockers (for example abiraterone or ketoconazole), anti-androgen (for example enzalutamide, apalutamide, darolutamide, Bicalutamide, nilutamide or flutamide), androgen synthesis inhibitor (for example abiraterone), estrogen or steroid (for example prednisone or dexamethasone); and/or indicates that a subject would benefit from from treatment with one or more alternative cancer treatment, for example a chemotherapy;
and/or wherein at least one target gene-region is selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 as defined in Table 1, and wherein the presence and/or alteration of one or more asCNA, and/or the presence of one or more somatic and/or germline mutations, in a target gene-region selected from the group consisting of AURKA, BRAF, CCND1, CDK12, CDK4, CDK6, CDKN1B, CDKN2A, CUL1, FBXW7, KRAS, MDM2, MDM4, MYC, MYCN, RB1 and TP53 as defined in Table 1, indicates that the subject would benefit from treatment with one or more of an ATR inhibitor (for example Berzosertib), CDK inhibitor (for example Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, and Pyrimidines; and in particular Flavopiridol, Palbociclib, ribociclib and abemaciclib) chemotherapy (for example a taxane (for example docetaxel or cabazitaxel), and c-Met inhibitors (for example cabozantinib)), WEE1 inhibitor (for example adavosertib), Aurora kinase inhibitor (for example Alisertib, ZM447439, hesperidin, and VX-680) or alkylating agent (for example nitrogen mustards (such as cyclophosphamide, chlormethine, uramustine, melphalan, chlorambucil, ifosfamide, and bendamustine), nitrosoureas (such as carmustine, lomustine, and streptozocin) and alkyl sulfonates (such as busulfan)), and/or indicates that the subject would benefit from ceasing or altering treatment with one or more hormonal agent, such as LHRH agonists, LHRH antagonists, anti-androgens, androgen synthesis inhibitors, estrogens and steroids;
and/or wherein at least one target gene-region is selected from the group consisting of ARID1A, CHD1, KMT2C, KMT2D and RYB as defined in Table 1, and wherein the presence and/or alteration of one or more asCNA, and/or the presence of one or more somatic and/or germline mutations, in a target gene-region selected from the group consisting of ARID1A, CHD1, KMT2C, KMT2D and RYBP as defined in Table 1, indicates that the subject would benefit from treatment with one or more of a DNA methyltransferase 1 (DNMT1) inhibitor (for example 5-azacitidine) and the histone deacetylase (HDAC) inhibitor (for example vorinostat and romidepsin).
and/or wherein at least one target gene-region is selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, and wherein the presence or alteration of one or more asCNA, and/or the presence of one or more somatic and/or germline mutations, in a target gene-region selected from the group consisting of ATM, ATR, BRCA1, BRCA2, CHD1, ERCC1, ERCC2, ERCC3, ERCC4, ERCC5, FANCA, FANCC, FANCD2, FANCE, FANCF, FANCG, MLH1, MSH2, MSH6, PALB2, RAD51B and RAD51C as defined in Table 1, indicates that the subject would benefit from treatment with one or more of a PARP inhibitor (for example olaparib, rucaparib, niraparib or talazoparib, Veliparib, Pamiparib, Rucaparib, and Veliparib; and in particular example olaparib, rucaparib, niraparib or talazoparib), ATR inhibitor (for example Berzosertib), CDK inhibitor (for example Flavopiridol (alvocidib), abemaciclib, ribociclib, Olomoucine, Roscovitine (Seliciclib), Purvalanol, Paullones, Butryolactone, Thio/oxoflavopiridols, Oxindoles, Aminothiazoles, Benzocarbazoles, and Pyrimidines; and in particular Flavopiridol, Palbociclib, ribociclib and abemaciclib), DNA-PK inhibitor (for example AZD7648, M3814, CC-122 and CC-115), immune checkpoint therapy (for example a PD-1 inhibitor (e.g. pembrolizumab, nivolumab, cemiplimab, or spartalizumab), PD-L1 inhibitor (e.g. atezolizumab, avelumab or durvalumab), or a CTLA-4 inhibitor (e.g. ipilimumab)), CHK1 inhibitor (for example V158411, PF-477736 and AZD7762), CHK2 inhibitor (for example CCT241533 and Aminopyridine 7), WEE1 inhibitor (for example adavosertib), platinum-based antineoplastic drug (for example cisplatin, carboplatin, oxaliplatin, nedaplatin, triplatin tetranitrate, picoplatin, satraplatin and phenanthriplatin, and in particular cisplatin, carboplatin, oxaliplatin, nedaplatin), or radionuclide or radiation therapy (for example radium-223 and PSMA-targeting radionuclide therapies (for example 225Ac-Labeled PSMA-617 or 177Lu-Labeled PSMA-617).
and/or, wherein at least one target gene-region is selected from the group consisting of ASXL1, CLU, CYLD, ERG_TMPRSS2, GNAS, IDH2, NFE2L2, NKX3-1, RUNX1, SPOP and ZFHX3 as defined in Table 1, and wherein the presence or alteration of one or more asCNA, and/or the presence of one or more somatic and/or germline mutations, in a target gene-region selected from the group consisting of ASXL1, CLU, CYLD, ERG_TMPRSS2, GNAS, IDH2, NFE2L2, NKX3-1, RUNX1, SPOP and ZFHX3 as defined in Table 1, indicates that the subject would benefit from treatment one or more of ATR inhibitor, CDK inhibitor, Chemotherapy, WEE1 inhibitor, Aurora kinase inhibitor, PARP inhibitor, DNA-PK inhibitor, immune checkpoint therapies (for example a PD-1 inhibitor, PD-L1 inhibitor, or a CTLA-4 inhibitor), CHK2 inhibitor, WEE1 inhibitor, platinum-based antineoplastic drug, radionuclide and radiation therapy, PI3K inhibitor, PORCN inhibitor, FZD antagonists/monoclonal antibody, or inhibitor of Wnt target genes.
and/or, wherein at least one target gene-region is selected from the group consisting of AKT1, AKT2, AKT3, MET, PIK3C, PIK3CB, PIK3R1 and PTEN as defined in Table 1, and wherein the presence or alteration of one or more asCNA, and/or the presence of one or more somatic and/or germline mutations, in a target gene-region selected from the group consisting of AKT1, AKT2, AKT3, MET, PIK3C, PIK3CB, PIK3R1 and PTEN as defined in Table 1, indicates that the subject would benefit from treatment with one or more of a PI3K inhibitor (for example idelalisib, copanlisib, duvelisib, alpelisib, umbralisib, dactolisib, voxtalisib, Taselisib, Idelalisib, Buparlisib, Duvelisib, and Copanlisib and in particular idelalisib, copanlisib, duvelisib, alpelisib, and umbralisib) or mTOR inhibitor (for example rapamycin, deforolimus, dactolisib, voxtalisib, temsirolimus, everolimus, sapanisertib, AZD8055, and AZD2014); and/or indicates that the subject would benefit from ceasing or altering treatment with one or more hormonal agent, such as LHRH agonists, LHRH antagonists, anti-androgens, androgen synthesis inhibitors, estrogens and steroids.
and/or, wherein at least one target gene-region is selected from the group consisting of APC, CTNNB1 and RNF43 as defined in Table 1, and wherein the presence or alteration of one or more asCNA, and/or the presence of one or more somatic and/or germline mutations, in a target gene-region selected from the group consisting of APC, CTNNB1 and RNF43 as defined in Table 1, indicates that the subject would benefit from treatment with one or more of a PORCN inhibitor (for example WNT974, ETC-1922159 and CGX1321), FZD antagonists/monoclonal antibody (for example Vantictumab, Ipafricept and OTSA101-DTPA-90Y), or inhibitor of Wnt target genes (for example SM08502).

19. The method of any preceding claim, wherein the asCNA is a balanced copy number gain, unbalanced copy number gain, a mono-allelic copy number loss, bi-allelic copy number loss, or a loss of heterozygosity other than mono-allelic deletion (for example a copy number-neutral loss of heterozygosity).

20. The method of any preceding claim, further comprising
I) providing a further biological sample obtained from the subject during or after the subject has undergone a treatment for cancer, wherein said sample comprises tumor DNA;
II) performing steps iii) to v) of any one of claims 1 to 19 using the further biological sample provided in step 1);
wherein the presence, absence, and/or alteration of one or more asCNA in the further biological sample comprising tumor DNA compared to the biological sample comprising tumor DNA indicates that the subject would benefit from treatment with one or more cancer treatments, has benefited, or is benefiting, from one or more cancer treatments and/or would benefit from ceasing or altering one or more cancer treatments.

21. The method of any preceding claim, wherein step ii) comprises detecting the presence of SNPs in the non-tumor DNA at:
at least 30, preferably at least 90, of the SNP loci defined in Table 1 for each of at least 5, at least 10, at least 20, at least 30, or all of the target gene-regions defined in Table 1;
and optionally at least 30, preferably at least 90, of the SNP loci defined in Table 2 for each of at least 5, at least 10, at least 20, at least 30, or all of the control gene-regions defined in Table 2.

22. A set of oligonucleotide probes suitable for use in the method of any one of claims 1 to 21, wherein the set of oligonucleotide probes are capable of hybridizing to:
at least 80% (or at least 50) of the SNP loci defined in Table 1 for each of at least 5 target gene-regions defined in Table 1,
and optionally at least 80% (or at least 50) of the SNP loci defined in Table 2 for each of at least 3 control gene-regions defined in Table 2.

23. A kit for use in the staging, classification, screening, monitoring, stratification, selecting treatment for, ascertaining whether treatment is working in, and/or prognostication of cancer in a biological sample obtained from a subject according to the method of any one of claims 1 to 21, said kit comprising the set of probes of claim 22.

24. An in vitro assay for staging, classification, screening, monitoring, stratification, selecting treatment for, ascertaining whether treatment is working in, and/or prognostication of a cancer in a subject, said in vitro assay comprising the method steps of:
a) providing a biological sample obtained from the subject, wherein said sample comprises tumor DNA, and a biological sample obtained from the subject, wherein said sample comprises genomic non-tumor DNA;
b) providing a set of probes, wherein said set of probes are capable of specifically hybridizing to:
at least 5% of the SNP loci defined in Table 1 for each of at least 5 target gene-regions defined in Table 1,
and optionally at least 5% of the SNP loci defined in Table 2 for each of at least 3 control gene-regions defined in Table 2;
c) contacting the biological sample comprising non-tumor and/or tumor DNA with the set of probes under conditions suitable for one or more of the probes to specifically hybridize to a SNP locus in the non-tumor and/or tumor DNA;
d) capturing the non-tumor DNA and/or tumor DNA in the biological sample that has hybridized to one or more of the probes, and determining the nucleotide sequence of the captured DNA;
e) analysing the nucleotide sequence of the captured non-tumor DNA to identify which of the SNPs present in the non-tumor DNA are informative SNPs (iSNPs) for the subject, wherein an iSNP is a SNP that is heterozygous in the non-tumor DNA for the subject;
f) analysing the nucleotide sequence of the captured tumor DNA to determine the allelic imbalance for each target gene-region, and optionally for each control gene-region, of the tumor DNA by reference to the iSNPs for the subject in each gene-region; and determining the copy number for each target gene-region, and optionally for each control gene-region, in the tumor DNA; and
g) analysing the allelic imbalance and copy number for each target gene-region, and optionally for each control gene-region, to determine the presence, absence, and/or alteration of one or more allele-specific copy number aberration (asCNA) at each target gene-region in the tumor DNA;
wherein the presence, absence, and/or alteration of one or more asCNA in the tumor DNA indicates that the subject would benefit from treatment with one or more cancer treatments, has benefited, or is benefiting, from one or more cancer treatments and/or would benefit from ceasing or altering one or more cancer treatments.

25. The assay of claim 24, wherein step d) further comprises amplifying the captured DNA using a polymerase chain reaction; and/or
wherein step f) comprises determining the nucleotide sequences of the amplified DNA by using a next generation sequencing technique selected from the group consisting of Polony sequencing, 454 pyrosequencing, Combinatorial probe anchor synthesis, SOLiD sequencing, Ion Torrent semiconductor sequencing, DNA nanoball sequencing, Heliscope single molecule sequencing, Single molecule real time (SMRT) sequencing, Nanopore DNA sequencing, Microfluidic Sanger sequencing and Illumina dye sequencing; and optionally wherein step f) further comprises a step of removing the duplicate nucleotide sequence reads from the sequencing data obtained for the captured DNA,

26. The assay of claim 25, wherein steps e) and f) are performed using the DNA derived from amplifying the DNA captured by one or more probes; and/or
wherein step f) further comprises aligning the nucleotide sequence reads for each amplified DNA molecule with a reference genome, for example the human G1Kv37 reference genome or genomic DNA sequence derived from a sample of white blood cells obtained from the subject.

27. The assay of any one of claims 24 to 26, wherein step b) comprises providing a set of probes, wherein said set of probes is capable of specifically hybridizing to:
at least 10%, at least 20%, at least 30%, at least 40%, at least 60%, or at least 80% of the SNP loci defined in Table 1 for each of at least 10, at least 25, at least 50, or all of the target gene-regions defined in Table 1; and/or
at least 30, preferably at least 90, of the SNP loci defined in Table 1 for each of at least 5, at least 10, at least 20, at least 30, or all of the target gene-regions defined in Table 1; and/or
optionally at least 10%, at least 20%, at least 30%, at least 40%, at least 60%, or at least 80% of the SNP loci defined in Table 1 for each of at least 10, at least 20, at least 30, or all of the control gene-regions defined in Table 2; and/or
and optionally at least 30, preferably at least 90, of the SNP loci defined in Table 2 for each of at least 5, at least 10, at least 20, at least 30, or all of the control gene-regions defined in Table 2.
